(19) United States
(12) Patent Application Publication
(10) Pub. No.: US 2026/0158147 A1
(43) Pub. Date: Jun. 11, 2026

(54) METHODS AND COMPOSITIONS FOR TARGETED DELIVERY OF THERAPEUTICS

(71) Applicant: Manifold Biotechnologies, Inc., Boston, MA (US)

(72) Inventors: Pierce Ogden, Jamaica Plain, MA (US); Kathleen Nudel, Boston, MA (US); Erik Swanson, Boston, MA (US); Salvatore Alioto, Waltham, MA (US); Alvaro Garcia, Arlington, MA (US); Karen Duffy, Somerville, MA (US); Alexander Reis, Jamaica Plain, MA (US); Glenn Gordon Hauk, Cambridge, MA (US); Naveen Khinduka Mehta, Watertown, MA (US); Marshall A. Case, Waltham, MA (US)

(21) Appl. No.: 19/205,127

(22) Filed: May 12, 2025

Related U.S. Application Data
(60) Provisional application No. 63/646,969, filed on May 13, 2024.
(60) Provisional application No. 63/676,361, filed on Jul. 27, 2024.
(60) Provisional application No. 63/703,625, filed on Oct. 4, 2024.
(60) Provisional application No. 63/710,935, filed on Oct. 23, 2024.
(60) Provisional application No. 63/758,738, filed on Feb. 14, 2025.
(60) Provisional application No. 63/780,105, filed on Mar. 28, 2025.

(51) Int. Cl. A61K 47/42; A61K 31/7105; A61K 31/711; A61K 38/00; C07K 14/00; C07K 16/00; C12N 15/11; C40B 30/04; C40B 40/10; C40B 70/00; G01N 33/68

(52) U.S. Cl. CPC A61K 47/42; A61K 31/7105; A61K 31/711; A61K 38/00; C07K 14/00; C07K 16/00; C12N 15/11; C40B 30/04; C40B 40/10; C40B 70/00; G01N 33/6803; C07K 2317/565

(57) ABSTRACT

Compositions, methods and systems for targeted delivery using one or more shuttles operably linked to one or more cargos (e.g., cargo polypeptides, e.g., cargo oligonucleotides) and subsequent quantification of an abundance of one or more cargos (e.g., proteins) in a mixture (e.g., a complex mixture (e.g., in vivo)) or in a specific cell, tissue, or organ of interest (e.g., in vivo) using barcodes (e.g., peptide barcodes), binders (e.g., polypeptide binders), and binding agents (e.g., phage) are provided herein.

### CROSS-REFERENCE TO RELATED APPLICATIONS

This application claims priority to and the benefit of U.S. Provisional Application No. 63/646,969, filed May 13, 2024, U.S. Provisional Application No. 63/676,361, filed Jul. 27, 2024, U.S. Provisional Application No. 63/703,625, filed Oct. 4, 2024, U.S. Provisional Application No. 63/710,935, filed Oct. 23, 2024, U.S. Provisional Application No. 63/758,738, filed Feb. 14, 2025, U.S. Provisional Application No. 63/780,105, filed Mar. 28, 2025, the disclosures of which are incorporated by reference herein in their entireties.

### SEQUENCE LISTING

The present specification makes reference to a Sequence Listing (submitted electronically as an .xml file named 2013703-0034_SL.xml on Feb. 20, 2026). The .xml file was generated on Jul. 8, 2025, and is 9,471,940 bytes in size. The entire contents of the Sequence Listing are herein incorporated by reference.

### BACKGROUND

Assessments of agents (e.g., cargo components encoding cargo polypeptides, delivery particles, shuttles, etc.) are central to much of molecular and pharmaceutical biology.

### SUMMARY

The present disclosure provides insights and technologies that achieve improved or otherwise desirable assessment of agents (e.g., shuttle agents, nucleic acids comprising and/or encoding shuttle agents, cargo agents (e.g., one or more nucleic acid sequence components, e.g., cargo polypeptide agents, e.g., cargo oligonucleotide agents), therapeutic agents, and/or in some embodiments delivery particles comprising such nucleic acids comprising and/or encoding shuttle agents, cargo agents, and/or therapeutic agents). Among other things, the present disclosure provides insights and technologies that achieve improved or otherwise desirable agents, which for example target specific tissues and/or organs of interest (e.g., brain). For example, the present disclosure provides insights and technologies that achieve improved delivery (e.g., using one or more shuttles as described herein) to one or more tissues and/or organs of interest (e.g., brain). For example, the present disclosure appreciates that one or more agents (e.g., shuttle agents, therapeutic agents, cargo agents) can be engineered for desirable properties (e.g., tropism, clearance, efficacy, reduced toxicity (e.g., reduced anemia), etc.) in target tissues and/or organs (e.g., brain). In some embodiments, desirable properties include increased target delivery to/across the blood brain barrier (“BBB”). The present disclosure appreciates that such engineering may be achieved using AI-based design. Other platforms that are limited to AI-based design and/or other in silico approaches often generate agents that perform with desirable properties in vitro but fail in vivo. However, the present disclosure provides insights and technologies that achieve such engineering using AI-based design and assess such designs (e.g., of shuttle agents and/or cargo agents and/or therapeutics agents) using a binder-barcode platform described herein, so as to generate agents with desirable properties both in vitro and more importantly in vivo. For example, among other things, a shuttle agent described herein comprises a structural feature (e.g., length, hydrophobicity, affinity, etc.) that has an effect (e.g., positive or negative) on cargo function.

Among other things, the present disclosure appreciates that many current technologies for targeted delivery (e.g., targeted delivery of cargo agents and/or therapeutic agents) to specific tissues and/or organs of interest, may typically be non-specific (e.g., may not be delivered to a specific tissue and/or organ of interest) and/or may be inefficient (e.g., may have reduced bioavailability to a specific tissue and/or organ of interest). Additionally, the present disclosure appreciates that many current technologies for assessing targeted delivery (e.g., targeted delivery of cargo agents and/or therapeutic agents) are slow and/or costly to perform or implement; many such assessments must be performed one at a time and are laborious.

Among other things, the present disclosure appreciates that many current technologies for assessing, and in particular for determining presence and/or abundance of, one or more agents of interest, may typically rely on mass spectroscopy and/or affinity (e.g., immuno-) detection. The present disclosure appreciates that many available affinity detection technologies are slow and/or costly to perform or implement; many such technologies must be performed one at a time and many are constrained, for example, by availability of fluorogenic substrates (e.g., that may be assessed by relevant technologies—e.g., light microscopy).

The present disclosure further appreciates that certain other technologies, such as DNA barcoding technologies, that are sometimes utilized to assess agents of interest, can also suffer disadvantages. DNA barcodes, for example, can lack stability and/or display undesirable immunogenicities, e.g., when utilized in vivo. The present disclosure appreciates that such technologies therefore can encounter problems, particularly for assessing agents (e.g., cargo agents) in complex environments (e.g., in vivo).

Among other things, the present disclosure encompasses the recognition of the source of certain problems with available technologies typically utilized to assess agents of interest, and in particular to assess cargo agents, shuttle agents, and/or delivery particles of interest. In particular, the present disclosure identifies the source of certain problems encountered by such technologies for assessment (e.g., detection and/or measurement of quantity, such as concentration; e.g., inabilities to detect and/or quantify functional information) of multiple agents, and in particular when such agents are present in a complex system (e.g., in a complex solution and/or in vivo).

Furthermore, the present disclosure provides certain technologies that achieve such assessments, in some embodiments with surprisingly high accuracy. Those skilled in the art will appreciate that a number of contexts exist in which detection and/or measurement (e.g., of a precise amount), of a plurality of agents within a complex system is desirable; moreover, those skilled in the art will appreciate the benefit of high accuracy in many such contexts.

Among other things, the present disclosure provides technologies that achieve detection and/or measurement (e.g., highly accurate and/or otherwise precise measurement) of one or more, and in some embodiments of a plurality of agents (e.g., nucleic acids comprising and/or encoding cargo agents, e.g., delivery particles comprising nucleic acids comprising and/or encoding cargo agents), including in complex systems (e.g., in vivo). In some embodiments, detected agent(s) may be or comprise cargo agents and/or forms thereof (e.g., aggregated; complexed; covalently modified such as by disulfide bond formation, glycosylation, pegylation, phosphorylation; truncated such as by proteolytic cleavage, etc.).

In some embodiments, detected agent(s) may be delivered via delivery particles (e.g., viral particles, virus-like particles, lipid-based particles, polymer-based particles, bead-based, metal-based, or polysaccharide-based particles of interest; e.g., of same and/or different types) in varying conditions (e.g., physiological conditions, e.g., target tissues of interest). In some embodiments, nucleic acids are disposed within delivery particles. In some embodiments, a nucleic acid sequence comprising (a) a cargo component that encodes a cargo polypeptide; (b) a barcode component wherein the cargo component is operably linked to the barcode component. In some embodiments, cargo components include other types of cargo as described herein. In some embodiments, detection of a cargo component associated with a barcode component can be used in turn to assess and/or quantify phenotypes of a delivery particle of interest (e.g., tropism, etc.).

In some embodiments, provided technologies are particularly useful or effective for assessment of therapeutic agents. For example, in some embodiments, provided technologies may be particularly useful for the assessment of one or more features (e.g., properties (e.g., concentration, localization, persistence, affinity, etc.)) of agent(s) of interest; in some such embodiments, relevant agent(s) may be characterized by one or more attributes appropriate or desirable for therapeutic use. For example, in some embodiments, provided technologies may be used to screen potential therapeutic agents (e.g., polypeptide entities) for one or more features (e.g., properties, attributes) suitable for therapeutic use. In some embodiments, features of potential therapeutic agent(s) may be measured one at a time. In some embodiments, two or more features of potential therapeutic agent(s) may be measured simultaneously. For example, in some embodiments, one or more therapeutic agents may be screened for an affinity to a target agent—yet other desirable properties for example molecular stability in a physiologically relevant environment are not yet known. In some embodiments, for example, one or more therapeutic agents may be screened for an affinity to a target agent, along with other desirable properties, for example molecular stability in a physiologically relevant environment.

The present disclosure appreciates that many current methods of polypeptide measurement rely on determining the abundance of light of a certain wavelength, or overall luminescence, such as western blot or ELISA (Towbin 1979, Engvall 1972). Due to constraints of visible light wavelength, these methods allow for only a small number of different polypeptides, often fewer than 4, to be measured at a single time within a single reaction (Elshal, 2006). The present disclosure appreciates that many applications, including drug discovery applications, would benefit from (and, in some cases, require) dramatically higher throughput.

The present disclosure further appreciates that nucleic acid sequencing technologies (e.g., DNA sequencing technologies) have been developed that can analyze billions of individual DNA molecules in a single experiment (Shendure, 2005). Various strategies have been developed to try to apply this massive throughput achievable with nucleic acid sequencing techniques to protein detection and measurement, in particular by tagging proteins with an attached piece of DNA (typically referred to as a “DNA barcode”), which may then be sequenced to indirectly detect the protein (Trads, 2017) or one or more features of the protein.

The present disclosure appreciates the power of applying high-throughput nucleic acid sequencing technologies to assessment of other agents, and in particular of cargo agents, but also identifies the source of certain problems associated with many approaches utilized to study polypeptides by attachment of DNA barcodes. For example, the present disclosure appreciates that modification of a protein by attachment of a DNA barcode can often alter its functionality (Trads, 2017), which can defeat the purpose of using the DNA barcode to assess a polypeptide.

Known techniques to quantify or screen a plurality of therapeutic moieties are described by WO2020097254 (Gordian Biotechnology). The present disclosure identifies the source of a problem with such approaches, however, and moreover provides certain advantages relative to them, including the ability to assess and/or quantify cargo polypeptides directly. Approaches such as those described by Gordian Biotechnology fail to describe such a feature and rely on cell-based analyses. Furthermore, as may be appreciated by a person of ordinary skill in the art, reading the present disclosure, cell-based analyses are limited by experimental complexity, number of outputs, require additional enrichment steps, and cost. In comparison, the present disclosure is not limited by such disadvantages since nucleic acid sequences that encode one or more polypeptide binders that are associated with one or more peptide barcodes can be sequenced and measured to quantify the cargo component without the need of additional analyses and/or enrichment steps.

Known techniques to quantify barcoded cargo polypeptides include those as presented in Egloff et al. (2019), that use mass spectrometry to determine the presence or absence of a protein sequence in a mixture (Egloff 2019). The present disclosure identifies the source of a problem with such approaches, however, and moreover provides certain advantages relative to them, including, for example, by using nucleic acids (e.g., DNA) for amplification of the original signal; approaches such as those described in Egloff et al. fail to include (or to benefit from) such a feature. Furthermore, as may be appreciated by a person of ordinary skill in the art, reading the present disclosure, mass spectrometry only reads out the mass-to-charge ratio of an associated sequence; thus methods using mass spectrometry are limited in their total throughput, since different sequences can have the same mass-to-charge ratio. By comparison, the present invention is not limited by such disadvantages since the nucleic acid sequence associated with one or more binding agents in turn associated with each barcode is sequenced and measured to determine and quantify the barcoded cargo polypeptide.

Other techniques available in the art use antibodies displayed on phage (Fab-phage) to determine presence of endogenous proteins expressed on cell surfaces (Pollock, 2018). In such methods, one Fab-phage is generated per endogenous protein (i.e., target protein to be assessed) and no barcodes are utilized. In contrast, the present technology envisions the use of engineered barcode sequences that are generalizable, such that they can be used to mark any protein, whether endogenous or exogenous to the context in which it is applied, and subsequently measured using one or more binding agents to which each barcode, and therefore each barcoded cargo polypeptide, is uniquely associated with (i.e., a “barcode fingerprint” as described elsewhere in this disclosure). Such complex association of one or more binding agents with a barcode is then measured and precise quantification of the associated protein is achieved, e.g., using a complex algorithm (i.e., ‘decoding’ as described elsewhere in this disclosure).

The present disclosure recognizes the ability of antigens displayed on phages to determine epitopes of antibodies within the blood to which the phages are able to bind (Mohan, 2018). However, this method is not able to determine the sequence of the antibody to which the antigen binds, and thus only provides limited information on any antibodies that specifically bind to the antigens displayed on phage. However, the present disclosure provides systems, compositions, and methods that provide the advantage of using generic barcodes with known affinities to one or more binders or binding agents, that can be used to tag any target(s) of interest in a complex mixture, including but not limited to blood, to determine and quantify the target(s).

The present disclosure, among other things, provides technologies that can achieve assessment (e.g., detection and/or quantification) of multiple agents (e.g., multiple nucleic acids comprising and/or encoding cargo agents, multiple delivery particles comprising nucleic acids encoding cargo agents, and/or a combination thereof) within a pool of such agents, using DNA sequencing without requiring (direct or indirect) covalent association of the DNA with the assessed agent, or otherwise constraining the assessed agent.

Described herein are peptide barcodes (also known as “barcodes”) and technologies to make and/or utilize them. In some embodiments, barcodes are utilized to mark cargos. Among other things, such an approach can achieve pooled measurement of cargos without amending non-polypeptide identifiers. In some embodiments, a peptide barcode is an amino acid polypeptide sequence. In some embodiments, a peptide barcode is contained within a cargo (e.g., a polypeptide (e.g., an antibody, e.g., a cell-surface antigen) to be measured; e.g., is endogenous to a cargo to be measured). In some embodiments, a peptide barcode is not contained with a cargo polypeptide (e.g., an antibody, e.g., a cell-surface antigen) to be measured; e.g., is exogenous to a cargo polypeptide to be measured). In some embodiments, a barcode, for example, is a sequence (e.g., a designed sequence) contained within a cargo (e.g., a cargo to be measured). In some embodiments, a cargo comprises a cargo polypeptide. In some embodiments, a barcode is associated (e.g., bound (e.g., covalently)) to the N terminus of a cargo polypeptide (e.g., a cargo polypeptide to be measured). In some embodiments, a barcode is associated (e.g., bound (e.g., covalently)) to the C terminus of a cargo polypeptide (e.g., a cargo polypeptide to be measured). In some embodiments, a barcode is associated (e.g., bound (e.g., covalently)) proximal to the N terminus (e.g., internal to a cargo polypeptide (e.g., a loop region that is proximal to the N terminus)) of a cargo polypeptide (e.g., a cargo polypeptide to be measured). In some embodiments, a barcode is associated (e.g., bound (e.g., covalently)) proximal to the C terminus e.g., internal to a cargo polypeptide (e.g., a loop region that is proximal to the C terminus)) of a cargo polypeptide (e.g., a cargo polypeptide to be measured).

Methods disclosed herein may use peptide barcodes that are designed to have varying lengths. In some embodiments, a peptide barcode may have a length ranging between 1-100, 5-50, 8-25, 9-25, or 9-15 amino acids. In some embodiments, a peptide barcode may have a length of at least 25 amino acids. In some embodiments, a peptide barcode may have a length of at most 8 amino acids. In some embodiments, a peptide barcode may have a length of 10 amino acids.

Barcode sequences as described herein may be reused, so as to be able to quantify different agents (e.g., nucleic acids comprising and/or encoding cargos of interest, e.g., delivery particles of interest, each comprising a nucleic acid encoding a cargo of interest) or mixture of agents (mixture of cargos of interest to be measured and/or mixture of delivery particles to be measured, e.g., via detection of a barcoded cargo). In some embodiments, a barcode is generated such that it can be easily reused between several different agents (e.g., nucleic acids comprising and/or encoding cargos of interest, e.g., delivery particles of interest, each comprising a nucleic acid encoding a cargo of interest) across different experiments.

Among other things, barcodes described herein are designed to be distinct from each other (e.g., unique). In some embodiments, a barcode is designed to have a distinct sequence (e.g., distinct from another barcode). For example, each barcode is designed to be distinct (e.g., unique) from every other barcode used in an experiment, such that each agent (e.g., nucleic acids comprising cargos to be measured, e.g., delivery particles comprising a nucleic acid comprising a cargo to be measured) is associated (e.g., operably linked) with at least one barcode, and each barcode (e.g., barcode with a specific sequence) is only associated with one cargo. As may be understood by a person of ordinary skill in the art, the diversity of barcodes contained within a pool is limited only by the possible diversity of amino acid sequences for a given barcode length. For example, for a barcode length ‘N’, there exists 20^{N} distinct amino acid barcode sequences of length N.

Methods described herein relate to the detection of one or more barcodes using a binding agent. In some embodiments, a barcode is contacted with a binding agent that is associated with or comprises a detectable nucleic acid. For example, in some embodiments, a binding agent may be or comprises a phage, a ribosome, mRNA, DNA, etc. In some embodiments, a binding agent is a phage with a binding motif on its surface (e.g., a polypeptide binder as described herein). In some embodiments, a binding agent comprises a detectable nucleic acid. In some embodiments, a binding agent expresses a detectable nucleic acid. In some embodiments, a binding agent expresses a detectable nucleic acid on (e.g., on a surface of) the binding agent (e.g., a binder). In some embodiments, a binder is a polypeptide. In some embodiments, a binder associates with a barcode (e.g., with known specificity and affinity). In some embodiments a binder associates with one or more barcodes (e.g., with different known specificities and affinities). In some embodiments, a binder is an antibody (e.g., expressed on a surface of a binding agent). In some embodiments, for example, to detect the presence of a specific (e.g., distinct) barcode, the present disclosure envisions the association of a distinct detectable nucleic acid (e.g., a DNA sequence, an RNA sequence, etc.) to a specific barcode. This is achieved through the contact of a binder, which may be expressed on (e.g., on a surface of) a binding agent that comprises the distinct detectable nucleic acid.

Described herein are binders. In some embodiments, a binder is a polypeptide. In some embodiments, for example, a binder is generated to have known specificity and affinity for a given barcode. In some embodiments, a binder is generated to have known specificity and affinity for one barcode. In some embodiments, a binder is generated to have known specificity and affinity for multiple (e.g., two or more, three or more, etc.) barcodes. In some embodiments, a binder is generated to have known specificity and affinity for at least one barcode. In some embodiments, a binder, for example, is expressed on the surface of a binding agent (e.g., a phage, a ribosome, etc.) using methods known to those skilled in the art.

Among other things, systems and methods described, for example, as described herein, identify the advantages of nucleic acid sequencing techniques and apply them effectively to protein detection and measurement methods. For example, methods described herein may use several binders, with known specificities and affinities to different barcodes, which can be expressed on binding agents and mixed together in a single pool. Upon mixing with a pool of barcoded cargo (i.e., cargo polypeptides, each associated with a barcode as described herein), a binder expressed on a binding agent binds to any given barcode in the pool with known but varying affinities. Such a spectrum of affinities of a binder to various barcodes is termed herein as a ‘Binder Fingerprint’. Conversely, a barcode may bind to any given binder in a pool of binders with known but varying affinities. Such a spectrum of affinities of a barcode to various binders is termed herein as a ‘Barcode Fingerprint’. Thus, the presence of specific barcoded cargos can be detected, for example, in a complex solution, by extracting and sequencing the associated nucleic acid (e.g., detectable nucleic acid (e.g., DNA sequence, RNA sequence, etc.)) of the population of binding agents (e.g., phage) bound to barcodes associated with cargos.

Other methods to use binders to identify polypeptide sequences have been developed. However, these methods encounter a number of challenges, including difficulty in generating and characterizing binders, and effectively decoding their binding to specifically identify polypeptides. Another limitation with previously developed binders is their non-specific binding that results in poor signal-to-noise ratios, thereby negatively affecting the accuracy of detection. In contrast, the present technology generates many binders rapidly (e.g., in about a week, about 2 weeks, about 3 weeks, about 4 weeks, about 1 month, about 2 months, about 3 months, about 4 months, about 5 months, about 6 months, or about 1 year). In some embodiments, for example, between about 100 to about 1000 binders may be generated rapidly. In some embodiments, between about 10 to about 1000 binders may be generated rapidly. In some embodiments, between about 10 to about 10,000 binders may be generated rapidly. In some embodiments, at least about 10,000 binders may be generated rapidly.

Binders as described herein are robust. Binders can bind to barcodes (e.g., with robust affinities to one or more barcodes) as described herein in a variety of conditions and/or environments. For example, binders as described herein can bind to barcodes (e.g., with robust affinities to one or more barcodes) in various complex environments (e.g., in blood, tissue, serum, plasma, etc.). Thus, binders of the present disclosure may be used to detect targets (e.g., a nucleic acid encoding a cargo of interest) in varying conditions (e.g., physiological conditions, e.g., target tissues of interest). Moreover, binders of the present disclosure may also be used to detect delivery particles (e.g., viral particles, virus-like particles, lipid-based particles, polymer-based particles, bead-based, or polysaccharide-based particles of interest) in varying conditions (e.g., physiological conditions, e.g., target tissues of interest).

Analogously, barcodes, as described herein, may be generated in a rapid and robust manner. In some embodiments, barcodes as described herein are specific to binders as described herein. In some embodiments, for example, between about 100 to about 2000 barcodes may be generated rapidly (e.g., in about a week, about 2 weeks, about 3 weeks, about 4 weeks, about 1 month, about 2 months, about 3 months, about 4 months, about 5 months, about 6 months, or about 1 year). In some embodiments, between about 10 to about 1000 barcodes may be generated rapidly. In some embodiments, between about 10 to about 10,000 barcodes may be generated rapidly. In some embodiments, at least about 10,000 barcodes may be generated rapidly.

Barcodes as described herein are robust. Barcodes can bind to binders (e.g., with robust affinities to one or more binders) as described herein in a variety of conditions and/or environments. For example, barcodes as described herein can bind to binders (e.g., with robust affinities to one or more binders) in various complex environments (e.g., in blood, tissue, serum, plasma, etc.). Thus, barcodes of the present disclosure may be used to detect targets (e.g., agents of interest) in varying conditions (e.g., physiological conditions). The present disclosure, therefore corrects for the disadvantages and defects of existing methods (e.g., non-specific binding, variable binding in different environments, etc.) by generating large numbers of robust binders and barcodes rapidly, which may be used in combination with computational methods (e.g., deconvolution methods) described herein, to allow for specific, well-characterized binder-barcode binding/association and accurate detection methods.

The present disclosure also envisions an ability to modify sequence(s) of one or more peptide barcode sequences such that they are readily distinguishable from each other, and/or from potential background protein sequence. Analogously, the present disclosure also envisions the ability to modify the sequence(s) of one or more polypeptide binder sequences such that they are readily distinguishable from each other, and/or from potential background protein sequence.

Among other things, the present invention as described herein provides methods of testing ‘n’ distinct protein candidates where n≥1, in a single assay or animal model. In some embodiments, a protein candidate is a therapeutic protein candidate. In some embodiments, multiple protein candidates are designed and each distinct protein candidate is associated with its own unique peptide barcode as described herein. Such barcoding has many advantages, including but not limited to injecting all protein candidates in a single injection into an assay and/or an animal in a cost- and time-efficient manner. Subsequently, a sample (e.g., tissue sample, serum sample, blood sample, extracellular sample, single cell sample etc.) from an injected animal may be obtained and barcodes extracted. In some embodiments, such extracted barcodes provide a measure of the relative abundance of protein candidates originally injected. For example, one or more extracted barcodes may be identified by contacting them with a pool of binders (e.g., expressed on a binding agent) known to bind to the barcodes originally bound to the protein candidates. Following binding of barcodes and binders, bound binding agents (e.g., phage) are selected and their detectable nucleic acid (e.g., DNA sequence, RNA sequence, etc.) extracted. In some embodiments, extracted nucleic acids are subjected to sequencing (e.g., next generation sequencing). The sequenced nucleic acid may then be used to identify the one or more barcodes they were designed to bind to, which along with the previously established information on binding affinities between various binder-barcode pairs may be used to identify and determine the relative abundance of each protein originally injected.

Also described herein, are methods used to translate nucleic acid counts, for example from a sequencing experiment, to relative or absolute protein quantifications. In some embodiments, nucleic acid sequences are counted and in silico translated into protein sequences. As is described herein, a nucleic acid sequence corresponds to a binder sequence, with established and characterized affinity for every barcode given in a pool. In some embodiments, binder counts are compared to a database of known propensities for binding to a single barcode. In some embodiments, binder counts are compared to a database of known propensities for binding to multiple barcodes (e.g., two or more, three or more, etc.). In some embodiments, for example in a sequencing experiment, relative proportions of binder counts are compared directly in order to determine relative proportions of barcodes and/or proteins associated with barcodes. In some embodiments, as may be known to a person of ordinary skill in the art, sequences (e.g., control sequences or accessory sequences) of known abundance (e.g., count, quantification, concentration, etc.) are utilized (e.g., added to the sequencing experiment) to determine an absolute abundance (e.g., count, quantification, concentration, etc.) for a given binder or binders, which may be used to estimate an absolute abundance (e.g., count, quantification, concentration, etc.) for a barcode or barcodes, and/or protein(s) associated with barcode(s) using either direct counts or a linear model as described herein.

In some embodiments, a nucleic acid comprises a cargo component which encodes a cargo polypeptide. In some embodiments, a cargo polypeptide is or comprises a therapeutic polypeptide. In some embodiments, a cargo component further comprises one or more sequence elements. In some embodiments, a cargo component is associated with (e.g., operably linked to) nucleotide sequences encoding barcodes as described herein.

Among other things, the present disclosure provides a peptide shuttle that has or comprises an amino acid sequence having at least 80%, 85%, 90%, 91%, 92%, 93%, 94%, 95%, 96%, 97%, 98%, 99%, or 100% identity to any one of SEQ ID NOs: 8813-8894.

Moreover, among other things, the present disclosure provides a shuttle component that has or comprises a nucleic acid sequence having at least 80%, 85%, 90%, 91%, 92%, 93%, 94%, 95%, 96%, 97%, 98%, 99%, or 100% identity to any one of SEQ ID NOs: 8753-8812.

Moreover, among other things, the present disclosure provides a polypeptide comprising: (a) a peptide shuttle; and (b) a cargo polypeptide. In some embodiments, a peptide shuttle has or comprises an amino acid sequence according to any one of SEQ ID NOs: 8813-8894. In some embodiments, a peptide shuttle is encoded by a nucleic acid sequence having at least 80%, 85%, 90%, 91%, 92%, 93%, 94%, 95%, 96%, 97%, 98%, 99%, or 100% identity to any one of SEQ ID NOs: 8753-8812. In some embodiments, a peptide shuttle is encoded by a nucleic acid sequence according to any one of SEQ ID NOs: 8753-8812.

In some embodiments, a shuttle is a shuttle component (e.g., nucleic acid shuttle).

In some embodiments, a peptide shuttle has a length of 20 to 200 amino acids. In some embodiments, a peptide shuttle has a length of 50 to 200 amino acids. In some embodiments, a peptide shuttle has a length of 100 to 130 amino acids. In some embodiments, a peptide shuttle is encoded by a nucleic acid sequence having a length of 60-600 nucleic acids. In some embodiments, a peptide shuttle is encoded by a nucleic acid sequence having a length of 150-600 nucleic acids. In some embodiments, a peptide shuttle is encoded by a nucleic acid sequence having a length of 300-390 nucleic acids.

In some embodiments, a peptide shuttle is operably linked to a cargo polypeptide. In some embodiments, a peptide shuttle is encoded by a shuttle component. In some embodiments, wherein a cargo polypeptide is encoded by a cargo component. In some embodiments, a cargo polypeptide encodes a therapeutic polypeptide.

Moreover, among other things, the present disclosure provides a polynucleotide comprising: (a) a shuttle component whose nucleotide sequence is or comprises a sequence encoding a peptide shuttle; and (b) a cargo component whose nucleotide sequence is or comprises a sequence encoding a cargo polypeptide.

In some embodiments, a shuttle component is operably linked to a cargo component.

In some embodiments, a polynucleotide further comprises: (c) a barcode component whose nucleotide sequence is or comprises a sequence encoding a peptide barcode characterized in that: (i) a peptide barcode has a length within a range of 1 to 100, 5 to 50, 8 to 25, 9 to 25, or 9 to 15 amino acids; and (ii) has been determined to bind specifically to a particular group of polypeptide binders within a set of binders.

In some embodiments, a shuttle component encodes a peptide sequence having at least 80%, 85%, 90%, 91%, 92%, 93%, 94%, 95%, 96%, 97%, 98%, 99%, or 100% identity to any one of SEQ ID NOs: 8813-8894. In some embodiments, a shuttle component encodes an amino acid sequence according to any one of SEQ ID NOs: 8813-8894.

In some embodiments, an operably linked shuttle component and cargo component encode a shuttle-cargo polypeptide, wherein an encoded shuttle-cargo polypeptide has or comprises an amino acid sequence having at least 80%, 85%, 90%, 91%, 92%, 93%, 94%, 95%, 96%, 97%, 98%, 99%, or 100% identity to any one of SEQ ID NOs: 8633-8752. In some embodiments, an encoded shuttle-cargo polypeptide has or comprises an amino acid sequence according to any one of SEQ ID NOs: 8633-8752. In some embodiments, a composition is or comprises a shuttle-cargo polypeptide, wherein shuttle-cargo polypeptide is encoded by a nucleic acid sequence having at least 80%, 85%, 90%, 91%, 92%, 93%, 94%, 95%, 96%, 97%, 98%, 99%, or 100% identity to any one of SEQ ID NOs: 8513-8632. In some embodiments, a composition is or comprises a shuttle-cargo polypeptide, wherein shuttle-cargo polypeptide is encoded by a nucleic acid sequence according to any one of SEQ ID NOs: 8513-8632.

In some embodiments, a polynucleotide has or comprises a nucleic acid sequence having at least 80%, 85%, 90%, 91%, 92%, 93%, 94%, 95%, 96%, 97%, 98%, 99%, or 100% identity to any one of SEQ ID NOs: 8513-8632. In some embodiments, a polynucleotide has or comprises a nucleic acid sequence according to any one of SEQ ID NOs: 8513-8632.

In some embodiments, a cargo component further comprises one or more sequence elements, or a complement thereof, selected from a group consisting of: a promoter, an enhancer, a silencer, an insulator, a transcriptional regulatory element, a translational regulatory element, a splice donor, a splice acceptor, a transcriptional terminator, a translational start site, a translational stop site, a packaging signal, an integration signal, and any combination thereof.

In some embodiments, a cargo component comprises an internal ribosome entry site (IRES). In some embodiments, a cargo component further encodes a cleavable moiety (e.g., a self-cleaving peptide (e.g., a 2A peptide)).

In some embodiments, a nucleic acid is or comprises DNA. In some embodiments, a nucleic acid is or comprises RNA.

In some embodiments, a cargo component further comprises one or more of a capping moiety, a 5′ untranslated region (UTR), 3′ UTR, a polyadenylation (polyA) tail, or a complement thereof, or any combination thereof.

In some embodiments, a cargo component, or a portion thereof, is codon-optimized.

Moreover, among other things, the present disclosure provides a composition comprising: (a) a shuttle (e.g., a peptide shuttle described herein); and (b) a cargo, wherein a shuttle is operably linked to a cargo.

In some embodiments, a cargo is or comprises a therapeutic polypeptide. In some embodiments, a cargo is or comprises a cargo polypeptide. In some embodiments, a cargo is or comprises an antibody or a fragment thereof or a variant thereof. In some embodiments, an antibody is or comprises rituximab, ocrelizumab, ofatumumab, ublituximab, obinutuzumab, aducanumab, lecanemab, gantenerumab, donanemab, prasinezumab, anti-BACE-1 antibody 6266, anti-BACE-1 antibody YW412.8.31, Lu AF82422, or MEDI1341 or a fragment thereof or a variant thereof. In some embodiments, a fragment is or comprises one or more sequences (e.g., VL, VH, CDR sequences) associated with rituximab, ocrelizumab, ofatumumab, ublituximab, obinutuzumab, aducanumab, lecanemab, gantenerumab, donanemab, prasinezumab, anti-BACE-1 antibody 6266, anti-BACE-1 antibody YW412.8.31, Lu AF82422, or MEDI1341.

In some embodiments, an antibody has or comprises an amino acid sequence having at least 80%, 85%, 90%, 91%, 92%, 93%, 94%, 95%, 96%, 97%, 98%, 99%, or 100% identity to any one of rituximab, ocrelizumab, ofatumumab, ublituximab, obinutuzumab, aducanumab, lecanemab, gantenerumab, donanemab, prasinezumab, anti-BACE-1 antibody 6266, anti-BACE-1 antibody YW412.8.31, Lu AF82422, or MEDI1341.

In some embodiments, a fragment has or comprises a sequence having at least 80%, 85%, 90%, 91%, 92%, 93%, 94%, 95%, 96%, 97%, 98%, 99%, or 100% identity to any one more sequences (e.g., VL, VH, CDR sequences) associated with rituximab, ocrelizumab, ofatumumab, ublituximab, obinutuzumab, aducanumab, lecanemab, gantenerumab, donanemab, prasinezumab, anti-BACE-1 antibody 6266, anti-BACE-1 antibody YW412.8.31, Lu AF82422, or MEDI1341.

In some embodiments, a cargo polypeptide further comprises a localizing moiety. In some embodiments, a localizing moiety is selected from a group consisting of: a secretory signal and an intracellular localization moiety. In some embodiments, a localizing moiety is selected from a group consisting of a secretory signal and an intracellular localization moiety.

In some embodiments, a cargo polypeptide further comprises an intermediate or a pro component. In some embodiments, a cargo polypeptide further comprises a tag moiety. In some embodiments, a cargo polypeptide further comprises a liganding moiety. In some embodiments, a cargo polypeptide further comprises a stability modifying moiety. In some embodiments, a cargo polypeptide further comprises a masking moiety. In some embodiments, a cargo polypeptide further comprises an allosteric modulation moiety.

In some embodiments, a localizing moiety, tag moiety, liganding moiety, stability modifying moiety, masking moiety, or an allosteric modulation moiety is cleavable.

In some embodiments, a cargo polypeptide is or comprises a wild-type (e.g., naturally occurring) polypeptide. In some embodiments, a cargo polypeptide is or comprises a variant polypeptide. In some embodiments, a variant polypeptide is a variant of a reference polypeptide, which reference polypeptide is or comprises a wild-type (e.g., naturally occurring) polypeptide (e.g., wherein a variant polypeptide comprises an amino acid sequence that is at least 70% identical to a reference polypeptide (e.g., at least 80%, at least 85%, at least 90%, at least 95%, at least 96%, at least 97%, at least 98%, at least 99% identical to each other)).

In some embodiments, a shuttle component is operably linked to a barcode component.

In some embodiments, a nucleotide sequence of a barcode component comprises, in order from 5′ to 3′ or 3′ to 5′, one or more of: (a) a first invariant sequence (e.g., a linker sequence or a payload sequence); (b) a variant sequence that is at least 9 nucleotides long; and (c) a second invariant sequence (e.g., a linker sequence, a stop codon, or a payload sequence).

In some embodiments, a variant sequence is at least 15, 24, 27, 45, 150, or 300, nucleotides long.

In some embodiments, a nucleotide sequence of a barcode component further comprises one or more of: (d) a sequence encoding a short helical motif; (e) a sequence encoding a disordered motif; (f) an invariant sequence linking a barcode component to a cargo component.

In some embodiments, a polypeptide further comprises (c) a peptide barcode characterized in that: (i) a peptide barcode has a length within a range of 1 to 100, 5 to 50, 8 to 25, 9 to 25, or 9 to 15 amino acids; and (ii) has been determined to bind specifically to a particular group of polypeptide binders within a set of binders.

In some embodiments, a peptide barcode has an amino acid sequence according to any one of SEQ ID NOs: 5347-8398. In some embodiments, a peptide barcode is encoded by a nucleic acid sequence according to any one of SEQ ID NOs: 1148-4199.

In some embodiments, a peptide barcode has a length of 8 to 25 amino acids. In some embodiments, a peptide barcode has a length of 10 amino acids.

In some embodiments, each polypeptide binder of a group of polypeptide binders has an amino acid sequence according to any one of SEQ ID NOs: 4200-5346. In some embodiments, each polypeptide binder of a group of polypeptide binders is encoded by a nucleic acid sequence according to any one of SEQ ID NOs: 1-1147.

In some embodiments, each polypeptide binder is expressed on a phage. In some embodiments, a phage is selected from M13, T4, T7, Lambda, and filamentous phage. In some embodiments, a phage is M13.

Among other things, the present disclosure provides a fusion polynucleotide, wherein a fusion polynucleotide encodes a polypeptide described herein.

Among other things, the present disclosure provides a plurality of delivery particles, wherein one or more of the delivery particles in a plurality comprises a nucleic acid described herein.

In some embodiments, a nucleic acid in each of the delivery particles is a same. In some embodiments, delivery particles comprise at least two different nucleic acids. In some embodiments, at least two different nucleic acids comprise different cargo components.

In some embodiments, delivery particles comprise cargo components encoding at least two different cargo polypeptides.

In some embodiments, a nucleic acid is disposed within a delivery particle. In some embodiments, a nucleic acid is disposed on a surface of a delivery particle.

In some embodiments, a polynucleotide encodes a barcoded shuttle-cargo polypeptide, wherein a barcoded shuttle-cargo polypeptide, or a characteristic portion thereof, is expressed on a surface of a delivery particle (e.g., a viral particle, a lipid-based particle [e.g., cell-produced or not cell-produced, a lipid nanoparticle (LNP), a liposome, a micelle, an extracellular vesicle (e.g., exosomes, microparticles, etc.)], a polymer-based particle (e.g., PGLA), a polysaccharide-based particle, etc.).

In some embodiments, delivery particles comprise at least two different shuttles.

In some embodiments, two different shuttles are variants of a reference shuttle, which reference shuttle is or comprises a reference sequence. In some embodiments, variants comprise amino acid sequences that are at least 70% identical to each other (e.g., at least 80%, at least 85%, at least 90%, at least 95%, at least 96%, at least 97%, at least 98%, at least 99% identical to each other).

In some embodiments, delivery particles comprise one or more associated (e.g., covalently or non-covalently) targeting moieties. In some embodiments, a one or more targeting moieties are of a same type. In some embodiments, a one or more targeting moieties are of different types.

In some embodiments, a plurality of delivery particles are substantially a same type of delivery particle. In some embodiments, plurality of delivery particles comprises two or more types of delivery particles.

In some embodiments, delivery particles are or comprise a viral particle. In some embodiments, delivery particles are or comprise two or more types of viral particles. In some embodiments, viral particles are or comprise one or more of AAV delivery particles, lentivirus delivery particles, adenovirus delivery particles, herpesvirus delivery particles, and anellovirus delivery particles. In some embodiments, an AAV delivery particles are or comprise two or more serotypes (e.g., AAV2, AAV5, AAV6, AAV8, AAV9, AAV.DJ, AAV.PHP, any variant thereof, or a combination thereof).

In some embodiments, a two or more types of delivery particles are or comprise two or more types of lipid-based particles (e.g., LNPs) (e.g., having different formulations).

In some embodiments, the present disclosure provides a population of delivery particles described herein.

Among other things, the present disclosure provides a library comprising a plurality of polynucleotides as described herein.

Among other things, the present disclosure provides a fusion polypeptide, wherein a fusion polypeptide is encoded by a polynucleotide described herein.

Among other things, the present disclosure provides a cell comprising a polynucleotide described herein, a library described herein, a plurality of delivery particles described herein, or a delivery particle described herein.

Among other things, the present disclosure provides a population of cells comprising a polynucleotide described herein, a library described herein, a plurality of delivery particles described herein, or a delivery particle described herein.

Among other things, the present disclosure provides a pharmaceutical composition comprising a peptide shuttle described herein, a shuttle component described herein, a polypeptide described herein, a polynucleotide described herein, a library as described herein, a plurality of delivery particles described herein, a delivery particle described herein, or a population of delivery particles described herein.

Among other things, the present disclosure provides a kit comprising a set of peptide shuttles, wherein each peptide shuttle of a set is described herein.

Among other things, the present disclosure provides a kit comprising a set of shuttle components, wherein each shuttle component of a set described herein.

Among other things, the present disclosure provides a kit comprising a set of polypeptides, wherein each polypeptide of a set is described herein.

Among other things, the present disclosure provides a kit comprising a set of polynucleotides, wherein each polynucleotide of a set is described herein.

Moreover, among other things, the present disclosure provides a method of identifying a candidate peptide shuttle comprising steps of: a) subjecting a population of barcoded peptide shuttles to an assessment, wherein each barcoded peptide shuttle comprises: (i) a peptide shuttle, and (ii) a peptide barcode, wherein a peptide shuttle is operably linked to a peptide barcode; b) separating those members of a population that satisfy an assessment from those that do not, so that a positive population or a negative population, or both, is identified; c) contacting a positive population, or a negative population, or each population separately from a other, with a set of binders which includes at least one binder specific for each barcode in a population; and d) determining which binders bind to a separated members, thereby determining which barcoded peptide shuttle are present in a contacted population(s).

Among other things, the present disclosure provides for a method for identifying a therapeutic polypeptide or a target polypeptide to treat a disease, disorder, or condition comprising steps of: a) subjecting a population of barcoded shuttle-cargo polypeptides to an assessment, wherein each barcoded shuttle-cargo polypeptide comprises: (i) a peptide shuttle, (ii) a peptide barcode, and (iii) a cargo polypeptide wherein a peptide shuttle is operably linked to a cargo polypeptide, and a cargo polypeptide is operably linked to a peptide barcode; b) separating those members of a population that satisfy an assessment from those that do not, so that a positive population or a negative population, or both, is identified; c) contacting a positive population, or a negative population, or each population separately from a other, with a set of binders which includes at least one binder specific for each barcode in a population; and d) determining which binders bind to a separated members, thereby determining which barcoded shuttle-cargo polypeptides are present in a contacted population(s).

Among other things, the present disclosure provides for a method of pharmacokinetic screening, a method comprising: a) administering a population of barcoded therapeutic candidate polypeptides, or characteristic portion thereof, to an animal, wherein each therapeutic candidate polypeptide comprises a specific peptide barcode and a specific shuttle; b) obtaining a sample from an animal; c) purifying one or more barcoded therapeutic candidate polypeptides from a sample; d) contacting a sample with a set of binders (e.g., binding agents with binders expressed on them) which includes at least one binder specific for each barcode in a sample; and e) determining (e.g., simultaneously) a relative amounts of each binder present in a sample to determine each barcoded therapeutic candidate polypeptides' pharmacokinetic properties, biodistribution, half-life, tissue-mediated drug disposition (TMDD), epitope properties, affinity properties, thermostability properties, pH sensitivity properties, or in vivo stability.

Among other things, the present disclosure provides for a method of treatment comprising: administering a therapeutic polypeptide or nucleic acid that encodes a therapeutic polypeptide, or characteristic portion thereof, that has been determined to satisfy an assessment by a process comprising steps of: a) subjecting a population of barcoded shuttle-cargo polypeptides to an assessment, wherein each barcoded shuttle-cargo polypeptide comprises: (i) a peptide shuttle, (ii) a peptide barcode, and (iii) a cargo polypeptide wherein a peptide shuttle is operably linked to a cargo polypeptide, and a cargo polypeptide is operably linked to a peptide barcode; b) separating those members of a population that satisfy an assessment from those that do not, so that a positive population or a negative population, or both, is identified; c) contacting a positive population, or a negative population, or each population separately from a other, with a set of binders which includes at least one binder specific for each barcode in a population; and d) determining which binders bind to a separated members, thereby determining which barcoded shuttle-cargo polypeptides are present in a contacted population(s); and e) identifying a therapeutic polypeptide from a barcoded shuttle-cargo polypeptides determined to be present in a contacted population(s).

Among other things, the present disclosure provides for a method of treatment comprising: administering a therapeutic polypeptide or nucleic acid that encodes a therapeutic polypeptide, or characteristic portion thereof, that has been determined to satisfy an assessment by a process comprising steps of: a) contacting a set of binders either with a first population, with a second population, or separately with each of a first and second populations of barcoded shuttle-cargo polypeptides, wherein each barcoded shuttle-cargo polypeptide comprises: (i) a peptide shuttle, (ii) a peptide barcode, and (iii) a cargo polypeptide wherein a peptide shuttle is operably linked to a cargo polypeptide, and a cargo polypeptide is operably linked to a peptide barcode, and wherein: i) each binder binds specifically to one barcode relative to a other barcodes; and ii) a set of binders, collectively, includes a binder specific for each of a barcodes in a first and second populations, wherein a first and second populations have been separated from one another based on performance in an assessment; b) determining which binders of a set bind to a member of a first population, a second population, or both, thereby determining which barcoded shuttle-cargo polypeptides are present in a contacted population(s); and c) identifying a therapeutic polypeptide from a barcoded shuttle-cargo polypeptides determined to be present in a contacted population(s).

Among other things, the present disclosure provides for a method of treatment comprising: administering a therapeutic polypeptide, or characteristic portion thereof, wherein a therapeutic polypeptide is identified from a population of barcoded shuttle-cargo polypeptides by a method described herein.

Among other things, the present disclosure provides for a method of treatment comprising: administering a therapeutic polypeptide, or characteristic portion thereof, wherein a therapeutic polypeptide is identified from a population of barcoded shuttle-cargo polypeptides by a method described herein.

In some embodiments, the present disclosure provides of a method that comprises treating a disease, disorder, or condition, wherein the disease, disorder, or condition is a neurodegenerative disease, disorder, or condition. In some embodiments, a neurodegenerative disease, disorder, or condition is AD, PD, MSA, or MS.

In some embodiments, a therapeutic polypeptide comprises a peptide shuttle.

In some embodiments, a peptide shuttle is cleaved after delivery of a therapeutic polypeptide to a target cell, tissue, or organ. In some embodiments, a target cell, tissue, or organ is or comprises a mammalian cell, tissue, or organ. In some embodiments, a target cell, tissue, or organ is or comprises a human cell, tissue, or organ. In some embodiments, a target cell, tissue, or organ is or comprises a brain cell, tissue, or organ.

In some embodiments, multiple samples are obtained from an animal.

In some embodiments, an animal is a model for a disease, disorder, or condition. In some embodiments, a disease, disorder, or condition is cancer, autoimmune, neurodegenerative, or a pathogenic (e.g., viral/bacterial) disease, disorder, or condition. In some embodiments, a disease, disorder, or condition is a neurodegenerative disease, disorder, or condition.

In some embodiments, a disease, disorder, or condition is brain cancer (e.g., glioblastoma, metastatic brain cancers), Alzheimer's Disease (AD), Parkinson's Disease (PD), Huntington's Disease (HD), Multiple Sclerosis (MS), Bell's palsy, Cerebral palsy, Progressive Supranuclear Palsy (PSP), epilepsy, Motor Neuron Disease (MND), Amyotrophic Lateral Sclerosis (ALS), spinal muscular atrophy, Multiple System Atrophy (MSA), ataxia (e.g., spinocerebellar, Friedreich), Frontotemporal Dementia (FTD), or Lewy body disease. In some embodiments, a disease, disorder, or condition is a neurodegenerative disease, disorder, or condition. In some embodiments, a neurodegenerative disease, disorder, or condition is AD, PD, MSA, or MS.

In some embodiments, a disease, disorder, or condition is cancer or metastatic cancer.

In some embodiments, purified therapeutic candidate polypeptides are a subset of barcoded therapeutic candidate polypeptides administered to an animal.

In some embodiments, a disease, disorder, or condition a sample is blood, tissue (e.g., brain tissue), an organ (e.g., brain), or a tumor (e.g., brain tumor).

Among other things, the present disclosure provides for a composition (e.g., pharmaceutical composition) comprising one or more therapeutic polypeptides, or characteristic portion thereof, wherein a one or more therapeutic polypeptides are identified from a population of barcoded shuttle-cargo polypeptides by a method described herein.

Among other things, the present disclosure provides for a composition (e.g., pharmaceutical composition) comprising one or more barcoded shuttle-cargo polypeptides, or characteristic portion thereof, wherein a one or more barcoded shuttle-cargo polypeptides are generated by a method described herein.

Among other things, the present disclosure provides for a composition (e.g., pharmaceutical composition) comprising one or more nucleic acids encoding one or more therapeutic polypeptides, or characteristic portion thereof, wherein therapeutic polypeptides are identified from a population of barcoded shuttle-cargo polypeptides by a method described herein.

Among other things, the present disclosure provides for a method of manufacturing a composition (e.g., pharmaceutical composition) comprising one or more therapeutic polypeptides, or characteristic portion thereof, wherein a one or more therapeutic polypeptides are identified from a population of barcoded shuttle-cargo polypeptides by a method described herein.

Among other things, the present disclosure provides for a method of manufacturing a composition (e.g., pharmaceutical composition) comprising one or more nucleic acids encoding one or more therapeutic polypeptides, or characteristic portion thereof, wherein therapeutic polypeptides are identified from a population of barcoded shuttle-cargo polypeptides by a method described herein.

In some embodiments, a composition is formulated for treating or preventing a disease, disorder, or condition, wherein the disease, disorder, or condition is brain cancer (e.g., glioblastoma, metastatic brain cancers), Alzheimer's Disease (AD), Parkinson's Disease (PD), Huntington's Disease (HD), Multiple Sclerosis (MS), Bell's palsy, Cerebral palsy, Progressive Supranuclear Palsy (PSP), epilepsy, Motor Neuron Disease (MND), Amyotrophic Lateral Sclerosis (ALS), spinal muscular atrophy, Multiple System Atrophy (MSA), ataxia (e.g., spinocerebellar, Friedreich), Frontotemporal Dementia (FTD), or Lewy body disease. In some embodiments, a composition is formulated for treating or preventing a disease, disorder, or condition, wherein the disease, disorder, or condition is a neurodegenerative disease, disorder, or condition. In some embodiments, a neurodegenerative disease, disorder, or condition is AD, PD, MSA, or MS.

These, and other aspects encompassed by the present disclosure, are described in more detail below and in the claims.

### BRIEF DESCRIPTION OF THE DRAWING

FIG. 1A is a schematic of barcoded cargo as described herein, according to an illustrative embodiment. It illustrates a barcoded cargo and the corresponding DNA encoding a barcoded cargo. LN refers to “linker N terminus” and LC refers to “linker C terminus”. In some embodiments, LN and LC sequences are constant and encode amino acids that connect the cargo to the barcode. In some embodiments, LN and LC sequences are constant and are nucleic acid sequences used for modular cloning of barcodes with different cargos. In some embodiments, LN and LC sequences are flanked by Type IIS restriction site sequences.

FIG. 1B is a schematic of barcoded cargo as described herein, according to an illustrative embodiment. It illustrates a nucleic acid sequence encoding a barcode and/or a barcoded cargo. LN refers to “linker N terminus” and LC refers to “linker C terminus”. In some embodiments, LN and LC sequences are constant and encode amino acids that connect the cargo to the barcode. In some embodiments, LN and LC sequences are constant and are nucleic acid sequences used for modular cloning of barcodes with different cargos. In some embodiments, LN and LC sequences are flanked by Type US restriction site sequences.

FIG. 2 is a schematic of a method to detect and/or quantify and/or characterize cargos (e.g., cargo polypeptides) in a pool using barcodes and binding agents as described herein, according to an illustrative embodiment. A library of barcoded cargo is contacted with a library of binding agents containing identifying DNA. A wash step is applied that removes binding agents that do not associate (e.g., link (e.g., form strong linkages)) to any of the barcoded cargo, while leaving only binding agents that associate with barcodes. Following the wash, a process of DNA sequencing is applied to associated binding agents. In some embodiments, sequencing may be performed using next-generation sequencing (NGS) (e.g., as operated by an Illumina sequencer). The relative abundances of DNA sequences are reported as a computer file (e.g., .fastq data). A computer algorithm is applied on the .fastq data combined with prior biophysical characterization of the binding agents to infer the abundance of each of barcoded cargo in a pool.

FIG. 3A is a schematic for capturing a barcode as described herein, so that it may be contacted by a binding agent as described herein, according to an illustrative embodiment. It illustrates a capture scaffold that may have a barcode associated with it (e.g., immobilized on its surface), and a binding agent (e.g., phage with binder expressed on its surface (e.g., with binder DNA in phage)) is contacted to characterize biophysical interaction. In some embodiments, the biophysical characterization is a measure of dissociation constant (Kd) between the binding agent and the peptide barcode.

FIG. 3B is a schematic for capturing a barcode as described herein, so that it may be contacted by a binding agent as described herein, according to an illustrative embodiment. It is a schematic of a barcode-binder platform as described herein, according to an illustrative embodiment. The schematic shows a magnetic bead with a bead binding domain conjugated to a universally tagged (e.g., HALO, Chitin BD, Avitag (Strep), etc.) barcoded cargo. To detect the captured barcoded cargo, a binding agent (e.g., phage expressing a binder on its surface (e.g., phage with binder DNA/lib)) with known affinity to the barcode is bound to the immobilized cargo. The DNA within the phage that encodes for the binder is then amplified and subjected to NGS to detect the cargo.

FIG. 3C is a schematic for capturing a barcode as described herein, so that it may be contacted by a binding agent as described herein, according to an illustrative embodiment. It is a schematic of a barcode-binder platform as described herein, according to an illustrative embodiment. The schematic shows a magnetic bead with an Fc/Protein A conjugated barcoded cargo. To detect the captured barcoded cargo, a binding agent (e.g., phage expressing a binder on its surface (e.g., phage with binder DNA/lib) with known affinity to the barcode is bound to the immobilized cargo. The DNA within the phage that encodes for the binder is then amplified and subjected to NGS to detect the cargo.

FIG. 4 is a schematic of a method to learn the barcode fingerprint of a given barcode as described herein, according to an illustrative embodiment A peptide barcode displayed on a capture scaffold is contacted with a library of binding agents containing identifying DNA. A wash step is applied that removes binding agents that do not associate (e.g., link (e.g., form strong linkages)) with any of the barcodes, while leaving only binding agents that do associate with barcodes. After the wash, a process of DNA sequencing is applied to associated binding agents. In some embodiments, sequencing may be performed using next-generation sequencing (NGS) (e.g., as operated by an Illumina sequencer). The relative abundances of DNA sequences are reported as a computer file (e.g., in .fastq format). A computer algorithm is applied on the .fastq data to computer a barcode fingerprint. This is a vector of the relative counts of the members of the binding agent library. The method of learning a barcode fingerprint can be repeated for any barcode to identify a unique fingerprint. In some embodiments, steps 1-4 of FIG. 4 may be repeated, each time starting with a focused binding agent library in order to improve the fingerprint, for a barcode with an existing fingerprint or a new barcode. In some embodiments, the focused binding agent library is made by oligonucleotide library synthesis.

FIG. 5 is a schematic of a method to use a fingerprint matrix of a set of barcodes to determine the relative abundance of a mixture of barcodes, according to an illustrative embodiment. A set of barcodes for which individual fingerprints have been determined are combined in a known ratio and displayed (e.g., on a scaffold) for subsequent contact with a binding agent library. A binding agent library is contacted with the set of barcodes and non-specific binding agents are washed away. The specific binding agents are quantified by NGS and reported as a mixed measurement computer file (e.g., in .fastq format). These data are provided to a computer algorithm that uses the mixed measurement to learn the relative scalings of readouts relative to the original fingerprints, and assembles the scaled fingerprints together into a scaled matrix. This scaled fingerprint matrix can then be used to quantify the relative abundance of barcoded cargos.

FIGS. 6A-6C show results of quantifying a complex mixture of barcodes. Up to 6 barcodes were pooled and then measured using the decoding method described herein. FIG. 6A shows the actual relative proportion of a given barcode (left panel) and the measured relative proportion of a given barcode (right panel). Rows are individual experimental conditions, columns are barcodes, color is measurements (100% barcode=white, 0% barcode=black). FIG. 6B shows a plot of measured concentration of barcodes against actual concentration of barcodes for all experiments compared across all barcodes. Across all experiments and mixtures, a pearson of 0.95 between measured and actual proportions was calculated. FIG. 6C shows a plot of NGS count values, normalized to counts per million, for each single barcode measurement as well as mixture that were used to predict the relative abundance of each barcode within the mixture. Rows are experiments, thus all values in a row are generated from a single .fastq file and columns are binding agents. FIG. 6C discloses SEQ ID NOS. 8895-8908, respectively, in order of appearance.

FIGS. 7A-7B show a schematic of a method and data obtained using the decoding method on cargo polypeptide with barcodes contained within internal regions of the polypeptide sequences (i.e., endogenous barcodes). The schematic shows results from a synthetic pooled barcode measurement assay. FIG. 7A shows two barcoded cargos (BC1 and BC2) combined at various known concentrations in different wells of a 96-well plate. Each mixture was subjected to contact with the same pool of binding agents and decoded as described herein. Each mixture was quantified and then compared to the known values of the barcoded cargos. FIG. 7B shows relative actual proportions (X axis) of each barcode correlate to relative measured proportions (Y axis) with a pearson of 0.96

FIGS. 8A-8C show a schematic of a method to detect cargo polypeptides in serum using the barcode-binder platform as described herein, according to an illustrative embodiment. The cargo polypeptides have barcodes contained within internal regions of the polypeptide sequences (i.e., endogenous barcodes). FIG. 8A shows the barcoded therapeutic antibody agents of interest (barcoded-mAbs) were mixed at known concentrations and then added to serum. The barcoded cargos were then purified, contacted with binding agents, and subjected to decoding. FIG. 8B shows the relative actual barcoded antibody proportion (left) and the relative measured antibody proportion (right) for 3 experimental conditions, with 3 replicates each. Rows correspond to experimental condition, columns to barcodes, and color of heat-map cell is a measure of the proportion of barcoded antibody present. FIG. 8C shows a scatterplot of all the data across all experimental conditions for all barcodes with a Spearman correlation of 0.926 across all experimental measurements.

FIG. 9A shows a schematic of the experiment provided in Examples 1, 2, and 8. Six unique barcodes (BC1, BC2, BC3, BC4, BC5, and BC6) were mixed at known proportions, contacted with binding agents, and subjected to decoding as described herein. Two barcodes were experimentally held out as negative controls, but prediction for these barcodes was allowed, thus allowing determination of background prediction.

FIG. 9B and FIG. 9C show data on accuracy of decoding procedure across a 10-fold range of concentrations for the 6 unique barcodes. FIG. 9B shows plot of actual data (input) and measured data obtained after decoding for one mixture of known barcode concentrations. Input known concentrations (left bar) are shown next to predictions/measured data (right bar) for each barcode across 3 replicates. FIG. 9C shows plots of actual data (input) and measured data obtained after decoding for five different mixtures (i.e., pools 1-5) of known barcode concentrations. Input known concentrations (left bar) are shown next to predictions/measured data (right bar) for each barcode across 3 replicates.

FIGS. 10A-10C show a method and data for determining the absolute concentration of a single test barcode as described herein. FIG. 10A shows a schematic of an experiment. A single test barcode was assayed at several concentrations, while a “spike-in” barcode (i.e., a reference barcode) was added to each assay mixture at a known concentration. The various concentrations of the test barcode were contacted with binding agents and decoding was performed as described herein. The prediction of the “spike-in” barcode was used to determine the absolute amount of the test barcode being measured. FIG. 10B shows a plot of the measured absolute quantities of the test barcode (right bar) compared to known input concentrations of the test barcode (left bar) for each titration of the test barcode. The Y-axis is the logarithm of the test barcode concentration in nanograms per milliliter (ng/mL). FIG. 10C shows the results of determination of absolute concentration for 6 different barcodes. Plots show known input concentrations (left bar) and measured concentration (right bar) for six (6) different barcodes.

FIG. 11 shows a method for determining the relative abundance of two polypeptides after injection in vivo, using the binder-barcode system described herein according to an illustrative embodiment. The figure shows a graphical depiction of experimental setup. In group 1 (top), mice were injected with 1 barcoded cargo. In group 2 (middle), 2 barcoded cargos were injected. In group 3 (bottom), no barcoded cargos were injected. For each of the three groups, at 24 hours, a serum sample was taken; the barcoded cargo(s) captured using binding agents as described herein, and subjected to decoding. The measured barcoded cargo concentrations (right bar) compared to known input concentrations (left bar) for each group are shown.

FIGS. 12A-12E show determination of twenty-four (24) barcodes contained within a single mixture. FIG. 12A shows a graphical depiction of the experiment. Of 24 total barcodes the algorithm can predict, 10 were present within a mixture at equal concentrations. The rest were held out from the pool, but prediction was computationally allowed. Three (3) separate pools, which cover all possible barcodes, were measured in replicate. FIG. 12B shows prediction for the first pool. Input concentration (left bar) and measured concentration (right bar) are displayed. FIG. 12C shows predictions across all three pools. As in B, input concentration is left bar and measured is right bar. FIG. 12D shows the barcode fingerprint for the 24 barcodes used to computationally determine the relative abundance of the barcodes within the 3 pools. Columns represent barcode fingerprints, and rows represent binding agent fingerprints. FIG. 12D discloses SEQ ID NOS. 8909-8910, 8909, 8911, 8909, 8908-8909, 8912, 8909, 8913, 8909, 8914, 8909, 8915-8920, 8917, 8921-8922, 8921, 8923-8926, 8925, 8927-8928, 8927, 8929, 8927, 8930, 8927, 8925, 8927, 8931-8948, 8908, 8948-8949, 8948, 8950-8970, 8969, 8971-8975, 8974, 8976-8979, 8978-8988, 8967, 8989, 8967, 8990, 8967, 8991, 8967, 8992, 8967, 8993-8997, 8996, 8998-9000, 8999, 9001, 8999, 9002, 8999, 9003-9011, 9010, 9012-9013, 8912, 9014-9015, 9014, 9016-9027, 8898, 9028-9037, 9036, 9038-9040, 9039, 9041, 9039, 9042, 9039, 9043-9047, 9046, 9048, 9046, 9049-9057, respectively, in order of appearance. FIG. 12E shows the binding agent counts from the three pools, used to computationally determine the proportion of the pools. Rows are the binding agent counts, columns are the pools, the cell is the binding agent count within a specific pool. FIG. 12E discloses SEQ ID NOS. 8909-8910, 8909, 8911, 8909, 8908-8909, 8912, 8909, 8913, 8909, 8914, 8909, 8915-8920, 8917, 8921-8922, 8921, 8923-8926, 8925, 8927-8928, 8927, 8929, 8927, 8930, 8927, 8925, 8927, 8931-8948, 8908, 8948-8949, 8948, 8950-8970, 8969, 8971-8975, 8974, 8976-8979, 8978-8988, 8967, 8989, 8967, 8990, 8967, 8991, 8967, 8992, 8967, 8993-8997, 8996, 8998-9000, 8999, 9001, 8999, 9002, 8999, 9003-9011, 9010, 9012-9013, 8912, 9014-9015, 9014, 9016-9027, 8898, 9028-9037, 9036, 9038-9040, 9039, 9041, 9039, 9042, 9039, 9043-9047, 9046, 9048, 9046, 9049-9057, respectively, in order of appearance.

FIG. 13A is a schematic of a method to detect and/or quantify and/or characterize fourteen (14) exemplary cargos (e.g., cargo polypeptides) in a pool using a binder-barcode platform as described herein. A library of barcoded cargo was contacted with a library of binding agents containing identifying DNA (“binder-barcode particles”). Binder-barcode particles were injected as a pooled library into wild-type (wt) BALB/c mice (n=3 per timepoint) in vivo. Blood was collected from individual mice at timepoints 30 min, 6 hours, 24 hours, and 48 hours, (n=3 per timepoint), and serum was extracted. Binder-barcode particles were captured and subjected to a decoding procedure as described herein.

FIG. 13B depicts plots showing clearance of fourteen (14) exemplary binder-barcode particles injected into wild-type (wt) BALB/c mice (n=3 per timepoint) in vivo. Data were collected at time points 30 min, 6 hours, 24 hours, and 48 hours as measured by a decoding procedure described herein. Y-axis is normalized to 100% of injection volume for each exemplary binder-barcode particle. Plots shown in FIG. 13B were measured simultaneously. Each plot contains exemplary binder-barcode particles that were characterized as having certain measurable phenotypes. The left plot shows clearance (% injection) of clinical controls with known properties. The middle plot shows clearance (% injection) of exemplary binder-barcode particles that were characterized has having slow clearance properties. The right plot shows clearance (% injection) of exemplary binder-barcode particles that were characterized as having fast clearance properties.

FIG. 14A is a schematic of a method to detect and/or quantify and/or characterize thirty-six (36) cargos (e.g., cargo polypeptides) in a pool using a binder-barcode platform as described herein. A library of barcoded cargo was contacted with a library of binding agents containing identifying DNA (“binder-barcode particles”). Binder-barcode particles were injected as a pooled library into tumor bearing NSG mice, which had been previously implanted with two tumor cell lines (“Tumor 1”, “Tumor 2”), (n=2-4 per timepoint) in vivo. Blood and tumor tissue was collected from individual mice at timepoints 30 min, 6 hours, 24 hours, and 48 hours, (n=3 per timepoint). Tissue was lysed using standard lysis buffer, and serum was separated from blood. Binder-barcode particles were captured and subjected to a decoding procedure as described herein.

FIG. 14B is a heat-map of data collected from thirty-six (36) exemplary binder-barcode particles using a decoding procedure described herein. Rows identify each exemplary binder-barcode particle tested in the present example. Columns indicate data for a mouse across each time point for serum, Tumor 1, or Tumor 2. Color intensity indicates relative units of drug as measured via a decoding procedure described herein. Color intensity indicates a normalized readout of relative concentration as measured via next generation sequencing (NGS).

FIG. 14C depicts plots of binder-barcode particles described by FIG. 14B using a decoding procedure described herein. A diversity of properties was simultaneously measured. For example, binder-barcode particle P14_A5 was rapidly cleared from serum, with minimal accumulation in Tumor 1 or Tumor 2, while binder-barcode particle P17_A10 was more slowly cleared and maintained in tumor 1 over time.

FIGS. 15A-15C depict plots showing ELISA quantitation of two groups of cargos (Group 1: cargo polypeptides with no barcode; Group 2: a pool of eight (8) binder-barcode particles where each particle includes the same cargo polypeptides used in Group 1, and each particle is barcoded with a different barcode) (FIG. 15A), quantification of Group 2 using a decoding procedure described herein (FIG. 15B), and a comparison of half-life measurements for Group 1 and Group 2 quantified using ELISA and a decoding procedure described herein, respectively (FIG. 15C).

FIG. 16A is a schematic of a method to detect and/or quantify and/or characterize thirty-five (35) cargos (e.g., cargo polypeptides) in distinct pools with different number of barcoded cargos at different concentrations using a binder-barcode platform as described herein.

FIG. 16B depicts a plot showing measured barcode level (arbitrary units) versus expected barcoded-cargo level (ng) generated by arraying ninety-six (96) distinct mixtures comprising 10-35 barcoded cargo with each barcoded cargo at a known concentration between 1 pg and 1 μg. Each data point in FIG. 16B represents a comparison between a known concentration of a binder-barcode particle from one of the ninety-six (96) distinct mixtures and a concentration determined by a decoding procedure described herein.

FIG. 17 depicts a schematic of an exemplary method that provides for high throughput cargo delivery, production, screening, identification, and/or characterization as described herein. Nucleic acids comprising (1) a cargo component whose nucleotide sequence is or comprises a sequence encoding a cargo polypeptide and (2) a barcode component whose nucleotide sequence is or comprises a sequence encoding a peptide barcode are disposed within one or more delivery particles and are administered to an animal (e.g., a mammal). Functional cargos are expressed in a tissue of interest. Decoding methods are used to determine cargos and/or delivery particles with desired properties.

FIG. 18 depicts a schematic of an exemplary method that provides for tracking and/or assessment and/or quantification of different nucleic acids encoding a cargo component disposed within different types of delivery particles, according to an embodiment of the present disclosure. Two exemplary nucleic acid constructs were designed: (1) a first nucleic acid comprising (a) a cargo component encoding a cargo polypeptide comprising a secretion signal peptide, and (b) a barcode component; and (2) a second nucleic acid comprising (a) a cargo component encoding a cargo polypeptide without a secretion signal peptide, and (b) a barcode component. Each nucleic acid design was disposed within different delivery particles (e.g., AAV delivery particles, e.g., AAV2, AAV9, AAV.PHPB) that exhibit different tissue tropisms. Delivery particles were administered into mice and decoded according to methods described herein.

FIGS. 19A-19C depict bar graphs showing high-throughput screening, identification, and/or quantification of two different cargo polypeptides (with or without a secretion signal peptide) delivered via different delivery particles (AAV2, AAV9, AAV.PHPB) across different tissue types (brain, liver, serum).

FIG. 20 depicts a schematic showing that high-throughput screening provides for screening of multiple cargos, formats, targets, and tissues simultaneously in different models.

FIG. 21 depicts octet biolayer interferometry (BLI) data that show respective dissociation of cargo polypeptides against the transferrin receptor (TfR).

FIG. 22 depicts ELISA data that show respective dissociation of cargo polypeptides against the transferrin receptor (TfR).

FIG. 23 depicts a schematic of an exemplary method showing that variant cargos of a previously detected, assessed, and/or characterized cargo (e.g., wild-type cargo) may be generated and subject to further detection, assessment, and/or characterization, for example, using methods as described herein. In some embodiments, such variant cargos may possess improved functionality (e.g., improved developability, improved expression, improved affinity, etc.).

FIGS. 24A-24B depicts a plot showing a high-throughput in vivo screen of brain shuttle candidates using the binder-barcode platform described herein. FIG. 24A shows anti-TfR VHHs with unique properties including: epitope, affinity, thermostability, and pH sensitivity, that were nominated for screening in vivo. FIG. 24B shows 239 anti-TfR VHHs that were simultaneously screened for abundance in vivo in sets of 15 to 96, at doses ranging from 0.5 to 1 mg/kg, depending on batch size, in brain, serum, and other tissue using the binder-barcode platform at 24 hours.

FIG. 25 depicts a plot showing PK analysis across brain, cell-free fraction (parenchyma), serum, and muscle tissues of select screened TfR brain shuttle candidates analyzed in a multiplexed experiment using the binder-barcode platform described herein.

FIG. 26 shows a general description of an exemplary in vivo screening method to identify molecules with enhanced properties (e.g., tissue targeting properties).

FIG. 27 shows a schematic of an in vivo screen for TfR-targeted VHH shuttles in a humanized TfR mouse according to an embodiment described herein.

FIG. 28 depicts a plot showing relative quantification of all TfR shuttles tested in vivo.

FIG. 29 depicts a plot showing comparison of relative brain concentration to relative serum concentration for each TfR shuttle tested in vivo.

FIGS. 30A-30F depicts plots showing pharmacokinetics (PK) for all measured shuttles selected for follow-up from in vivo screens in various tissues of interest. FIG. 30A depicts plots showing PK for all measured shuttles in brain. FIG. 30B depicts plots showing PK for all measured shuttles in serum. FIG. 30C depicts plots showing pharmacokinetics PK for all measured shuttles in parenchyma. FIG. 30D depicts plots showing PK for all measured shuttles in muscle. FIG. 30E depicts plots showing area under curve (AUC) for brain and parenchyma tissues for TfR validation. FIG. 30F depicts plots showing comparison of AUC in serum to brain fractions for measured shuttles.

FIG. 31 shows a schematic of an overview of AI-based iteration methods, for example using Protein Language Models (PLMs) according to an embodiment described herein.

FIG. 32 shows a description of an exemplary algorithm used for AI-based sequence iteration.

FIG. 33 depicts plots showing output of in vivo screen highlighting improved shuttle variants.

FIG. 34 depicts plots showing fitness landscapes for in vitro versus in vivo measurements.

FIG. 35 depicts plots showing PK validation of improved brain uptake for shuttle TfR-1409-51 (columns 1-3 from left) and a shuttle-cargo fusion protein (e.g., a shuttle-cargo polypeptide) aBACE1-TfR-1409-51 that depletes aβ40 in brain (column 4).

FIG. 36 depicts plots showing plasma PK for exemplary aBACE-shuttle constructs described herein. Data in FIG. 36 highlights improved PK performance for aBACE shuttled by TfR-1409-51.

FIG. 37 depicts plots showing brain IgG concentration of exemplary aBACE1 constructs described herein. Data in FIG. 37 highlights increased concentration for aBACE1 with monovalent TfR-1409-51 shuttle.

FIG. 38 shows micrographs of immunofluorescent staining of shuttled aBACE1 in brain at 24 hrs.

FIGS. 39A-39B depicts plots showing PK for TfR-shuttled lecanemab variants. FIG. 39A depicts plots showing PK for TfR-shuttled lecanemab variants in brain, muscle, and serum (X-axis shows time in hours and Y-axis shows PK concentration in nM). FIG. 39B shows comparison of computed AUC values in serum to brain fractions for shuttled lecanemab variants.

FIGS. 40A-40C depicts plots showing results of in vivo screening and assessment of shuttle variants to varied targets using a binder-barcode platform as described herein. FIG. 40A shows schematic of an exemplary experimental method for shuttle target screening described herein. FIG. 40B shows brain concentration versus serum concentration for a subset of screened shuttle variants designed to target to different targets. FIG. 40C shows brain or parenchyma versus serum concentration for a subset of screened CD98 shuttles.

FIG. 41 shows a table listing affinities for shuttle-cargo fusion proteins (e.g., shuttle-cargo polypeptides) comprising lecanemab and shuttle variants directed to CD98.

FIGS. 42A-42C depicts plots showing PK data for shuttle-cargo fusion proteins (e.g., shuttle-cargo polypeptides) comprising lecanemab and shuttle variants directed to different targets. FIG. 42A depicts plots showing PK data for shuttle-cargo fusion proteins for brain tissue (X-axis shows time in hours and Y-axis shows PK concentration in nM). FIG. 42B depicts plots showing PK data for shuttle-cargo fusion proteins for serum (X-axis shows time in hours and Y-axis shows PK concentration in nM). FIG. 42C shows AUC values for shuttle-cargo fusion proteins to un-shuttled lecanemab.

FIG. 43 depicts plots showing aBeta binding with mAbs (gantenerumab and lecanemab) associated with brain shuttle VHHs.

FIGS. 44A-44D depicts plots showing anti-CD20 ADCC with mAbs fused to brain shuttle VHHs. FIG. 44A depicts plots related to rituximab and ublituximab. FIG. 44B depicts plots related to obinutuzumab and ofatumumab. FIG. 44C depicts plots related to ocrelizumab. FIG. 44D depicts plots related to ocrelizumab that is Fc-enhanced.

FIG. 45 depicts plots showing results of a transferrin (Tf) competition ELISA.

FIG. 46 depicts plots showing PK for various shuttle-cargo fusion proteins (e.g., shuttle-cargo polypeptides) comprising lecanemab and shuttle variants directed to TfR tested in the cerebrospinal fluid (CSF) of Cynomolgus monkeys (X-axis shows time in days and Y-axis shows PK concentration in nM).

FIGS. 47A-47D depict plots showing multiplexed in vivo screening data of shuttle candidates. The diagonal lines with percentages denote brain/serum ratios. FIG. 47A depicts plots related to protein-barcoded anti-TfR1 nanobodies screened in multiplex in humanized TfR1 mice (n=3), with takedown and analysis of serum and whole-brain tissue conducted at 24 hrs post-injection. A total of 250 total molecules were screened and those nanobodies detected in brain are shown. FIG. 47B depicts plots related to protein-barcoded anti-CD98hc nanobodies screened in multiplex in humanized CD98hc mice (n=3), with takedown and analysis of serum and whole-brain tissue conducted at 72 hrs post-injection. FIG. 47C depicts plots related to 295 protein-barcoded nanobodies directed to additional unique (e.g., not previously known) targets (n=7) screened in multiplex in C57 mice, with takedown and analysis of serum and whole-brain tissue conducted at 72 hrs post-injection. Each color represents a unique (e.g., not previously known) target. FIG. 47D depicts plots related to 30 protein-barcoded nanobodies against FLT1 screened in multiplex in C57 mice (n=3), with takedown and analysis of serum and whole-brain tissue conducted at 72 hrs post-injection. Three putative shuttle hits (green stars) were identified with a brain/serum ratio greater than 1%, while the remaining FLT1 shuttle candidates showed little/no brain exposure (red circles). Shuttle candidates against other targets (open circles) are left for reference.

FIG. 48 depicts plots showing in vivo PK validation of TfR1 and CD98hc shuttles with a common anti-amyloid beta (Aβ) antibody payload. Shown are shuttled and unshuttled anti-aβ mAbs with timepoints measured at 1, 6, 24, and 72 hours post-injection. The left plot shows kinetics of shuttle TfR-1409-51 with anti-aβ measured in humanized TfR1 mice. The right plot shows kinetics of shuttle CD98-11 with anti-aβ measured in humanized CD98hc mice.

FIG. 49 depicts plots showing in vivo PK validation of a unique (e.g., not previously known) target FLT1 using shuttle FLT1-49 in C57 mice. Shown are shuttled and unshuttled anti-aβ mAbs with timepoints measured at 1, 6, 24, and 72 hours post-injection. The left plot shows quantitation of FLT1-49 in whole-brain. The right plot shows quantitation of FLT1-49 in parenchymal-extracted tissues.

FIG. 50 depicts plots showing brain uptake and functional activity of an anti-BACE1 antibody alone or shuttled by MB-TfR-1409-51 versus benchmarks in humanized TfR1 mice at 24 hrs post-injection. Column 1 from left shows format of unshuttled anti-BACE1 control mAb and the shuttled MB-TfR-1409-51 construct. Column 2 from left shows levels of antibody for each construct measured in whole brain using ELISA. Column 3 from left shows levels of antibody for each construct measured in plasma using ELISA. Column 4 from left shows levels of Aβ40 for each construct measured in brain using ELISA.

FIG. 51 shows micrographs of immunofluorescence imaging of an anti-aβ antibody alone versus MB-TfR-1409-51 and multiple benchmark shuttles. Each construct was assayed in humanized TfR mice at 5 mg/kg dose and imaged using constant exposure time at 4600 ms (Cy3). The micrographs show the in vivo biodistribution of constructs in mouse hippocampus sections stained for IgG at 6 and 24 hrs post-injection. The lower plot shows four of the shuttled and unshuttled antibodies shown in the micrographs, in an ELISA readout for total IgG concentration in the brain (nM) at 1, 6, 24, and 72 hours. MB-TfR-1409-51 is differentiated by faster and more total uptake in the brain relative to the Benchmark Shuttle B and unshuttled controls tested.

FIG. 52 shows a summary of exemplary TfR, CD98, and FLT1 expression profiles. Such profiles show that FLT1 and CD98 are supportive of improved tolerability and efficacy for shuttled anti-aβ cargos.

FIGS. 53A-53B depict methodology related to conjugation of an antisense oligonucleotide to a shuttle in accordance with embodiments of the present disclosure. FIG. 53A depicts a schematic for an exemplary ASO conjugation strategy. Shuttles appended to Fc molecules are conjugated to functionalized antisense oligonucleotides (ASOs) by a two-step process, followed by a final purification step. FIG. 53B shows oligo conjugation and size exclusion chromatography of ASO conjugated shuttles. (Left) Control molecules and TfR1409-51 run on non-reducing SDS-PAGE, stained for protein (Coomassie) and DNA (SYBR gold), before and after ASO conjugation. (Right) Chromatogram from size exclusion chromatography of Fc construct after ASO conjugation to remove excess ASO.

FIG. 54 depicts a plot showing MALAT1 lncRNA knockdown in TfR expressing HEK293 cells by shuttle conjugated ASOs. qRT-PCR measured fold change of MALAT1 after treatment with unshuttled control (Fc:ASO); ASO alone; TfR1409-51 (Shuttle-Fc:ASO); Benchmark #1 (Benchmark #1:ASO). Normalized to untreated control cells and GAPDH. qRT-PCR conducted with TaqMan probes.

FIG. 55 depicts a plot showing nomination of exemplary receptor targets for brain shuttling with more favorable gene expression profiles.

FIG. 56 depicts a plot showing reticulocyte counts after injection with shuttled Anti-aβ or unshuttled Anti-aβ.

FIG. 57 shows immunofluorescence imaging of an exemplary antibody alone versus with shuttle FLT1-49.

FIG. 58 depicts plots showing levels of an anti-aβ mAb alone or fused to shuttle CD98-11 in serum and whole brain.

FIG. 59 depicts plots showing levels of an anti-aβ mAb alone or fused to shuttle FLT1-49 in serum and brain.

FIG. 60 depicts plots showing levels of an anti-aβ mAb alone or fused to shuttle FLT1-49 in serum and brain.

FIG. 61 shows representative 3D whole brain images generated using light sheet fluorescence microscopy (LSFM) and whole-organ immunohistochemistry (iDISCO) following treatment with an anti-aβ mAb alone (unshuttled), versus fused to shuttle CD98-11, or a TfR-shuttled aβ control.

FIG. 62 shows representative 3D whole brain images generated using light sheet fluorescence microscopy (LSFM) and whole-organ immunohistochemistry (iDISCO) following treatment with an anti-aβ mAb alone (unshuttled), versus fused to shuttle FLT1-49, or a TfR-shuttled aβ control.

FIG. 63 shows representative 2D sections from magnified areas of whole brains scans acquired using Light sheet fluorescence microscopy (LSFM) following treatment with an anti-aβ mAb alone (unshuttled), versus fused to shuttle FLT1-49, or a TfR-shuttled aβ control.

FIGS. 64A-64C depict plots showing levels of an IgG either alone (unshuttled) versus fused to shuttle FLT1-49 (parent) or an affinity-optimized variant FLT1-49-41.

FIG. 65 depicts a plot showing reticulocyte counts after injection with shuttled Anti-aβ or unshuttled Anti-aβ.

FIG. 66 depicts a plot showing gene expression profiles of exemplary receptor targets across various cell types and tissues.

FIG. 67 depicts a table showing acute symptoms in mice following treatment with shuttled Anti-aβ or unshuttled Anti-aβ.

FIG. 68 shows representative immunofluorescence images from brain sections twenty-four hours post-injection of either unshuttled Anti-aβ or FLT1-49-shuttled Anti-aβ.

FIG. 69 depicts plots showing pharmacokinetic (PK) profiles of selected TfR shuttles (blue) vs two unshuttled controls (gray and black) across serum brain tissue, and corresponding brain to serum ratio as measured in non-human primates.

FIG. 70 depicts plots showing pharmacokinetic (PK) profiles of exemplary TfR shuttles (blue) vs two unshuttled controls (gray and black) across serum, brain tissue, and corresponding brain to serum ratio as measured in humanized TfR mice.

FIG. 71 depicts plots showing pharmacokinetic (PK) profiles of exemplary CD98 shuttles (blue) vs two unshuttled controls (gray and black) across serum, brain tissue, and corresponding brain to serum ratio as measured in humanized CD98 mice.

FIG. 72 depicts plots showing pharmacokinetic (PK) profiles of exemplary FLT1 shuttles (blue) vs 2 unshuttled controls (gray and black) across serum, brain tissue, and corresponding brain to serum ratio as measured in humanized FLT1 mice.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administer: The term “administer” or “administering”, when used herein typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference”]. In some embodiments, affinity may be assessed relative to a contemporaneous reference; in some embodiments, affinity may be assessed relative to a historical reference. Typically, when affinity is assessed relative to a reference, it is assessed under comparable conditions.

Agent: In general, the term “agent”, as used herein, is used to refer to an entity (e.g., for example, a lipid, metal, nucleic acid, polypeptide, polysaccharide, small molecule, etc, or complex, combination, mixture or system [e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Animal: as used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a cargo [e.g., a detectable moiety, a therapeutic moiety, a catalytic moiety, etc], or other pendant group [e.g., poly-ethylene glycol, etc.]

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a cargo [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Barcode, Barcode component, or Barcode peptide or Peptide barcode: As used herein, the term “barcode” refers to a sequence (nucleic acid or amino acid), which associates (e.g., covalently or non-covalently) with a cargo as described herein. In some embodiments, a nucleic acid comprises a “barcode component” encoding a peptide barcode. In some embodiments, a barcode component is operably linked to a cargo component that encodes a cargo polypeptide. In some embodiments, a peptide barcode is linked to cargo polypeptide. As described herein a barcode associates with a binder with known specificity and affinity. In some embodiments, a barcode binds to a specific antibody-agent. In some embodiments, a barcode may be contained within a specific cargo of interest. In some embodiments, a barcode may be terminal to a specific cargo of interest. In some embodiments, a barcode may be synthetic. In some embodiments, a barcode may be designed. For example, a barcode sequence may be ordered as a DNA polynucleotide and cloned into a cargo of interest using methods of molecular cloning known to a person of ordinary skill in the art.

Binder: As used herein, the term “binder” or “binder moiety” refers to a polypeptide sequence, which associates with a barcode with known specificity and affinity. In some embodiments, a binder is or comprises an antibody agent. In some embodiments, a binder is expressed on a surface of a binding agent. In some embodiments, a binder may bind to one or more barcodes.

Binding: It will be understood that the term “binding” or “bind”, as used herein, typically refers to a non-covalent association between or among two or more entities. “Direct” binding involves physical contact between entities or moieties, indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Binding agent: In general, the term “binding agent” is used herein to refer to any entity that binds to a target of interest as described herein (e.g., a barcode, a barcoded target, etc.). In many embodiments, a binding agent of interest is one that binds specifically with its target in that it discriminates its target from other potential binding partners in a particular interaction context. In general, a binding agent may be or comprise an entity of any chemical class (e.g., polymer, non-polymer, small molecule, polypeptide, carbohydrate, lipid, nucleic acid, etc) or biological class (e.g., bacteria, phage, ribosome, mRNA, DNA, etc.). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding moiety partner. In some embodiments, binding agents are or comprise phages. In some embodiments, binding agents are or comprise polypeptides (including, e.g., antibodies or antibody fragments). In some embodiments, binding agents are or comprise small molecules. In some embodiments, binding agents are or comprise nucleic acids. In some embodiments, binding agents are or comprise aptamers. In some embodiments, binding agents are polymers; in some embodiments, binding agents are not polymers. In some embodiments, binding agents are non-polymeric in that they lack polymeric moieties. In some embodiments, binding agents are or comprise carbohydrates. In some embodiments, binding agents are or comprise lectins. In some embodiments, binding agents are or comprise peptidomimetics. In some embodiments, binding agents are or comprise scaffold proteins. In some embodiments, binding agents are or comprise mimeotopes. In some embodiments, binding agents are or comprise stapled peptides. In certain embodiments, binding agents are or comprise nucleic acids, such as DNA or RNA.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow, blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Cargo, Cargo component, or Cargo polypeptide: As used herein, the term “cargo” refers to a payload, which may be associated (e.g., covalently or non-covalently) to a barcode and/or a shuttle. In some embodiments, a cargo comprises a nucleic acid (referred to herein as a “cargo component”) encoding a cargo polypeptide. In some embodiments, a cargo is or comprises a cargo component (e.g., that encodes a cargo polypeptide). In some embodiments, a cargo is or comprises a cargo polypeptide (e.g., encoded by a cargo component). In some embodiments, a cargo component is operably linked to a nucleic acid referred to herein as a “barcode component”. In some embodiments, a barcode component encodes a peptide barcode. In some embodiments, a peptide barcode is linked (e.g., covalently and/or non-covalently) to a cargo polypeptide. In some embodiments, a cargo component is operably linked to a nucleic acid referred to herein as a “shuttle component”. In some embodiments, a shuttle component encodes a peptide shuttle (or “transporter”). In some embodiments, a peptide shuttle is linked (e.g., covalently and/or non-covalently) to a cargo polypeptide. In some embodiments, a peptide shuttle is linked (e.g., covalently and/or non-covalently) to a peptide barcode. In some embodiments, a cargo polypeptide is detected in a pool of polypeptides. In some embodiments, a cargo polypeptide is an unmodified polypeptide that is to be detected in a pool of polypeptides without association of a peptide barcode. In some embodiments, a cargo polypeptide is a modified polypeptide that is to be detected in a pool of polypeptides. In some embodiments, a cargo polypeptide may not be associated with a barcode (e.g., a peptide barcode). In some embodiments, a cargo polypeptide may not be associated with a shuttle (e.g., a peptide shuttle). In some embodiments, a cargo comprises one or more sequences (nucleic acid sequence or amino acid sequence) that modify expression of a cargo polypeptide. In some embodiments, such one or more sequences are associated (directly or indirectly) with a barcode throughout a period of assessment of cargo polypeptides, as described herein.

CDR: As used herein, “CDR” refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

Characteristic portion: As used herein, the term “characteristic portion,” in the broadest sense, refers to a portion of a substance whose presence (or absence) correlates with presence (or absence) of a particular feature, attribute, or activity of the substance. In some embodiments, a characteristic portion of a substance is a portion that is found in a given substance and in related substances that share a particular feature, attribute or activity, but not in those that do not share the particular feature, attribute or activity. In some embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some embodiments, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence dement: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of a polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share a sequence element.

Combination therapy: As used herein, the term “combination therapy” refers to those situations in which a subject is simultaneously exposed to two or more therapeutic regimens (e.g., two or more therapeutic agents). In some embodiments, two or more agents may be administered simultaneously. In some embodiments, two or more agents may be administered sequentially. In some embodiments, two or more agents may be administered in overlapping dosing regimens.

Comparable. As used herein, the term “comparable” refers to two or more agents, entities, situations, sets of conditions, that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Decoding: As used herein, the term “decoding”, refers to a laboratory and/or bioinformatics process of identifying and quantifying a unique set of amino acids within a barcode. In some embodiments, such identification and quantification is achieved using nucleic acid (e.g., DNA) counts form a sequencing experiment and measuring an abundance of binder counts. In some embodiments, previously measured fingerprints (e.g., binder fingerprint or barcode fingerprint) are used to determine the relationship between an unknown barcode mixture, which is being decoded, for example, by comparing to a previously known mixture's binder counts, across binders with known and varying affinities to several barcodes within the pool.

Designed. As used herein, the term “designed” refers to an agent (i) whose structure is or was selected by the hand of man; (ii) that is produced by a process requiring the hand of man; and/or (iii) that is distinct from natural substances and other known agents.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Engineered: In general, the term “engineered” refers to the aspect of having been manipulated by the hand of man. For example, in some embodiments, a small molecule may be considered to be engineered if its structure and/or production is designed and/or implemented by the hand of man. Analogously, in some embodiments, a polynucleotide may be considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first sequence (e.g., coding sequence) but not in operative association with a second sequence (e.g., coding sequence), is linked by the hand of man so that it is operatively associated with the second sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, expression products of an engineered polynucleotide, and/or progeny of an engineered polynucleotide or cell are typically still referred to as “engineered” even though the actual manipulation was performed on a prior entity.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fingerprint: As used herein, the term “fingerprint” refers to the counts of one or more unknown agents that a known agent may bind to or be associated with. In some embodiments, a fingerprint may be for a known barcode or barcode mixture. In some embodiments, a fingerprint may be for a known binder or binder mixture. For example, in some embodiments, a fingerprint (e.g., barcode fingerprint) may refer to the counts of one or more binders (e.g., determined through sequencing analysis) to bind specifically to a known barcode or barcode mixture. That is, in some embodiments, a fingerprint for a barcode refers to the counts of one or more binders, some of which may have high affinity for the barcode, and some of which may have low affinity for the barcode. In some embodiments, a fingerprint may be used in the decoding process, which process is used to determine the relative or absolute abundance of a given barcode within a pool of barcodes. As is understood to a person of ordinary skill in the art a fingerprint may be determined for a known barcode or barcode mixture, or for a known binder or binder mixture. For example, in some embodiments, a fingerprint (e.g., binder fingerprint) may refer to the counts of one or more barcodes (e.g., determined through sequencing analysis) that bind specifically to a known binder or binder mixture. That is, in some embodiments, a fingerprint for a binder refers to the counts of one or more barcodes, some of which may have high affinity for the binder, and some of which may have low affinity for the binder. Accordingly, a fingerprint may also be used in the decoding process, in some embodiments, to determine the relative or absolute abundance of a given binder within a pool of binders.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the whole.

Human: In some embodiments, a human is an embryo, a fetus, an infant, a child, a teenager, an adult, or a senior citizen.

Identity, identical: As used herein, the terms “identity” or “identical” refers to overall relatedness between polymeric molecules, e.g., between nucleic acid molecules (e.g., DNA molecules and/or RNA molecules) and/or between polypeptide molecules. Methods for calculation of a percent identity as between two provided sequences are known in the art. Calculation of a percent identity of two nucleic acid or polypeptide sequences, for example, can be performed by aligning two sequences (or a complement of one or both sequences) for optimal comparison purposes (e.g., gaps can be introduced in one or both of a first and a second sequences for optimal alignment and non-identical sequences can be disregarded for comparison purposes). Nucleotides or amino acids at corresponding positions are then compared. When a position in the first sequence is occupied by a same residue (e.g., nucleotide or amino acid) as a corresponding position in the second sequence, then molecules are identical at that position. Percent identity between two sequences is a function of a number of identical positions shared by the sequences and, optionally, taking into account a number of gaps and a length of each gap, which may need to be introduced for optimal alignment of two sequences. Comparison of sequences and determination of percent identity between two sequences can be accomplished using a computational algorithm, such as BLAST (basic local alignment search tool).

Improve, increase, inhibit or reduce: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment. In some embodiments, “improve”, “increase”, “inhibit”, “reduce” may be referred to collectively as “modify”.

Invariant sequence: As used herein, the term “invariant sequence” indicates a sequence that is substantially identical in a library of nucleic acids. In some embodiments, each nucleic acid comprises, among other things, a barcode component. As an example, in some embodiments, a barcode component may further comprise one or more of: (1) a nucleic acid sequence encoding a short helical motif, (2) a nucleic acid encoding a disordered motif, and (3) an invariant sequence linking the barcode component to the cargo component. A nucleic acid sequence encoding a short helical motif and a nucleic acid encoding a disordered motif may each respectively vary across the library of nucleic acids. In contrast, each invariant sequence in a pool of nucleic acids is substantially identical.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Library: The term “library” as used herein refers to a mixture of one or more distinct molecules. In some embodiments, all elements of a library share one or more common components. In some embodiments, all elements of a library share no common components. In some embodiments, one or more elements of a library are distinguished by one or more unique components. In some embodiments, as may be apparent from the context, a library may refer to a mixture of binding agents. In some embodiments, a library may be a phage library. In some embodiments, for example, a phage library may consist of phage with distinct binders displayed on (e.g., on a surface) of the phage and encapsulating DNA encoding for this binder within the phage. In some embodiments, a library may refer to a mixture of barcoded cargo proteins. In some embodiments, a library may refer to a mixture of barcodes (e.g., peptide barcodes).

Linker: as used herein, is used to refer to that portion of a multi-element agent that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a polyptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) known in the art (see e.g., Holliger, P., et al. (1993) Proc. Natl. Acad. Sci. USA 90:6444-6448; Poljak, R J., et al. (1994) Structure 2: 1 121-1123).

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with coding elements of interest; in some embodiments, control elements act in trans to or otherwise at a from the functional element of interest. In some embodiments, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some embodiments, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame. In some embodiments, a cargo component is operably linked to a barcode component.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition in which an active agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some embodiments, a pharmaceutical composition may be formulated for administration via injection either in a particular organ or compartment, e.g., directly into an ear, or systemic, e.g., intravenously. In some embodiments, a formulation may be or comprise drenches (aqueous or non-aqueous solutions or suspensions), tablets, boluses, powders, granules, pastes, capsules, powders, etc. In some embodiments, an active agent may be or comprise an isolated, purified, or pure compound.

Polypeptide: As used herein refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide. In some embodiments, a polypeptide may be a protein.

Pro Component: As used herein, the term “pro component” refers to an inactive component. In some embodiments, a pro component once expressed can take an active form, for example, to have an intended effect. In some embodiments, a pro component may be expressed in vitro. In some embodiments, a pro component may be expressed in vivo. In some embodiments, a pro component may be expressed in a tissue (e.g., of an animal (e.g., a mammal)).

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain l-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Regulatory Element: As used herein, the term “regulatory element” or “regulatory sequence” refers to non-coding regions of DNA that regulate, in some way, expression of one or more particular genes. In some embodiments, such genes are apposed or “in the neighborhood” of a given regulatory element. In some embodiments, such genes are located quite far from a given regulatory element. In some embodiments, a regulatory element impairs or enhances transcription of one or more genes. In some embodiments, a regulatory element may be located in cis to a gene being regulated. In some embodiments, a regulatory element may be located in trans to a gene being regulated. For example, in some embodiments, a regulatory sequence refers to a nucleic acid sequence which is regulates expression of a gene product operably linked to a regulatory sequence. In some such embodiments, this sequence may be an enhancer sequence and other regulatory elements which regulate expression of a gene product.

Sample: As used herein, the term “sample” typically refers to an aliquot of material obtained or derived from a source of interest. In some embodiments, as would be appreciated from the context by a person of ordinary skill in the art, the term “sample” may be used interchangeably with terms like “mixture”, or “complex mixture”, or “complex sample”. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise cells, serum, extracellular matrix, CSF, and/or combinations or component(s) thereof. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Specific: The term “specific”, when used herein with reference to an agent having an activity, is understood by those skilled in the art to mean that the agent discriminates between potential target entities or states. For example, in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Subject: As used herein, the term “subject” refers to an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a therapeutic protein.

Variant: As used herein, the term “variant” refers to a version of something, e.g., a gene sequence, that is different, in some way, from another version. To determine if something is a variant, a reference version is typically chosen and a variant is different relative to that reference version. In some embodiments, a variant can have the same or a different (e.g., increased or decreased) level of activity or functionality than a wild type sequence. For example, in some embodiments, a variant can have improved functionality as compared to a wild-type sequence if it is, e.g., mutated to confer reduced toxicity in a cell. As another example, in some embodiments, a variant can have improved functionality as compared to a wild-type sequence if it is, e.g., mutated to confer improved protein production in a cell. As another example, a “variant polypeptide” as used herein is a variant polypeptide that comprises one or more mutations relative to a reference polypeptide.

### DETAILED DESCRIPTION

### I. Barcoded Cargos

Methods and systems to generate and use barcodes and barcoded cargo are described herein. In some embodiments, a cargo polypeptide is encoded by a cargo component. In some embodiments, a peptide barcode is encoded by a barcode component. In some embodiments, cargo components are operably linked to barcode components. Other exemplary cargos are described throughout the present disclosure.

Among other things, the present disclosure provides for methods used to detect and/or characterize cargos. In some embodiments, methods disclosed herein are used to detect and/or characterize cargo polypeptides (e.g., therapeutic polypeptides) encoded by cargo components. In some embodiments, methods disclosed herein are used to detect and/or characterize therapeutic or non-therapeutic polypeptides. In some embodiments, methods disclosed herein are used to detect and/or characterize cargos by tagging them with barcodes (e.g., barcoded cargo components). In some embodiments, methods disclosed herein are used to detect and/or characterize cargos in vitro. In some embodiments, methods disclosed herein are used to detect and/or characterize cargos in vivo. In some embodiments, methods disclosed herein are used to detect and/or characterize a cargo. In some embodiments, methods disclosed herein are used to detect and/or characterize multiple (e.g., two or more, three or more, four or more, etc.) cargos.

i. Barcodes

In some embodiments, a barcode is or comprises an amino acid sequence. In some embodiments, a barcode is or comprises an amino acid sequence that occurs in nature. In some embodiments, a barcode is or comprises an amino acid sequence that does not occur in nature. In some embodiments, a barcode is or comprises an amino acid sequence that is synthetic. In some embodiments, a barcode comprises naturally occurring amino acids. In some embodiments, a barcode comprises non-naturally occurring amino acids (e.g., modified amino acids). In some embodiments, a barcode is or comprises a peptide barcode.

Barcodes of the present disclosure can be of varying lengths. For example, in some embodiments, a barcode may have a length ranging between 1 and 100 amino acids. In some embodiments, a barcode may have a length ranging between 5 and 50 amino acids. In some embodiments, a barcode may have a length ranging between 8 and 25 amino acids. In some embodiments, a barcode may have a length ranging between 9 and 25 amino acids. In some embodiments, a barcode may have a length ranging between 9 and 15 amino acids. In some embodiments, a barcode may have a length of 1, 2, 3, 4, 5, 6, 7, 8, 9, 10, 11, 12, 13, 14, 15, 16, 17, 18, 19, 20, 21, 22, 23, 24, or 25 amino acids. In some embodiments, a barcode may have a length of at least 5 amino acids. In some embodiments, a barcode may have a length of at most 100 amino acids.

Barcodes, as described herein may be available in a library in different formats. For example, in some embodiments a barcode as described herein may be described as a nucleic acid sequence. In other instance, a barcode as described herein may be described as an amino acid sequence. A person of ordinary skill in the art will appreciate that barcodes described in one format may be converted to another format using basic biological principles. Accordingly, barcodes described as nucleic acid sequences may be translated into proteins, which may be used to detect the presence or absence of a cargo (e.g., cargo polypeptide) in a mixture. Such a translated barcode is referred to herein as a peptide barcode.

Accordingly, barcodes of the present disclosure when described using nucleic acids may have lengths different from amino acid sequence lengths disclosed in the paragraph above. For example, in some embodiments, a barcode may have a length ranging between 3 and 300 nucleotides. In some embodiments, a barcode may have a length ranging between 15 and 150 nucleotides. In some embodiments, a barcode may have a length ranging between 24 and 75 nucleotides. In some embodiments, a barcode may have a length ranging between 27 and 75 nucleotides. In some embodiments, a barcode may have a length ranging between 27 and 45 nucleotides. In some embodiments, a barcode may have a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nucleotides. In some embodiments, a barcode may have a length of at least 15 nucleotides. In some embodiments, a barcode may have a length of at most 300 nucleotides.

Barcodes of the present disclosure may have one or more properties. In some embodiments, a barcode may be naturally occurring. In some embodiments, a barcode may not be naturally occurring (e.g., synthetic). In some embodiments, a barcode may have relatively no effect on cargo function. For example, in some embodiments, tagging a cargo (e.g., a cargo component) with a barcode as described herein does not alter or change relatively the function of the tagged cargo. In some embodiments, a barcode may have an effect (e.g., positive or negative) on cargo function. For example, in some embodiments, tagging a cargo (e.g., a cargo component) with a barcode as described herein may alter or change relatively a function (e.g., half-life (e.g., longer half-life), enhance targeting to specific tissue, etc.) of the tagged cargo. In some embodiments, a barcode may not elicit an immune response (e.g., an IgG response, a complement response, etc.). In some embodiments, barcodes are orthogonal to each other. In some embodiments, barcodes are not orthogonal to each other.

Barcodes of the present disclosure may be associated to various positions of a cargo. For example, in some embodiments, a barcode may be associated (e.g., covalently or non-covalently) to a suitable (e.g., desirable) position on a cargo. In some embodiments, a barcode may be associated (e.g., covalently or non-covalently) to a less-suitable or non-suitable (e.g., non-desirable) position on a cargo. In some embodiments, a peptide barcode may be associated (e.g., covalently or non-covalently) to a suitable position on a cargo polypeptide. For example, in some embodiments, a barcode may be associated (e.g., covalently or non-covalently) to an N-terminus of a cargo polypeptide. In some embodiments, a barcode may be associated (e.g., covalently or non-covalently) to a C-terminus of a cargo polypeptide. In some embodiments, a barcode may be associated (e.g., covalently or non-covalently) to a non-terminal position on a cargo polypeptide (e.g., side chain). In some embodiments, a barcode may be associated (e.g., covalently or non-covalently) to a less-suitable or non-suitable (e.g., non-desirable) position on a cargo polypeptide.

Among other things, barcodes (e.g., peptide barcodes or barcode components encoding peptide barcodes) of the present disclosure may be flanked by additional sequences (e.g., nucleic acid sequences, amino acid sequences, etc.). In some embodiments, a barcode may be flanked by additional sequences on a barcode's 5′ end. In some embodiments, a barcode may be flanked by additional sequences on a barcode's 3′ end. In some embodiments, a barcode may be flanked by additional sequences on a barcode's 3′ and 5′ end. In some embodiments, an additional sequence may be a primer binding site, a protease-cleavable linker sequence, a sortase site, a transglutaminase site, a restriction endonuclease recognition sequence, a restriction enzyme site (e.g., a cleavage site), a sequence that encodes an amino acid sequence, a sequence that does not encode an amino acid sequence, an amino acid sequence, or a nucleic acid sequence. For example, in some embodiments, a barcode may be flanked by nucleic acid sequences encoding an amino acid sequence. In some embodiments, a barcode may be flanked by nucleic acid sequences that does not encode an amino acid sequence. In some embodiments, a barcode may be flanked by amino acid sequences. In some embodiments, a peptide barcode may be flanked by amino acid sequences (e.g., Glycine-Serine (GS), e.g., other linker amino acid sequences). Analogously, in some embodiments, a barcode component encoding a peptide barcode of the present disclosure may be flanked by additional sequences (e.g., nucleic acid sequences, amino acid sequences, etc.). In some embodiments, a barcode component encoding a peptide barcode may be flanked by nucleic acid sequences on a 5′ end. In some embodiments, a barcode component encoding a peptide barcode may be flanked by nucleic acid sequences on a 3′ end. In some embodiments, a barcode component encoding a peptide barcode may be flanked by nucleic acid sequences on a 3′ and 5′ end. In some embodiments, a barcode component encoding a peptide barcode may be flanked by nucleic acid sequences encoding an amino acid sequence comprising a Glycine-Serine (GS). In some embodiments, a barcode component encoding a peptide barcode may be flanked by nucleic acid sequences encoding an amino acid sequence comprising a linker amino acid sequence as described herein.

In some embodiments, a barcode may be flanked by restriction endonuclease recognition sequences. In some embodiments, a barcode may be flanked by restriction endonuclease recognition sequences on a barcode's 5′ end. In some embodiments, a barcode may be flanked by restriction endonuclease recognition sequences on a barcode's 3′ end. In some embodiments, barcode may be flanked by restriction endonuclease recognition sequences on a barcode's 3′ and 5′ end. In some embodiments, a nucleic acid encoding a peptide barcode may be flanked by restriction endonuclease recognition sequences. In some embodiments, a nucleic acid encoding a peptide barcode may be flanked by restriction endonuclease recognition sequences on a 5′ end. In some embodiments, a nucleic acid encoding a peptide barcode may be flanked by restriction endonuclease recognition sequences on a 3′ end. In some embodiments, a nucleic acid encoding a peptide barcode may be flanked by restriction endonuclease recognition sequences on a 3′ and 5′ end. In some embodiments, a restriction endonuclease recognition sequence may be recognized by one or more restriction enzymes (e.g., BsaI, BsmBI, BbsI, SapI, etc.). In some embodiments, restriction endonuclease recognition sequences are Type I, Type II, or Type IIs restriction endonuclease recognition sequences. Such recognition sequences, for example, may be used to produce universal overhangs that may be used in cloning peptide barcodes into different locations of various cargo. Such flexibility allows a barcode to be used to detect different cargo polypeptides in different experiments.

In some embodiments, a barcode component encoding a barcode (e.g., a peptide barcode) may be associated with (e.g., attached to, linked to) a second cargo component encoding a cargo polypeptide (e.g., a cargo polypeptide of interest). Such nucleic acid sequences, for example, may be translated to form barcoded cargos (e.g., barcoded cargo polypeptides, barcoded shuttle-cargo polypeptides). In some embodiments, a barcode component encoding a barcode (e.g., a peptide barcode) is separate from a cargo component encoding a cargo polypeptide (e.g., a cargo polypeptide of interest). For such nucleic acid sequences, for example, a barcode component encoding a peptide barcode may be translated separately from a cargo component sequence encoding a cargo polypeptide, and subsequently attached using one or more methods known in the art to join distinct amino acid sequences (e.g., using linkers).

Barcodes of the present disclosure may be associated (e.g., directly or indirectly attached) to cargos so as to form barcoded cargos (or barcoded cargo components as described herein). For example, in some embodiments, each barcode sequence (e.g., peptide barcode sequence) may be associated to only one cargo of interest (e.g., cargo polypeptide of interest) within a mixture. In some embodiments, each barcode sequence may be associated to more than one cargo of interest (e.g., cargos with different sequences) within a mixture. In some embodiments, multiple (e.g., two or more, three or more, four or more, etc.) barcode sequences may be associated to one cargo of interest within a mixture. For example, in some embodiments, one or more barcode sequences may be associated to various different positions on a given cargo—such a setup may be useful in, for example, in studying and identifying the stability and/or cleavage of such barcoded cargos. In some embodiments, each cargo in a mixture is a unique sequence (e.g., each cargo has a different sequence from every other cargo in the mixture). In some embodiments, each cargo in a mixture is a non-unique sequence.

Various methods and parameters may be used to select suitable barcodes for a given cargo. For example, stability of a barcoded cargo is key in determining if a cargo may be tagged by said barcode. In some embodiments, a barcode may be tagged to a specific cargo across different experiments. In some embodiments, a barcode may be tagged to different cargos in different experiments. For example, in some embodiments, a barcode may be tagged to two or more, three or more, four or more, ten or more, 100 or more, 1000 or more, or 10,000 or more different cargos across different experiments.

In some embodiments, a barcode may be associated with only one cargo in a given experiment. In some embodiments, a barcode may be associated with multiple cargos in a given experiment. For example, in some embodiments, one or more barcodes are associated with multiple cargos (i.e., a barcode is tagged to multiple cargos) in a mixture, such that each cargo is associated with a unique set of barcodes within the mixture. That is, each cargo may be associated with a unique “pattern” of barcodes in the mixture. Analogously, in some embodiments, several cargos may be associated with the same barcode.

Among other things, barcodes described herein are designed to have a distinct (i.e., unique) sequence. In some embodiments, a barcode is designed to have a distinct sequence (e.g., distinct from another barcode). For example, each barcode is designed to be distinct (e.g., unique) from every other barcode used in an experiment, such that each cargo (e.g., protein to be measured) is attached to at least one barcode, and each barcode (e.g., barcode with a specific sequence) is only attached to one cargo. As may be understood by a person of ordinary skill in the art, the diversity of barcodes contained within a pool is limited only by the possible diversity of amino acid sequences for a given barcode length. For example, for a barcode length ‘N’, there exists 20^{N} distinct amino acid barcode sequences of length N (if only unmodified/naturally occurring amino acids are used). That is, for a barcode length of 15, the theoretical limit is 20¹⁵, or 3.2768×10¹⁹.

In some embodiments, barcodes, as described herein, can be designed and/or developed through machine-learning methods.

Example of barcodes according to various embodiments of the present disclosure are listed in Table 1 and Table 2. In some embodiments, a barcode (e.g., peptide barcode) is or comprises an amino acid sequence selected from SEQ ID NOs: 5347-8398. In some embodiments, a barcode (e.g., peptide barcode) is encoded by a sequence that is or comprises a nucleic acid sequence selected from SEQ ID NOs: 1148-4199.

ii. Cargo Polypeptides

Methods and systems disclosed herein may be used for detection of one or more cargos as described herein.

In one aspect, systems and methods disclosed herein may be used for detecting a cargo (e.g., cargo polypeptide) in a mixture. Specifically, barcodes disclosed herein tagged to a cargo (e.g., barcoded cargo component) in a mixture and used to detect said cargo in the mixture. In some embodiments, each cargo is different from every other cargo in a mixture. In some embodiments, each cargo in a mixture is different from every other cargo in a mixture by at least one amino acid. In some embodiments, each cargo in a mixture is different from every other cargo in a mixture by two or more amino acids. In some embodiments, a cargo (e.g., in a mixture) may be tagged with a barcode. In some embodiments, each cargo (e.g., in a mixture) may be tagged with a same barcode. In some embodiments, each cargo (e.g., in a mixture) may be tagged with different barcode. In some embodiments, a cargo (e.g., in a mixture) may be tagged with a barcode that is different from every other barcode (e.g., associated with other cargos) in a mixture by at least one amino acid. In some embodiments, a cargo (e.g., in a mixture) may be tagged with a barcode that is different from every other barcode (e.g., associated with other cargos) in a mixture by two or more amino acids.

As discussed elsewhere in the specification, a cargo may be tagged with different barcodes (e.g., in different mixtures, different experiments, etc.). For example, as noted above, in some embodiments, each barcode sequence may be associated (e.g., covalently or non-covalently) with only one cargo of interest within a mixture. In some embodiments, each barcode sequence may be associated (e.g., covalently or non-covalently) with more than one cargo of interest (e.g., cargos with different sequences) within a mixture. In some embodiments, multiple (e.g., two or more, three or more, four or more, etc.) barcode sequences may be attached to one cargo of interest within a mixture. For example, in some embodiments, one or more barcode sequences may be attached to various different positions on a given cargo—such a setup may be useful in, for example, in studying and identifying the stability of such barcoded cargos. In some embodiments, each cargo in a mixture is a unique sequence (e.g., each cargo has a different sequence from every other cargo in the mixture). In some embodiments, each cargo in a mixture is a non-unique sequence.

In some embodiments, a cargo may be tagged to a specific barcode across different experiments. In some embodiments, a cargo may be tagged to different barcodes in different experiments. For example, in some embodiments, a cargo may be tagged to two or more, three or more, four or more, ten or more, 100 or more, 1000 or more, or 10,000 or more different barcodes across different experiments.

In some embodiments, a cargo may be associated with only one barcode in a given experiment. In some embodiments, a cargo may be associated with multiple barcodes in a given experiment. For example, in some embodiments, one or more barcodes (e.g., in a mixture) are associated with multiple cargos (i.e., a barcode is tagged to multiple cargos) in a mixture, such that each cargo is associated with a unique set of barcodes within the mixture. That is, each cargo may be associated with a unique “pattern” of barcodes in the mixture. In some embodiments, several cargos may be associated with the same barcode.

Among other things, the present disclosure provides for nucleic acids comprising, for example, a cargo component encoding a cargo polypeptide of interest. In some embodiments, a cargo polypeptide has a therapeutic function. In some embodiments, a cargo polypeptide does not have a therapeutic function (e.g., may aid another cargo with a therapeutic function). For example, possible cargo polypeptides which one may wish to screen as drugs, such as monoclonal antibodies, single domain antibodies, enzymes, bispecific antibodies, or any other cargo polypeptide which may have therapeutic function.

In some embodiments, a cargo polypeptide further comprises a targeting moiety. In some embodiments, a targeting moiety targets a cargo polypeptide to a location of interest (e.g., a cell of interest, a tissue of interest, an organ of interest). In some embodiments, a targeting moiety targets a cargo polypeptide to a cell-receptor agent of interest. In some embodiments, a cell-receptor agent targeted by the targeting moiety is referred to herein as a “portal”. In some embodiments, a targeting moiety is expressed on a surface of a delivery particle described herein. Targeting moieties are known in the art.

In some embodiments, a cargo polypeptide further comprises a localizing moiety. In some embodiments, a localizing moiety is a secretion peptide signal. In some embodiments, a localizing moiety is a nuclear localization signal. Other localizing moieties are known in the art.

In some embodiments, a cargo polypeptide further comprises a pro component. In some embodiments, a pro component, as described herein, refers to an inactive component that, once expressed in a tissue of interest, takes an active form so that it exhibits an intended effect. For example, pro components include moieties such as carboxylic, hydroxyl, amine, or phosphate/phosphonate groups. In some embodiments, pro components may be activated once exposed to environmental conditions such as pH, presence (or absence) of an agent, etc.

In some embodiments, a cargo polypeptide further comprises a tag moiety. In some embodiments, a tag moiety comprises a detectable moiety. Tag moieties are known in the art.

In some embodiments, a cargo polypeptide further comprises a liganding moiety. In some embodiments, a liganding moiety targets a cargo polypeptide to a tissue of interest. In some embodiments, a liganding moiety targets a cargo polypeptide to a target agent within a cell, tissue, or organ (e.g., in vivo). In some embodiments, a liganding moiety targets a cargo polypeptide to a target agent on a surface of a cell, tissue, or organ (e.g., in vivo). In some embodiments, a cargo polypeptide further comprises a stability modifying moiety. In some embodiments, a cargo polypeptide further comprises a masking moiety. In some embodiments, a cargo polypeptide further comprises an allosteric modulation moiety.

In some embodiments, a liganding moiety is or comprises a targeting moiety. In some embodiments, a targeting moiety is or comprises a liganding moiety.

In some embodiments, a targeting moiety, as described herein, can be designed and/or developed through machine-learning methods. In some embodiments, a liganding moiety, as described herein, can be designed and/or developed through machine-learning methods.

In some embodiments, a cargo is or comprises a therapeutic agent. In some embodiments, a therapeutic agent is or comprises a nucleic acid. In some embodiments, a therapeutic agent is or comprises a polysaccharide. In some embodiments, a therapeutic agent is or comprises a small molecule. In some embodiments, a therapeutic agent is or comprises a peptide or polypeptide. In some embodiments, a cargo is or comprises an antibody. In some embodiments, a cargo is or comprises an antibody associated with a targeting moiety, as described herein. In some embodiments, a cargo is or comprises an antibody associated with a liganding moiety, as described herein. In some embodiments, a cargo is or comprises an antibody drug conjugate (ADC). In some embodiments, a cargo is or comprises an ADC associated with a targeting moiety, as described herein. In some embodiments, a cargo is or comprises an ADC associated with a liganding moiety, as described herein.

Examples of cargos according to various embodiments of the present disclosure are listed in Table 3 and Table 4. In some embodiments, a cargo is or comprises an antibody. In some embodiments, a cargo is or comprises one or more variable light (VL) chains, variable heavy (VH) chains, and/or complementarity-determining regions (CDRs) of an antibody. In some embodiments, a cargo is or comprises an anti-CD20 antibody (i.e., anti-CD20 Abs). In some embodiments, a cargo is or comprises an anti-amyloid Beta (Aβ) antibody (i.e., anti-Aβ Abs, anti-Aβ oligomers, anti-Ab Abs or anti-Ab oligomers). In some embodiments, a cargo is or comprises an aBACE-1 antibody (i.e., aBACE-1 or anti-BACE-1 (i.e., an antibody that targets BACE-1)).

In some embodiments, a cargo is or comprises one or more sequences (e.g., VL, VH, CDR sequences) associated with an antibody. In some embodiments, a cargo is or comprises one or more sequences (e.g., VL, VH, CDR sequences) associated with rituximab, ocrelizumab, ofatumumab, ublituximab, obinutuzumab, aducanumab, lecanemab, gantenerumab, donanemab, prasinezumab, trontinemab, anti-BACE-1 antibody 6266, anti-BACE-1 antibody YW412.8.31, Lu AF82422, or MEDI1341.

In some embodiments, a cargo is operably linked to a shuttle. In some embodiments, a cargo component is operably linked to a nucleic acid referred to herein as a “shuttle component”. In some embodiments, a shuttle component is or comprises a nucleic acid sequence that encodes a peptide shuttle. In some embodiments, a peptide shuttle is linked (e.g., covalently and/or non-covalently) to a cargo polypeptide (e.g., to form a shuttle-cargo polypeptide). In some embodiments, a cargo polypeptide may not be associated with a shuttle (e.g., a peptide shuttle). Examples of shuttle-cargo polypeptides according to various embodiments of the present disclosure are listed in Table 5.

In some embodiments, a cargo polypeptide is or comprises a wild-type (e.g., naturally occurring) polypeptide. In some embodiments, a cargo polypeptide is or comprises a variant polypeptide (e.g., a variant cargo polypeptide). In some embodiments, a variant polypeptide is a variant of a reference polypeptide, which reference polypeptide is or comprises a wild-type (e.g., naturally occurring) polypeptide. In some embodiments, a variant polypeptide is or comprises at least one mutation relative to a reference polypeptide (e.g., a wild-type polypeptide).

Lengthy table referenced here
US20260158147A1-20260611-T00001
Please refer to the end of the specification for access instructions.

Lengthy table referenced here
US20260158147A1-20260611-T00002
Please refer to the end of the specification for access instructions.

Lengthy table referenced here
US20260158147A1-20260611-T00003
Please refer to the end of the specification for access instructions.

In some embodiments, a variant cargo polypeptide is associated with (e.g., operably linked to) a barcode, as described herein (i.e., a barcoded variant cargo polypeptide). In some embodiments, a variant cargo polypeptide possesses improved functionality (e.g., reduced toxicity (e.g., reduced anemia), improved pharmacokinetic measures (e.g., dissociation constant (Kd), improved biophysical properties, etc.) relative to a reference polypeptide (e.g., a wild-type polypeptide).

In some embodiments, cargos, as described herein, can be designed and/or developed through machine-learning methods. In some embodiments, cargo polypeptides, as described herein, can be designed and/or developed through machine-learning methods. For example, in some embodiments, a cargo polypeptide (e.g., comprising a targeting moiety or a liganding moiety as described herein) can be designed and/or developed (e.g., may be refined through multiple iterations) through machine-learning methods.

An assessment of pharmacokinetic (PK) properties is a key criteria in the nomination of therapeutic leads, but typically occurs in the later stages of drug discovery and only for a limited number of candidates. The binder-barcode platform described herein allows to characterize PK of many therapeutic candidates earlier in drug discovery.

In some embodiments, cargos, designed and/or developed through machine-learning methods possess improved functionality (e.g, reduced toxicity (e.g., reduced anemia), improved pharmacokinetic (PK) measures (e.g., dissociation constant (Kd), improved biophysical properties, epitope properties, affinity properties, thermostability properties, pH sensitivity properties, etc.) relative to a reference cargo (e.g., a wild-type cargo). In some embodiments, cargo polypeptides, designed and/or developed through machine-learning methods possess improved functionality (e.g, reduced toxicity (e.g., reduced anemia), improved pharmacokinetic (PK) measures (e.g., dissociation constant (Kd), improved biophysical properties, epitope properties, affinity properties, thermostability properties, pH sensitivity properties, etc.) relative to a reference cargo polypeptide (e.g., a wild-type cargo polypeptide). In some embodiments, shuttle-cargo polypeptides, designed and/or developed through machine-learning methods possess improved functionality (e.g, reduced toxicity (e.g., reduced anemia), improved pharmacokinetic (PK) measures (e.g., dissociation constant (Kd), improved biophysical properties, epitope properties, affinity properties, thermostability properties, pH sensitivity properties, etc.) relative to a reference shuttle-cargo polypeptide (e.g., a wild-type shuttle-cargo polypeptide).

iii. Cargo Oligonucleotides

In some embodiments, a cargo is or comprises a nucleic acid. In some embodiments, a cargo is or comprises an oligonucleotide. In some embodiments, a cargo is or comprises an antibody associated with (e.g., covalently, non-covalently) an oligonucleotide. In some embodiments, a cargo is or comprises an antibody associated with an oligonucleotide that is associated with a targeting moiety, as described herein. In some embodiments, a cargo is or comprises an antibody associated with an oligonucleotide that is associated with a liganding moiety, as described herein.

In some embodiments, an oligonucleotide comprises DNA. In some embodiments, an oligonucleotide comprises RNA. In some embodiments, an oligonucleotide comprises DNA and RNA. In some embodiments, an oligonucleotide comprises or is an RNA interference (RNAi) molecule. In some embodiments a cargo is or comprises an RNAi targeting an RNA. In some embodiments, a cargo is or comprises an RNAi targeting a non-coding RNA (ncRNA). In some embodiments a cargo is or comprises an RNAi targeting a long non-coding RNA (lncRNA) In some embodiments, a cargo is or comprises an RNAi targeting a coding RNA (cRNA or mRNA). In some embodiments, an RNAi molecule is or comprises an antisense oligonucleotide (ASO). In some embodiments, an RNAi molecule is or comprises an shRNA. In some embodiments, an RNAi molecule is or comprises an miRNA. In some embodiments, an oligonucleotide comprises or is a gRNA. In some embodiments, an oligonucleotide comprises or is an siRNA. In some embodiments, an oligonucleotide comprises or is a DNA interference (DNAi) molecule.

iv. Linkers

Among other things, systems and methods described herein may use linkers. In some embodiments, a cargo as described herein and a barcode as described herein are separated by a linker. In some embodiments, linkers (L) provide distance between a cargo (P) and a barcode (b). That is, structurally a barcoded cargo, in some embodiments, may have a sequence of P-L-b. This, for example, may contribute to folding characteristics, cargo functionality, and/or cargo stability.

In some embodiments, linkers may be nucleic acids. In some embodiments, linkers may be amino acids. Linkers as described herein may have varying lengths. For example, in some embodiments, a linker may have a length of at least 3 amino acids. In some embodiments, a linker may have a length of between 1 and 50 amino acids (e.g., between 1 and 30 amino acids). In some embodiments, for example, a linker is or comprises a sequence GGGS (SEQ ID NO: 9059).

In some embodiments, linkers of the present invention may be cleaved upon treatment. For example, in some embodiments, a linker may comprise one or more motifs that may be cleaved upon treatment.

In some embodiments, linkers of the present invention may be resistant to cleavage. In some embodiments, linkers of the present invention may be resistant to cleavage in assays. In some embodiments, linkers of the present invention may be resistant to cleavage in vivo.

In one aspect, linkers may be used to tag barcodes. In some embodiments, each linker sequence is associated with a distinct barcode sequence. For example, in some embodiments, a linker sequence may be used as a unique tag associated with a distinct barcode sequence (e.g., nucleic acid sequence) in a mixture. That is, in some embodiments, such a linker may be used to amplify an associated barcode sequence. For example, in some embodiments, such a linker may be used as a primer to amplify an associated barcode sequence. Subsequently, in some embodiments, an amplified linker may be used to isolate an associated barcode sequence, allowing for retrieval of the barcode sequence (e.g., nucleic acid sequence) from a given linker-barcode pair. In some embodiments, a linker-barcode pair may be subject to DNA sequencing for identification of the barcode sequence.

In some embodiments, a nucleic acid sequence encoding for a linker-barcode pair may be used to associate (e.g., link) the linker-barcode pair to a new cargo.

### II. Binders and Binding Agents

i. Binders

In some embodiments, a binder (i.e., a binder moiety) is or comprises a nucleic acid sequence. In some embodiments, a binder is or comprises a nucleic acid sequence that occurs in nature. In some embodiments, a binder is or comprises a nucleic acid sequence that does not occur in nature. In some embodiments, a binder is or comprises a nucleic acid sequence that is synthetic. In some embodiments, a binder comprises naturally occurring nucleic acids. In some embodiments, a binder comprises non-naturally occurring nucleic acids (e.g., modified nucleic acids).

In some embodiments, a binder nucleic acid sequence is or comprises a sequence that encodes for a polypeptide sequence. For example, in some embodiments, a binder nucleic acid sequence may contain a region, which encodes for a polypeptide sequence conferring high affinity and/or specificity for a given barcode (e.g., peptide barcode). In some embodiments, a binder nucleic acid sequence is or comprises a sequence that encodes for an antibody. In some embodiments, a binder nucleic acid sequence is or comprises a sequence that encodes for a fragment of an antibody. In some embodiments, a binder nucleic acid sequence is or comprises a sequence that encodes for a single-chain variable Fragment (scFv). As maybe known to those of ordinary skill in the art, a scFv is a fusion protein of the variable regions of the heavy (V_{H}) and light chains (V_{L}) of immunoglobulins. In some embodiments, a V_{H} and V_{L} chain may be connected with a short linker peptide (e.g., linker of about 5-50 amino acids in length, 10-25 amino acids in length, etc.).

In some embodiments, for example, a binder is generated to have known specificity and affinity for a given barcode. In some embodiments, a binder is generated to have known specificity and affinity for one barcode. In some embodiments, a binder is generated to have known specificity and affinity for multiple (e.g., two or more, three or more, etc.) barcodes. In some embodiments, a binder is generated to have known specificity and affinity for at least one barcode. In some embodiments, a binder, for example, is expressed on the surface of a binding agent (e.g., a phage, a ribosome, etc.) using methods known to those skilled in the art.

In some embodiments, a binder associates with a barcode (e.g., with known specificity and affinity).

In some embodiments, a binder is or comprises a polypeptide sequence that occurs in nature. In some embodiments, a binder is or comprises a polypeptide sequence that does not occur in nature. In some embodiments, a binder is or comprises a polypeptide sequence that is synthetic. In some embodiments, a binder comprises naturally occurring amino acids. In some embodiments, a binder comprises non-naturally occurring amino acids (e.g., modified amino acids).

Binders of the present invention may be of varying lengths. For example, in some embodiments, a binder may have a length ranging between 5 to 1000 amino acids. In some embodiments, a binder may have a length ranging between 5 to 800 amino acids. In some embodiments, a binder may have a length ranging between 6 to 500 amino acids. In some embodiments, a binder may have a length ranging between 10 to 400 amino acids. In some embodiments, a binder may have a length ranging between 5 to 500 amino acids. In some embodiments, a binder may have a length ranging between 5 to 1000 amino acids. In some embodiments, a binder may have a length of 10 amino acids. In some embodiments, a binder may have a length of at least 5 amino acids. In some embodiments, a binder may have a length of at most 1000 amino acids.

Binders, as described herein may be available in a library in different formats. For example, in some embodiments a binder as described herein may be described as a nucleic acid sequence. In other instance, a binder as described herein may be described as an amino acid sequence. A person of ordinary skill in the art will appreciate that binders described in one format may be converted to another format using basic biological principles. Accordingly, binders described as nucleic acid sequences may be translated into proteins, which may be used to detect the presence or absence of a cargo (e.g., barcoded cargo (e.g., barcoded cargo polypeptide, barcoded shuttle-cargo polypeptide)) in a mixture. Such a translated binder is referred to herein as a polypeptide binder or polypeptide binder moiety.

Accordingly, binders of the present disclosure when described using nucleic acids may have lengths different from amino acid sequence lengths disclosed in the paragraph above. For example, in some embodiments, a binder may have a length ranging between 15 to 3000 nucleotides. In some embodiments, a binder may have a length ranging between 15 to 2400 nucleotides. In some embodiments, a binder may have a length ranging between 24 to 1500 nucleotides. In some embodiments, a binder may have a length ranging between 30 to 1200 nucleotides. In some embodiments, a binder may have a length of 30 nucleotides. In some embodiments, a binder may have a length of at least 15 nucleotides. In some embodiments, a binder may have a length of at most 3000 nucleotides.

Binders of the present disclosure may have one or more specific properties. In some embodiments, a binder may be naturally occurring. In some embodiments, a binder may not be naturally occurring (e.g., synthetic). In some embodiments, a binder may not elicit an immune response (e.g., an IgG response, a complement response, etc.).

Among other things, binders (e.g., polypeptide binders, nucleic acids encoding binders) of the present disclosure, like barcodes discussed above, may be flanked by additional sequences (e.g., nucleic acid sequences, amino acid sequences, etc.). In some embodiments, a binder may be flanked by additional sequences on a binder's 5′ end. In some embodiments, a binder may be flanked by additional sequences on a binder's 3′ end. In some embodiments, a binder may be flanked by additional sequences on a binder's 3′ and 5′ end. In some embodiments, an additional sequence may be a primer binding site, a protease-cleavable linker sequence, a sortase site, a transglutaminase site, a restriction endonuclease recognition sequence, a restriction enzyme site (e.g., a cleavage site), a sequence that encodes an amino acid sequence, a sequence that does not encode an amino acid sequence, an amino acid sequence, or a nucleic acid sequence.

In one aspect of the present invention, a binder nucleic acid sequence may be associated with (e.g., attached to, linked to) another nucleic acid sequence. For example, in some embodiments, a binder nucleic acid sequence may be associated with a nucleic acid sequence encoding one or more genes. In some embodiments, a binder nucleic acid sequence may be associated with a nucleic acid sequence encoding one or more genes of a phage (e.g., m13). In some embodiments, a binder nucleic acid sequence may be associated with a nucleic acid sequence encoding a polypeptide. In some embodiments, a binder nucleic acid sequence may be associated with a nucleic acid sequence encoding a polypeptide of a phage (e.g., m13 gene3 protein). The binder-gene3 protein fusion can be expressed and incorporated into m13 phage.

Among other things, binders described herein are designed to have a distinct (i.e., unique) sequence. In some embodiments, a binder is designed to have a distinct sequence (e.g., distinct from another binder). For example, each binder is designed to be distinct from every other binder used in an experiment (i.e., to be unique).

In some embodiments, binders, as described herein, can be designed and/or developed through machine-learning methods.

In one aspect of the present invention, binders bind to barcodes or barcoded cargos, as described herein, with high specificity and high affinity. In some embodiments, a barcode or barcoded cargo (e.g., barcoded cargo polypeptide to be measured, a barcoded shuttle-cargo polypeptide to be measured, a barcoded shuttle peptide to be measured) binds to one binder, and each binder (e.g., binder with a specific sequence) binds to one barcode or barcoded cargo. In some embodiments, a barcode or barcoded cargo (e.g., barcoded cargo polypeptide to be measured, a barcoded shuttle-cargo polypeptide to be measured, a barcoded shuttle peptide to be measured) binds to at least one binder. In some embodiments, each binder (e.g., binder with a specific sequence) binds to at least one barcode or barcoded cargo. In some embodiments, multiple binders (e.g., with different sequences (e.g., polypeptide sequences)) bind to a single barcode. In some embodiments, multiple barcodes (e.g., with different sequences (e.g., peptide sequences)) bind to a single binder.

Example of binders according to various embodiments of the present disclosure are listed in Table 1 and Table 2. In some embodiments, a binder (e.g., polypeptide binder) is or comprises an amino acid sequence selected from SEQ ID NOs: 4200-5346. In some embodiments, a binder (e.g., polypeptide binder) is encoded by a sequence that is or comprises a nucleic acid sequence selected from SEQ ID NOs: 1-1147.

iii. Binding Agents

Methods described herein relate to the detection of one or more barcodes using a binding agent. In some embodiments, a binding agent is associated with or comprises a detectable nucleic acid. In some embodiments, a binding agent expresses a detectable nucleic acid. In some embodiments, a binding agent expresses a detectable nucleic acid on its surface (e.g., a binder). In some embodiments, a binding agent expresses an antibody on its surface.

In some embodiments, for example, to detect the presence of a specific (e.g., distinct) barcode, the present invention envisions the association of a distinct detectable nucleic acid (e.g., a DNA sequence, an RNA sequence, etc.) to a specific barcode. This is achieved through contacting a barcode with a binding agent. In some embodiments, one or more barcodes may be contacted with a binding agent. In some embodiments, one or more binding agents may be contacted with a barcode.

In some embodiments, a binding agent may be or comprises a phage, a ribosome, mRNA, DNA etc. In some embodiments, a binding agent is a phage. In some embodiments, a binding agent is may be a M13 phage, T4 phage, T7 phage, Lambda phage, or filamentous phage. In some embodiments, a binding agent is may be a M13 phage.

Binders as disclosed herein may be expressed on binding agents using methods known in the art. For example, a person of ordinary skill in the art may be able to express a nucleic acid encoding a polypeptide binder on (e.g., on a surface) of a phage using techniques and methods available in the art.

### III. Shuttles

In some embodiments, a shuttle is or comprises a polypeptide sequence. In some embodiments, a shuttle is or comprises polypeptide sequence that occurs in nature. In some embodiments, a shuttle is or comprises a polypeptide sequence that does not occur in nature. In some embodiments, a shuttle is or comprises a polypeptide sequence that is synthetic. In some embodiments, a shuttle comprises naturally occurring amino acids. In some embodiments, a shuttle comprises non-naturally occurring amino acids (e.g., modified amino acids). In some embodiments, a shuttle is or comprises a peptide shuttle.

Shuttles of the present disclosure can be of varying lengths. For example, in some embodiments, a shuttle may have a length ranging between 1 and 500 amino acids. In some embodiments, a shuttle may have a length ranging between 1 and 400 amino acids. In some embodiments, a shuttle may have a length ranging between 1 and 300 amino acids. In some embodiments, a shuttle may have a length ranging between 1 and 200 amino acids. In some embodiments, a shuttle may have a length ranging between 5 and 200 amino acids. In some embodiments, a shuttle may have a length ranging between 20 and 200 amino acids. In some embodiments, a shuttle may have a length ranging between 50 and 200 amino acids. In some embodiments, a shuttle may have a length ranging between 20 and 175 amino acids. In some embodiments, a shuttle may have a length ranging between 20 and 150 amino acids. In some embodiments, a shuttle may have a length ranging between 100 and 150 amino acids. In some embodiments, a shuttle may have a length ranging between 100 and 130 amino acids. In some embodiments, a shuttle may have a length of 1, 2, 3, 4, 5, 6, 7, 8, 9, 10, 11, 12, 13, 14, 15, 16, 17, 18, 19, 20, 21, 22, 23, 24, 25, 50, 100, 105, 110, 115, 120, 125, 130, 135, 140, 145, or 150 amino acids. In some embodiments, a shuttle may have a length of at least 5 amino acids. In some embodiments, a shuttle may have a length of at most 150 amino acids.

Shuttles, as described herein may be available in different formats. For example, in some embodiments a shuttle as described herein may be described as a nucleic acid sequence. In other instances, a shuttle as described herein may be described as an amino acid sequence. A person of ordinary skill in the art will appreciate that shuttles described in one format may be converted to another format using basic biological principles. Accordingly, in some embodiments, shuttles described as nucleic acid sequences may be translated into proteins, which may be used to deliver a cargo (e.g., cargo polypeptide) to a cell, tissue, or organ of interest. In some embodiments, shuttles described as nucleic acid sequences may be translated into proteins, which may be used to transport a cargo (e.g., cargo polypeptide) to a cell, tissue, or organ of interest. In some embodiments, shuttles described as nucleic acid sequences may be translated into proteins, which may be used to “shuttle” (or “transport”) a cargo (e.g., cargo polypeptide) to a cell, tissue, or organ of interest. In some embodiments, shuttles described as nucleic acid sequences may be translated into proteins, which may be used to unidirectionally “shuttle” a cargo (e.g., cargo polypeptide) to a cell, tissue, or organ of interest. Such a translated shuttle, for example, may be referred to herein as a peptide shuttle or a shuttle.

In some embodiments, shuttles comprising a nucleic acid sequence may be used to deliver a cargo (e.g., cargo nucleic acid) to a cell, tissue, or organ of interest, without first undergoing translation. In some embodiments, shuttles comprising a nucleic acid sequence may be used to transport a cargo (e.g., cargo nucleic acid) to a cell, tissue, or organ of interest, without first undergoing translation. In some embodiments, shuttles comprising a nucleic acid sequence may be used to “shuttle” (or “transport”) a cargo (e.g., cargo nucleic acid) to a cell, tissue, or organ of interest, without first undergoing translation. In some embodiments, shuttles comprising a nucleic acid sequence may be used to unidirectionally “shuttle” a cargo (e.g., cargo nucleic acid) to a cell, tissue, or organ of interest, without first undergoing translation.

In some embodiments, a shuttle comprising an amino acid sequence may be used to deliver, transport, or shuttle a cargo that is of a different format from said shuttle (e.g., a heterologous cargo). That is, in some embodiments, a peptide shuttle comprising an amino acid sequence may be used to deliver, transport, or shuttle a cargo comprising a nucleic acid, a polysaccharide, a small molecule, or a combination thereof (i.e., a heterologous cargo). In some embodiments, a shuttle comprising an amino acid sequence may be used to deliver, transport, or shuttle a cargo (e.g., a cargo nucleic acid, a cargo polysaccharide, a cargo small molecule). In some embodiments, a shuttle comprising a nucleic acid sequence may be used to deliver, transport, or shuttle a cargo that is of a different format from said shuttle (e.g., a heterologous cargo). That is, in some embodiments, shuttle comprising a nucleic acid sequence may be used to deliver, transport, or shuttle a heterologous cargo (e.g., a cargo polypeptide, a cargo polysaccharide, a cargo small molecule).

Accordingly, shuttles of the present disclosure when described using nucleic acids may have lengths different from amino acid sequence lengths disclosed in the paragraph above. For example, in some embodiments, a shuttle may have a length ranging between 3 and 1500 nucleotides. In some embodiments, a shuttle may have a length ranging between 3 and 1200 nucleotides. In some embodiments, a shuttle may have a length ranging between 3 and 900 nucleotides. In some embodiments, a shuttle may have a length ranging between 3 and 600 nucleotides. In some embodiments, a shuttle may have a length ranging between 15 and 600 nucleotides. In some embodiments, a shuttle may have a length ranging between 60 and 600 nucleotides. In some embodiments, a shuttle may have a length ranging between 150 and 600 nucleotides. In some embodiments, a shuttle may have a length ranging between 60 and 525 nucleotides. In some embodiments, a shuttle may have a length ranging between 60 and 450 nucleotides. In some embodiments, a shuttle may have a length ranging between 300 and 450 nucleotides. In some embodiments, a shuttle may have a length ranging between 300 and 390 nucleotides. In some embodiments, a shuttle may have a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 150, 300, 315, 330, 345, 360, 375, 390, 405, 420, 435, or 450 nucleotides. In some embodiments, a shuttle may have a length of at least 15 nucleotides. In some embodiments, a shuttle may have a length of at most 450 nucleotides.

Shuttles of the present disclosure may have one or more properties. In some embodiments, a shuttle may be naturally occurring. In some embodiments, a shuttle may not be naturally occurring (e.g., synthetic). In some embodiments, a shuttle may have relatively no effect on cargo function. For example, in some embodiments, tagging a cargo (e.g., a cargo component) with a shuttle as described herein does not alter or change relatively the function of the tagged cargo. In some embodiments, a shuttle may have an effect (e.g., positive or negative) on cargo function. In some embodiments, a shuttle may comprise a structural feature (e.g., length, hydrophobicity, affinity, sequence motif, etc.) that has an effect (e.g., positive or negative) on cargo function. For example, in some embodiments, tagging a cargo (e.g., a cargo component) with a shuttle as described herein may alter or change relatively a function (e.g., half-life (e.g., longer half-life), toxicity (e.g., anemia), enhance targeting to specific tissue, etc.) of the tagged cargo. For example, in some embodiments, toxicity (e.g., anemia) may occur due to reticulocyte killing caused by sub-optimal shuttles (e.g., shuttle moieties). In some embodiments, a shuttle may not elicit an immune response (e.g., an IgG response, a complement response, etc.). In some embodiments, shuttles are orthogonal to each other. In some embodiments, shuttles are not orthogonal to each other. In some embodiments, shuttles (e.g., peptide shuttles) enhance targeting to a specific cell, tissue, or organ of interest. In some embodiments, shuttles (e.g., peptide shuttles) enhance targeting to a brain cell, brain tissue, or brain of an organism. In some embodiments, shuttles (e.g., peptide shuttles) do not enhance targeting to a specific cell, tissue, or organ of interest. In some embodiments, shuttles (e.g. peptide shuttles) enhance targeting across the blood brain barrier. In some embodiments, a shuttle (e.g., a shuttle moiety) is or comprises an antibody. In some embodiments, a shuttle (e.g., a shuttle moiety) is or comprises a variant or a fragment of an antibody. For example, in some embodiments, a shuttle as described herein is a VHH-based (i.e., nanobody-based) shuttle. In some embodiments, a shuttle as described herein is a Fabs-based shuttle. In some embodiments, a shuttle as described herein is scFv-based.

Shuttles of the present disclosure may be associated to various positions of a cargo. For example, in some embodiments, a shuttle (e.g., a shuttle moiety) may be associated (e.g., covalently or non-covalently) to a suitable (e.g., desirable) position on a cargo. In some embodiments, a shuttle may be associated (e.g., covalently or non-covalently) to a less suitable or non-suitable (e.g., non-desirable) position on a cargo. In some embodiments, a peptide shuttle may be associated (e.g., covalently or non-covalently) to a suitable position on a cargo. In some embodiments, a peptide shuttle may be associated (e.g., covalently or non-covalently) to a suitable position on a cargo polypeptide. For example, in some embodiments, a shuttle (e.g., a peptide shuttle) may be associated (e.g., covalently or non-covalently) to an N-terminus of a cargo polypeptide. In some embodiments, a shuttle (e.g., a peptide shuttle) may be associated (e.g., covalently or non-covalently) to a C-terminus of a cargo polypeptide. In some embodiments, a shuttle (e.g., a peptide shuttle) may be associated (e.g., covalently or non-covalently) to a non-terminal position on a cargo polypeptide (e.g., side chain). In some embodiments, a shuttle (e.g., a peptide shuttle) may be associated (e.g., covalently or non-covalently) to a less suitable or non-suitable (e.g., non-desirable) position on a cargo polypeptide.

Shuttles of the present disclosure may be associated to various positions of a therapeutic agent. For example, in some embodiments, a shuttle (e.g., a shuttle moiety) may be associated (e.g., covalently or non-covalently) to a suitable position on a therapeutic agent. In some embodiments, a shuttle may be associated (e.g., covalently or non-covalently) to a non-suitable (e.g., non-desirable) position on a therapeutic agent. In some embodiments, a peptide shuttle may be associated (e.g., covalently or non-covalently) to a suitable position on a therapeutic agent. In some embodiments, a peptide shuttle may be associated (e.g., covalently or non-covalently) to a suitable position on a therapeutic polypeptide. For example, in some embodiments, a shuttle (e.g., a peptide shuttle) may be associated (e.g., covalently or non-covalently) to an N-terminus of a therapeutic polypeptide. In some embodiments, a shuttle (e.g., a peptide shuttle) may be associated (e.g., covalently or non-covalently) to a C-terminus of a therapeutic polypeptide. In some embodiments, a shuttle (e.g., a peptide shuttle) may be associated (e.g., covalently or non-covalently) to a non-terminal position on a therapeutic polypeptide (e.g., side chain). In some embodiments, a shuttle (e.g., a peptide shuttle) may be associated (e.g., covalently or non-covalently) to a less suitable or non-suitable (e.g., non-desirable) position on a therapeutic polypeptide.

Among other things, shuttles (e.g., peptide shuttles or shuttle components encoding peptide shuttles) of the present disclosure may be flanked by additional sequences (e.g., nucleic acid sequences, amino acid sequences, etc.). In some embodiments, a shuttle may be flanked by additional sequences on a shuttle's 5′ end. In some embodiments, a shuttle may be flanked by additional sequences on a shuttle's 3′ end. In some embodiments, a shuttle may be flanked by additional sequences on a shuttle's 3′ and 5′ end. In some embodiments, an additional sequence may be a primer binding site, a protease-cleavable linker sequence, a sortase site, a transglutaminase site, a restriction endonuclease recognition sequence, a restriction enzyme site (e.g., a cleavage site), a sequence that encodes an amino acid sequence, a sequence that does not encode an amino acid sequence, an amino acid sequence, or a nucleic acid sequence. For example, in some embodiments, a shuttle may be flanked by nucleic acid sequences encoding an amino acid sequence. In some embodiments, a shuttle may be flanked by nucleic acid sequences that does not encode an amino acid sequence. In some embodiments, a shuttle may be flanked by amino acid sequences. In some embodiments, a peptide shuttle may be flanked by amino acid sequences (e.g., Glycine-Serine (GS), e.g., other linker amino acid sequences). Analogously, in some embodiments, a shuttle component encoding a peptide shuttle of the present disclosure may be flanked by additional sequences (e.g., nucleic acid sequences, amino acid sequences, etc.). In some embodiments, a shuttle component encoding a peptide shuttle may be flanked by nucleic acid sequences on a 5′ end. In some embodiments, a shuttle component encoding a peptide shuttle may be flanked by nucleic acid sequences on a 3′ end. In some embodiments, a shuttle component encoding a peptide shuttle may be flanked by nucleic acid sequences on a 3′ and 5′ end. In some embodiments, a shuttle component encoding a peptide shuttle may be flanked by nucleic acid sequences encoding an amino acid sequence comprising a Glycine-Serine (GS). In some embodiments, a shuttle component encoding a peptide shuttle may be flanked by nucleic acid sequences encoding an amino acid sequence comprising a linker amino acid sequence as described herein.

In some embodiments, a shuttle may be flanked by restriction endonuclease recognition sequences. In some embodiments, a shuttle may be flanked by restriction endonuclease recognition sequences on a shuttle's 5′ end. In some embodiments, a shuttle may be flanked by restriction endonuclease recognition sequences on a shuttle's 3′ end. In some embodiments, a shuttle may be flanked by restriction endonuclease recognition sequences on a shuttle's 3′ and 5′ end. In some embodiments, a nucleic acid encoding a peptide shuttle may be flanked by restriction endonuclease recognition sequences. In some embodiments, a nucleic acid encoding a peptide shuttle may be flanked by restriction endonuclease recognition sequences on a 5′ end. In some embodiments, a nucleic acid encoding a peptide shuttle may be flanked by restriction endonuclease recognition sequences on a 3′ end. In some embodiments, a nucleic acid encoding a peptide shuttle may be flanked by restriction endonuclease recognition sequences on a 3′ and 5′ end. In some embodiments, a restriction endonuclease recognition sequence may be recognized by one or more restriction enzymes (e.g., BsaI, BsmBI, BbsI, SapI, etc.). In some embodiments, restriction endonuclease recognition sequences are Type I, Type II, or Type IIs restriction endonuclease recognition sequences. Such recognition sequences, for example, may be used to produce universal overhangs that may be used in cloning peptide shuttles into different locations of various cargo. Such flexibility allows a shuttle to be used to detect different cargo polypeptides in different experiments.

In some embodiments, a shuttle (e.g., a peptide shuttle) is cleavable. In some embodiments, a shuttle may be cleaved using an enzyme. In some embodiments, a shuttle (e.g., a peptide shuttle) may be cleaved using a protease. In some embodiments, a shuttle (e.g., a peptide shuttle) is cleaved spontaneously. For example, in some embodiments, a shuttle (e.g., a peptide shuttle) is cleaved spontaneously in a specific cell, tissue, or organ of interest (e.g., a brain cell, tissue, or organ). In some embodiments, a shuttle (e.g., a peptide shuttle) is non-cleavable.

In some embodiments, a shuttle component encoding a shuttle (e.g., a peptide shuttle) may be associated with (e.g., attached to, linked to) a second cargo component encoding a cargo polypeptide (e.g., a cargo polypeptide of interest). Such nucleic acid sequences, for example, may be translated to form shuttle-cargos (e.g., shuttle-cargo polypeptides). In some embodiments, a shuttle component encoding a shuttle (e.g., a peptide shuttle) is separate from a cargo component encoding a cargo polypeptide (e.g., a cargo polypeptide of interest). For such nucleic acid sequences, for example, a shuttle component encoding a peptide shuttle may be translated separately from a cargo component sequence encoding a cargo polypeptide, and subsequently attached using one or more methods known in the art to join distinct amino acid sequences (e.g., using linkers).

Shuttles (e.g., shuttle moieties) of the present disclosure may be associated (e.g., directly or indirectly attached) to cargos so as to form shuttle-cargos (or shuttle-cargo components as described herein). For example, in some embodiments, each shuttle sequence (e.g., peptide shuttle sequence) may be associated to only one cargo of interest (e.g., cargo polypeptide of interest) within a mixture. In some embodiments, each shuttle sequence may be associated to more than one cargo of interest (e.g., cargos with different sequences) within a mixture. In some embodiments, multiple (e.g., two or more, three or more, four or more, etc.) shuttle sequences may be associated to one cargo of interest within a mixture. For example, in some embodiments, one or more shuttle sequences may be associated to various different positions on a given cargo—such a setup may be useful in, for example, in studying and identifying the stability and/or cleavage of such shuttle-cargos. In some embodiments, each cargo in a mixture is a unique sequence (e.g., each cargo has a different sequence from every other cargo in the mixture). In some embodiments, each cargo in a mixture is a non-unique sequence.

In some embodiments, a shuttle (e.g., a shuttle moiety), as described herein, can be designed and/or developed through machine-learning methods. In some embodiments, shuttles, designed and/or developed through machine-learning methods possess improved functionality (e.g., reduced toxicity (e.g., reduced anemia), improved pharmacokinetic (PK) measures (e.g., dissociation constant (Kd), improved biophysical properties, epitope properties, affinity properties, thermostability properties, pH sensitivity properties, etc.) relative to a reference shuttle (e.g., a standard shuttle).

Various methods and parameters may be used to select suitable shuttles for a given cargo. For example, stability of a shuttle-cargo is key in determining if a cargo may be tagged (e.g., associated (e.g., covalently or non-covalently)) by said shuttle. In some embodiments, a shuttle may be tagged to a specific cargo across different experiments. In some embodiments, a shuttle may be tagged to different cargos in different experiments. For example, in some embodiments, a shuttle may be tagged to two or more, three or more, four or more, ten or more, 100 or more, 1000 or more, or 10,000 or more different cargos across different experiments.

In some embodiments, a shuttle may be associated with only one cargo in a given experiment. In some embodiments, a shuttle may be associated with multiple cargos in a given experiment. For example, in some embodiments, one or more shuttles are associated with multiple cargos (i.e., a shuttle is tagged to multiple cargos) in a mixture, such that each cargo is associated with a unique set of shuttles within the mixture. That is, each cargo may be associated with a unique “pattern” of shuttles in the mixture. Analogously, in some embodiments, several cargos may be associated with the same shuttle.

Among other things, shuttles described herein are designed to have a distinct (i.e., unique) sequence. In some embodiments, a shuttle is designed to have a distinct sequence (e.g., distinct from another shuttle). For example, each shuttle is designed to be distinct (e.g., unique) from every other shuttle used in an experiment, such that each cargo (e.g., protein to be measured) is attached to at least one shuttle, and each shuttle (e.g., shuttle with a specific sequence) is only attached to one cargo. As may be understood by a person of ordinary skill in the art, the diversity of shuttles contained within a pool is limited only by the possible diversity of amino acid sequences for a given shuttle length. For example, for a shuttle length ‘N’, there exists 20^{N} distinct amino acid shuttle sequences of length N (if only unmodified/naturally occurring amino acids are used). That is, for a shuttle length of 100, the theoretical limit is 20¹⁰⁰, or 1.26762×10¹³⁰.

In some embodiments, shuttles, as described herein, can be designed and/or developed through machine-learning methods.

Example of shuttles according to various embodiments of the present disclosure are listed in Table 6 and Table 7. In some embodiments, a shuttle (e.g., peptide shuttle) is or comprises an amino acid sequence selected from SEQ ID NOs: 8813-8894. In some embodiments, a shuttle (e.g., peptide shuttle) is encoded by a sequence that is or comprises a nucleic acid sequence selected from SEQ ID NOs: 8753-8812. In some embodiments, a shuttle (e.g., a shuttle component) is or comprises a nucleic acid sequence selected from SEQ ID NOs: 8753-8812. In some embodiments, a shuttle (e.g., peptide shuttle) is or comprises an amino acid sequence having 80%, 85%, 90%, 91%, 92%, 93%, 94%, 95%, 96%, 97%, 98% or 99% identity to a sequence selected from SEQ ID NOs: 8813-8894. In some embodiments, a shuttle (e.g., peptide shuttle) is encoded by a sequence that is or comprises a nucleic acid sequence having 80%, 85%, 90%, 91%, 92%, 93%, 94%, 95%, 96%, 97%, 98% or 99% identity to a sequence selected from SEQ ID NOs: 8753-8812. In some embodiments, a shuttle (e.g., a shuttle component) is or comprises a nucleic acid sequence having 80%, 85%, 90%, 91%, 92%, 93%, 94%, 95%, 96%, 97%, 98% or 99% identity to a sequence selected from SEQ ID NOs: 8753-8812.

TABLE 6
Table providing exemplary list of shuttle nucleic acid sequences

| SEQ ID NO. | ID | NUCLEIC ACID SEQUENCE |
|---|---|---|
| (SEQ ID NO. | TfR-1409 | CAGGTGCAACTGGTCGAGTCCGGCGGAGGCGTCGTGCAACCTG |
| 8753) | | GCAGGAGCCTCAGACTCTCCTGTGCTGCTAGCGGCTTTTCTTT |
| | | TAGCAATTTCGGAATGAGCTGGGTACGACAGGCACGCGGAAAA |
| | | GGATTGGAGTGGGTTTCCTCAACTGGATCTGACGGCACTGCGT |
| | | ACTATGCTGACTCCGTGAAAGGAAGGTTCACTATTTCTAGGGA |
| | | TAACGCGAAGAATACACTTTATTTGCAGATGAACTCTCTGCGA |
| | | GCTGAGGACACCGCGGTGTACTACTGCGCCAGAGCCCCAAGTA |
| | | GACACGGGTTCGACTACTGGGGCCAAGGAACCCIGGTTACAGT |
| | | CAGCAGC |
| (SEQ ID NO. | TfR-1409- | CAGGTGCAACTGGTCGAGTCCGGCGGAGGCGTCGTGCAACCTG |
| 8754) | 51 | GCAGGAGCCTCAGACTCTCCTGTGCTGCTAGCGGCTTCAGCTT |
| | | CAGCAATTTCGGCATGAGCTGGGTGCGTCAGGCCCGAGGGAAA |
| | | GGCTTGGAATGGGTCAGTTCCATCGGGAGCGACGGACGAGCTT |
| | | ACTACGCTGACAGCGTGAAGGGTCGGTTTACTATCAGCAGAGA |
| | | TAACGCCAAGAACACTCTGTACCTGCAAATGAATTCTCTGAGG |
| | | GCAGAGGATACTGCCGTGTACTATTGTGCCAGGGCGCCTTCAA |
| | | GGCACGGATTTGACTACTGGGGCCAAGGAACCCTGGTTACAGT |
| | | CAGCAGC |
| (SEQ ID NO. | TfR-1409-6 | CAGGTGCAACTGGTCGAGTCCGGCGGAGGCGTCGTGCAACCTG |
| 8755) | | GCAGGAGCCTCAGACTCTCCTGTGCTGCTAGCGGCTTCAGCTT |
| | | CAGCAATTTCGGCATGAGCTGGGTGCGTCAGGCCCGAGGGAAA |
| | | GGCTTGGAATGGGTCAGTTCCGTAGGGAGCGACGGAACCGCTT |
| | | ACTACGCTGACAGCGTGAAGGGTCGGTTTACTATCAGCAGAGA |
| | | TAACGCCAAGAACACTCTGTACCTGCAAATGAATTCTCTGAGG |
| | | GCAGAGGATACTGCCGTGTACTATTGTGCCAGGGCGCCTTCAA |
| | | GGCACGGATTTGACTACTGGGGCCAAGGAACCCTGGTTACAGT |
| | | CAGCAGC |
| (SEQ ID NO. | TfR-1409- | CAGGTGCAACTGGTCGAGTCCGGCGGAGGCGTCGTGCAACCTG |
| 8756) | 25 | GCAGGAGCCTCAGACTCTCCTGTGCTGCTAGCGGCTTCAGCTT |
| | | CAGCAATTTCGGCATGAGCTGGGTGCGTCAGGCCCCCGGGAAA |
| | | GGCTTGGAATGGGTCAGTTCCACCGGGAGCGACGGAACCGCTT |
| | | ACTACGCTGACAGCGTGAAGGGTCGGTTTACTATCAGCAGAGA |
| | | TAACGCCAAGAACACTCTGCACCTGCAAATGAATTCTCTGAGG |
| | | GCAGAGGATACTGCCGTGTACTATTGTGCCAGGGCGCCTTCAA |
| | | GGCACGGATTTGACTACTGGGGCCAAGGAACCCTGGTTACAGT |
| | | CAGCAGC |
| (SEQ ID NO. | TfR-455 | CAGGTGCAACTGGTCGAGTCCGGCGGAGGCGTCGTGCAACCTG |
| 8757) | | GCAGGAGCCTCAGACTCTCCTGTGCTGCTAGCGGCTTCGCTGT |
| | | CGATGCTAGCGATATGGGCTGGTTCCGCCAAGCCCCAGGCAAA |
| | | GGACGAGAACTGGTCTCATCTATCTCCAGCGATAACGACACCT |
| | | ATTACGCAGACAGCGTGGAGGGACGCTTCACCATTAGTCGTGA |
| | | CAATGCTAAGCGGATGGTCTACCTGCAGATGAATTCACTGAGG |
| | | GCTGAAGATACAGCTGTGTACTACTGCGCTAGCGGTAGATTTC |
| | | TGGAGCCTGATTACTGGGGCCAAGGAACCCTGGTTACAGTCAG |
| | | CAGC |
| (SEQ ID NO. | TfR-299 | CAGGTGCAACTGGTCGAGTCCGGCGGAGGCGTCGTGCAACCTG |
| 8758) | | GCAGGAGCCTCAGACTCTCCTGTGCTGCTAGCGGCTTCACCTT |
| | | TGACGACTCAGACATGGGTTGGTTTAGGCAGGCTCCTGGTAAA |
| | | GGCCGGGAGCTGGTCGCTGCTATCAACACAGGAGGAGGAAAGA |
| | | CCTACTATGCTGCAACAGTCGAAGGACGGTTTACAATATCCAG |
| | | GGATAACGCCAAGCGCATGGTGTATTTGCAGATGAACTCCCTT |
| | | AGAGCTGAGGACACCGCAGTATACTATTGCGCCTCAGGGCGAT |
| | | TTTTAGAACCCGACTACTGGGGCCAAGGAACCCTGGTTACAGT |
| | | CAGCAGC |
| (SEQ ID NO. | TfR-891 | CAGGTGCAACTGGTCGAGTCCGGCGGAGGCGTCGTGCAACCTG |
| 8759) | | GCAGGAGCCTCAGACTCTCCTGTGCTGCTAGCGGCCTGACATT |
| | | CGATGATAGCGATATGGGGTGGTTTCGCCAGGCTCCTGGTAAA |
| | | GAGCGTGAAAGAGTATCCACTATATCTAGTGACGGTACCCCAC |
| | | ACTATGATGACTCAGTCAAAGGCCGCTTTACCATTAGCAGAGA |
| | | CAATAGCAAAAATACTCTGTACCTTCAGATGAATTCACTTCGA |
| | | GCGGAAGACACTGCCGTGTACTACTGTGCAAGACGCGATGGTG |
| | | GCAGCTGGAATTTCGACTATTGGGGCCAAGGAACCCTGGTTAC |
| | | AGTCAGCAGC |
| (SEQ ID NO. | TfR-732 | CAGGTGCAACTGGTCGAGTCCGGCGGAGGCGTCGTGCAACCTG |
| 8760) | | GCAGGAGCCTCAGACTCTCCTGTGCTGCTAGCGGCTTTACCTT |
| | | CGATGGATCCGACATTGCATGGGTCAGGAGAGCCCCAGGAAAA |
| | | GGAGAGGAGTGGGTGAGTACCATCAGTTCAGACGGCACTGTGT |
| | | ACTACAGTGACCCTGTGAAGGGAAGATTTACAATCTCTGCTGA |
| | | TACATCAAAGAACACTGCATATCTGCAAATGAACAGTCTTAGG |
| | | GCAGAAGACACCGCAGTGTACTACTGTGCCATCAGCTCTTGGT |
| | | ACGGCGACTTCGACTACTGGGGCCAAGGAACCCTGGTTACAGT |
| | | CAGCAGC |
| (SEQ ID NO. | TfR-734 | CAGGTGCAACTGGTCGAGTCCGGCGGAGGCGTCGTGCAACCTG |
| 8761) | | GCAGGAGCCTCAGACTCTCCTGTGCTGCTAGCGGCCATATTCT |
| | | GAATTATTGGTATATGGGTTGGGTAAGACGTGCTCCCGGAAAA |
| | | GGAGAAGAGTGGGTCGCIGGCATATCCCCGGGGGGGACCGTCA |
| | | CGGTCTATGCCGACTCAGTGAAGGGAAGGTTCACTATAAGCAG |
| | | AGATAATTCCAAGAACACTCTGTACCTCCAGATGAACTCACTG |
| | | AGGGCAGAGGATACGGCTGTGTACTACTGTGCTAGAGGCGGCC |
| | | AGGGTGGTGACGCTTGGGCCTTCGACTATTGGGGCCAAGGAAC |
| | | CCTGGTTACAGTCAGCAGC |
| (SEQ ID NO. | TfR-358 | CAGGTGCAACTGGTCGAGTCCGGCGGAGGCGTCGTGCAACCTG |
| 8762) | | GCAGGAGCCTCAGACTCTCCTGTGCTGCTAGCGGCTTCGGCGT |
| | | GGCCTCAAGCGACATGGGTTGGTTCAGGCAGGCCCCTGGAAAG |
| | | GGAAGGGAGCTGGTGGCCAGTATTAATACTGACGGGTCAACGA |
| | | TCTACGCGGAGTCTGTGGAGGGCAGGTTCACCATCTCAAGAGA |
| | | CAACGCCAAGAGGATGGTCTACCTCCAGATGAACTCTTTGCGG |
| | | GCCGAAGATACTGCTGTGTATTACTGCGCCGCATACGATACCA |
| | | GCGCATACCACTCTCCTTTCGACTACTGGGGCCAAGGAACCCT |
| | | GGTTACAGTCAGCAGC |
| (SEQ ID NO. | TfR-85 | CAGGTGCAACTGGTCGAGTCCGGCGGAGGCGTCGTGCAACCTG |
| 8763) | | GCAGGAGCCTCAGACTCTCCTGTGCTGCTAGCGGCTACACCTT |
| | | CCGCTCAGCCAGTATGGGGTGGGTTCGGAGGGCCCCTGGCAAG |
| | | GGTGAGGAGTGGGTGGCCGTGATAGACAGTGAGGGACATACTA |
| | | TCTACGCTGACTCTGTAAAAGGGCGGTTTACTATAAGCGCCGA |
| | | TACCTCAAAAAATACTGCATATCTCCAGATGAACTCCCTCAGA |
| | | GCCGAGGATACTGCCGTGTATTATTGTGCGAAGGATATTTATG |
| | | GTGACTATGGCGGTCCCGACTATTGGGGCCAAGGAACCCTGGT |
| | | TACAGTCAGCAGC |
| (SEQ ID NO. | TfR-448 | CAGGTGCAACTGGTCGAGTCCGGCGGAGGCGTCGTGCAACCTG |
| 8764) | | GCAGGAGCCTCAGACTCTCCTGTGCTGCTAGCGGCACCCGACG |
| | | TCCTAACTACGTTACATGGTTCAGACAAGCTCCACTGAACGAG |
| | | CGACAAGTCGTCGCCGGCATTGACAGCGCCGGCACCACTACGT |
| | | ACGCCGATTCTGTCAAGGGCAGGTTCACTATCAGCCAGGATAA |
| | | CGCGAAAAACACTGTATATCTGCAGATGAACAGTCTGCGTGCG |
| | | GAGGATACAGCCGTTTACTACTGTGCTTCAGGAATTTCTTCCT |
| | | CATCCTTCTATCCATTCGACTCTTGGGGCCAAGGAACCCTGGT |
| | | TACAGTCAGCAGC |
| (SEQ ID NO. | TfR-1573 | CAGGTGCAACTGGTCGAGTCCGGCGGAGGCGTCGTGCAACCTG |
| 8765) | | GCAGGAGCCTCAGACTCTCCTGTGCTGCTAGCGGCTTTAACTT |
| | | TGACGACAGTGACATGGGGTGGTTTCGCCAAGCGCCAGGGAAA |
| | | GGTAGAGAGTTGGTCTCCTCAATCGCATCTGACGGGGCGACAT |
| | | ATTACGCGGACTCCGTGGAGGGCCGGTTTACCATCTCCCGGGA |
| | | TAATGCAAAGAGGATGGTGTACCTGCAAATGAATTCCCTCCGA |
| | | GCTGAAGATACAGCTGTCTACTACTGTGCTCGAGATAGTCCGT |
| | | CATTGCGAGCCTTTGACTCCTGGGGCCAAGGAACCCTGGTTAC |
| | | AGTCAGCAGC |
| (SEQ ID NO. | TfR-1417 | CAGGTGCAACTGGTCGAGTCCGGCGGAGGCGTCGTGCAACCTG |
| 8766) | | GCAGGAGCCTCAGACTCTCCTGTGCTGCTAGCGGCACCACCTA |
| | | TTCTCGCCAATACTGTATGGGATGGGCGAGGCAGGCCCCAGGT |
| | | AAGGGCCTGGAATGGGTCAGCTGTATTGACGACGATGGGAATA |
| | | CCTATTACGCCGATAGCGTTAAGGGTAGATTTACTATCAGCAG |
| | | AGATAATAGTAAGAACACACTCTATCTCCAGATGAATTCTCTG |
| | | CGCGCCGAAGATACTGCAGTGTACTACTGTGCTAGAGACCCTG |
| | | GCCTCAGCGGAGACTTTGATTATTGGGGCCAAGGAACCCTGGT |
| | | TACAGTCAGCAGC |
| (SEQ ID NO. | TfR-1090 | CAGGTGCAACTGGTCGAGTCCGGCGGAGGCGTCGTGCAACCTG |
| 8767) | | GCAGGAGCCTCAGACTCTCCTGTGCTGCTAGCGGCATTACCTA |
| | | CCGGGGGCCATGCATGGGGTGGTATCGCCAGGCGCCCGGGAAA |
| | | GGGCTTGAGTGGGTTGCGACTCTCGACTCTGACGGGAGCACCA |
| | | CTTATGCGGATTCTGTGAAAGGACGGTTTACGATCTCTAGGGA |
| | | TAACAGCAAAAATACACTGTATCTGCAGATGAATAGCCTCCGC |
| | | GCAGAGGATACTGCGGTGTATTACTGCGCAATGTATATGACAG |
| | | GCGGAGGGGGTGTAGCATTTGATTACTGGGGCCAAGGAACCCT |
| | | GGTTACAGTCAGCAGC |
| (SEQ ID NO. | TfR-1415 | CAGGTGCAACTGGTCGAGTCCGGCGGAGGCGTCGTGCAACCTG |
| 8768) | | GCAGGAGCCTCAGACTCTCCTGTGCTGCTAGCGGCTTTACCTT |
| | | CTCCAAATACGCTATGAACTGGGCCAGACAAGCGCCGGGCAAG |
| | | GGCCTCGAGTGGGTAGCGTTTATCGATACGGCCGGCAGGACCA |
| | | TCTATGCCGATTCAGTGAAAGGCAGATTTACCATTTCCCGGGA |
| | | TAACTCAAAGAATACCTTGTACCTTCAAATGAATTCTCTGAGA |
| | | GCTGAAGATACCGCCGTCTATTATTGCGCCCCTAGCTACGGCG |
| | | ATTCCGGAGATTACTTCGACTATTGGGGCCAAGGAACCCTGGT |
| | | TACAGTCAGCAGC |
| (SEQ ID NO. | CD98-11 | CAGGTGCAACTGGTCGAGTCCGGCGGAGGCGTCGTGCAACCTG |
| 8769) | | GCAGGAGCCTCAGACTCTCCTGTGCTGCTAGCGGCTTCCCTTT |
| | | TTCTAGCTACGGAATGGGATGGTTCCGTCAGGCTCCAGGCAAG |
| | | GAGCGAGAGGGTGTTGCAGGAGTGAATACGGGGGGAAGAACAT |
| | | CCTATGCAGAGTCCGTAAAGGGGCGATTCACCATCTCCCGGGA |
| | | CAATACACGCAACACACTTTACTTACAGATGAATAGCCTTCGT |
| | | GCCGAAGATACCGCCGTGTATTACTGTGCTAGAATACGGGCGG |
| | | CCGGCACAGTGGCGCCCTACTTCGACTACTGGGGCCAAGGAAC |
| | | CCTGGTTACAGTCAGCAGC |
| (SEQ ID NO. | CD98-74 | CAGGTGCAACTGGTCGAGTCCGGCGGAGGCGTCGTGCAACCTG |
| 8770) | | GCAGGAGCCTCAGACTCTCCTGTGCTGCTAGCGGCTACGTCGC |
| | | TGGTTACGTGGGATGGTACAGGCAGGCTCCAGGTAAGGATAGG |
| | | GAGGGCGTAGCTGCTATCGCCAGTGATGGGATGACAGCCTACG |
| | | CCCATAGTGTAAAAGGCAGGTTCACCATCTCCAGAGATAATAG |
| | | CAAAAACACTCTGTACCTTCAGATGAATTCTCTCAGGGCTGAA |
| | | GATACTGCTGTGTATTACTGTGCGTCCGGTTACTGCTCAGGGT |
| | | CAGGCTGCTACTTCGACAGTTGGGGCCAAGGAACCCTGGTTAC |
| | | AGTCAGCAGC |
| (SEQ ID NO. | CD98-82 | CAGGTGCAACTGGTCGAGTCCGGCGGAGGCGTCGTGCAACCTG |
| 8771) | | GCAGGAGCCTCAGACTCTCCTGTGCTGCTAGCGGCTTCCGATA |
| | | TAATAGCTACGCCATGGCCTGGGCACGTCAGGCTCCAGGCAAG |
| | | GGACTTGAGTGGGTTGCAGCCATCGCCGGGGACGGAAGAACAG |
| | | ATTATGCAGACTCCGTAAAGGGGCGATTCACCATCTCCCGGGA |
| | | CAATAGTAAAAACACACTTTACTTACAGATGAATAGCCTTCGT |
| | | GCCGAAGATACCGCCGTGTATTACTGTGCTAGAGAGGATACGG |
| | | CCGCCTTCGACTACTGGGGCCAAGGAACCCTGGTTACAGTCAG |
| | | CAGC |
| (SEQ ID NO. | CD98-87 | CAGGTGCAACTGGTCGAGTCCGGCGGAGGCGTCGTGCAACCTG |
| 8772) | | GCAGGAGCCTCAGACTCTCCTGTGCTGCTAGCGGCTACCGATC |
| | | TAATGGGTACTACATGGGATGGTTCCGTCAGGCTAGTGGCAAG |
| | | GAGCGAGAGGGTGTTGCAGTGATCGATAGCGGGGGAAGTACAT |
| | | CCTATGCAGACTCCGTAAAGGGGCGATTCACCATCTCCCTCGA |
| | | CAATGCCAAAAACACACTTTACTTACAGATGAATAGCCTTCGT |
| | | GCCGAAGATACCGCCGTGTATTACTGTGCTAGAGGGGTAAAGC |
| | | GGTATGGAGACGGGGGCACAGACCTCTACTATTACTTCGACTA |
| | | TTGGGGCCAAGGAACCCTGGTTACAGTCAGCAGC |
| (SEQ ID NO. | CD98-96 | CAGGTGCAACTGGTCGAGTCCGGCGGAGGCGTCGTGCAACCTG |
| 8773) | | GCAGGAGCCTCAGACTCTCCTGTGCTGCTAGCGGCTACACCTT |
| | | ACCTATGAGCACCGCATGGGCGAGGCAAGCACCTGGAAAAGGG |
| | | CTAGAATGGGTTGCTGCAATCGCCTCCGACGGGAGAACAGACT |
| | | ATGCTGACTCAGTCAAGGGGAGGTTTACCATTTCGCGAGATAA |
| | | CTCCAAGAATACAGTGTATCTGCAAATGAATTCACTGCGCGCT |
| | | GAAGATACTGCCGTCTACTATTGCGCCACTCTGTGGTGGTACG |
| | | GCGGCAGAACGTTCGACAGTTGGGGCCAAGGAACCCTGGTTAC |
| | | AGTCAGCAGC |
| (SEQ ID NO. | CD98-39 | CAGGTGCAACTGGTCGAGTCCGGCGGAGGCGTCGTGCAACCTG |
| 8774) | | GCAGGAGCCTCAGACTCTCCTGTGCTGCTAGCGGCTTCACCTT |
| | | TGATGACTTCGACATGGGATGGTACCGTCAGGCTCCAGGCAAG |
| | | GACCGAGAGGGTGTTGCAGTGATCCGGAGCGACGGAATCCTGG |
| | | CCTATAGTGACTCCGTAAAGGGGCGATTCACCATCTCCCGGGA |
| | | CAATAGTAAAAACACAGTCTACTTACAGATGAATAGCCTTCGT |
| | | GCCGAAGATACCGCCGTGTATTACTGTGCTAGAAGCCGGAGCA |
| | | ACTATAGTAGCGACGCCTACGCCTACTACTGGTTCGACGTGTG |
| | | GGGCCAAGGAACCCTGGTTACAGTCAGCAGC |
| (SEQ ID NO. | CD98-47 | CAGGTGCAACTGGTCGAGTCCGGCGGAGGCGTCGTGCAACCTG |
| 8775) | | GCAGGAGCCTCAGACTCTCCTGTGCTGCTAGCGGCGGATTTGA |
| | | TGATGCAGACGCCGAGTGGGGGTGGGCTAGGCAAGCACCTGGG |
| | | AAGGGCCTCGAATGGGTGGCCGCCATCGCGGGGGACGGAAGAA |
| | | CCGATTACGCCGGCTCAGTGAAGGGCCGGTTTACCATTTCCAG |
| | | GGATAACTCTAAGAACACTGTGTATCTTCAAATGAACTCCTTA |
| | | CGGGCTGAAGACACCGCAGTCTACTATTGTGCCAAGAATGGGC |
| | | AGTCAGGATTCAGCTTCGACCCCTGGGGCCAAGGAACCCTGGT |
| | | TACAGTCAGCAGC |
| (SEQ ID NO. | CD98-53 | CAGGTGCAACTGGTCGAGTCCGGCGGAGGCGTCGTGCAACCTG |
| 8776) | | GCAGGAGCCTCAGACTCTCCTGTGCTGCTAGCGGCTACACCTT |
| | | ACCTATGAGCACCGCATGGGCGAGGCAAGCACCTGGAAAAGGG |
| | | CTAGAATGGGTTGCTGCAATCGCCTCCGACGGGAGAACAGACT |
| | | ATGCTGACTCAGTCAAGGGGAGGITTACCATTTCGCGAGATAA |
| | | CTCCAAGAATACAGTGTATCTGCAAATGAATTCACTGCGCGCT |
| | | GAAGATACTGCCGTCTACTATTGCGCCTCTCTGAGCCGGGGGA |
| | | CCTATAGTAGCGGGTGGAAGTGGGACGACTATTGGGGCCAAGG |
| | | AACCCTGGTTACAGTCAGCAGC |
| (SEQ ID NO. | CD98-62 | CAGGTGCAACTGGTCGAGTCCGGCGGAGGCGTCGTGCAACCTG |
| 8777) | | GCAGGAGCCTCAGACTCTCCTGTGCTGCTAGCGGCTTCACCTT |
| | | TTCTAGCTACGCCATGTCATGGTTCCGTCAGGCTCTGGGCAAG |
| | | GACCGAGAGGGTGTTGCAGCCATCGATAGCGACGGAACAACAT |
| | | CCTATGGAGAGTCCGTAAAGGGGCGATTCACCATCTCCCGGGA |
| | | CAATAGTAAAAACACAGTCTACTTACAGATGAATAGCCTTCGT |
| | | GCCGAAGATACCGCCGTGTATTACTGTGCTAGAGCGTGGGGGG |
| | | TAGGCGACGACTACTACAGAATGGACTACTGGGGCCAAGGAAC |
| | | CCTGGTTACAGTCAGCAGC |
| (SEQ ID NO. | CD98-68 | CAGGTGCAACTGGTCGAGTCCGGCGGAGGCGTCGTGCAACCTG |
| 8778) | | GCAGGAGCCTCAGACTCTCCTGTGCTGCTAGCGGCTACACCGC |
| | | TACTAACTACGCCATGGGATGGGTGCGTAGGGCTCCAGGCAAG |
| | | GGAGAAGAGTGGGTTGCAACAATCGATAGCGACGGAAGAGCAT |
| | | CCTATGCAGACTCCGTAAAGGGGCGATTCACCATCTCCGCCGA |
| | | CACTAGTAAAAACACAGCTTACTTACAGATGAATAGCCTTCGT |
| | | GCCGAAGATACCGCCGTGTATTACTGTGGTAGACTGCAGTACT |
| | | CATCAGGATGGTACTCAGACTACIGGGGCCAAGGAACCCTGGT |
| | | TACAGTCAGCAGC |
| (SEQ ID NO. | CD98-80 | CAGGTGCAACTGGTCGAGTCCGGCGGAGGCGTCGTGCAACCTG |
| 8779) | | GCAGGAGCCTCAGACTCTCCTGTGCTGCTAGCGGCTTCTCCTT |
| | | TGATGACCTGGACATGGGATGGGCACGTCAGGCTCCAGGCAAG |
| | | GGACTTGAGTGGGTTGCAACAATCACCAACGGGGACTACACAC |
| | | GGTATAGTGACTCCGTAAAGGGGCGATTCACCATCTCCCGGGA |
| | | CAATAGTAAAAACACACTTTACTTACAGATGAATAGCCTTCGT |
| | | GCCGAAGATACCGCCGTGTATTACTGTGCTAGAGACCTCAGCG |
| | | GCTCACTGTACTTCGACTACTGGGGCCAAGGAACCCTGGTTAC |
| | | AGTCAGCAGC |
| (SEQ ID NO. | CD98-112 | CAGGTGCAACTGGTCGAGTCCGGCGGAGGCGTCGTGCAACCTG |
| 8780) | | GCAGGAGCCTCAGACTCTCCTGTGCTGCTAGCGGCTACACCGT |
| | | TTCTAACAACTGCATGGGATGGTTCCGTCAGGCTCCAGGCAAG |
| | | GAGCGAGAGTTAGTTGCAGIGATCGGCAGCGACGGACACGCAA |
| | | CCTATGCAGCCTCCGTAGAGGGGCGATTCACCATCTCCCGGGA |
| | | CAATGCCAAAAGGATGGTCTACTTACAGATGAATAGCCTTCGT |
| | | GCCGAAGATACCGCCGTGTATTACTGTGCTAGAGCGCACCCGC |
| | | GGGGCAGTGGGAGCTACTACTTCGACTACTGGGGCCAAGGAAC |
| | | CCTGGTTACAGTCAGCAGC |
| (SEQ ID NO. | CD98-3 | CAGGTGCAACTGGTCGAGTCCGGCGGAGGCGTCGTGCAACCTG |
| 8781) | | GCAGGAGCCTCAGACTCTCCTGTGCTGCTAGCGGCTTCCGATA |
| | | TAATAGCTACGCCATGGCCTGGGCACGTCAGGCTCCAGGCAAG |
| | | GGAGAAGAGTGGGTTGCAGCCATCGATGGGGACGGAACAACAG |
| | | ATTATGCAGACTCCGTAAAGGGGCGATTCACCATCTCCGCCGA |
| | | CACTAGTAAAAACACAGTCTACTTACAGATGAATAGCCTTCGT |
| | | GCCGAAGATACCGCCGTGTATTACTGTGCTACAGTGAAGAAGT |
| | | TCTGGAACGGGTACGCCTTCGACTCATGGGGCCAAGGAACCCT |
| | | GGTTACAGTCAGCAGC |
| (SEQ ID NO. | CD98-138 | CAGGTGCAACTGGTCGAGTCCGGCGGAGGCGTCGTGCAACCTG |
| 8782) | | GCAGGAGCCTCAGACTCTCCTGTGCTGCTAGCGGCTTCCCTTT |
| | | TGGTGGGTACTCGGTGGGATGGTTCCGTCAGGCTCCAGGCAAG |
| | | GAGCGAGAGGGTGTTGCAGTGATCGATAGGGACGGAAGTACAA |
| | | CCTATGCAGACTCCGTAAAGGGGCGATTCACCATCTCCCGGGA |
| | | CAATGCCAAAGACACACTTTACTTACAGATGAATAGCCTTCGT |
| | | GCCGAAGATACCGCCGTGTATTACTGTGCTAGAGGGGGCAGGA |
| | | AGGGCCTGGGGTGGTACTACTTCGACTACTGGGGCCAAGGAAC |
| | | CCTGGTTACAGTCAGCAGC |
| (SEQ ID NO. | CD98-35 | CAGGTGCAACTGGTCGAGTCCGGCGGAGGCGTCGTGCAACCTG |
| 8783) | | GCAGGAGCCTCAGACTCTCCTGTGCTGCTAGCGGCTTCTCCTT |
| | | TTCTAGCTACCCCATGACCTGGGTGCGTCAGGCTCCAGGCAAG |
| | | GGACCCGAGTGGGTTAGTATGATCAATAGGGACGGAAGTACAT |
| | | ATTATGGAGACTCCGTAAAGGGGCGATTCGCCATCTCCCGGGA |
| | | CAATGCCAAAAACACACTTTACTTACAGATGAATAGCCTTCGT |
| | | GCCGAAGATACCGCCGTGTATTACTGTGCTAGAGACGGCGGGT |
| | | CATATGACATGGACGTATGGGGCCAAGGAACCCTGGTTACAGT |
| | | CAGCAGC |
| (SEQ ID NO. | CD98-59 | CAGGTGCAACTGGTCGAGTCCGGCGGAGGCGTCGTGCAACCTG |
| 8784) | | GCAGGAGCCTCAGACTCTCCTGTGCTGCTAGCGGCTACACCTT |
| | | ACCTATGAGCACCGCATGGGCGAGGCAAGCACCTGGAAAAGGG |
| | | CTAGAATGGGTTGCTGCAATCGCCTCCGACGGGAGAACAGACT |
| | | ATGCTGACTCAGTCAAGGGGAGGTTTACCATTICGCGAGATAA |
| | | CTCCAAGAATACAGTGTATCTGCAAATGAATTCACTGCGCGCT |
| | | GAAGATACTGCCGTCTACTATTGCGCCACTCTGAGCATGGCGC |
| | | GGAATAGTGACTACTGGGGCCAAGGAACCCTGGTTACAGTCAG |
| | | CAGC |
| (SEQ ID NO. | CD98-7 | CAGGTGCAACTGGTCGAGTCCGGCGGAGGCGTCGTGCAACCTG |
| 8785) | | GCAGGAGCCTCAGACTCTCCTGTGCTGCTAGCGGCTTCACCAT |
| | | TTCTGGGTACACCATGCACTGGTTCCGTCAGGCTCCAGGCAAG |
| | | GGACTTGAGTGGGTTGCAAGTATCGATACGGGGGGAGACACAA |
| | | CCTATCTGCCCTCCGTAAAGGGGCGATTCACCATCTCCCGGGA |
| | | CAATAGTAAAAACACACTTTACTTACAGATGAATAGCCTTCGT |
| | | GCCGAAGATACCGCCGTGTATTACTGTGCTACAGACCGGAACG |
| | | GCGGCGGAGCGTTCGACGTGTGGGGCCAAGGAACCCTGGTTAC |
| | | AGTCAGCAGC |
| (SEQ ID NO. | CD98-71 | CAGGTGCAACTGGTCGAGTCCGGCGGAGGCGTCGTGCAACCTG |
| 8786) | | GCAGGAGCCTCAGACTCTCCTGTGCTGCTAGCGGCTTCATTTC |
| | | TACTTACTACATGAGTTGGGTGAGGCAAGCACCTGGAAAAGGG |
| | | CCCGAATGGGTTTCTACAACAGGAGCCGACGGGACAACATACT |
| | | ATGCTGACTCAGTCAACGGGAGGTTTACCGTCTCGCAGGATAA |
| | | CGCTAAGAATACAGTGTATCTGCAAATGAATTCACTGCGCGCT |
| | | GAAGATACTGCCGTCTACTATTGCGCCCGCTACGAGGATGGGC |
| | | CCGGCTTCGACATATGGGGCCAAGGAACCCTGGTTACAGTCAG |
| | | CAGC |
| (SEQ ID NO. | CD98-84 | CAGGTGCAACTGGTCGAGTCCGGCGGAGGCGTCGTGCAACCTG |
| 8787) | | GCAGGAGCCTCAGACTCTCCTGTGCTGCTAGCGGCTACACCTT |
| | | ACCTATGAGCATGGGATGGTACAGGCAAGCACCTGGAAAAGAC |
| | | CGGGAAGGCGTTGCTGCAATCGCCGGCGACGGGAGAACAGACT |
| | | ATGCTGACTCAGTCAAGGGGAGGTTTACCATTICGCGAGATAA |
| | | CTCCAAGAATACACTGTATCTGCAAATGAATTCACTGCGCGCT |
| | | GAAGATACTGCCGTCTACTATTGCGCCCGCGGAGCGGATTGGA |
| | | ACTATGGATACTGGTACTTCGACCTCTGGGGCCAAGGAACCCT |
| | | GGTTACAGTCAGCAGC |
| (SEQ ID NO. | CD98-9 | CAGGTGCAACTGGTCGAGTCCGGCGGAGGCGTCGTGCAACCTG |
| 8788) | | GCAGGAGCCTCAGACTCTCCTGTGCTGCTAGCGGCTTCCCTTT |
| | | TGGTGGGTACTCGGTGGGATGGTTCCGTCAGGCTCCAGGCAAG |
| | | GAGCGAGAGGGTGTTGCAGTGATCGATAGGGACGGAAGTACAA |
| | | CCTATGCAGACTCCGTAAAGGGGCGATTCACCATCTCCCGGGA |
| | | CAATGCCAATGACACACTTTACTTACAGATGAATAGCCTTCGT |
| | | GCCGAAGATACCGCCGTGTATTACTGTGCTAAGAGCGCCAAGT |
| | | ACTATTACGAGGGGTCATACTACTACGTGCATTTCGACTACTG |
| | | GGGCCAAGGAACCCTGGTTACAGTCAGCAGC |
| (SEQ ID NO. | CD98-94 | CAGGTGCAACTGGTCGAGTCCGGCGGAGGCGTCGTGCAACCTG |
| 8789) | | GCAGGAGCCTCAGACTCTCCTGTGCTGCTAGCGGCTTCACCAC |
| | | TACTACATACACCATGGGATGGTTCCGTCAGGCTCCAGGCAAG |
| | | GGACGAGAGTTAGITGCAGTGATCGATAGCGACGGAGACACAG |
| | | CCTATGCAGAGTCCGTAGAGGGGCGATTCACCATCTCCCGGGA |
| | | CAATGCCAAAAGGATGGTCTACTTACAGATGAATAGCCTTCGT |
| | | GCCGAAGATACCGCCGTGTATTACTGTGCTAGAGACAAGGGGG |
| | | GCTATGGAGACTACCCCTTCTTCCTCTTCGATTACTGGGGCCA |
| | | AGGAACCCTGGTTACAGTCAGCAGC |
| (SEQ ID NO. | ALK1-13 | CAGGTGCAACTGGTCGAGTCCGGCGGAGGCGTCGTGCAACCTG |
| 8790) | | GCAGGAGCCTCAGACTCTCCTGTGCTGCTAGCGGCTTCACCTT |
| | | TTCTAGCTACGCCATGTCATGGTTCCGTCAGGCTCCAGGCAAG |
| | | GGACGAGAGTTAGTTGCAGCCATCGATAGCGACGGAAGTACAT |
| | | CCTATGCAGACTCCGTAGAGGGGCGATTCACCATCTCCCGGGA |
| | | CAATGCCAAAAGGATGGTCTACTTACAGATGAATAGCCTTCGT |
| | | GCCGAAGATACCGCCGTGTATTACTGTGCTAGAGTGGATTTCT |
| | | ACTATTACTACATGGACGTGTGGGGCCAAGGAACCCTGGTTAC |
| | | AGTCAGCAGC |
| (SEQ ID NO. | FLT1-39 | CAGGTGCAACTGGTCGAGTCCGGCGGAGGCGTCGTGCAACCTG |
| 8791) | | GCAGGAGCCTCAGACTCTCCTGTGCTGCTAGCGGCGCCACCTC |
| | | TAATACATACTACATGGGATGGTTCCGTCAGGCTCCAGGCAAG |
| | | GAGCGAGAGGGTGTTGCAACAATCCGGAGGGACGACCTGACAG |
| | | CCTATACAGACTCCGTAAAGGGGCGATTCACCATCTCCCGGGA |
| | | CAATACAAAAAACACACTTTACTTACAGATGAATAGCCTTCGT |
| | | GCCGAAGATACCGCCGTGTATTACTGTGCTAGACTGGATAGCA |
| | | ACGGCTACAGCTACTGGGGCCAAGGAACCCTGGTTACAGTCAG |
| | | CAGC |
| (SEQ ID NO. | FLT1-4 | CAGGTGCAACTGGTCGAGTCCGGCGGAGGCGTCGTGCAACCTG |
| 8792) | | GCAGGAGCCTCAGACTCTCCTGTGCTGCTAGCGGCAGATCCAA |
| | | TGAAAACTACTTCCTGGCCTGGTACCGTCAGGCTCCAGGCAAG |
| | | GACCGAGAGGGTGTTGCAGTGATCTATAGCGACGGAAAGACAG |
| | | TCTATGCAGACTCCGTAAAGGGGCGATTCACCATCTCCCGGGA |
| | | CAATAGTAAAAACACACTTTACTTACAGATGAATAGCCTTCGT |
| | | GCCGAAGATACCGCCGTGTATTACTGTACTGAGTACGGCTTCA |
| | | AGAATGGATACGCGTTCGACGCCTGGGGCCAAGGAACCCTGGT |
| | | TACAGTCAGCAGC |
| (SEQ ID NO. | FLT1-49 | CAGGTGCAACTGGTCGAGTCCGGCGGAGGCGTCGTGCAACCTG |
| 8793) | | GCAGGAGCCTCAGACTCTCCTGTGCTGCTAGCGGCGCCACCTC |
| | | TAATACATACTACATGGGATGGTTCCGTCTGGCTCCAGGCAAG |
| | | GAGCGAGAGGGTGTTGCAAGTATCTATACGCAGGACGGAAGAA |
| | | CCGCTTACGCCGATTCAGTGAAGGGCCGGTTTACCATTTCCGA |
| | | GGATAACGCTAAGAACACTGTGTATCTTCAAATGAACTCCTTA |
| | | CGGGCTGAAGACACCGCAGTCTACTATTGTGCCAGGATCGACA |
| | | TCGATGGATACGCGTACTGGGGCCAAGGAACCCTGGTTACAGT |
| | | CAGCAGC |
| (SEQ ID NO. | FLT1-6 | CAGGTGCAACTGGTCGAGTCCGGCGGAGGCGTCGTGCAACCTG |
| 8794) | | GCAGGAGCCTCAGACTCTCCTGTGCTGCTAGCGGCAGTTCCGA |
| | | TGAAATCTACGCCATGGCCTGGTTCCGTCAGGCTCCAGGCAAG |
| | | GGACGAGAGTTAGTTGCAGCCATCGAAAACGACGGAACAACAA |
| | | CCTATGCACACTCCGTAAAGGGGCGATTCACCATCTCCGCCGA |
| | | CACTAGTAAAAACACAGCTTACTTACAGATGAATAGCCTTCGT |
| | | GCCGAAGATACCGCCGTGTATTACTGTGCTAGAGACTCAACGT |
| | | CATGGTGGTACTTCGACCTGTGGGGCCAAGGAACCCTGGTTAC |
| | | AGTCAGCAGC |
| (SEQ ID NO. | IGF1R-17 | CAGGTGCAACTGGTCGAGTCCGGCGGAGGCGTCGTGCAACCTG |
| 8795) | | GCAGGAGCCTCAGACTCTCCTGTGCTGCTAGCGGCCCATCCGC |
| | | TTCTGCAAAGGCCATGGCCTGGTTCCGTCAGTCTGTGGGCAAG |
| | | GAGCGAGAGGGTATTGCAGCCATCCGGCACGACGGAGGAGAGA |
| | | CCTGGTACGCCGCCTCAGTGAAGGGCCGGTTTACCATTTCCAG |
| | | GGATAACGCTAAGAACACTCTGTATCTTCAAATGAACTCCTTA |
| | | CGGGCTGAAGACACCGCAGTCTACTATTGTGTGAGGGATCACG |
| | | GCGGCGCCAGGGACATCTGGGGCCAAGGAACCCTGGTTACAGT |
| | | CAGCAGC |
| (SEQ ID NO. | IGF1R-37 | CAGGTGCAACTGGTCGAGTCCGGCGGAGGCGTCGTGCAACCTG |
| 8796) | | GCAGGAGCCTCAGACTCTCCTGTGCTGCTAGCGGCCTGTCCGT |
| | | TTCTGACAGCTCGATGGCCTGGTTCCGTCAGCGTCCAGGCCAG |
| | | GAGCGAGAGGGTATTGCAGCCATCGATACGGGGGACGGAAGTA |
| | | CCTATTACCTCAATTCAGTGGAGGGCCGGTTTACCATTTCCCA |
| | | CGATAACGCTAAGAACACTCTGTATCTTCAAATGAACTCCTTA |
| | | CGGGCTGAAGACACCGCAGTCTACTATTGTGTGAGGCAGCAGC |
| | | GGGATGGACTGGTGAATTTCTGGGGCCAAGGAACCCTGGTTAC |
| | | AGTCAGCAGC |
| (SEQ ID NO. | INSULINR- | CAGGTGCAACTGGTCGAGTCCGGCGGAGGCGTCGTGCAACCTG |
| 8797) | 87 | GCAGGAGCCTCAGACTCTCCTGTGCTGCTAGCGGCTTCACCGT |
| | | TGGTGCAAGGGCCATGGCCTGGTACCGTCAGGCTCCAGGCAAG |
| | | GACCGAGAGGGTGTTGCAGCCATCGATAGCGACGGAATCGCAT |
| | | CCTATGCAGACTCCGTAAAGGGGCGATTCACCATCTCCCGGGA |
| | | CAATGCCAAAAACACACTTTACTTACAGATGAATAGCCTTCGT |
| | | GCCGAAGATACCGCCGTGTATTACTGTGCTAGAGTGTCAGGGT |
| | | CATATGGAGACTACTTCGACTACTGGGGCCAAGGAACCCTGGT |
| | | TACAGTCAGCAGC |
| (SEQ ID NO. | INSULINR- | CAGGTGCAACTGGTCGAGTCCGGCGGAGGCGTCGTGCAACCTG |
| 8798) | 96 | GCAGGAGCCTCAGACTCTCCTGTGCTGCTAGCGGCTACACCTT |
| | | TGATGAGAGCGACATGGGATGGTTCCGTCAGGCTCCAGGCAAG |
| | | GGACGAGAGTTAGTTAGTACAATCTCCCCGGACGGAACAACAT |
| | | ATTATGCAGACTCCGTAGAGGGGCGATTCACCATCTCCCGGGA |
| | | CAATGCCAAAAGGATGGTCTACTTACAGATGAATAGCCTTCGT |
| | | GCCGAAGATACCGCCGTGTATTACTGTGCTAGATGGCGGAGGT |
| | | CATGGTTCGACACGTGGGGCCAAGGAACCCTGGTTACAGTCAG |
| | | CAGC |
| (SEQ ID NO. | LDLR-13 | CAGGTGCAACTGGTCGAGTCCGGCGGAGGCGTCGTGCAACCTG |
| 8799) | | GCAGGAGCCTCAGACTCTCCTGTGCTGCTAGCGGCTACACCGT |
| | | TGCTACATACTTCATCGGATGGTTCCGTCAGGCTCCAGGCAAG |
| | | GCCCGAGAGGGTGTTGCAACAATCACCAGCGACGGAAGAACAA |
| | | AATATGCAGACTCCGTAAAGGGGCGATTCACCATCTCCCGGGA |
| | | CAATGCCAAAAACACAGTCTACTTACAGATGAATAGCCTTCGT |
| | | GCCGAAGATACCGCCGTGTATTACTGTGCTAGTAGGTCAATAG |
| | | CCGCCGCCGGGGAGGGCTACTGGGGCCAAGGAACCCTGGTTAC |
| | | AGTCAGCAGC |
| (SEQ ID NO. | LRP1C2-46 | CAGGTGCAACTGGTCGAGTCCGGCGGAGGCGTCGTGCAACCTG |
| 8800) | | GCAGGAGCCTCAGACTCTCCTGTGCTGCTAGCGGCTACCCTTC |
| | | TTGTATGGGGTGGGCACGGCAGGCACCTGGGAAAGGATTGGAG |
| | | TGGGTCGCCTGTATTGAGAGAAGAGGCAGGATACACTACGCAG |
| | | CCTCTGTGAAGGGCCGGTTCACGATTTCGCGAGACAATTCCAA |
| | | GAATACATTGTACCTGCAAATGAATTCCTTGCGGGCAGAAGAT |
| | | ACCGCTGTTTACTATTGCGCTAGCCAGGATAGTAGCTCATACG |
| | | ACGCCTTCGACATATGGGGCCAAGGAACCCTGGTTACAGTCAG |
| | | CAGC |
| (SEQ ID NO. | LRP1C3-24 | CAGGTGCAACTGGTCGAGTCCGGCGGAGGCGTCGTGCAACCTG |
| 8801) | | GCAGGAGCCTCAGACTCTCCTGTGCTGCTAGCGGCGGAGCCTA |
| | | TCGTAAGTACTACATGGGATGGTTCCGTCAGGCTCCAGGCAAG |
| | | GGACGAGAGTTAGTTGCAGCCATCGAAAGCGACGGAACAACAG |
| | | ATTATGCAGACTCCGTAGAGGGGCGATTCACCATCTCCCGGGA |
| | | CAATGCCAAAAGGATGGTCTACTTACAGATGAATAGCCTTCGT |
| | | GCCGAAGATACCGCCGTGTATTACTGTGCTAGTGTGATCGGGG |
| | | GCAATAGTGAGGACTACTGGGGCCAAGGAACCCTGGTTACAGT |
| | | CAGCAGC |
| (SEQ ID NO. | LRP1C4-33 | CAGGTGCAACTGGTCGAGTCCGGCGGAGGCGTCGTGCAACCTG |
| 8802) | | GCAGGAGCCTCAGACTCTCCTGTGCTGCTAGCGGCGACGCCAA |
| | | TGAATTCGAGTGGATCGGATGGTACCGTCAGGCTCCAGGCAAG |
| | | GGACGAGAGTTAGTTGCAAGAATCGGCAGCAGCGGACCAAGAT |
| | | ATGCTGACTCAGTCGAGGGGAGGTTTACCATTICGCGAGATAA |
| | | CTCCAAGAATACACTGTATCTGCAAATGAATTCACTGCGCGCT |
| | | GAAGATACTGCCGTCTACTATTGCGCCGCTACACACTGTAGCT |
| | | TCCTCACAACGAGCGTAGCAGGCTGCACAGATGACAAGTACTG |
| | | GGGCCAAGGAACCCTGGTTACAGTCAGCAGC |
| (SEQ ID NO. | SCARB1-3 | CAGGTGCAACTGGTCGAGTCCGGCGGAGGCGTCGTGCAACCTG |
| 8803) | | GCAGGAGCCTCAGACTCTCCTGTGCTGCTAGCGGCTTCACCTT |
| | | AGGTACAAACTGCATGGCCTGGTTCCGTCAGGCTCCAGGCAAG |
| | | GAGCGAGAGCGTGTTGCAAGTATCGGCAGCGACGGAAGAACAT |
| | | ATTATGCAGACTCCGTAAAGGGGCGATTCACCATCTCCCGGGA |
| | | CAATAGTAAAAACACACTTTACTTACAGATGAATAGCCTTCGT |
| | | GCCGAAGATACCGCCGTGTATTACTGTGCTAGAGCGCCCGGGA |
| | | TCCAGCTGTGGAAGTTCGACTACTGGGGCCAAGGAACCCTGGT |
| | | TACAGTCAGCAGC |
| (SEQ ID NO. | SCARB1- | CAGGTGCAACTGGTCGAGTCCGGCGGAGGCGTCGTGCAACCTG |
| 8804) | 49 | GCAGGAGCCTCAGACTCTCCTGTGCTGCTAGCGGCTTCACCAA |
| | | TTCTAAGACGTACCTGGGATGGGTGCGTAGGGCTCCAGGCAAG |
| | | GGACTTGAGTGGGTTGCAGCCATCGCCCCGAGCACAGGAAGTA |
| | | CCTATTACGACGATTCAGTGAAGGGCCGGTTTACCATTTCCAG |
| | | GGATAACTCTAAGAACACTCTGTATCTTCAAATGAACTCCTTA |
| | | CGGGCTGAAGACACCGCAGTCTACTATTGTGCCAGCCAGGGGT |
| | | CATCATGGTACGAGAATGACTACTGGGGCCAAGGAACCCTGGT |
| | | TACAGTCAGCAGC |
| (SEQ ID NO. | IGF1R-55 | CAGGTGCAACTGGTCGAGTCCGGCGGAGGCGTCGTGCAACCTG |
| 8805) | | GCAGGAGCCTCAGACTCTCCTGTGCTGCTAGCGGCTTCCGATT |
| | | TGATGACTACGCCATGGGATGGTTCCGTCAGGCTCCAGGCAAG |
| | | GAGCGAGAGGGTGTTGCAGGATACAAAAGCGACGGAAGTACAA |
| | | CCTATGCAGACTCCGTAAAGGGGCGATTCACCATCTCCCGGGA |
| | | CAATAGTAAAAACACACTTTACTTACAGATGAATAGCCTTCGT |
| | | GCCGAAGATACCGCCGTGTATTACTGTGCTAGTAGGGCCCTGT |
| | | ACGGCGGAAAGCCGTTCGTGATCTGGGGCCAAGGAACCCTGGT |
| | | TACAGTCAGCAGC |
| (SEQ ID NO. | LRP1C4-31 | CAGGTGCAACTGGTCGAGTCCGGCGGAGGCGTCGTGCAACCTG |
| 8806) | | GCAGGAGCCTCAGACTCTCCTGTGCTGCTAGCGGCAGTCCTGA |
| | | TCGTAGGGCGTGCATGGGATGGTACCGTCAGGCTCCAGGCAAG |
| | | GAGCGAGAGCGTGTTGCAGAGATCGATACGGACGGAAACACAT |
| | | CCTATGCAGACTCCGTAAAGGGGCGATTCACCATCTCCCGGGA |
| | | CAATAGTAAAAACACAGTCTACTTACAGATGAATAGCCTTCGT |
| | | GCCGAAGATACCGCCGTGTATTACTGTGCTCTGCAGCCCGGGT |
| | | GCCCCCCAGCGACGCTCTTCTACTGGAGAAAAACAGGATTCGG |
| | | TAACTGGGGCCAAGGAACCCTGGTTACAGTCAGCAGC |
| (SEQ ID NO. | LRP1C4-80 | CAGGTGCAACTGGTCGAGTCCGGCGGAGGCGTCGTGCAACCTG |
| 8807) | | GCAGGAGCCTCAGACTCTCCTGTGCTGCTAGCGGCTTCACCTT |
| | | TGATGACACGGACATGGGATGGTTCCGTCAGGCTCCAGGCAAG |
| | | GAGCGAGAGCGTGTTAGTACAATCAATAGCGACGGAGGAACAA |
| | | CCTATTACGCCGATTCAGTGAAGGGCCGGTTTACCATTTCCAG |
| | | GGATAACTCTAAGAACACTCTGTATCTTCAAATGAACTCCTTA |
| | | CGGGCTGAAGACACCGCAGTCTACTATTGTGCCAGGGCCAGGG |
| | | TAACCGACGCGGGGTGGCAGTACTTCGACTATTGGGGCCAAGG |
| | | AACCCTGGTTACAGTCAGCAGC |
| (SEQ ID NO. | ALK1-15 | CAGGTGCAACTGGTCGAGTCCGGCGGAGGCGTCGTGCAACCTG |
| 8808) | | GCAGGAGCCTCAGACTCTCCTGTGCTGCTAGCGGCTTCACCTT |
| | | TTCTGACTACTACATCGGATGGTTCCGTCAGGCTCCAGGCAAG |
| | | GAGCGAGAGCGTGTTGCACAGATCGATAAGAACGGAGTGACAA |
| | | ATTATGCAGACGCCGTAAAGGGGCGATTCACCATCTCCCGGGA |
| | | CAATAGTAAAAACACACTTTACTTACAGATGAATAGCCTTCGT |
| | | GCCGAAGATACCGCCGTGTATTACTGTGCTGGAGTGGATGCGT |
| | | TCAATTACATGGAGGTATGGGGCCAAGGAACCCTGGTTACAGT |
| | | CAGCAGC |
| (SEQ ID NO. | IGF1R-13 | CAGGTGCAACTGGTCGAGTCCGGCGGAGGCGTCGTGCAACCTG |
| 8809) | | GCAGGAGCCTCAGACTCTCCTGTGCTGCTAGCGGCGACTTTAA |
| | | TGATGCAAGGGCCGAGTGGGGGTGGTTTAGGCAAGCACCTGGG |
| | | AAGGGCCGGGAATTGGTGGCCAGTCTCGCGAGCGACGGAAGTA |
| | | CCATTTACGCCAATCCCGTGGAGGGCCGGTTTACCATTTCCAG |
| | | GGATAACGCTAAGAACACTCTGTATCTTCAAATGAACTCCTTA |
| | | CGGGCTGAAGACACCGCAGTCTACTATTGTGCCAGGTCAGGGG |
| | | CCTCAAGTAGCTGGTACTGGTTCGACTACTGGGGCCAAGGAAC |
| | | CCTGGTTACAGTCAGCAGC |
| (SEQ ID NO. | IGF1R-96 | CAGGTGCAACTGGTCGAGTCCGGCGGAGGCGTCGTGCAACCTG |
| 8810) | | GCAGGAGCCTCAGACTCTCCTGTGCTGCTAGCGGCAGTATTCA |
| | | TAATAGGAGGCACATGGGATGGTTCCGTCAGGCTGCAGGCAAG |
| | | GAGCGAGAGGGTGTTGCAGCCATCGATATAGCGGGAAACACAC |
| | | GGATTAGTGACTCCGTAAAGGGGCGATTCACCATCTCCCATGA |
| | | CAAAGACGAAAACAGACTTTACTTACAGATGAATAGCCTTCGT |
| | | GCCGAAGATACCGCCGTGTATTACTGTGCTATCCAGTCAAGCG |
| | | ACGGCAGAGGGACGGTATACTGGGGCCAAGGAACCCTGGTTAC |
| | | AGTCAGCAGC |
| (SEQ ID NO. | INSULINR- | CAGGTGCAACTGGTCGAGTCCGGCGGAGGCGTCGTGCAACCTG |
| 8811) | 86 | GCAGGAGCCTCAGACTCTCCTGTGCTGCTAGCGGCAGACGAGG |
| | | TAATAACTACTCGATGGCCTGGTACCGTCAGGCTCCAGGCAAG |
| | | GACCGAGAGGGTGTTGCAGCCATCGATAGCGACGGAGACACAT |
| | | CCTATGCAGACCCCGTAAAGGGGCGATTCACCATCTCCCGGGA |
| | | CAATAGTAAAAACACACTTTACTTACAGATGAATAGCCTTCGT |
| | | GCCGAAGATACCGCCGTGTATTACTGTGCTGTGGCGACCGGGG |
| | | AGTATAGAAGGTACTTCGACGTATGGGGCCAAGGAACCCTGGT |
| | | TACAGTCAGCAGC |
| (SEQ ID NO. | LRP1C3-93 | CAGGTGCAACTGGTCGAGTCCGGCGGAGGCGTCGTGCAACCTG |
| 8812) | | GCAGGAGCCTCAGACTCTCCTGTGCTGCTAGCGGCTTCGCCTT |
| | | TACTAAGTACCACATGTCATGGTTCCGTCAGGCTCCAGGCAAG |
| | | GGACGAGAGTTAGTTAGTCACATCTCCGCGGGGGGAAGAACAT |
| | | ATTATAGTGACTCCGTAGAGGGGCGATTCACCATCTCCCGGGA |
| | | CAATGCCAAAAGGATGGTCTACTTACAGATGAATAGCCTTCGT |
| | | GCCGAAGATACCGCCGTGTATTACTGTGTTAAGGAGTCAAGGC |
| | | CCCACAGTTACGGGGACCACTGGGGCCAAGGAACCCTGGTTAC |
| | | AGTCAGCAGC |

TABLE 7
Table providing exemplary list of shuttle amino acid sequences

| SEQ ID NO. | ID | AMINO ACID SEQUENCE |
|---|---|---|
| (SEQ ID NO. | TfR-1409 | QVQLVESGGGVVQPGRSLRLSCAASGFSFSNFGMSWVRQARGK |
| 8813) | | GLEWVSSTGSDGTAYYADSVKGRFTISRDNAKNTLYLQMNSLR |
| | | AEDTAVYYCARAPSRHGFDYWGQGTLVTVSS |
| (SEQ ID NO. | TfR-1409- | QVQLVESGGGVVQPGRSLRLSCAASGFSFSNEGMSWVRQARGK |
| 8814) | 51 | GLEWVSSIGSDGRAYYADSVKGRFTISRDNAKNTLYLQMNSLR |
| | | AEDTAVYYCARAPSRHGFDYWGOGTLVTVSS |
| (SEQ ID NO. | TfR-1409-6 | QVQLVESGGGVVQPGRSLRLSCAASGFSESNEGMSWVRQARGK |
| 8815) | | GLEWVSSVGSDGTAYYADSVKGRFTISRDNAKNTLYLQMNSLR |
| | | AEDTAVYYCARAPSRHGFDYWGQGTLVTVSS |
| (SEQ ID NO. | TfR-1409- | QVQLVESGGGVVQPGRSLRLSCAASGFSFSNFGMSWVRQAPGK |
| 8816) | 25 | GLEWVSSTGSDGTAYYADSVKGRFTISRDNAKNTLHLQMNSLR |
| | | AEDTAVYYCARAPSRHGFDYWGQGTLVTVSS |
| (SEQ ID NO. | TfR-455 | QVQLVESGGGVVQPGRSLRLSCAASGFAVDASDMGWFRQAPGK |
| 8817) | | GRELVSSISSDNDTYYADSVEGRFTISRDNAKRMVYLQMNSLR |
| | | AEDTAVYYCASGRFLEPDYWGQGTLVTVSS |
| (SEQ ID NO. | TfR-299 | QVQLVESGGGVVQPGRSLRLSCAASGFTFDDSDMGWFRQAPGK |
| 8818) | | GRELVAAINTGGGKTYYAATVEGRFTISRDNAKRMVYLQMNSL |
| | | RAEDTAVYYCASGRFLEPDYWGQGTLVTVSS |
| (SEQ ID NO. | TfR-891 | QVQLVESGGGVVQPGRSLRLSCAASGLTEDDSDMGWFRQAPGK |
| 8819) | | ERERVSTISSDGTPHYDDSVKGRFTISRDNSKNTLYLQMNSLR |
| | | AEDTAVYYCARRDGGSWNEDYWGQGTLVTVSS |
| (SEQ ID NO. | TfR-732 | QVQLVESGGGVVQPGRSLRLSCAASGFTFDGSDIAWVRRAPGK |
| 8820) | | GEEWVSTISSDGTVYYSDPVKGRFTISADTSKNTAYLQMNSLR |
| | | AEDTAVYYCAISSWYGDEDYWGQGTLVTVSS |
| (SEQ ID NO. | TfR-734 | QVQLVESGGGVVQPGRSLRLSCAASGHILNYWYMGWVRRAPGK |
| 8821) | | GEEWVAGISPGGTVTVYADSVKGRFTISRDNSKNTLYLQMNSL |
| | | RAEDTAVYYCARGGQGGDAWAFDYWGOGTLVTVSS |
| (SEQ ID NO. | TfR-358 | QVQLVESGGGVVQPGRSLRLSCAASGFGVASSDMGWFRQAPGK |
| 8822) | | GRELVASINTDGSTIYAESVEGRFTISRDNAKRMVYLQMNSLR |
| | | AEDTAVYYCAAYDTSAYHSPFDYWGQGTLVTVSS |
| (SEQ ID NO. | TfR-85 | QVQLVESGGGVVQPGRSLRLSCAASGYTERSASMGWVRRAPGK |
| 8823) | | GEEWVAVIDSEGHTIYADSVKGRFTISADTSKNTAYLQMNSLR |
| | | AEDTAVYYCAKDIYGDYGGPDYWGOGTLVTVSS |
| (SEQ ID NO. | TfR-448 | QVQLVESGGGVVQPGRSLRLSCAASGTRRPNYVTWERQAPLNE |
| 8824) | | ROVVAGIDSAGTTTYADSVKGRFTISQDNAKNTVYLQMNSLRA |
| | | EDTAVYYCASGISSSSFYPFDSWGQGTLVTVSS |
| (SEQ ID NO. | TfR-1573 | QVQLVESGGGVVQPGRSLRLSCAASGENEDDSDMGWFRQAPGK |
| 8825) | | GRELVSSIASDGATYYADSVEGRFTISRDNAKRMVYLQMNSLR |
| | | AEDTAVYYCARDSPSLRAFDSWGQGTLVTVSS |
| (SEQ ID NO. | TfR-1417 | QVQLVESGGGVVQPGRSLRLSCAASGTTYSRQYCMGWARQAPG |
| 8826) | | KGLEWVSCIDDDGNTYYADSVKGRFTISRDNSKNTLYLQMNSL |
| | | RAEDTAVYYCARDPGLSGDFDYWGQGTLVTVSS |
| (SEQ ID NO. | TfR-1090 | QVQLVESGGGVVQPGRSLRLSCAASGITYRGPCMGWYRQAPGK |
| 8827) | | GLEWVATLDSDGSTTYADSVKGRFTISRDNSKNTLYLQMNSLR |
| | | AEDTAVYYCAMYMTGGGGVAFDYWGQGTLVTVSS |
| (SEQ ID NO. | TfR-1415 | QVQLVESGGGVVQPGRSLRLSCAASGFTFSKYAMNWARQAPGK |
| 8828) | | GLEWVAFIDTAGRTIYADSVKGRFTISRDNSKNTLYLQMNSLR |
| | | AEDTAVYYCAPSYGDSGDYFDYWGQGTLVTVSS |
| (SEQ ID NO. | CD98-11 | QVQLVESGGGVVQPGRSLRLSCAASGFPFSSYGMGWFRQAPGK |
| 8829) | | EREGVAGVNTGGRTSYAESVKGRFTISRDNTRNTLYLQMNSLR |
| | | AEDTAVYYCARIRAAGTVAPYFDYWGQGTLVTVSS |
| (SEQ ID NO. | CD98-74 | QVQLVESGGGVVQPGRSLRLSCAASGYVAGYVGWYRQAPGKDR |
| 8830) | | EGVAAIASDGMTAYAHSVKGRFTISRDNSKNTLYLQMNSLRAE |
| | | DTAVYYCASGYCSGSGCYFDSWGQGTLVTVSS |
| (SEQ ID NO. | CD98-82 | QVQLVESGGGVVQPGRSLRLSCAASGFRYNSYAMAWARQAPGK |
| 8831) | | GLEWVAAIAGDGRTDYADSVKGRFTISRDNSKNTLYLQMNSLR |
| | | AEDTAVYYCAREDTAAFDYWGQGTLVTVSS |
| (SEQ ID NO. | CD98-87 | QVQLVESGGGVVQPGRSLRLSCAASGYRSNGYYMGWFRQASGK |
| 8832) | | EREGVAVIDSGGSTSYADSVKGRFTISLDNAKNTLYLQMNSLR |
| | | AEDTAVYYCARGVKRYGDGGTDLYYYFDYWGQGTLVTVSS |
| (SEQ ID NO. | CD98-96 | QVQLVESGGGVVQPGRSLRLSCAASGYTLPMSTAWARQAPGKG |
| 8833) | | LEWVAAIASDGRTDYADSVKGRFTISRDNSKNTVYLQMNSLRA |
| | | EDTAVYYCATLWWYGGRTFDSWGQGTLVTVSS |
| (SEQ ID NO. | CD98-39 | QVQLVESGGGVVQPGRSLRLSCAASGFTEDDEDMGWYRQAPGK |
| 8834) | | DREGVAVIRSDGILAYSDSVKGRFTISRDNSKNTVYLQMNSLR |
| | | AEDTAVYYCARSRSNYSSDAYAYYWEDVWGQGTLVTVSS |
| (SEQ ID NO. | CD98-47 | QVQLVESGGGVVQPGRSLRLSCAASGGFDDADAEWGWARQAPG |
| 8835) | | KGLEWVAAIAGDGRTDYAGSVKGRFTISRDNSKNTVYLQMNSL |
| | | RAEDTAVYYCAKNGQSGFSFDPWGQGTLVTVSS |
| (SEQ ID NO. | CD98-53 | QVQLVESGGGVVQPGRSLRLSCAASGYTLPMSTAWARQAPGKG |
| 8836) | | LEWVAAIASDGRTDYADSVKGRFTISRDNSKNTVYLQMNSLRA |
| | | EDTAVYYCASLSRGTYSSGWKWDDYWGQGTLVTVSS |
| (SEQ ID NO. | CD98-62 | QVQLVESGGGVVQPGRSLRLSCAASGFTFSSYAMSWFRQALGK |
| 8837) | | DREGVAAIDSDGTTSYGESVKGRFTISRDNSKNTVYLQMNSLR |
| | | AEDTAVYYCARAWGVGDDYYRMDYWGQGTLVTVSS |
| (SEQ ID NO. | CD98-68 | QVQLVESGGGVVQPGRSLRLSCAASGYTATNYAMGWVRRAPGK |
| 8838) | | GEEWVATIDSDGRASYADSVKGRFTISADTSKNTAYLQMNSLR |
| | | AEDTAVYYCGRLQYSSGWYSDYWGQGTLVTVSS |
| (SEQ ID NO. | CD98-80 | QVQLVESGGGVVQPGRSLRLSCAASGFSFDDLDMGWARQAPGK |
| 8839) | | GLEWVATITNGDYTRYSDSVKGRFTISRDNSKNTLYLQMNSLR |
| | | AEDTAVYYCARDLSGSLYFDYWGQGTLVTVSS |
| (SEQ ID NO. | CD98-112 | QVQLVESGGGVVQPGRSLRLSCAASGYTVSNNCMGWFRQAPGK |
| 8840) | | ERELVAVIGSDGHATYAASVEGRETISRDNAKRMVYLQMNSLR |
| | | AEDTAVYYCARAHPRGSGSYYFDYWGQGTLVTVSS |
| (SEQ ID NO. | CD98-3 | QVQLVESGGGVVQPGRSLRLSCAASGFRYNSYAMAWARQAPGK |
| 8841) | | GEEWVAAIDGDGTTDYADSVKGRFTISADTSKNTVYLQMNSLR |
| | | AEDTAVYYCATVKKFWNGYAFDSWGQGTLVTVSS |
| (SEQ ID NO. | CD98-138 | QVQLVESGGGVVQPGRSLRLSCAASGFPFGGYSVGWFRQAPGK |
| 8842) | | EREGVAVIDRDGSTTYADSVKGRFTISRDNAKDTLYLQMNSLR |
| | | AEDTAVYYCARGGRKGLGWYYFDYWGQGTLVTVSS |
| (SEQ ID NO. | CD98-35 | QVQLVESGGGVVQPGRSLRLSCAASGFSFSSYPMTWVRQAPGK |
| 8843) | | GPEWVSMINRDGSTYYGDSVKGRFAISRDNAKNTLYLQMNSLR |
| | | AEDTAVYYCARDGGSYDMDVWGQGTLVTVSS |
| (SEQ ID NO. | CD98-59 | QVQLVESGGGVVQPGRSLRLSCAASGYTLPMSTAWARQAPGKG |
| 8844) | | LEWVAAIASDGRTDYADSVKGRFTISRDNSKNTVYLQMNSLRA |
| | | EDTAVYYCATLSMARNSDYWGQGTLVTVSS |
| (SEQ ID NO. | CD98-7 | QVQLVESGGGVVQPGRSLRLSCAASGFTISGYTMHWFRQAPGK |
| 8845) | | GLEWVASIDTGGDTTYLPSVKGRFTISRDNSKNTLYLQMNSLR |
| | | AEDTAVYYCATDRNGGGAFDVWGOGTLVTVSS |
| (SEQ ID NO. | CD98-71 | QVQLVESGGGVVQPGRSLRLSCAASGFISTYYMSWVRQAPGKG |
| 8846) | | PEWVSTTGADGTTYYADSVNGRFTVSQDNAKNTVYLQMNSLRA |
| | | EDTAVYYCARYEDGPGFDIWGQGTLVTVSS |
| (SEQ ID NO. | CD98-84 | QVQLVESGGGVVQPGRSLRLSCAASGYTLPMSMGWYRQAPGKD |
| 8847) | | REGVAAIAGDGRTDYADSVKGRFTISRDNSKNTLYLQMNSLRA |
| | | EDTAVYYCARGADWNYGYWYFDLWGOGTLVTVSS |
| (SEQ ID NO. | CD98-9 | QVQLVESGGGVVQPGRSLRLSCAASGFPEGGYSVGWFRQAPGK |
| 8848) | | EREGVAVIDRDGSTTYADSVKGRFTISRDNANDTLYLQMNSLR |
| | | AEDTAVYYCAKSAKYYYEGSYYYVHFDYWGOGTLVTVSS |
| (SEQ ID NO. | CD98-94 | QVQLVESGGGVVQPGRSLRLSCAASGFTTTTYTMGWFRQAPGK |
| 8849) | | GRELVAVIDSDGDTAYAESVEGRETISRDNAKRMVYLQMNSLR |
| | | AEDTAVYYCARDKGGYGDYPFFLEDYWGQGTLVTVSS |
| (SEQ ID NO. | ALK1-13 | QVQLVESGGGVVQPGRSLRLSCAASGFTFSSYAMSWFROAPGK |
| 8850) | | GRELVAAIDSDGSTSYADSVEGRETISRDNAKRMVYLQMNSLR |
| | | AEDTAVYYCARVDFYYYYMDVWGQGTLVTVSS |
| (SEQ ID NO. | FLT1-39 | QVQLVESGGGVVQPGRSLRLSCAASGATSNTYYMGWFRQAPGK |
| 8851) | | EREGVATIRRDDLTAYTDSVKGRFTISRDNTKNTLYLQMNSLR |
| | | AEDTAVYYCARLDSNGYSYWGOGTLVTVSS |
| (SEQ ID NO. | FLT1-4 | QVQLVESGGGVVQPGRSLRLSCAASGRSNENYFLAWYRQAPGK |
| 8852) | | DREGVAVIYSDGKTVYADSVKGRFTISRDNSKNTLYLQMNSLR |
| | | AEDTAVYYCTEYGFKNGYAFDAWGQGTLVTVSS |
| (SEQ ID NO. | FLT1-49 | QVQLVESGGGVVQPGRSLRLSCAASGATSNTYYMGWFRLAPGK |
| 8853) | | EREGVASIYTQDGRTAYADSVKGRFTISEDNAKNTVYLQMNSL |
| | | RAEDTAVYYCARIDIDGYAYWGOGTLVTVSS |
| (SEQ ID NO. | FLT1-6 | QVQLVESGGGVVQPGRSLRLSCAASGSSDEIYAMAWFROAPGK |
| 8854) | | GRELVAAIENDGTTTYAHSVKGRFTISADTSKNTAYLQMNSLR |
| | | AEDTAVYYCARDSTSWWYFDLWGQGTLVTVSS |
| (SEQ ID NO. | IGF1R-17 | QVQLVESGGGVVQPGRSLRLSCAASGPSASAKAMAWFRQSVGK |
| 8855) | | EREGIAAIRHDGGETWYAASVKGRFTISRDNAKNTLYLQMNSL |
| | | RAEDTAVYYCVRDHGGARDIWGQGTLVTVSS |
| (SEQ ID NO. | IGF1R-37 | QVQLVESGGGVVQPGRSLRLSCAASGLSVSDSSMAWFRQRPGQ |
| 8856) | | EREGIAAIDTGDGSTYYLNSVEGRFTISHDNAKNTLYLQMNSL |
| | | RAEDTAVYYCVRQQRDGLVNFWGOGTLVTVSS |
| (SEQ ID NO. | INSULINR- | QVQLVESGGGVVQPGRSLRLSCAASGFTVGARAMAWYRQAPGK |
| 8857) | 87 | DREGVAAIDSDGIASYADSVKGRFTISRDNAKNTLYLQMNSLR |
| | | AEDTAVYYCARVSGSYGDYFDYWGQGTLVTVSS |
| (SEQ ID NO. | INSULINR- | QVQLVESGGGVVQPGRSLRLSCAASGYTEDESDMGWFRQAPGK |
| 8858) | 96 | GRELVSTISPDGTTYYADSVEGRETISRDNAKRMVYLQMNSLR |
| | | AEDTAVYYCARWRRSWFDTWGQGTLVTVSS |
| (SEQ ID NO. | LDLR-13 | QVQLVESGGGVVQPGRSLRLSCAASGYTVATYFIGWFRQAPGK |
| 8859) | | AREGVATITSDGRTKYADSVKGRFTISRDNAKNTVYLQMNSLR |
| | | AEDTAVYYCASRSIAAAGEGYWGQGTLVTVSS |
| (SEQ ID NO. | LRP1C2-46 | QVQLVESGGGVVQPGRSLRLSCAASGYPSCMGWARQAPGKGLE |
| 8860) | | WVACIERRGRIHYAASVKGRFTISRDNSKNTLYLQMNSLRAED |
| | | TAVYYCASODSSSYDAFDIWGQGTLVTVSS |
| (SEQ ID NO. | LRP1C3-24 | QVQLVESGGGVVQPGRSLRLSCAASGGAYRKYYMGWFROAPGK |
| 8861) | | GRELVAAIESDGTTDYADSVEGRETISRDNAKRMVYLQMNSLR |
| | | AEDTAVYYCASVIGGNSEDYWGOGTLVTVSS |
| (SEQ ID NO. | LRP1C4-33 | QVQLVESGGGVVQPGRSLRLSCAASGDANEFEWIGWYRQAPGK |
| 8862) | | GRELVARIGSSGPRYADSVEGRFTISRDNSKNTLYLQMNSLRA |
| | | EDTAVYYCAATHCSFLTTSVAGCTDDKYWGQGTLVTVSS |
| (SEQ ID NO. | SCARB1-3 | QVQLVESGGGVVQPGRSLRLSCAASGFTLGTNCMAWFRQAPGK |
| 8863) | | ERERVASIGSDGRTYYADSVKGRFTISRDNSKNTLYLQMNSLR |
| | | AEDTAVYYCARAPGIQLWKEDYWGQGTLVTVSS |
| (SEQ ID NO. | SCARBI- | QVQLVESGGGVVQPGRSLRLSCAASGFTNSKTYLGWVRRAPGK |
| 8864) | 49 | GLEWVAAIAPSTGSTYYDDSVKGRFTISRDNSKNTLYLQMNSL |
| | | RAEDTAVYYCASQGSSWYENDYWGQGTLVTVSS |
| (SEQ ID NO. | IGF1R-55 | QVQLVESGGGVVQPGRSLRLSCAASGFREDDYAMGWFRQAPGK |
| 8865) | | EREGVAGYKSDGSTTYADSVKGRFTISRDNSKNTLYLQMNSLR |
| | | AEDTAVYYCASRALYGGKPFVIWGQGTLVTVSS |
| (SEQ ID NO. | LRP1C4-31 | QVQLVESGGGVVQPGRSLRLSCAASGSPDRRACMGWYROAPGK |
| 8866) | | ERERVAEIDTDGNTSYADSVKGRFTISRDNSKNTVYLQMNSLR |
| | | AEDTAVYYCALQPGCPPATLFYWRKTGEGNWGQGTLVTVSS |
| (SEQ ID NO. | LRPIC4-80 | QVQLVESGGGVVQPGRSLRLSCAASGFTEDDTDMGWFRQAPGK |
| 8867) | | ERERVSTINSDGGTTYYADSVKGRFTISRDNSKNTLYLQMNSL |
| | | RAEDTAVYYCARARVTDAGWQYFDYWGOGTLVTVSS |
| (SEQ ID NO. | ALK1-15 | QVQLVESGGGVVQPGRSLRLSCAASGFTFSDYYIGWFRQAPGK |
| 8868) | | ERERVAQIDKNGVTNYADAVKGRFTISRDNSKNTLYLQMNSLR |
| | | AEDTAVYYCAGVDAFNYMEVWGQGTLVTVSS |
| (SEQ ID NO. | IGF1R-13 | QVQLVESGGGVVQPGRSLRLSCAASGDENDARAEWGWFRQAPG |
| 8869) | | KGRELVASLASDGSTIYANPVEGRFTISRDNAKNTLYLQMNSL |
| | | RAEDTAVYYCARSGASSSWYWFDYWGQGTLVTVSS |
| (SEQ ID NO. | IGF1R-96 | QVQLVESGGGVVQPGRSLRLSCAASGSIHNRRHMGWFRQAAGK |
| 8870) | | EREGVAAIDIAGNTRISDSVKGRFTISHDKDENRLYLQMNSLR |
| | | AEDTAVYYCAIQSSDGRGTVYWGOGTLVTVSS |
| (SEQ ID NO. | INSULINR- | QVQLVESGGGVVQPGRSLRLSCAASGRRGNNYSMAWYROAPGK |
| 8871) | 86 | DREGVAAIDSDGDTSYADPVKGRFTISRDNSKNTLYLQMNSLR |
| | | AEDTAVYYCAVATGEYRRYFDVWGQGTLVTVSS |
| (SEQ ID NO. | LRP1C3-93 | QVQLVESGGGVVQPGRSLRLSCAASGFAFTKYHMSWFRQAPGK |
| 8872) | | GRELVSHISAGGRTYYSDSVEGRETISRDNAKRMVYLQMNSLR |
| | | AEDTAVYYCVKESRPHSYGDHWGQGTLVTVSS |
| (SEQ ID NO. | TfR-1687 | QVQLVESGGGVVQPGRSLRLSCAASGVTFDEVDMGWARQAPGK |
| 8873) | | GLEWVSRIASDGRTYYTDSVKGRFTISRDNAKRMVYLQMNSLR |
| | | AEDTAVYYCTRRTIWGDAFDIWGQGTLVTVSS |
| (SEQ ID NO. | TfR-1704 | QVQLVESGGGVVQPGRSLRLSCAASGYSPAKYAMGWARQAPGK |
| 8874) | | GLEWVATIGRDGVAFYADSVKGRFTISRDNSKNIVYLQMNSLR |
| | | AEDTAVYYCAKGDYGDFQHYYLDYWGQGTLVTVSS |
| (SEQ ID NO. | TfR-1744 | QVQLVESGGGVVQPGRSLRLSCAASGFTFSNNWMHWFRQAPGK |
| 8875) | | GRELVAIVDDDGDPTYAPSVEGRFTISRDNSKNTLYLQMNSLR |
| | | AEDTAVYYCASSAGSYYYYGMDVWGQGTLVTVSS |
| (SEQ ID NO. | TfR-1715 | QVQLVESGGGVVQPGRSLRLSCAASGFTIRDSDMGWFRQAPGK |
| 8876) | | GRELVSTITSGGTTWYADSVEGRETISRDNAKRMVYLQMNSLR |
| | | AEDTAVYYCARSYNWNFYGMDVWGQGTLVTVSS |
| (SEQ ID NO. | TfR-1728 | QVQLVESGGGVVQPGRSLRLSCAASGFTFDDTDMGWFRQAPGK |
| 8877) | | ERERVSSISVDGNTWYSESVKGRFTISRDNSKNTVYLQMNSLR |
| | | AEDTAVYYCARGRMGSSWYWYFDLWGQGTLVTVSS |
| (SEQ ID NO. | TfR-1651 | QVQLVESGGGVVQPGRSLRLSCAASGIVEGSHDMGWARQAPGK |
| 8878) | | GLEWVATLTKAGQTLYTDTVKGRFTISADTSKNTAYLQMNSLR |
| | | AEDTAVYYCASDSRRDGWLDSWGOGTLVTVSS |
| (SEQ ID NO. | TfR1_6wrv | KKKYKITVTVPGKKEEEVIEVEEKDLTDVLTQKALEAAKKWGV |
| 8879) | 68_7_9 | KSGVKAEAEELE |
| (SEQ ID NO. | TfR-1409- | QVQLVESGGGVVQPGRSLRLSCAASGFSFSNFGMSWVRQARGK |
| 8880) | 51-105 | GLEWVSSIGSDGRAYYADSVKHRFTISRDNAKNTLYLQMNSLR |
| | | AEDTAVYYCARAPSRHGFDYWGQGTLVTVSS |
| (SEQ ID NO. | TfR-1409- | QVQLVESGGGVVQPGRSLRLSCAASGFSFSNFGMSWVRQARGK |
| 8881) | 51-G55I | GLEWVSSIGSDIRAYYADSVKGRFTISRDNAKNTLYLQMNSLR |
| | | AEDTAVYYCARAPSRHGFDYWGQGTLVTVSS |
| (SEQ ID NO. | TfR-1746 | QVQLVESGGGVVQPGRSLRLSCAASGFTFSNYAMTWERQAPGK |
| 8882) | | GRELVSAINGAGVTYYADSVEGRETISRDNAKRMVYLQMNSLR |
| | | AEDTAVYYCARASYYDFWSGYEDYYGMDVWGQGTLVTVSS |
| (SEQ ID NO. | TfR-1765 | QVQLVESGGGVVQPGRSLRLSCAASGFSFNDADMGWARQAPGK |
| 8883) | | GLEWVSTITADGSPYYSDSVKGRETISRDNSKNTLYLQMNSLR |
| | | AEDTAVYYCARAAGRSIGWFDPWGQGTLVTVSS |
| (SEQ ID NO. | TfR-1679 | QVQLVESGGGVVQPGRSLRLSCAASGAGYRPTTMGWFRQAPGK |
| 8884) | | ERERVAAISASGVSTNYADSVKGRFTISRDNSKNTLYLQMNSL |
| | | RAEDTAVYYCARAITPGGYFDSWGQGTLVTVSS |
| (SEQ ID NO. | TfR-7063 | QVQLVESGGGVVQPGRSLRLSCAASGFSFSNFGMSWVRQARGK |
| 8885) | | GLEWVSSIGSDGRAYYADSVEGRETISRDNAKNTLYLQMNSLR |
| | | AEDTAVYYCARAPSRHGFDYWGQGTLVTVSS |
| (SEQ ID NO. | TfR-1409- | QVQLVESGGGVVQPGRSLRLSCAASGFSFSNFGMSWVROARGK |
| 8886) | 51-A60M | GLEWVSSIGSDGRAYYMDSVKGRETISRDNAKNTLYLQMNSLR |
| | | AEDTAVYYCARAPSRHGFDYWGQGTLVTVSS |
| (SEQ ID NO. | CD98-62-10 | QVQLVESGGGVVQPGRSLRLSCAASGFTFSSYAMSWFRQALGK |
| 8887) | | DREGVAAIDSDGGTSYGESVKGRETISRDNSKNTVYLQMNSLR |
| | | AEDTAVYYCARAAPVGDDYYRMDYWGQGTLVTVSS |
| (SEQ ID NO. | CD98-7-2 | QVQLVESGGGVVQPGRSLRLSCAASGFTISGYSMHWFRQAPGK |
| 8888) | | GLEWVASIDTGGDRTYAPAVKGRFTISRDNSKNTLYLQMNSVR |
| | | AEDTAVYYCATDRNGGGAFDVWGQGTLVTVSS |
| (SEQ ID NO. | CD98-7-34 | QVQLVESGGGVVQPGRSLRLSCAASGFTISGYTMHWFRQAPGK |
| 8889) | | GLEWVASIDTGGDTTYSPSVKGRFTISRDNSSNTLYLQMNSLR |
| | | AEDTAVYYCATDRNGGGAFDVWGQGTLVTVSS |
| (SEQ ID NO. | CD98-7-6 | QVQLVESGGGVVQPGRSLRLSCAASGFTISGYTMHWFRQAPGK |
| 8890) | | GLEWVASDDTGGDTTYLPSVKRRFTISRDNSKNTLYLQMNSLR |
| | | AEDTAVYYCATDRNGGGAFDVWGOGTLVTVSS |
| (SEQ ID NO. | FLT1-49-11 | QVQLVESGGGVVQPGRSLRLSCAASGATSNTYGMGWFRLAPGK |
| 8891) | | EREGVASIYTODGRTAYADSVKGRFTISEDNAKNTVYLQMNSL |
| | | RAEDTAVYYCARIDIDGYAYWGQGTLVTVSS |
| (SEQ ID NO. | FLT1-49-31 | QVQLVESGGGVVQPGRSLRLSCAASGATSNTYYMGWFRLAPGK |
| 8892) | | EREGVASIYTODGRTAVADSVKGRFTISEDNAKNTVYLQMNSL |
| | | RAEDTAVYYCARIDIDGYAYWGQGTLVTVSS |
| (SEQ ID NO. | FLT1-49-51 | QVQLVESGGGVVQPGRSLRLSCAASGATSNTYYMGWFRLAPGK |
| 8893) | | EREGVASIYTODGRTAYIKSVKGRFTISEDNAKNTVYLQMNSL |
| | | RAEDTAVYYCARIDIDGYAYWGQGTLVTVSS |
| (SEQ ID NO. | FLT-1-49-41 | QVQLVESGGGVVQPGRSLRLSCAASGATSNTYYMGWFRLAPGK |
| 8894) | | EREGVASIYTODGRTAYADSVKGRFTISEDNAKNTVYLQMNSL |
| | | RAEDTAVYYCARHDIDGYVYWGQGTLVTVSS |

### IV. Production

i. Production of Barcodes

Disclosed herein are methods and systems for the production of barcodes for use in systems and methods of the present disclosure. In some embodiments, barcodes, as described herein, may be generated rapidly (e.g., in about a week, about 2 weeks, about 3 weeks, about 4 weeks, about 1 month, about 2 months, about 3 months, about 4 months, about 5 months, about 6 months, or about 1 year). In some embodiments, for example, between about 100 to about 1,000 barcodes may be generated rapidly. In some embodiments, between about 10 to about 1000 barcodes may be generated rapidly. In some embodiments, between about 10 to about 10,000 barcodes may be generated rapidly. While large numbers of barcodes, as described herein, may be generated rapidly, such barcodes are also robust, in that barcodes generated using the methods disclosed herein may bind specifically and with different affinities to a known set of binders.

In accordance with various embodiments, barcodes as described herein may be synthesized using a nucleic acid (e.g., oligonucleotide) array. In some embodiments, barcodes as described herein may be synthesized using a DNA array. In some embodiments, nucleic acids (e.g., oligonucleotides) of a nucleic acid array are expressed into barcodes. In some embodiments, barcodes as described herein may be synthesized using nucleic acid library. In some embodiments, a nucleic acid library is synthesized using a nucleic acid array. In some embodiments, nucleic acids (e.g., oligonucleotides) of a nucleic acid library are expressed into barcodes.

In some embodiments, a barcode nucleic acid library comprises about 1 or more, about 2 or more, about 3 or more, about 4 or more, about 5 or more, about 10 or more, about 50 or more, about 100 or more, about 200 or more, about 300 or more, about 400 or more, about 500 or more, about 600 or more, about 700 or more, about 800 or more, about 900 or more, about 1000 or more, about 2000 or more, about 3000 or more, about 4000 or more, or about 5000 or more potential barcodes. In some embodiments, a nucleic acid library comprises one or more potential barcode sequences. Such potential barcode sequences may be screened for functionality as peptide barcodes (i.e., after translation of potential barcode nucleic acid sequences) using one or more methods described herein.

Barcodes of the present disclosure may be screened for one or more specific properties. In some embodiments, a barcode may be screened for specific binding (e.g., specificity, binding affinity) to a binder. In some embodiments, a barcode may be screened for specific binding to one or more binders. In some embodiments, a barcode may be screened for specific binding to at least a binder. In some embodiments, a barcode may be screened for specific binding to at most a binder. In some embodiments, a barcode may be screened for specific binding to multiple binders.

As may be understood by a person of ordinary skill in the art, a barcode is designed to be distinct (i.e., unique (e.g., have a unique sequence)) in a pool of barcodes. Such distinction may be achieved, in some embodiments, by changing one or more amino acids in a barcode. In some embodiments, a barcode is distinct from other barcodes in a pool of barcodes by 1 amino acid. In some embodiments, a barcode is distinct from other barcodes in a pool of barcodes by at least 1 amino acid. In some embodiments, a barcode is distinct from other barcodes in a pool of barcodes by at most 1 amino acid. In some embodiments, a barcode is distinct from other barcodes in a pool of barcodes by 2, 3, 4, 5, 6, 7, 8, 9, 10, 11, 12, 13, 14, 15, 16, 17, 18, 19, 20, 21, 22, 23, 24, or 25 amino acids. In some embodiments, a barcode is distinct from other barcodes in a pool of barcodes by at least 2 amino acids. In some embodiments, a barcode is distinct from other barcodes in a pool of barcodes by at most 50 amino acids.

ii. Production of Barcoded Cargos

Barcoded cargos in accordance with the present invention may be produced in various ways. In some embodiments, cargo-barcode nucleic acid sequence pairs may be inserted into a plasmid to allow for expression in different expression systems (e.g., protein expression systems). In some embodiments, at least one cargo-barcode nucleic acid sequence pair is inserted into a plasmid. In some embodiments, at least two cargo-barcode nucleic acid sequence pairs are inserted into a plasmid. In some embodiments, at least three cargo-barcode nucleic acid sequence pairs are inserted into a plasmid. In some embodiments, one or more cargo-barcode nucleic acid sequence pairs are inserted into a plasmid.

In some embodiments, a cargo-barcode nucleic acid sequence may comprise additional sequences. In some embodiments, a cargo-barcode nucleic acid sequence may comprise additional nucleic acid sequences. In some embodiments, a cargo-barcode nucleic acid sequence may comprise a universal motif sequence. In some embodiments, a cargo-barcode nucleic acid sequence may comprise at least one universal motif sequence. In some embodiments, a cargo-barcode nucleic acid sequence may comprise at least two universal motif sequences. In some embodiments, a cargo-barcode nucleic acid sequence may comprise two or more universal motif sequences.

In some embodiments, at least one cargo-barcode nucleic acid sequences in a pool of cargo-barcode nucleic acid sequences may comprise a universal motif sequence. In some embodiments, all cargo-barcode nucleic acid sequences in a pool of cargo-barcode nucleic acid sequences may comprise a universal motif sequence.

Different plasmids may be used to produce technologies described herein. In some embodiments, a plasmid is a DNA plasmid. In some embodiments, a plasmid is an RNA plasmid. In some embodiments, a plasmid is a fertility F-plasmid. In some embodiments, a plasmid is a resistance plasmid. In some embodiments, a plasmid is a virulence plasmid. In some embodiments, a plasmid is a degradative plasmid. In some embodiments, a plasmid is a Col plasmid.

Different hosts (e.g., host cell, host cell line, etc.) may be used to produce technologies described herein. In some embodiments, a host is a mammalian host. In some embodiments, a host is a non-mammalian host. In some embodiments, a host is an insect. In some embodiments, a host is a bacteria. In some embodiments, a host is E. coli.

In some embodiments, a cargo-barcode pair is expressed in vitro. In some embodiments, a cargo-barcode pair is expressed in vivo. In some embodiments, a cargo-barcode pair is expressed from RNA. In some embodiments, a cargo-barcode pair is expressed from transcribed RNA. In some embodiments, a cargo-barcode pair is expressed from DNA. In some embodiments, a cargo-barcode pair is expressed using protein components (e.g., required for protein translation).

After expression of barcoded cargo constructs, constructs may be purified from the pool. In some embodiments, purification may be performed using a universal motif. In some embodiments, purification may be performed using HIS tag, FLAG tag, HALO tag, SNAP tag, Avitag, Twin strep tag, or any other tag based method of protein purification known in the art.

iii. Production of Binders

Disclosed herein are methods and systems for the production of binders for use in systems and methods of the present disclosure. In some embodiments, binders, as described herein, may be generated rapidly (e.g., in about a week, about 2 weeks, about 3 weeks, about 4 weeks, about 1 month, about 2 months, about 3 months, about 4 months, about 5 months, about 6 months, or about 1 year). In some embodiments, for example, between about 100 to about 1000 binders may be generated rapidly. In some embodiments, between about 10 to about 1000 binders may be generated rapidly. In some embodiments, between about 10 to about 10,000 binders may be generated rapidly. While large numbers of binders, as described herein, may be generated rapidly, such binders are also robust, in that binders generated using the methods disclosed herein may bind specifically and with different affinities to a known set of barcodes.

In some embodiments, a binder nucleic acid library comprises about 1 or more, about 2 or more, about 3 or more, about 4 or more, about 5 or more, about 10 or more, about 50 or more, about 100 or more, about 200 or more, about 300 or more, about 400 or more, about 500 or more, about 600 or more, about 700 or more, about 800 or more, about 900 or more, about 1000 or more, about 2000 or more, about 3000 or more, about 4000 or more, or about 5000 or more potential binders. In some embodiments, a nucleic acid library comprises one or more potential binder sequences. Such potential binder sequences may be screened for functionality as polypeptide binders (i.e., after translation of potential nucleic acid binder sequences) using one or more methods described herein.

Binders in accordance with the present invention may be produced in various ways. In some embodiments, a binder nucleic acid sequence may be inserted into a plasmid to allow for expression in different expression systems. In some embodiments, at least one binder nucleic acid sequence is inserted into a plasmid. In some embodiments, at least two binder nucleic acid sequences are inserted into a plasmid. In some embodiments, at least three binder nucleic acid sequences are inserted into a plasmid. In some embodiments, one or more binder nucleic acid sequences are inserted into a plasmid.

In some embodiments, a binder nucleic acid sequence is attached to one or more genes. In some embodiments, a binder nucleic acid sequence is attached to one or more genes prior to insertion into a plasmid. In some embodiments, a binder nucleic acid sequence is attached to one or more genes after insertion into a plasmid. In some embodiments, a binder nucleic acid sequence is attached to a bacteriophage gene. In some embodiments, a binder nucleic acid sequence is attached to an m13 bacteriophage gene. In some embodiments, a binder nucleic acid sequence is attached to gene 3 (i.e., that encodes for gene 3 protein) of m13 bacteriophage.

In some embodiments, plasmids (e.g., containing binder sequences, containing binder and bacteriophage sequences, etc.) may be transformed into a host. In some embodiments, plasmids may be transformed into a host and expressed. In some embodiments, plasmids are transformed into a bacterium. In some embodiments, plasmids are transformed into f. coli.

In some embodiments, expression of plasmids results in phage production. In some embodiments, expression of plasmids results in display of a binder on a surface of a phage. In some embodiments, expression of plasmids results in display of two binders on a surface of a phage. In some embodiments, expression of plasmids results in display of at least one binder on a surface of a phage. In some embodiments, expression of plasmids results in display of one or more binders on a surface of a phage. In some embodiments, expression of plasmids results in display of one or more binders on one or more surfaces of a phage. In some embodiments, expression of plasmids results in display of at least one binder on one or more surfaces of a phage.

Following phage production, the resulting pool may be purified to determine the presence of one or more polypeptide binders. In some embodiments, purification may be performed using a universal motif. In some embodiments, purification may be performed using HIS tag, FLAG tag, HALO tag, SNAP tag, Avitag, Twin strep tag, or any other tag based method of protein purification known in the art.

In some embodiments, a purified binder pool may be highly diverse. In some embodiments, a purified binder pool may not be highly diverse. In some embodiments, a purified binder pool is subjected to screening methods to select binders of interest.

Binders of the present disclosure may be screened for one or more specific properties. In some embodiments, a binder may be screened for specific binding to a barcode. In some embodiments, a binder may be screened for specific binding to one or more barcodes. In some embodiments, a binder may be screened for specific binding to at least a barcode. In some embodiments, a binder may be screened for specific binding to at most a barcode. In some embodiments, a binder may be screened for specific binding to multiple barcodes.

As may be understood by a person of ordinary skill in the art, a binder is designed to be distinct (i.e., unique (e.g., have a unique sequence)) in a pool of binders. Such distinction may be achieved, in some embodiments, by changing one or more amino acids in a binder. In some embodiments, a binder is distinct from other binders in a pool of binders by 1 amino acid. In some embodiments, a binder is distinct from other binder in a pool of binders by at least 1 amino acid. In some embodiments, a binder is distinct from other binder in a pool of binders by at most 1 amino acid. In some embodiments, a binder is distinct from other binder in a pool of binders by 2, 3, 4, 5, 6, 7, 8, 9, 10, 11, 12, 13, 14, 15, 16, 17, 18, 19, 20, 21, 22, 23, 24, or 25 amino acids. In some embodiments, a binder is distinct from other binder in a pool of binders by at least 2 amino acids. In some embodiments, a binder is distinct from other binder in a pool of binders by at most 1000 amino acids.

iv. Production of Shuttles

Disclosed herein are methods and systems for the production of shuttles for use in systems and methods of the present disclosure. In some embodiments, shuttles, as described herein, may be generated rapidly (e.g., in about a week, about 2 weeks, about 3 weeks, about 4 weeks, about 1 month, about 2 months, about 3 months, about 4 months, about 5 months, about 6 months, or about 1 year). In some embodiments, for example, between about 100 to about 1,000 shuttles may be generated rapidly. In some embodiments, between about 10 to about 1000 shuttles may be generated rapidly. In some embodiments, between about 10 to about 10,000 shuttles may be generated rapidly. While large numbers of shuttles, as described herein, may be generated rapidly, such shuttles are also robust, in that shuttles generated using the methods disclosed herein may bind specifically and with different affinities to different targets.

In accordance with various embodiments, shuttles as described herein may be synthesized using a nucleic acid (e.g., oligonucleotide) array. In some embodiments, shuttles as described herein may be synthesized using a DNA array. In some embodiments, nucleic acids (e.g., oligonucleotides) of a nucleic acid array are expressed into shuttles. In some embodiments, shuttles as described herein may be synthesized using nucleic acid library. In some embodiments, a nucleic acid library is synthesized using a nucleic acid array. In some embodiments, nucleic acids (e.g., oligonucleotides) of a nucleic acid library are expressed into shuttles.

In some embodiments, a shuttle nucleic acid library comprises about 1 or more, about 2 or more, about 3 or more, about 4 or more, about 5 or more, about 10 or more, about 50 or more, about 100 or more, about 200 or more, about 300 or more, about 400 or more, about 500 or more, about 600 or more, about 700 or more, about 800 or more, about 900 or more, about 1000 or more, about 2000 or more, about 3000 or more, about 4000 or more, or about 5000 or more potential shuttles. In some embodiments, a nucleic acid library comprises one or more potential shuttle sequences. Such potential shuttle sequences may be screened for functionality as peptide shuttles (i.e., after translation of potential shuttle nucleic acid sequences) using one or more methods described herein.

Shuttles of the present disclosure may be screened for one or more specific properties. In some embodiments a shuttle may be screened for delivery to a specific cell, tissue, or organ of interest (e.g., targeted delivery). In some embodiments a shuttle may be screened for delivery to one or more specific cells, tissues, or organs of interest. In some embodiments a shuttle may be screened for delivery to at least a specific cell, tissue, or organ of interest. In some embodiments a shuttle may be screened for delivery to at most a specific cell, tissue, or organ of interest. In some embodiments a shuttle may be screened for delivery to more than one (e.g., multiple) specific cell, tissue, or organ of interest.

In some embodiments, a shuttle may be screened for specific binding (e.g., specificity, binding affinity) to a target. In some embodiments, a shuttle may be screened for specific binding to one or more targets. In some embodiments, a shuttle may be screened for specific binding to at least a target. In some embodiments, a shuttle may be screened for specific binding to at most a target. In some embodiments, a shuttle may be screened for specific binding to multiple targets.

As may be understood by a person of ordinary skill in the art, a shuttle is designed to be distinct (i.e., unique (e.g., have a unique sequence)) in a pool of shuttles. Such distinction may be achieved, in some embodiments, by changing one or more amino acids in a shuttle. In some embodiments, a shuttle is distinct from other shuttles in a pool of shuttles by 1 amino acid. In some embodiments, a shuttle is distinct from other shuttles in a pool of shuttles by at least 1 amino acid. In some embodiments, a shuttle is distinct from other shuttles in a pool of shuttles by at most 1 amino acid. In some embodiments, a shuttle is distinct from other shuttles in a pool of shuttles by 2, 3, 4, 5, 6, 7, 8, 9, 10, 11, 12, 13, 14, 15, 16, 17, 18, 19, 20, 21, 22, 23, 24, or 25 amino acids. In some embodiments, a shuttle is distinct from other shuttles in a pool of shuttles by at least 2 amino acids. In some embodiments, a shuttle is distinct from other shuttles in a pool of shuttles by at most 50 amino acids. In some embodiments, a shuttle is distinct from other shuttles in a pool of shuttles by at most 100 amino acids. In some embodiments, a shuttle is distinct from other shuttles in a pool of shuttles by at most 150 amino acids.

v. Production of Shuttle-Cargos

The present disclosure contemplates that in some embodiments, a cargo as described herein is operably linked to a shuttle (e.g., a shuttle moiety (e.g., a peptide shuttle)) as described herein. In some embodiments, a cargo is operably linked to a shuttle to form shuttle-cargos.

Shuttle-cargos in accordance with the present invention may be produced in various ways. In some embodiments, shuttle-cargo nucleic acid sequence pairs may be inserted into a plasmid to allow for expression in different expression systems (e.g., protein expression systems). In some embodiments, at least one shuttle-cargo nucleic acid sequence pair is inserted into a plasmid. In some embodiments, at least two shuttle-cargo nucleic acid sequence pairs are inserted into a plasmid. In some embodiments, at least three shuttle-cargo nucleic acid sequence pairs are inserted into a plasmid. In some embodiments, one or more shuttle-cargo nucleic acid sequence pairs are inserted into a plasmid.

In some embodiments, a shuttle-cargo nucleic acid sequence may comprise additional sequences. In some embodiments, a shuttle-cargo nucleic acid sequence may comprise additional nucleic acid sequences. In some embodiments, a shuttle-cargo nucleic acid sequence may comprise a universal motif sequence. In some embodiments, a shuttle-cargo nucleic acid sequence may comprise at least one universal motif sequence. In some embodiments, a shuttle-cargo nucleic acid sequence may comprise at least two universal motif sequences. In some embodiments, a shuttle-cargo nucleic acid sequence may comprise two or more universal motif sequences.

In some embodiments, at least one shuttle-cargo nucleic acid sequence in a pool of shuttle-cargo nucleic acid sequences may comprise a universal motif sequence. In some embodiments, all shuttle-cargo nucleic acid sequences in a pool of shuttle-cargo nucleic acid sequences may comprise a universal motif sequence.

Different plasmids may be used to produce technologies described herein. In some embodiments, a plasmid is a DNA plasmid. In some embodiments, a plasmid is an RNA plasmid. In some embodiments, a plasmid is a fertility F-plasmid. In some embodiments, a plasmid is a resistance plasmid. In some embodiments, a plasmid is a virulence plasmid. In some embodiments, a plasmid is a degradative plasmid. In some embodiments, a plasmid is a Col plasmid.

Different hosts (e.g., host cell, host cell line, etc.) may be used to produce technologies described herein. In some embodiments, a host is a mammalian host. In some embodiments, a host is a non-mammalian host. In some embodiments, a host is an insect. In some embodiments, a host is a bacteria. In some embodiments, a host is E. coli.

In some embodiments, a shuttle-cargo pair is expressed in vitro. In some embodiments, a shuttle-cargo pair is expressed in vivo. In some embodiments, a shuttle-cargo pair is expressed from RNA. In some embodiments, a shuttle-cargo pair is expressed from transcribed RNA. In some embodiments, a shuttle-cargo pair is expressed from DNA. In some embodiments, a shuttle-cargo pair is expressed using protein components (e.g., required for protein translation).

After expression of shuttle-cargo constructs, constructs may be purified from the pool. In some embodiments, purification may be performed using a universal motif. In some embodiments, purification may be performed using HIS tag, FLAG tag, HALO tag, SNAP tag, Avitag, Twin strep tag, or any other tag based method of protein purification known in the art.

The present disclosure appreciates that in some embodiments a cargo is or comprises a therapeutic agent. For example, in some embodiments, a cargo is or comprises an antibody. In some embodiments, a cargo is or comprises one or more components of an antibody (e.g., one or more variable light (VL) chains, variable heavy (VH) chains, and/or complementarity- determining regions (CDRs) of an antibody). In some embodiments, a cargo is or comprises an antibody drug conjugate (ADC). In some embodiments, a cargo is or comprises an oligonucleotide. In some embodiments, a cargo is or comprises an antibody associated with (e.g., covalently, non-covalently) an oligonucleotide.

Shuttles (e.g., a peptide shuttle) as disclosed herein may be associated (e.g., covalently, non-covalently) with a cargo as described herein (e.g., a cargo polypeptide) through various methods. Shuttles (e.g., a peptide shuttle) as disclosed herein may be associated with a therapeutic agent as described herein (e.g., a therapeutic polypeptide) through various methods. In some embodiments, for example, methods by which shuttles as described herein may be associated with (i) cargos as described herein, or (ii) therapeutic agents as described herein include, but are not limited to, recombinant fusion, click chemistry, sortase-based methods, transglutaminase-based methods, lysine/NHS conjugation, and cysteine/maleimide conjugation. As may be apparent to those of ordinary skill in the art reading the specification, many other methods may be used to associate shuttles with cargos and/or shuttles with therapeutic agents.

### V. Characterization

i. Samples

As described elsewhere in the present disclosure, a sample may be a biological sample. In some embodiments, a sample may contain one or more barcoded cargos. In some embodiments, a sample may contain one or more barcoded shuttle-cargos. In some embodiments, a sample may contain one or more barcoded cargo polypeptides. In some embodiments, a sample may contain one or more barcoded shuttle-cargo polypeptides.

In some embodiments, a sample is derived from an organism. In some embodiments, a sample is derived from an animal. In some embodiments, a sample is derived from an animal model of disease. In some embodiments, a sample is derived from a non-mammal. In some embodiments, a sample is derived from a mammal (e.g., a rodent, a mouse, a rat, a rabbit, a monkey, a dog, a cat, a sheep, cattle, a primate, and/or a pig). In some embodiments, a sample is derived from a mouse. In some embodiments, a sample is derived from a human. In some embodiments, a sample is derived from cells (e.g., in vitro). In some embodiments, a sample is a human cell line.

In some embodiments, a sample may be purified. In some embodiments, a sample may not be purified.

In some embodiments, a sample is obtained from cells that was treated with barcoded cargos. In some embodiments, a sample is obtained from cells that was not treated with barcoded cargos. In some embodiments, a sample is obtained from an animal that was treated with barcoded cargos. In some embodiments, a sample is obtained from an animal that was not treated with barcoded cargos. For example, in some embodiments, a sample is obtained from a human that was treated with barcoded cargo polypeptides (e.g., barcoded shuttle-cargo polypeptides).

In some embodiments, a sample is obtained from cells that was genetically modified. In some embodiments, a sample is obtained from cells that was modified by gene therapy. In some embodiments, a sample is obtained from cells that was genetically modified to include one or more barcoded cargos. In some embodiments, a sample is obtained from cells that was genetically modified to express a barcoded cargos. In some embodiments, a sample is obtained from cells that was genetically modified to include one or more barcodes. In some embodiments, a sample is obtained from cells that was genetically modified to express a barcodes. In some embodiments, a sample is obtained from cells that was genetically modified to include one or more binders. In some embodiments, a sample is obtained from cells that was genetically modified to express a binders.

In some embodiments, a sample is obtained from an animal that was genetically modified. In some embodiments, a sample is obtained from an animal that was modified by gene therapy. In some embodiments, a sample is obtained from an animal that was genetically modified to include one or more barcoded cargos. In some embodiments, a sample is obtained from an animal that was genetically modified to express a barcoded cargos. In some embodiments, a sample is obtained from an animal that was genetically modified to include one or more barcodes. In some embodiments, a sample is obtained from an animal that was genetically modified to express a barcodes. In some embodiments, a sample is obtained from an animal that was genetically modified to include one or more binders. In some embodiments, a sample is obtained from an animal that was genetically modified to express a binders.

ii. Fingerprints

Among other things, systems and methods described herein identify the advantages of nucleic acid sequencing techniques and apply them effectively to protein detection and measurement methods. For example, methods described herein may use several binders, with known binding specificities and affinities to different barcodes, that can be expressed on binding agents and mixed together in a single pool. Upon mixing with a pool of barcoded cargo polypeptides (i.e., proteins, each associated with a barcode as described herein (e.g., barcoded shuttle-cargo polypeptides)), each binder expressed on a binding agent binds to a one or more barcodes in the pool with known but varying affinities. Such a spectrum of affinities for a given barcode to one or more binders results in a distinct distribution of binder counts for a given barcode that can be determined through NGS, and is termed herein a ‘Barcode Fingerprint’. In some embodiments, the collective barcode fingerprints for a set of barcodes is termed herein a ‘Fingerprint Matrix’. Analogously, a spectrum of affinities of a binder to various (e.g., one or more) barcodes is termed herein as a ‘Binder Fingerprint’. In some embodiments, using the provided technologies the presence of a barcoded cargo polypeptide(s) or a barcoded shuttle-cargo polypeptide(s) can be detected, for example, in a complex solution, by extracting and sequencing the associated nucleic acid (e.g., detectable nucleic acid (e.g., DNA sequence, RNA sequence, etc.)) of the population of binding agents (e.g., phage) that bind to the barcoded cargo polypeptide(s) or a barcoded shuttle-cargo polypeptide(s). That is, for example, in some embodiments, the presence of a protein in a complex solution is determined not through a single binder, but through a specific combination of multiple binders that bind to a barcode associated with said protein in fixed, known proportions.

Fingerprints, as disclosed herein, have many advantages. In some embodiments, a fingerprint approach of detection allows for reduction of noise. For example, the use of multiple binders to detect a barcode in a complex solution introduces a redundancy into the detection methods that in turn reduces signal noise. Additionally, another advantage of the “fingerprint” approach is that partial non-specificities in the binders (e.g., to barcodes other than the barcode of interest to be detected) can be tolerated and compensated for by the computational prediction methods.

In some embodiments, binder sequences may be modified in order to change a fingerprint. In some embodiments, binder sequences may be modified in order to improve a fingerprint.

A barcode fingerprint, as described herein, for a given barcode may include affinity information of a given barcode to one or more binders. In some embodiments, a barcode fingerprint may include affinity information of a given barcode to one binder. In some embodiments, a barcode fingerprint may include affinity information of a given barcode to at least one binder. In some embodiments, a barcode fingerprint may include affinity information of a given barcode to 2, 3, 4, 5, 10, 20, 25, 50, 100, 200, 300, 400, 500, 600, 700, 800, 900, 1000, 2000, 3000, 4000, 5000, 6000, 7000, 8000, 9000, or 10,000 or more binders. In some embodiments, a barcode fingerprint may include affinity information of a given barcode to at most 10,000 binders.

A binder fingerprint, as described herein, for a given binder may include affinity information of a given binder to one or more barcodes. In some embodiments, a binder fingerprint may include affinity information of a given binder to one barcode. In some embodiments, a binder fingerprint may include affinity information of a given binder to at least one barcode. In some embodiments, a binder fingerprint may include affinity information of a given binder to 2, 3, 4, 5, 10, 20, 25, 50, 100, 200, 300, 400, 500, 600, 700, 800, 900, 1000, 2000, 3000, 4000, 5000, 6000, 7000, 8000, 9000, or 10,000 or more barcodes. In some embodiments, a binder fingerprint may include affinity information of a given binder to at most 10,000 barcodes.

As discussed herein, in some embodiments, multiple barcode fingerprints for a set of barcodes may be grouped together and is termed herein a ‘Fingerprint Matrix’. In some embodiments, a fingerprint matrix may comprise one barcode fingerprint. In some embodiments, a fingerprint matrix may comprise at least one barcode fingerprint. In some embodiments, a fingerprint matrix may comprise 2, 3, 4, 5, 10, 20, 25, 50, 100, 200, 300, 400, 500, 600, 700, 800, 900, 1000, 2000, 3000, 4000, 5000, 6000, 7000, 8000, 9000, or 10,000 or more barcode fingerprints. In some embodiments, a fingerprint matrix may comprise at most 10,000 barcode fingerprints.

The technologies described herein allow for the generation and characterization of unique fingerprints for each barcode. This allows, for example, availability of methods of cargo (i.e., target (e.g., protein)) detection that may not require orthogonality between barcode-binder pairs. In some embodiments, barcode-binder pairs may be orthogonal. In some embodiments, barcode-binder pairs may not be orthogonal. As may be evident to a person of ordinary skill in the art, barcode-binder pairs as described herein provide the advantage of being more robust, as the availability of unique fingerprints makes non-specific binding less of a concern, a major advantage in complex environments (e.g., serum, blood, etc.).

iii. Decoding

### Analysis

A key component of the invention is the method used to deduce relative or absolute protein concentrations from the DNA sequencing of binders. In the invention, the DNA sequences are translated in silico into amino acid sequences corresponding to each binder and tabulated to yield a table of binder counts. The binder count table measured for any given barcode in isolation is henceforth known as a “fingerprint” of a barcode. When applying the invention to an unknown mixture of barcoded cargos, the relative or absolute abundance of individual barcodes is determined by comparing the binder count table to the predetermined fingerprints of the individual barcodes and applying a computational prediction method described below. In some embodiments, the binder count table of a mixture of m unknown barcodes is assumed to be a linear combination of their respective fingerprints; the coefficients of the linear combination are inferred through least-squares fitting of the equation A x=b, where A is an n-by-m matrix of fingerprints, b is a length-n vector of binder counts, and x is an undetermined length-m vector of the abundances of each of the barcodes. In some embodiments, the abundances of each of the barcodes is inferred using a Bayesian method, whereby a suitable prior probability distribution over the barcode abundances is assumed, a likelihood ratio of the observed count table given barcode abundances is calculated from a model of the uncertainties in the experimental system, and a posterior probability distribution is inferred the product of the prior with the likelihood ratio. In some embodiments, the posterior distribution is estimated using Monte Carlo sampling methods. In some embodiments, the maximum of the posterior distribution is determined with a computational optimization procedure. In some embodiments, the binder count table is assumed to be a non-linear function of the abundances of various barcodes to account for saturation of particular barcode-binder interactions or competition between distinct barcodes or distinct binders.

In some embodiments, relative proportions of binder counts are compared directly in order to determine relative proportions of barcodes. In some embodiments, sequences of known abundance are mixed into the experiment, and utilized to determine the absolute abundance of a given binder, which is used to estimate an absolute concentration for a barcode.

### VI. Nucleic Acids

i. Cargo Nucleic Acid

Among other things, the present disclosure provides nucleic acids, e.g., that can be disposed within a delivery particle as described herein. Nucleic acids according to the present disclosure include all those known in the art, including cosmids, plasmids (e.g., naked or contained in liposomes) and viral constructs (e.g., lentiviral, retroviral, adenoviral, and adeno-associated viral constructs) that incorporate a nucleic acid encoding a cargo polypeptide, or characteristic portion thereof. Those of skill in the art will be capable of selecting suitable constructs, as well as cells, for making any of the polynucleotides described herein. In some embodiments, a nucleic acid is a plasmid (i.e., a circular DNA molecule that can autonomously replicate inside a cell). In some embodiments, a nucleic acid can be a cosmid (e.g., pWE or sCos series).

In some embodiments, a cargo nucleic acid (e.g., a cargo component) is or comprises a wild-type (e.g., naturally occurring) nucleic acid. In some embodiments, a cargo nucleic acid (e.g., a cargo component) is or comprises a variant nucleic acid (e.g., a variant cargo nucleic acid). In some embodiments, a variant nucleic acid is a variant of a reference nucleic acid, which reference nucleic acid is or comprises a wild-type (e.g., naturally occurring) nucleic acid (e.g., a nucleic acid encoding a wild-type polypeptide). In some embodiments, a variant nucleic acid is or comprises at least one mutation relative to a reference nucleic acid (e.g., a wild-type nucleic acid (e.g., a nucleic acid encoding a wild-type polypeptide)).

In some embodiments, a variant cargo nucleic acid (e.g., a variant cargo component) is associated with (e.g., operably linked to) a barcode, as described herein (i.e., a barcoded variant cargo nucleic acid). In some embodiments, a variant cargo nucleic acid possesses improved functionality (e.g., reduced toxicity (e.g., reduced anemia), improved pharmacokinetic measures (e.g., dissociation constant (Kd), improved biophysical properties, improved developability, improved expression, etc.) relative to a reference nucleic acid (e.g., a wild-type nucleic acid (e.g., a nucleic acid encoding a wild-type polypeptide)).

### 1. Viral Nucleic Acid

In some embodiments, a nucleic acid is a viral construct. In some embodiments, a viral construct is a lentivirus, retrovirus, adenovirus, or adeno-associated virus construct. In some embodiments, a nucleic acid is an adeno-associated virus (AAV) construct (see, e.g., Asokan et al., Mol. Ther. 20: 699-7080, 2012, which is incorporated in its entirety herein by reference). In some embodiments, a viral construct is an adenovirus construct. In some embodiments, a viral construct may also be based on or derived from an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference to each of the publications in its entirety.

In some embodiments, a nucleic acid is a viral construct and can have a total number of nucleotides of up to 10 kb. In some embodiments, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, a nucleic acid is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb

In some embodiments, a nucleic acid is an adenovirus construct and can have a total number of nucleotides of up to 8 kb. In some embodiments, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the nucleic acids described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some embodiments, a promoter can be a native promoter, a constitutive promoter, an inducible promoter, and/or a tissue-specific promoter. Non-limiting examples of control sequences are described herein.

In some embodiments, the present disclosure provides for a cargo component that further comprises one or more sequence elements, or the complement thereof, selected from the group consisting of: a promoter, an enhancer, a silencer, an insulator, a transcriptional regulatory element, a translational regulatory element, a splice donor, a splice acceptor, a transcriptional terminator, a translational start site, a translational stop site, a packaging signal, an integration signal, inverted terminal repeats (ITRs), and any combination thereof. Exemplary sequence elements are described herein.

### 2. Plasmid

In some embodiments, a nucleic acid (e.g., cargo nucleic acid) is or comprises a plasmid. In some embodiments, a nucleic acid is a DNA plasmid. In some embodiments, a nucleic acid is an RNA plasmid. In some embodiments, a plasmid is able to replicate independently in a cell. In some embodiments, a plasmid comprises an origin of replication sequence. In some embodiments, a plasmid is a nanoplasmid.

Nucleic acids provided herein can be of different sizes. In some embodiments, a nucleic acid is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, a nucleic acid is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, the present disclosure provides for a plasmid comprising a cargo component that further comprises one or more sequence elements, or the complement thereof, selected from the group consisting of: a promoter, an enhancer, a silencer, an insulator, a transcriptional regulatory element, a translational regulatory element, a splice donor, a splice acceptor, a transcriptional terminator, a translational start site, a translational stop site, a packaging signal, an integration signal, inverted terminal repeats (ITRs), and any combination thereof. Exemplary sequence elements are described herein.

### 3. RNA

In certain embodiments, the disclosed compositions comprise nucleic acids. In some embodiments, nucleic acids are RNAs. In some embodiments, nucleic acids comprise modified nucleic acids. In some embodiments, nucleic acids comprise modified RNAs. Among other things, the present disclosure describes that selection and combination of nucleic acids as described herein impacts characteristics of cargo nucleic acid such as stability and ionizability.

### A. Modified RNAs

In certain embodiments, the disclosed compositions and/or nucleic acids comprise modified nucleic acids, including modified RNAs.

Modified nucleosides or nucleotides can be present in an RNA, for example a mRNA. A mRNA comprising one or more modified nucleosides or nucleotides, for example, is called a “modified” RNA to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.”

Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Certain embodiments comprise a 5′ end modification to an mRNA or nucleic acid. Certain embodiments comprise a 3′ end modification to an mRNA or nucleic acid. A modified RNA can contain 5′ end and 3′ end modifications. A modified RNA can contain one or more modified residues at non-terminal locations. In certain embodiments, an mRNA includes at least one modified residue.

Unmodified nucleic acids can be prone to degradation by, e.g., intracellular nucleases or those found in serum. For example, nucleases can hydrolyze nucleic acid phosphodiester bonds. Accordingly, in one aspect the RNAs (e.g., mRNAs) described herein can contain one or more modified nucleosides or nucleotides, e.g., to introduce stability toward intracellular or serum-based nucleases. The term “innate immune response” includes a cellular response to exogenous nucleic acids, including single stranded nucleic acids, which involves the induction of cytokine expression and release, particularly the interferons, and cell death.

Accordingly, in some embodiments, RNA or nucleic acids in the disclosed the disclosed compositions, preparations, nanoparticles, and/or nanomaterials comprise at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the RNA or nucleic acid more stable (e.g., resistant to nuclease digestion) than the wild-type or naturally occurring version of the RNA or nucleic acid. As used herein, the terms “stable” and “stability” as such terms relate to the nucleic acids of the present invention, and particularly with respect to the RNA, refer to increased or enhanced resistance to degradation by, for example nucleases (i.e., endonucleases or exonucleases) which are normally capable of degrading such RNA. Increased stability can include, for example, less sensitivity to hydrolysis or other destruction by endogenous enzymes (e.g., endonucleases or exonucleases) or conditions within the target cell or tissue, thereby increasing or enhancing the residence of such RNA in the target cell, tissue, subject and/or cytoplasm. The stabilized RNA molecules provided herein demonstrate longer half-lives relative to their naturally occurring, unmodified counterparts (e.g., the wild-type version of the mRNA). Also contemplated by the terms “modification” and “modified” as such terms related to the mRNA of the LNP compositions disclosed herein are alterations which improve or enhance translation of mRNA nucleic acids, including for example, the inclusion of sequences which function in the initiation of protein translation (e.g., the Kozac consensus sequence). (Kozak, M., Nucleic Acids Res 15 (20): 8125-48 (1987), the contents of which are hereby incorporated by reference herein in its entirety).

In some embodiments, an RNA or nucleic acid of the disclosed compositions, preparations, nanoparticles, and/or nanomaterials disclosed herein have undergone a chemical or biological modification to render it more stable. Exemplary modifications to an RNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring RNA, for example, covalent modifications such as the introduction of modified nucleotides, (e.g., nucleotide analogs, or the inclusion of pendant groups which are not naturally found in such RNA molecules).

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution. Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### 4. Other

In certain embodiments, cargo nucleic acids comprise other components. In some embodiments, cargo nucleic acids comprise one or more of components such as promoters, enhancers, untranslated regions (UTRs), internal ribosome entry sites (IRES), splice sites, polyadenylation sequences, sequences comprising destabilization domains, reporter sequences or elements, and/or other additional sequences. Among other things, the present disclosure describes that selection and combination of one or more of the components as described herein impacts characteristics of nucleic acids such as stability, expression, localization and tropism.

### A. Promoters

In some embodiments, a nucleic acid comprises a promoter. The term “promoter” refers to a DNA sequence recognized by enzymes/proteins that can promote and/or initiate transcription of an operably linked gene (e.g., a nucleic acid encoding a cargo polypeptide). For example, a promoter typically refers to, e.g., a nucleotide sequence to which an RNA polymerase and/or any associated factor binds and from which it can initiate transcription. Thus, in some embodiments, a nucleic acid (e.g., disposed within a delivery particle) comprises a promoter operably linked to one of the non-limiting example promoters described herein.

In some embodiments, a promoter is an inducible promoter, a constitutive promoter, a mammalian cell promoter, a viral promoter, a chimeric promoter, an engineered promoter, a tissue-specific promoter, or any other type of promoter known in the art. In some embodiments, a promoter is a RNA polymerase II promoter, such as a mammalian RNA polymerase II promoter. In some embodiments, a promoter is a RNA polymerase III promoter, including, but not limited to, a HI promoter, a human U6 promoter, a mouse U6 promoter, or a swine U6 promoter. A promoter will generally be one that is able to promote transcription in a cell, tissue, organ, organoid, or organism of interest. In some embodiments, a promoter is a mammalian cell-specific promoter.

A variety of promoters are known in the art, which can be used herein. Non-limiting examples of promoters that can be used herein include: human EF1α, human cytomegalovirus (CMV) (U.S. Pat. No. 5,168,062, which is incorporated in its entirety herein by reference), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein H (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2K b, HSP70, proliferin, tumor necrosis factor, thyroid stimulating hormone a gene, immunoglobulin light chain, T-cell receptor, HlA DQa and DQ, interleukin-2 receptor, MHC class II, MHC class II HlA-DRa, muscle creatine kinase, prealbumin (transthyretin), elastase 1, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007, each of which is incorporated in its entirety herein by reference. In some embodiments, a promoter is the CMV immediate early promoter. In some embodiments, the promoter is a CAG promoter or a CAG/CBA promoter. The term “constitutive” promoter refers to a nucleotide sequence that, when operably linked with a nucleic acid encoding a cargo polypeptide, causes RNA to be transcribed from the nucleic acid in a cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985, which is incorporated in its entirety herein by reference), the SV40 promoter, the dihydrofolate reductase promoter, the beta-actin promoter, the phosphoglycerol kinase (PGK) promoter, and the EFl-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionein (MT) promoter, the dexamethasone (Dex)-inducible mouse mammary tumor virus (MMTV) promoter, the T7 polymerase promoter system (WO 98/10088, which is incorporated in its entirety herein by reference); the ecdysone insect promoter (see, e.g., No et al, Proc. Natl. Acad Sci. US.A. 93:3346-3351, 1996, which is incorporated in its entirety herein by reference), the tetracycline-repressible system (see, e.g., Gossen et al, Proc. Natl. Acad Sci. US.A. 89:5547-5551, 1992, which is incorporated in its entirety herein by reference), the tetracycline-inducible system (see, e.g., Gossen et al, Science 268:1766-1769, 1995; and Harvey et al, Curr. Opin. Chem. Biol. 2:512-518, 1998, each of which is incorporated in their entirety herein by reference), the RU486-inducible system (see, e.g., Wang et al, Nat. Biotech. 15:239-243, 1997; and Wang et al, Gene Ther. 4:432-441, 1997, each of which is incorporated in their entirety herein by reference), and the rapamycin-inducible system (see, e.g., Magari et al. J Clin. Invest. 100:2865-2872, 1997, which is incorporated in its entirety herein by reference).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter).

In some embodiments, regulatory and/or control sequences impart tissue-specific gene expression capabilities. In some cases, tissue-specific regulatory and/or control sequences bind tissue-specific transcription factors that induce transcription in a tissue-specific manner.

In some embodiments, provided nucleic acids comprise a promoter sequence selected from a CAG, a CBA, a CMV, or a CB7 promoter.

### B. Enhancers

In some instances, a construct can include an enhancer sequence. The term “enhancer” refers to a nucleotide sequence that can increase the level of transcription of a nucleic acid encoding a protein of interest (e.g., a cargo polypeptide). Enhancer sequences (generally 50-1500 bp in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and/or a SV40 enhancer.

### C. Flanking Untranslated Regions, 5′ UTRs and 3′ UTRs

In some embodiments, any of the nucleic acids described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, kits, or methods as described herein to enhance or otherwise modulate the expression of a cargo polypeptide.

Natural 5′ UTRs include a sequence that plays a role in translation initiation, in some embodiments, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding.

In some embodiments, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (see, e.g., Chen et al., Mal. Cell. Biol. 15:5777-5788, 1995; Chen et al., Mal. Cell Biol. 15:2010-2018, 1995, each of which is incorporated herein by reference in its entirety): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding a cargo polypeptide. In other embodiments, AREs can be removed or mutated to increase the intracellular stability and thus increase translation and production of a cargo polypeptide.

In other embodiments, non-ARE sequences may be incorporated into the 5′ or 3′ UTRs. In some embodiments, introns or portions of intron sequences may be incorporated into the flanking regions of the polynucleotides in any of the constructs, compositions, kits, and methods provided herein. Incorporation of intronic sequences may increase protein production as well as mRNA levels.

### D. Internal Ribosome Entry Sites (IRES)

In some embodiments, a nucleic acid comprising a cargo component can include an internal ribosome entry site (IRES). An IRES forms a complex secondary structure that allows translation initiation to occur from any position with an mRNA immediately downstream from where the IRES is located (see, e.g., Pelletier and Sonenberg, Mal. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV) (see e.g., Alberts, Molecular Biology of the Cell, Garland Science, 2002; and Hellen et al., Genes Dev. 15(13):1593-612, 2001, each of which is incorporated in its entirety herein by reference).

In some embodiments, the IRES sequence that is incorporated into a construct that encodes a cargo polypeptide, or a C-terminal portion of a cargo polypeptide is the foot and mouth disease virus (FMDV) 2A sequence. The Foot and Mouth Disease Virus 2A sequence is a small peptide (approximately 18 amino acids in length) that has been shown to mediate the cleavage of polyproteins (see, e.g., Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999, each of which is incorporated in its entirety herein by reference). The cleavage activity of the 2A sequence has previously been demonstrated in artificial systems including plasmids and gene therapy constructs (AAV and retroviruses) (see, e.g., Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy II: 1921-1931, 2000; and Klump et al., Gene Therapy 8:811-817, 2001, each of which is incorporated in its entirety herein by reference).

An IRES can be utilized in a delivery particle described herein. In some embodiments, a nucleic acid encoding a C-terminal portion of a cargo polypeptide can include a polynucleotide internal ribosome entry site (IRES). In some embodiments, an IRES can be part of a composition comprising more than one nucleic acid. In some embodiments, an IRES is used to produce more than one cargo polypeptide from a single gene transcript.

### E. Splice Sites

In some embodiments, any of the nucleic acids provided herein can include splice donor and/or splice acceptor sequences, which are functional during RNA processing occurring during transcription. In some embodiments, splice sites are involved in trans-splicing.

### F. Polyadenylation Sequences

In some embodiments, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (see, e.g., Molecular Biology of the Cell, Third Edition by B. Alberts et al., Garland Publishing, 1994, which is incorporated herein by reference in its entirety). In some embodiments, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) (SEQ ID NO: 9063) added to the pre-mRNA through the action of an enzyme, polyadenylate polymerase. In some embodiments, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (see, e.g., Woychik et al., Proc. Natl. Acad Sci. US.A. 81(13):3944-3948, 1984; U.S. Pat. No. 5,122,458, each of which is incorporated herein by reference in its entirety), mouse-β-globin, mouse-α-globin (see, e.g., Orkin et al., EMBO J 4(2):453-456, 1985; Thein et al., Blood 71(2):313-319, 1988, each of which is incorporated herein by reference in its entirety), human collagen, polyoma virus (see, e.g., Batt et al., Mal. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (see, e.g., Szymanski et al., Mal. Therapy 15(7):1340-1347, 2007, which is incorporated herein by reference in its entirety), the group consisting of SV40 poly(A) site, such as the SV40 late and early poly(A) site (see, e.g., Schek et al., Mal. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some embodiments, a poly(A) signal sequence can be a synthetic polyadenylation site (see, e.g., the pCl-neo expression construct of Promega that is based on Levitt el al, Genes Dev. 3(7):1019-1025, 1989, which is incorporated herein by reference in its entirety). In some embodiments, a poly(A) signal sequence is the polyadenylation signal of soluble neuropilin-1 (sNRP) (AAATAAAATACGAAATG (SEQ ID NO: 9058)) (see, e.g., WO 05/073384, which is incorporated herein by reference in its entirety). In some embodiments, a poly(A) signal sequence comprises or consists of the SV40 poly(A) site.

### G. Destabilization Domains

In some embodiments, any of the nucleic acids provided herein can optionally include a sequence encoding a destabilizing domain (“a destabilizing sequence”) for temporal control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

Additional examples of destabilizing sequences are known in the art. In some embodiments, the destabilizing sequence is a FK506- and rapamycin-binding protein (FKBP12) sequence, and the stabilizing ligand is Shield-1 (Shld1) (see, e.g., Banaszynski et al. (2012) Cell 126(5): 995-1004, which is incorporated in its entirety herein by reference). In some embodiments, a destabilizing sequence is a DHFR sequence, and a stabilizing ligand is trimethoprim (TMP) (see, e.g., Iwamoto et al. (2010) Chem Biol 17:981-988, which is incorporated in its entirety herein by reference).

In some embodiments, a destabilizing sequence is a FKBP12 sequence, and a presence of nucleic acids carrying the FKBP12 gene in a subject cell (e.g., a cell of interest (e.g., a glial cell, a liver cell, a tumor cell, etc.)) is detected by Western blotting. In some embodiments, a destabilizing sequence can be used to verify the temporally-specific activity of delivery particles described herein.

### H. Reporter Sequences or Elements

In some embodiments, nucleic acids provided herein can optionally include a sequence encoding a reporter polypeptide and/or protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, a reporter sequence is the LacZ gene, and the presence of a construct carrying the LacZ gene in a mammalian cell (e.g., a cell of interest (e.g., a glial cell, a liver cell, a tumor cell, etc.)) is detected by assays for beta-galactosidase activity. When the reporter is a fluorescent protein (e.g., green fluorescent protein) or luciferase, the presence of a construct carrying the fluorescent protein or luciferase in a mammalian cell (e.g., a cell of interest (e.g., a glial cell, a liver cell, a tumor cell, etc.)) may be measured by fluorescent techniques (e.g., fluorescent microscopy or FACS) or light production in a luminometer (e.g., a spectrophotometer or an IVIS imaging instrument). In some embodiments, a reporter sequence can be used to verify the tissue-specific targeting capabilities and tissue-specific promoter regulatory and/or control activity of any of the constructs described herein.

In some embodiments, a reporter sequence is a FLAG tag (e.g., a 3×FLAG tag), and the presence of a construct carrying the FLAG tag in a mammalian cell (e.g., a cell of interest (e.g., a glial cell, a liver cell, a tumor cell, etc.)) is detected by protein binding or detection assays (e.g., Western blots, immunohistochemistry, radioimmunoassay (RIA), mass spectrometry).

### I. Additional Sequences

In some embodiments, nucleic acids of the present disclosure may comprise a T2A element or sequence. In some embodiments, nucleic acids of the present disclosure may include one or more cloning sites. In some such embodiments, cloning sites may not be fully removed prior to manufacturing for administration to a subject. In some embodiments, cloning sites may have functional roles including as linker sequences, or as portions of a Kozak site. As will be appreciated by those skilled in the art, cloning sites may vary significantly in primary sequence while retaining their desired function.

### J. Inverted Terminal Repeat Sequences (ITRs)

In some embodiments, a delivery particle is an AAV delivery particle. AAV derived nucleic sequences of a construct typically comprises the cis-acting 5′ and 3′ ITRs (see, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs can also aid in efficient encapsidation of an AAV construct in an AAV delivery particle.

An rAAV delivery particle (e.g., an AAV2 delivery particle) of the present disclosure can comprise a nucleic acid comprising a cargo component encoding a cargo polypeptide and associated elements flanked by a 5′ and a 3′ AAV ITR sequences. In some embodiments, an ITR is or comprises about 145 nucleic acids. In some embodiments, all or substantially all of a sequence encoding an ITR is used. An AAV ITR sequence may be obtained from any known AAV, including presently identified mammalian AAV types. In some embodiments an ITR is an AAV2 ITR

An example of a construct molecule employed in the present disclosure is a “cis-acting” construct containing a transgene, in which the selected transgene sequence and associated regulatory elements are flanked by 5′ or “left” and 3′ or “right” AAV ITR sequences. 5′ and left designations refer to a position of an ITR sequence relative to an entire construct, read left to right, in a sense direction. For example, in some embodiments, a 5′ or left ITR is an ITR that is closest to a promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some embodiments, a 3′ or right ITR is an ITR that is closest to a polyadenylation sequence (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

### VII. Delivery Particles

Among other things, the present disclosure provides delivery particles. In some embodiments, a delivery particle is a viral particle, a lipid-based particle [(e.g., cell-produced or not cell-produced), a lipid nanoparticle (LNP), a liposome, a micelle, an extracellular vesicle (e.g., exosomes, microparticles, etc.)], a polymer-based particle (e.g., PGLA), a polysaccharide-based particle, etc. In some embodiments, delivery particles as described herein comprise nucleic acids In some embodiments, a nucleic acid described herein is disposed within a delivery particle. In some embodiments, a nucleic acid described herein is associated (e.g., covalently or non-covalently) with a surface of delivery particle. In some embodiments, a nucleic acid comprises, among other things, a cargo component encoding a cargo polypeptide, that, when expressed, is expressed on a surface of a delivery particle.

i. Virions:

Among other things, the present disclosure provides virions that comprise a nucleic acid and a capsid as described herein. In some embodiments, virions are delivery particles that comprise a nucleic acid comprising a cargo component encoding a cargo polypeptide or characteristic portion thereof described herein, and a capsid described herein. An exemplary delivery particle is an AAV delivery particle. An exemplary delivery particle is a lentivirus delivery particle. However, other delivery particles may be used.

In some embodiments, a delivery particle is an AAV delivery particle. AAV delivery particles that comprise a nucleic acid comprising a cargo component encoding a cargo polypeptide or characteristic portion thereof described herein, and a capsid described herein. In some embodiments, AAV delivery particles can be described as having a serotype, which is a description of the construct strain and the capsid strain. For example, in some embodiments an AAV delivery particle may be described as AAV2, wherein the particle has an AAV2 capsid and a construct that comprises characteristic AAV2 Inverted Terminal Repeats (ITRs). In some embodiments, an AAV delivery particle may be described as a pseudotype, wherein the capsid and construct are derived from different AAV strains, for example, AAV2/9 would refer to an AAV delivery particle that comprises a construct utilizing the AAV2 ITRs and an AAV9 capsid.

### 1. AAV Construct

The present disclosure provides nucleic acids that comprise a cargo component encoding a cargo polypeptide or characteristic portion thereof. In some embodiments described herein, a nucleic acid that comprises a cargo component encoding a cargo polypeptide or characteristic portion thereof can be disposed within an AAV delivery particle.

In some embodiments, a nucleic acid comprises one or more components derived from or modified from a naturally occurring AAV genomic construct. In some embodiments, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV9 construct, an AAV2.7m8 construct, an AAV8BP2 construct, an AAV293 construct, an AAV.DJ construct, or AAV Anc80 construct. In some embodiments, an rAAV Anc80 capsid is an rAAV Anc80L65 capsid. Additional exemplary AAV constructs that can be used herein are known in the art (see, e.g., Kanaan et al., Mol. Ther. Nucleic Acids 8:184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which is incorporated in its entirety herein by reference).

In some embodiments, provided nucleic acids comprise a cargo component, e.g., encoding a cargo polypeptide, one or more regulatory and/or control sequences, and optionally 5′ and 3′ AAV derived inverted terminal repeats (ITRs). In some embodiments wherein a 5′ and 3′ AAV derived ITR is utilized, the polynucleotide construct may be referred to as a recombinant AAV (rAAV) construct. In some embodiments, provided rAAV constructs are packaged into an AAV capsid to form an AAV delivery particle.

In some embodiments, AAV derived sequences (which are comprised in a polynucleotide construct) typically include the cis-acting 5′ and 3′ ITR sequences (see, e.g., B. J. Carter, in “Handbook of Parvoviruses,” ed., P. Tijsser, CRC Press, pp. 155 168, 1990, which is incorporated herein by reference in its entirety). Typical AAV2-derived ITR sequences are about 145 nucleotides in length. In some embodiments, at least 80% of a typical ITR sequence (e.g., at least 85%, at least 90%, or at least 95%) is incorporated into a construct provided herein. The ability to modify these ITR sequences is within the skill of the art. (see, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated in its entirety by reference). In some embodiments, any of the coding sequences and/or constructs described herein are flanked by 5′ and 3′ AAV ITR sequences. The AAV ITR sequences may be obtained from any known AAV, including presently identified AAV types.

In some embodiments, nucleic acids described in accordance with this disclosure and in a pattern known to the art (see, e.g., Asokan et al., Mol. Ther. 20: 699-7080, 2012, which is incorporated herein by reference in its entirety) are typically comprised of, a coding sequence or a portion thereof, at least one and/or control sequence, and optionally 5′ and 3′ AAV inverted terminal repeats (ITRs). In some embodiments, provided constructs can be packaged into a capsid to create an AAV delivery particle. An AAV delivery particle may be delivered to a selected target cell. In some embodiments, provided constructs comprise an additional optional coding sequence that is a nucleic acid sequence (e.g., inhibitory nucleic acid sequence), heterologous to the nucleic sequences, which encodes a polypeptide, protein, functional RNA molecule (e.g., miRNA, miRNA inhibitor) or other gene product, of interest. In some embodiments, a nucleic acid coding sequence is operatively linked to and/or control components in a manner that permits coding sequence transcription, translation, and/or expression in a cell of a target tissue.

In some embodiments, a nucleic acid is an rAAV nucleic acid. In some embodiments, an rAAV nucleic acid can include at least 500 bp, at least 1 kb, at least 1.5 kb, at least 2 kb, at least 2.5 kb, at least 3 kb, at least 3.5 kb, at least 4 kb, or at least 4.5 kb. In some embodiments, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some embodiments, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the nucleic acids described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some embodiments, a promoter can be a native promoter, a constitutive promoter, an inducible promoter, and/or a tissue-specific promoter. Non-limiting examples of control sequences are described herein.

### 2. AAV Capsids

The present disclosure provides one or more nucleic acids disposed with an AAV capsid. In some embodiments, an AAV capsid is from or derived from an AAV capsid of an AAV2, 3, 4, 5, 6, 7, 8, 9, 10, DJ, PHP-B, rh8, rh10, rh39, rh43 or Anc80 serotype, or one or more hybrids thereof. In some embodiments, an AAV capsid is from an AAV ancestral serotype

As provided herein, any combination of AAV capsids and AAV nucleic acids (e.g., comprising AAV ITRs) may be used in recombinant AAV (rAAV) particles of the present disclosure. For example, wild type or variant AAV2 ITRs and Anc80 capsid, wild type or variant AAV2 ITRs and AAV6 capsid, etc. In some embodiments of the present disclosure, an AAV delivery particle is wholly comprised of AAV2 components (e.g., capsid and ITRs are AAV2 serotype). In some embodiments, an AAV delivery particle is an AAV2/6, AAV2/8 or AAV2/9 particle (e.g., an AAV6, AAV8 or AAV9 capsid with an AAV construct having AAV2 ITRs).

ii. Lipid-Based Delivery Particles

Among other things, the present disclosure provides for compositions, preparations, and/or delivery particles that comprise lipids (e.g., lipid-based delivery particles). In some embodiments, lipid-based delivery particles are produced by a cell. In some embodiments, lipid-based delivery particles are not produced by a cell. The present invention provided for lipid-based delivery particles that may be of various types. In some embodiments, lipid-based delivery particles may be lipid nanoparticles (LNPs). In some embodiments, lipid-based delivery particles may be liposomes. In some embodiments, lipid-based delivery particles may be micelles. In some embodiments, lipid-based delivery particles may be extracellular vesicles (e.g., exosomes).

### 1. Lipid Nanoparticles (LNPs)

In some embodiments, the present disclosure provides for compositions, preparations, and/or delivery particles that comprise lipid nanoparticles. In some embodiments, lipid nanoparticles comprise one or more components. In some embodiments, lipid nanoparticles comprise one or more components such as compounds, ionizable lipids, sterols, conjugate-linker lipids, and phospholipids. Among other things, the present disclosure describes that selection and combination of one or more of the components as described herein impacts characteristics of lipid nanoparticles such as diameter, pKa, stabilization, and ionizability.

Among other things, the present disclosure describes that selection and combination of one or more of the components as described herein impacts functional activity of lipid nanoparticles such as tropism, stabilization, and drug delivery efficacy. For example, the present disclosure describes that a combination of components may better suit delivery of nucleic acids comprising a cargo (e.g., a nucleic acid encoding a cargo polypeptide) as described herein. In some embodiments, a cargo comprises RNA. In some embodiments, a cargo comprises DNA.

In some embodiments, lipid nanoparticles comprise one or more compounds as described herein. In some embodiments, lipid nanoparticles comprise one or more ionizable lipids as described herein. In some embodiments, lipid nanoparticles comprise one or more sterols as described herein. In some embodiments, lipid nanoparticles comprise one or more conjugate-linker lipids as described herein. In some embodiments, lipid nanoparticles comprise one or more phospholipids as described herein.

### A. Ionizable Lipids

Among other things, the present disclosure describes compositions, preparations, delivery particles, and/or methods that comprise one or more ionizable lipids as described herein.

Among other things, the present disclosure describes that different ratios of ionizable lipids influence one or more functional activities such as desired tropisms, stabilization, and drug delivery efficacy of compositions, preparations, nanoparticles, and/or nanomaterials described herein. For example, in some embodiments, compositions, preparations delivery particles, and methods can be used to identify an amount of ionizable lipid that is useful and/or critical to functional activity of lipid nanoparticles such as desired tropisms, stabilization, and drug delivery efficacy as described herein. In some embodiments, compositions, preparations delivery particles, and methods can be used to identify characteristic features of an ionizable lipid that is useful and/or critical to functional activity of lipid nanoparticles such as desired tropisms, stabilization, and drug delivery efficacy as described herein.

### B. Sterols

Among other things, the present disclosure describes compositions, preparations, delivery particles, and/or methods that comprise one or more sterols as described herein.

In some embodiments, a sterol is a cholesterol, or a variant or derivative thereof. In some embodiments, a cholesterol is modified. In some embodiments, a cholesterol is an oxidized cholesterol. In some embodiments, a cholesterol is esterified cholesterol. Unmodified cholesterol can be acted upon by enzymes to form variants that are side-chain or ring oxidized. In some embodiments, a cholesterol can be oxidized on the beta-ring structure or on the hydrocarbon tail structure. In some embodiments, a sterol is a phytosterol. Exemplary sterols that are considered for use in the disclosed lipid nanoparticles include but are not limited to 25-hydroxycholesterol (25-OH), 20α-hydroxycholesterol (20α-OH), 27-hydroxycholesterol, 6-keto-5α-hydroxycholesterol, 7-ketocholesterol, 7β-hydroxycholesterol, 7α-hydroxycholesterol, 7β-25-dihydroxycholesterol, beta-sitosterol, stigmasterol, brassicasterol, campesterol, or combinations thereof. In some embodiments, a side-chain oxidized cholesterol can enhance cargo delivery relative to other cholesterol variants. In some embodiments, a cholesterol is an unmodified cholesterol. In some embodiments, a cholesterol is campesterol.

For example, in some embodiments, compositions, preparations delivery particles, and methods can be used to identify an amount of sterol that is useful and/or critical to functional activity of lipid nanoparticles such as desired tropisms, stabilization, and drug delivery efficacy as described herein. In some embodiments, compositions, preparations delivery particles, and methods can be used to identify characteristic features of a sterol that is useful and/or critical to functional activity of lipid nanoparticles such as desired tropisms, stabilization, and drug delivery efficacy as described herein.

### C. Conjugate-Linker Lipids

Among other things, the present disclosure describes compositions, preparations, delivery particles, and/or methods that comprise one or more conjugate-linker lipids as described herein.

In some embodiments, a conjugate-linker lipid is or comprises a polyethylene glycol (PEG)-lipid or PEG-modified lipid. In some embodiments, PEG or PEG-modified lipids may be alternately referred to as PEGylated lipids or PEG-lipids. Inclusion of a PEGylating lipid can be used to enhance lipid nanoparticle colloidal stability in vitro and circulation time in vivo. In some embodiments, the PEGylation is reversible in that the PEG moiety is gradually released in blood circulation. Exemplary PEG-lipids include but are not limited to PEG conjugated to saturated or unsaturated alkyl chains having a length of C6-C20. PEG-modified phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. For example, in some embodiments, a PEG lipid may be PEG-c-DOMG, PEG-DMG, PEG-DLPE, PEG-DMPE, PEG-DPPE, PEG-DSG or a PEG-DSPE lipid.

In some embodiments, a conjugate-linker lipid comprises a polyethylene glycol lipid. In some embodiments, the conjugate-linker lipid comprises DiMystyrlGlycerol (DMG), 1,2-Dipalmitoyl-rac-glycerol, methoxypolyethylene Glycol (DPG-PEG), or 1,2-Distearoyl-rac-glycero-3-methylpolyoxyethylene (DSG-PEG). In some embodiments, a conjugate-linker lipid has an average molecular mass from about 500 Da to about 5000 Da. In some embodiments, a conjugate-linker lipid has an average molecular mass of about 2000 Da.

For example, in some embodiments, compositions, preparations delivery particles, and methods can be used to identify an amount of conjugate-linker lipid that is useful and/or critical to functional activity of lipid nanoparticles such as desired tropisms, stabilization, and drug delivery efficacy as described herein. In some embodiments, compositions, preparations delivery particles, and methods can be used to identify characteristic features of a conjugate-linker lipid that is useful and/or critical to functional activity of lipid nanoparticles such as desired tropisms, stabilization, and drug delivery efficacy as described herein.

### D. Phospholipids

Among other things, the present disclosure describes compositions, preparations, delivery particles, and/or methods that comprise one or more phospholipids as described herein.

In some embodiments, one or more phospholipids may assemble into one or more lipid bilayers. In some embodiments, one or more phospholipids may include a phospholipid moiety. In some embodiments, one or more phospholipids may include one or more fatty acid moieties. In some embodiments, one or more phospholipids may include a phospholipid moiety and one or more fatty acid moieties. In some embodiments, a phospholipid moiety includes but is not limited to phosphatidyl choline, phosphatidyl ethanolamine, phosphatidyl glycerol, phosphatidyl serine, phosphatidic acid, 2-lysophosphatidyl choline, and sphingomyelin. In some embodiments, a fatty acid moiety includes but is not limited to lauric acid, myristic acid, myristoleic acid, palmitic acid, palmitoleic acid, stearic acid, oleic acid, linoleic acid, alphalinolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

Exemplary phospholipids include but are not limited to 1,2-distearoyl-snglycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycerophosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycerophosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoy 1-sn-glycero-3-phosphocholine (OChemsPC), 1-hexadecyl snglycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2 oleoylphosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or combinations thereof. In some embodiments, a phospholipid is DSPC. In some embodiments, a phospholipid is DMPC.

In some embodiments, the phospholipid comprises 1,2-dioleoyl-sn-glycero-3-phosphoethanolamine-N-(succinyl) (succinyl PE), 1,2-distearoyl-sn-glycero-3-phosphocholine (DSPC), cholesterol, 1,2-distearoyl-sn-glycero-3-phosphoethanolamine (DSPE), 1,2-dipalmitoyl-sn-glycero-3-phosphoethanolamine-N-(succinyl) (succinyl-DPPE), 1,2-dioleoyl-sn-glycero-3-phosphoethanolamine (DOPE), 1,2-dimyristoyl-sn-glycero-3-phosphocholine (DMPC), 1,2-dipalmitoyl-sn-glycero-3-phosphocholine (DPPC), or a combination thereof.

For example, in some embodiments, compositions, preparations delivery particles, and methods can be used to identify an amount of phospholipid that is useful and/or critical to functional activity of lipid nanoparticles such as desired tropisms, stabilization, and drug delivery efficacy as described herein. In some embodiments, compositions, preparations delivery particles, and methods can be used to identify characteristic features of a phospholipid that is useful and/or critical to functional activity of lipid nanoparticles such as desired tropisms, stabilization, and drug delivery efficacy as described herein.

### E. Diameter

Among other things, the present disclosure describes compositions and/or delivery particles that have an average hydrodynamic diameter from about 30 to about 220 nm. In some embodiments, compositions, preparations, nanoparticles, and/or nanomaterials described herein have an average hydrodynamic diameter that is about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, or any range having endpoints defined by any two of the aforementioned values. For example, in some embodiments, compositions, preparations, nanoparticles, and/or nanomaterials described herein have an average hydrodynamic diameter from between 50 nm to 200 nm.

In some embodiments, lipid nanoparticles described herein have an average hydrodynamic diameter from about 30 to about 220 nm. In some embodiments, lipid nanoparticles described herein have an average hydrodynamic diameter that is about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, or any range having endpoints defined by any two of the aforementioned values. For example, in some embodiments, lipid nanoparticles described herein have an average hydrodynamic diameter from between 50 nm to 200 nm.

### F. Methods of Manufacturing LNPs

Methods of manufacturing lipid nanoparticles are known in the art. In some embodiments, the described compositions, preparations, and/or delivery particles are manufactured using microfluidics. For instance, exemplary methods of using microfluidics to form lipid nanoparticles are described by Leung, A. K. K, et al., J Phys Chem, 116:18440-18450 (2012), Chen, D., et al., J Am Chem Soc, 134:6947-6951 (2012), and Belliveau, N. M., et al., Molecular Therapy—Nucleic Acids, 1: e37 (2012), the disclosures of which are hereby incorporated by reference in their entireties. Additionally, exemplary methods as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures of each of which are herein incorporated by reference in their entirety for the purposes described herein.

Briefly, a cargo, such as a cargo described herein, is prepared in a first buffer solution. Other lipid nanoparticle components (such as ionizable lipid, conjugate-linker lipids, cholesterol, and phospholipid) are prepared in a second buffer solution. In some embodiments, a syringe pump introduces the two solutions into a microfluidic device. The two solutions come into contact within the microfluidic device to form lipid nanoparticles encapsulating the cargo.

For example, in some embodiments, cationic lipids, neutral lipids (e.g., DSPC, and/or cholesterol) and polymer-conjugated lipids can be solubilized in ethanol at a pre-determined molar ratio (e.g., ones described herein). In some embodiments, lipid nanoparticles (lipid nanoparticle) are prepared at a total lipid to polyribonucleotides weight ratio of approximately 10:1 to 30:1. In some embodiments, such polyribonucleotides can be diluted to 0.2 mg/mL in acetate buffer.

In some embodiments, using an ethanol injection technique, a colloidal lipid dispersion comprising polyribonucleotides can be formed as follows: an ethanol solution comprising lipids, such as cationic lipids, neutral lipids, and polymer-conjugated lipids, is injected into an aqueous solution comprising polyribonucleotides (e.g., ones described herein).

In some embodiments, lipid and polyribonucleotide solutions can be mixed at room temperature by pumping each solution at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous polyribonucleotides. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

In some embodiments, RNA-encapsulated lipid nanoparticles can be processed through filtration.

In some embodiments, particle size and/or internal structure of lipid nanoparticles (with or without RNAs) may be monitored by appropriate techniques such as, e.g., small-angle X-ray scattering (SAXS) and/or transmission electron cryomicroscopy (CryoTEM).

### 2. Extracellular Vesicles (EVs)

As described herein, an extracellular vesicle (EV) is a lipid-bound vesicle-like structure. In some embodiments, EVs have a membrane. In some embodiments, EVs have a membrane that is a double layer membrane (e.g., a lipid bilayer). In some embodiments, EVs have a membrane that originates from a cell. In some embodiments, EVs have a membrane that originates from the plasma membrane of a cell.

The term extracellular vesicle encompasses exosomes, microvesicles, membrane microparticles, ectosomes, blebs or apoptotic bodies. In some embodiments, an EV is classified as an exosome, microvesicle, membrane microparticle, ectosome, bleb or apoptotic body based on the origin of formation.

In some embodiments, EVs are substantially transparent. In some embodiments, EVs are substantially spherical.

### A. Populations

In some embodiments, an EV has a diameter within a range of 50 to 1000 nm. In some embodiments, an EV has a diameter within a range of 50 to 750 nm. In some embodiments, an EV has a diameter within a range of 50 to 500 nm. In some embodiments, an EV has a diameter within a range of 50 to 300 nm. In some embodiments, an EV has a diameter within a range of 50 to 200 nm. In some embodiments, an EV has a diameter within a range of 50 to 150 nm. In some embodiments, an EV has a diameter within a range of 100 to 1000 nm. In some embodiments, an EV has a diameter within a range of 100 to 750 nm. In some embodiments, an EV has a diameter within a range of 100 to 500 nm. In some embodiments, an EV has a diameter within a range of 100 to 300 nm. In some embodiments, an EV has a diameter within a range of 100 to 200 nm. In some embodiments, an EV has a diameter of at least 100 nm. In some embodiments, an EV has a diameter of at most 300 nm.

A population of EVs (e.g., as present in a composition, pharmaceutical composition, medicament, preparation or otherwise) will comprise EVs with a range of diameters. In some embodiments, the median diameter of EVs within a population is 50, 60, 70, 80, 90, 100, 110, 120, 130, 140, 150, 160, 170, 180, 190, 200, 210, 220, 230, 240, 250, 260, 270, 280, 290, or 300 nm (±1, 2, 3, 4, 5, 6, 7, 8, 9 or 10 nm). In some embodiments, the mean diameter of EVs within a population is 50, 60, 70, 80, 90, 100, 110, 120, 130, 140, 150, 160, 170, 180, 190, 200, 210, 220, 230, 240, 250, 260, 270, 280, 290, or 300 nm (+1, 2, 3, 4, 5, 6, 7, 8, 9 or 10 nm).

A population of EVs may comprise at least 10, 100, 1000, 104, 105, 106, 107, 108, 109, 1010, 1011, 1012, 1013 or 1014 EVs. A population of EVs may comprise at least 10, 100, 1000, 104, 105, 106, 107, 108, 109, 1010, 1011, 1012, 1013 or 1014 EVs per mL of carrier.

### B. Production

In some embodiments, an extracellular vesicle is derived from a cell into the extracellular environment. An extracellular vesicle may be derived from various cell types under both pathological and physiological conditions. In some embodiments, EVs have a similar composition to the cell from which it is derived from. In some embodiments, an EV is produced from outward budding and fission of cellular membrane. An EV may be produced via a natural process or a chemically-induced or enhance process.

In some embodiments, EVs are derived from cells that are contacted with a vesicle-inducing agent. A vesicle-inducing agent may be calcium ionophore, lysophosphatidic acid (LPA), or phorbol-12-myristat-13-acetate (PMA).

In some embodiments, EVs are derived from human cells, or cells of human origin. In some embodiments, EVs are derived from cells that are not modified (e.g., transduced, transfected, infected, or otherwise modified). In some embodiments, EVs are derived from cells that are ex vivo.

In some embodiments, EVS are derived from hematopoietic cells. In some embodiments, EVs are derived from immune cells. For example, EVs may be derived from red blood cells, white blood cells, cancer cells, stem cells, dendritic cells, macrophages, or other cell types.

### 3. Liposomes

In some embodiments, the present disclosure provides for compositions, preparations, and/or delivery particles that comprise liposomes.

The present disclosure contemplates liposomes capable of attaching and releasing nucleic acids, nucleic acid conjugates, polypeptides, and/or fusion proteins as described herein. Liposomes are microscopic spherical lipid bilayers surrounding an aqueous core that are made from amphiphilic molecules such as phospholipids. For example, a liposome may trap a nucleic acid between the hydrophobic tails of the phospholipid micelle. In some embodiments, water soluble agents can be entrapped in the core and lipid-soluble agents can be dissolved in the shell-like bilayer.

Those skilled in the art will appreciate that, in some embodiments liposomes allow water soluble and water insoluble chemicals to be used together in a medium without the use of surfactants or other emulsifiers. In some embodiments, liposomes can form spontaneously. For example, liposomes can form by forcefully mixing phospholipids in aqueous media. In some embodiments, liposomes can be formed, for example, when water soluble compounds are dissolved in an aqueous solution capable of hydrating phospholipids. Upon formation of the liposomes, these water soluble compounds are trapped within the aqueous liposomal center. The liposome wall, being a phospholipid membrane, holds fat soluble materials such as oils.

In some embodiments, liposomes provide controlled release of incorporated compounds. In some embodiments, liposomes do not provide controlled release of incorporated compounds. In addition, in some embodiments, liposomes can be coated with water soluble polymers such as polyethylene glycol to increase the pharmacokinetic half-life. Also contemplated is an ultra high-shear technology to refine liposome production, resulting in stable, unilamellar (single layer) liposomes having specifically designed structural characteristics. These unique properties of liposomes allow the simultaneous storage of normally immiscible compounds and the capability of their controlled release.

The present disclosure contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

Also contemplated is a delivery particle comprising liposomes that provides controlled release of at least one cargo as described herein. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

Compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

iii. Carbohydrate-Based Delivery Particles

The present disclosure provides for compositions, preparations, and/or delivery particles that comprise carbohydrate-based delivery particles (e.g., polysaccharide-based delivery particles).

A variety of carbohydrate-based delivery particles are known in the art. Non-limiting examples of carbohydrate-based delivery particles comprise, but are not limited to, chitosan, hyaluronic acid, dextran, arabinogalactan, starch, cyclodextrin, cycloamylose, pullulan, inulin, cellulose, hemicellulose, alginic acid, chondroitin sulfate, heparin, and gums (e.g., natural polysaccharide gums (e.g., Guar gum, xanthum gum, gum Arabic, carrageenan gum, pectin, etc.)) (see, e.g., Barclay, T. G., et al, Carbohydrate Polymer, 2019 Oct. 1; 221; 94-112, which is incorporated in its entirety herein by reference).

As may be appreciated by a skilled person, carbohydrate-based delivery particles are advantageous for cargo delivery as they have relatively high cargo loading. In some embodiments, a cargo may be released from a carbohydrate-based delivery particle by one or more external stimuli. In some embodiments, a cargo may be released from a carbohydrate-based delivery particle without one or more external stimuli.

In some embodiments, carbohydrate-based delivery particles are microspheres as described herein. In some embodiments, carbohydrate-based delivery particles are microparticles as described herein. In some embodiments, carbohydrate-based delivery particles are nanoparticles as described herein. In some embodiments, carbohydrate-based delivery particles are microcapsules as described herein. For example, microsphere, microparticle, nanoparticle, and microcapsule construction as described for polymer-based delivery particles may also be used to construct carbohydrate-based delivery particles as described herein.

Carbohydrate-based delivery particles may be constructed in a number of ways. In some embodiments, for example, a carbohydrate-based delivery particle may be constructed using polysaccharide chemistry. For example, a skilled person may appreciate that carbohydrate-based delivery particles as described herein may be constructed by exploiting functional groups in the polysaccharide. In some embodiments, a carbohydrate-based delivery particle as described herein may be constructed by exploiting a hydroxyl. In some embodiments, a carbohydrate-based delivery particle as described herein may be constructed by exploiting a carboxylic acid. In some embodiments, a carbohydrate-based delivery particle as described herein may be constructed by exploiting an amino group. The chemical nature of these groups makes them useful in the formation of carbohydrate-based delivery particles and binding of cargos (e.g., a cargo polypeptide, a shuttle-cargo polypeptide, a therapeutic polypeptide, nucleic acids encoding a cargo polypeptide, nucleic acids encoding a shuttle-cargo polypeptide, nucleic acids encoding a therapeutic polypeptide, nucleic acids encoding a barcoded cargo polypeptide, nucleic acids encoding a barcoded shuttle-cargo polypeptide, nucleic acids encoding a barcode, nucleic acids encoding a binder, etc.). In some embodiments, formation of carbohydrate-based delivery particles and binding of cargos is or comprises non-covalent interactions. In some embodiments, formation of carbohydrate-based delivery particles and binding of cargos is or comprises covalent linking reactions.

In some embodiments, carbohydrate-based delivery particles may self-assemble. In some embodiments, carbohydrate-based delivery particles may not self-assemble. In some embodiments, carbohydrate-based delivery particles may self-assemble spontaneously. In some embodiments, carbohydrate-based delivery particles may not self-assemble spontaneously. In some embodiments, carbohydrate-based delivery particles as described herein, may self-assemble to form, for example, hydrogels. In some embodiments, carbohydrate-based delivery particles as described herein, may self-assemble to not form hydrogels. In some embodiments, carbohydrate-based delivery particles as described herein, may self-assemble to form semicrystalline microparticles. In some embodiments, carbohydrate-based delivery particles as described herein, may self-assemble to form crystalline microparticles. In some embodiments, carbohydrate-based delivery particles as described herein, may self-assemble to form hydrophobically modified polysaccharides. In some embodiments, carbohydrate-based delivery particles as described herein, may self-assemble by ionic crosslinking. In some embodiments, carbohydrate-based delivery particles as described herein, may self-assemble by ionic assembly.

In some embodiments, carbohydrate-based delivery particles may be constructed by chemical crosslinking. In some embodiments, carbohydrate-based delivery particles may be constructed using covalent crosslinks. In some embodiments, carbohydrate-based delivery particles may be constructed using non-covalent crosslinks. In some embodiments, carbohydrate-based delivery particles may be constructed using click chemistry crosslinking. In some embodiments, carbohydrate-based delivery particles may be constructed using disulphide bridges. In some embodiments, carbohydrate-based delivery particles may be constructed using multifunctional reagents. In some embodiments, carbohydrate-based delivery particles may be constructed using alkenyl modification.

Sizes of carbohydrate-based delivery particles are generally tunable and may be engineered to be of different sizes. In some embodiments, carbohydrate-based delivery particles, as described herein, may be between 10 nm and 1000 nm in diameter. In some embodiments, carbohydrate-based delivery particles, as described herein, may be between 1 μm and 1000 μm.

iv. Polymer-Based Delivery Particles

The present disclosure provides for compositions, preparations, and/or delivery particles that comprise polymer-based delivery particles.

For example, in some embodiments, delivery particles described herein comprise poly(lactide-co-glycolide), aliphatic polyesters including, but not limited to, poly-glycolic acid and poly-lactic acid, hyaluronic acid, polyurethanes, polyacrylic acids, pseudo-poly(amino acids), polyhydroxybutyrate-related copolymers, polyanhydrides, polymethylmethacrylate, poly(ethylene oxide), lecithin and phospholipids.

In some embodiments, polymer-based delivery particles are microspheres as described herein. In some embodiments, polymer-based delivery particles are microparticles as described herein. In some embodiments, polymer-based delivery particles are microcapsules as described herein.

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (ProLease™, Alkermes: Cambridge, Mass.). For example, a freeze-dried medium comprising at least one therapeutic agent is homogenized in a suitable solvent and sprayed to manufacture microspheres in the range of 20 to 90 μm. Techniques are then followed that maintain sustained-release integrity during phases of purification, encapsulation, and storage. Scott et al., Improving Protein Therapeutics With Sustained Release Formulations, Nature Biotechnology, Volume 16:153-157 (1998). Modification of the microsphere composition by the use of biodegradable polymers can provide an ability to control the rate of nucleic acid release. Miller et al., Degradation rates of Oral Resorbable Implants, Polylactates and Polyglycolates: Rate Modification and Changes in PLA/PGA Copolymer Ratios, J. Biomed. Mater. Res., Vol. 11:711-719 (1977).

Alternatively, a sustained- or controlled-release microsphere preparation is prepared using an in-water drying method, where an organic solvent solution of a biodegradable polymer metal salt is first prepared. Subsequently, a dissolved or dispersed medium of nucleic acid(s) is added to the biodegradable polymer metal salt solution. The weight ratio of nucleic acid(s) to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and nucleic acid(s) is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and nucleic acid mixture are: i) phase separation during a gradual addition of a coacervating agent; ii) an in-water drying method or phase separation method, where an antiflocculant is added to prevent particle agglomeration and iii) by a spray-drying method.

Also contemplated in the disclosure is a medium comprising a microsphere or microcapsule capable of delivering controlled release of a nucleic acid for a duration of approximately between 1 day and 6 months. The microsphere or microparticle may be colored to allow the user the ability to see the medium clearly as it is dispensed. The microsphere or microcapsule may be clear. The microsphere or microparticle may be impregnated with a radio-opaque fluoroscopic dye.

Controlled-release microcapsules may be produced by using known encapsulation techniques such as centrifugal extrusion, pan coating and air suspension. Such microspheres and/or microcapsules can be engineered to achieve desired release rates. For example, Oliosphere™ (Macromed) is a controlled-release microsphere system. These particular microspheres are available in uniform sizes ranging between 5-500 μm and composed of biocompatible and biodegradable polymers. Specific polymer compositions of a microsphere can control the nucleic acid release rate such that custom-designed microspheres are possible, including effective management of the burst effect. ProMaxx™ (Epic Therapeutics, Inc.) is a protein-matrix delivery system. The system is aqueous in nature and is adaptable to standard pharmaceutical delivery models. In particular, ProMaxx™ are bioerodible protein microspheres that deliver both small and macromolecular drugs, and may be customized regarding both microsphere size and desired release characteristics.

A microsphere or microparticle may comprise a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated nucleic acid, therefore, will be selected for the pH environment in which dissolution is desired and stored in a pH preselected to maintain stability. Examples of pH sensitive material useful as encapsulants are Eudragit™ L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. Lipids may comprise the inner coating of the microcapsules. In these compositions, these lipids may be, but are not limited to, partial esters of fatty acids and hexitiol anhydrides, and edible fats such as triglycerides. Lew C. W, Controlled-Release pH Sensitive Capsule And Adhesive System And Method. U.S. Pat. No. 5,364,634 (herein incorporated by reference).

The present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a nucleic acid is directly bound to the surface of the microparticle or is indirectly attached via a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled-release medium is also empirically determined by the final density of glutaraldehyde-spacer crosslinks.

The present invention contemplates microparticles formed by spray-drying a composition comprising fibrinogen or thrombin with a nucleic acid. Preferably, these microparticles are soluble and the selected protein (i.e., fibrinogen or thrombin) creates the walls of the microparticles. Consequently, the nucleic acids are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant, thereby releasing the incorporated compound into the immediate surrounding area.

One having skill in the art will understand that the shape of the microspheres need not be exactly spherical, only to be small particles capable of being administered by an appropriate route to the site or subject of interest. Microparticles may be comprised of a biocompatible and/or biodegradable material selected from the group consisting of polylactide, polyglycolide and copolymers of lactide/glycolide (PLGA), hyaluronic acid, modified polysaccharides and any other well-known material.

v. Other Delivery Particles

In some embodiments, delivery particles, as contemplated herein. may be any delivery particle known in the art. For example, in some embodiments, delivery particles may be beads, metal-based (e.g., Au, Si, Zn, Fe) particles, micelles, etc.

vi. Production Nucleic Acids and Cells

Among other things, the present disclosure provides for production nucleic acids, e.g., from which a viral delivery particle as described herein is produced. Moreover, among other things, the present disclosure provides for production cells that contain and/or produce nucleic acids used to produce a viral delivery particle as described herein.

### 1. Production Nucleic Acids and Cells for Viral Delivery Particles

For example, in some embodiments, a viral delivery particle is an AAV delivery particle. AAV delivery particles, and related AAV systems and AAV constructs are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is incorporated in its entirety herein by reference). Methods for generating and using AAV constructs are described, for example, in U.S. Pat. Nos. 5,139,941, 4,797,368 and PCT filing application US2019/060328, each of which is incorporated in its entirety herein by reference.

Methods for obtaining viral constructs are known in the art. For example, to produce AAV constructs, the methods typically involve culturing a host cell which contains a nucleic acid sequence encoding an AAV capsid protein or fragment thereof; a functional rep gene; a recombinant AAV construct composed of AAV inverted terminal repeats (ITRs) and a coding sequence; and/or sufficient helper functions to permit packaging of the recombinant AAV construct into the AAV capsid proteins.

In some embodiments, components to be cultured in a host cell to package an AAV construct in an AAV capsid may be provided to the host cell in trans. Alternatively, any one or more components (e.g., recombinant AAV construct, rep sequences, cap sequences, and/or helper functions) may be provided by a stable host cell that has been engineered to contain one or more such components using methods known to those of skill in the art. In some embodiments, such a stable host cell contains such component(s) under the control of an inducible promoter. In some embodiments, such component(s) may be under the control of a constitutive promoter. In some embodiments, a selected stable host cell may contain selected component(s) under the control of a constitutive promoter and other selected component(s) under the control of one or more inducible promoters. For example, a stable host cell may be generated that is derived from HEK293 cells (which contain E1 helper functions under the control of a constitutive promoter), but that contain the rep and/or cap proteins under the control of inducible promoters. Other stable host cells may be generated by one of skill in the art using routine methods.

Recombinant AAV construct, rep sequences, cap sequences, and helper functions required for producing an AAV of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N.Y., which is incorporated in its entirety herein by reference). Similarly, methods of generating AAV particles are well known and any suitable method can be used with the present disclosure (see, e.g., K. Fisher et al, J. Virol., 70:520-532 (1993) and U.S. Pat. No. 5,478,745, which are incorporated in their entirety herein by reference).

In some embodiments, recombinant AAVs may be produced using a triple transfection method (e.g., as described in U.S. Pat. No. 6,001,650, which is incorporated in its entirety herein by reference). In some embodiments, recombinant AAVs are produced by transfecting a host cell with a recombinant AAV construct (comprising a coding sequence) to be packaged into AAV particles, an AAV helper function construct, and an accessory function construct. An AAV helper function construct encodes “AAV helper function” sequences (i.e., rep and cap), which function in trans for productive AAV replication and encapsidation. In some embodiments, the AAV helper function construct supports efficient AAV construct production without generating any detectable wild type AAV particles (i.e., AAV particles containing functional rep and cap genes). Non-limiting examples of constructs suitable for use with the present disclosure include pHLP19 (see, e.g., U.S. Pat. No. 6,001,650, which is incorporated in its entirety herein by reference) and pRep6cap6 construct (see, e.g., U.S. Pat. No. 6,156,303, which is incorporated in its entirety herein by reference). An accessory function construct encodes nucleotide sequences for non-AAV derived viral and/or cellular functions upon which AAV is dependent for replication (i.e., “accessory functions”). Accessory functions may include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAVs are separated from contaminating virus. Other systems do not require infection with helper virus to recover the AAV—the helper functions (i.e., adenovirus E1, E2a, VA, and E4 or herpesvirus UL5, UL5, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

Other types of viral delivery particles can be produced in accordance with embodiments described herein.

### 2. Production Nucleic Acids and Cells for Extracellular Vesicle Delivery Particles

Among other things, the present disclosure provides for production of nucleic acids, e.g., from which an extracellular vesicle delivery particle (EV) as described herein is produced. Moreover, among other things, the present disclosure provides for production cells that contain and/or produce nucleic acids used to produce an extracellular vesicle delivery particle as described herein.

EVs, e.g., exosomes, of the present disclosure can be produced from a cell grown in vitro or a body fluid of a subject. When exosomes are produced from in vitro cell culture, various producer cells, e.g., HEK293 cells, CHO cells, C2C12, and MSCs, can be used. In some aspects, producer cells can be selected from HEK293 cells, HEK293S cells, HEK293SF cells, Chinese Hamster Ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells, dendritic cells, macrophages, B cells, mast cells, neutrophils, Kupffer-Browicz cells, PER.C6 cells, Induced pluripotent stem cells (iPSCs), or C2C12 cells. In some aspects, the producer cells are stem cells.

In some embodiments, producer cells can be genetically and/or pharmacologically modified to reduce gene and/or protein function in a cholesterol biosynthetic pathway, as described herein. In some aspects, modified producer cells can be further modified (e.g., genetically) to comprise exogenous sequences encoding a protein to produce EVs described herein. Genetically-modified producer cells can contain the exogenous sequence by transient or stable transformation. Nucleic acids encoding cargo polypeptides can be transformed as a plasmid. Nucleic acids encoding cargo polypeptides can be stably integrated into a genomic sequence of a producer cell, at a targeted site or in a random site. In some embodiments, a stable cell line is generated for production of lumen-engineered exosomes. In some embodiments modified producer cells comprise a recruiting polypeptide. In some embodiments, a recruiting polypeptide comprises a nucleic acid binding moiety. In some embodiments, a recruiting polypeptide comprises a membrane-associating moiety. In some embodiments, a recruiting polypeptide comprises a nucleic acid binding moiety and a membrane-associating moiety.

Nucleic acids encoding cargo polypeptides can be inserted into a genomic sequence of a producer cell, located within, upstream (5′-end) or downstream (3′-end) of an endogenous sequence encoding an exosome protein. Various methods known in the art can be used for the introduction of nucleic acids encoding cargo polypeptides into a producer cell. For example, cells modified using various gene editing methods (e.g., methods using a homologous recombination, transposon-mediated system, loxP-Cre system, CRISPR/Cas9 or TALEN) are within the scope of the present disclosure.

In some embodiments, the present disclosure provides a cell composition comprising modified cells that produce EVs, wherein modified cells have a reduced gene and/or protein function in a cholesterol biosynthetic pathway. In some embodiments, reduced gene and/or protein comprises protein in a cholesterol biosynthesis comprises one or more genes selected from 3-hydroxy-3-methylglutaryl-coenzyme A reductase (HMGCR), sterol regulatory element-binding protein 2 (SREBF2), Squalene epoxidase (SQLE), or 7-Dehydrocholesterol reductase (DHCR7) or a protein encoded by the gene.

In some embodiments, the present disclosure provides producer cells for use in the methods described herein. In some aspects, producer cells are prepared according to the methods described herein.

Also disclosed are methods for delivering a nucleic acid as described herein to a cell (e.g., a target cell). Methods may include contacting a cell with exosomes disclosed herein. Targeted exosomes may comprise a ligand at the N-terminus of a fusion protein that targets the exosomes to target cells. As such, a ligand may be present on the surface of the exosome where the ligand binds specifically to a receptor on the surface of the target cells. Cargo components may include a candidate therapeutic nucleic acid or a therapeutic nucleic acid for treating a disease or disorder when a cargo component is delivered to the target cells (e.g., a hybrid RNA comprising a miRNA, shRNA, mRNA, ncRNA, or any combination of any of these RNAs fused to the RNA-motif that binds to the RNA-binding domain of the fusion protein). Exosomes may be formulated as a pharmaceutical composition for treating the disease or disorder.

### VIII. Systems

i. Expression Systems

Numerous expression systems exist that comprise at least a part or all of the compositions discussed herein. Prokaryote- and/or eukaryote-based systems can be employed to produce nucleic acid sequences, or their cognate cargo polypeptides, proteins and peptides. For example, component components, barcode components, and related cargo polypeptides and peptide barcodes may utilize an expression system, such as an inducible or constitutive expression system. Many such systems are commercially and widely available.

For example, an insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems, other examples of expression systems include STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

ii. Cells, Tissues, Organs, Organoids, Organisms

Among other things, the present disclosure provides for expression of cargo polypeptides in cells (e.g., eukaryotic cells, e.g., prokaryotic cells), tissues, organs, organoids, or organisms. In some embodiments, cells, tissues, organs, organoids, or organisms are mammalian cells, tissues, organs, organoids, or organisms. Nucleic acids, or cargo polypeptides encoded by nucleic acids, described herein may be comprised within a cell, tissue, organ, organoid, or organism. Nucleic acids may be in a eukaryotic cell, such as a mammalian cell or a plant cell. Mammalian cells, tissues, organs, organoids, or organisms may be of human, non-human primate, bovine, porcine, rodent or mouse origin. Cells, tissues, organs, organoids, or organisms may be of non-mammalian origin, such as poultry, fish, or shrimp.

A mammalian cell, tissue, organ, organoid, or organism may be of human or non-human mammalian origin, e.g., primate, bovine, ovine, porcine, canine, rodent, Leporidae such as monkey, cow, sheep, pig, dog, rabbit, rat or mouse. A cell, tissue, organ, organoid, or organism may be of non-mammalian origin such as poultry bird (e.g., chicken), vertebrate fish (e.g., salmon) or shellfish (e.g., oyster, clam, lobster, shrimp). A cell, tissue, organ, organoid, or organism may be or comprise a tumor cell, tissue, organ, or organoid. A cell, tissue, organ, or organoid may be or comprise a benign cell, tissue, organ, or organoid. A cell, tissue, organ, or organoid may be or comprise a kidney cell, tissue, organ, or organoid. A cell, tissue, organ, or organoid may be or comprise an adipose cell, tissue, organ, or organoid. A cell, tissue, organ, or organoid may be or comprise a brain cell, tissue, organ, or organoid. A cell, tissue, organ, or organoid may be or comprise a liver cell, tissue, organ, or organoid. A cell, tissue, organ, or organoid may be a blood cell, tissue, organ, or organoid. A cell, tissue, organ, or organoid may be or comprise a skin cell, tissue, organ, organoid, or organism. A cell, tissue, organ, or organoid may be or comprise a muscle cell, tissue, organ, or organoid. A cell, tissue, organ, or organoid may be or comprise a cardiac cell, tissue, organ, or organoid. A cell, tissue, organ, or organoid may be an ocular cell, tissue, organ, or organoid. A cell, tissue, organ, or organoid may be or comprise a nerve cell, tissue, organ, organoid, or organism. A cell, tissue, organ, or organoid may be a parenchyma cell, tissue, organ, or organoid. A cell, tissue, organ, or organoid may be or comprise an intestinal cell, tissue, organ, or organoid. A cell, tissue, organ, or organoid may be or comprise a lung cell, tissue, organ, or organoid. A cell, tissue, organ, or organoid may be or comprise an immune cell, tissue, organ, or organoid. Populations of cells may be or comprise one or more cells described herein, or any other cell type present in the tissue, organ, organoid or organism of interest.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Some vectors may employ control sequences that allow it to be replicated and/or expressed in both prokaryotic and eukaryotic cells. One of skill in the art would further understand the conditions under which to incubate all of the above-described host cells to maintain them and to permit replication of a vector. Also understood and known are techniques and conditions that would allow large-scale production of vectors, as well as production of the nucleic acids encoded by vectors and their cognate polypeptides, proteins, or peptides.

### IX. Use

i. Assessing Cargos

Technologies described herein may be used to detect, assess, and/or characterize cargos (e.g., cargo polypeptides (e.g., variant cargo polypeptides); cargo nucleic acids (e.g., variant cargo nucleic acids)). In some embodiments, provided technologies may be used, for example, to assay cargos in complex environments (e.g., serum, blood, tissue, etc.). In some embodiments, cargos may be nucleic acids encoding cargo polypeptides. In some embodiments, cargos may be nucleic acids encoding candidate therapeutic polypeptides or therapeutic polypeptides.

As described herein, a cargo may be associated with a barcode (i.e., a barcoded cargo). In some embodiments, a barcoded cargo may be assayed using binding agents (e.g., phages with binders expressed on them) using methods as described herein. In some embodiments, a barcoded cargo may be captured (e.g., using affinity reagents) on a surface (e.g., beads or plates). In some embodiments, a barcoded cargo may be immobilized for barcode assaying. In some embodiments, a barcoded cargo is contacted with one or more binders and subject to decoding as described herein.

In some embodiments, cargos may be detected, assessed, and/or characterized in vitro. In some embodiments, cargos may be detected, assessed, and/or characterized in vivo.

In some embodiments, cargos that were previously detected, assessed, and/or characterized may be pooled together and subject to further detection, assessment, and/or characterization, for example, using methods as described herein. In some embodiments, such further detection, assessment, and/or characterization may be performed in vitro. In some embodiments, such further detection, assessment, and/or characterization may be performed in vivo.

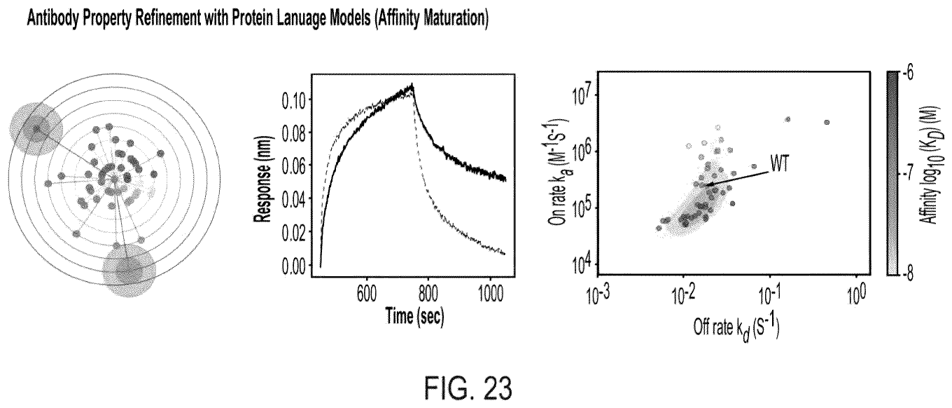

In some embodiments, cargos that were previously detected, assessed, and/or characterized may be subject to mutagenesis (e.g., random mutagenesis, targeted mutagenesis, or using machine learning (e.g., pre-trainer large language model)). In some embodiments, for example, such mutated cargos (e.g., variant cargos (e.g., variant cargo nucleic acids, variant cargo polypeptides) may be pooled together and subject to further detection, assessment, and/or characterization, for example, using methods as described herein. FIG. 23 depicts a schematic of such an exemplary method. FIG. 23 shows that variants of a previously detected, assessed, and/or characterized cargo may be generated and subject to further detection, assessment, and/or characterization, for example, using methods as described herein. In some embodiments, such iterative methods, for example, could result in a variant cargo that for example shows improved or enhanced functionality (e.g., improved developability, expression, affinity, etc.). In some embodiments, such further detection, assessment, and/or characterization may be performed in vitro. In some embodiments, such further detection, assessment, and/or characterization may be performed in vivo.

In some embodiments, cargos may be disposed within one or more delivery particles as described herein.

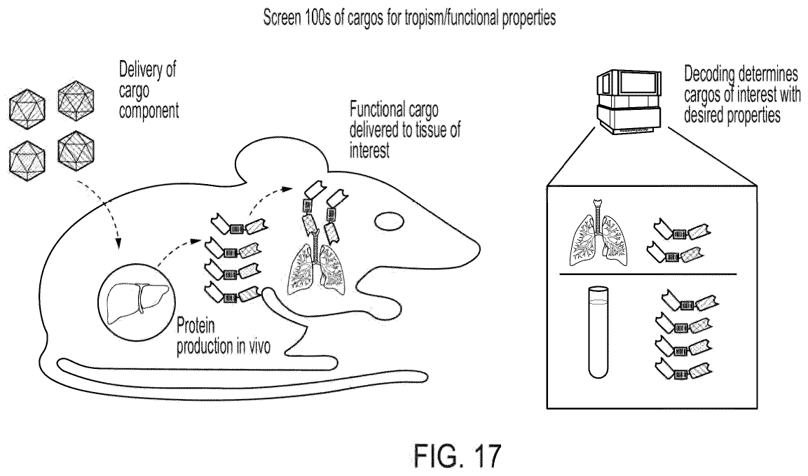

For example, FIG. 17 depicts a schematic of an exemplary method that provides for high throughput cargo delivery, production, screening, identification, and/or characterization as described herein. In some embodiments, nucleic acids comprising (1) a cargo component whose nucleotide sequence is or comprises a sequence encoding a cargo polypeptide and (2) a barcode component whose nucleotide sequence is or comprises a sequence encoding a peptide barcode are disposed within one or more delivery particles and are administered to an animal (e.g., a mammal). In some embodiments, functional cargos are expressed in a tissue of interest, and decoding methods are used to determine cargos and/or delivery particles with desired properties.

In some embodiments, methods described herein determine simultaneous in vivo assessment of phenotypes of cargos in multiple tissues. In some embodiments, a phenotype includes biodistribution information related to cargos. In some embodiments, a phenotype includes pharmacokinetic (clearance) information of cargos. In some embodiments, a phenotype includes half-life information of cargos. In some embodiments, a phenotype includes tissue-mediated drug disposition (TMDD) of cargos. In some embodiments, a phenotype includes properties related to in vivo stability of cargos. In some embodiments, multiple phenotypes of cargos may be determined simultaneously.

In some embodiments, methods described herein determine simultaneous in vivo tracking and/or assessment and/or quantification of different cargos. In some embodiments, methods described herein determine simultaneous in vivo tracking and/or assessment and/or quantification of different types of delivery particles.

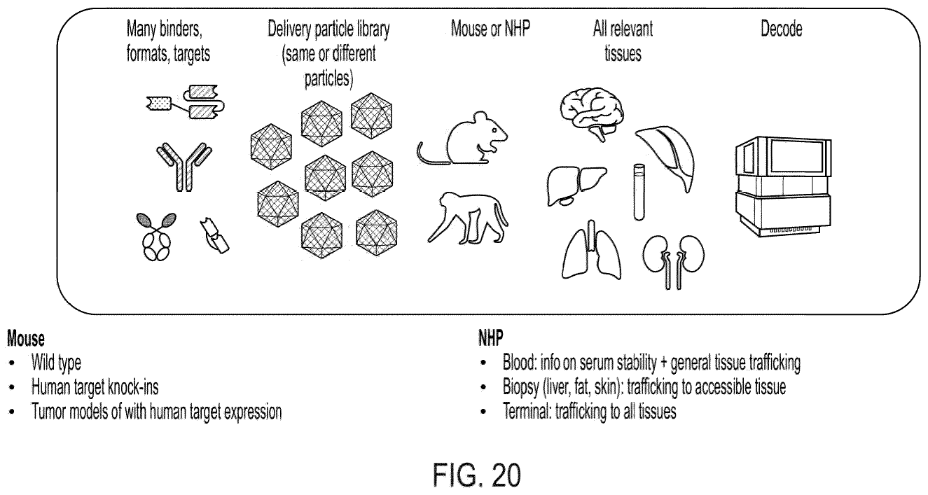

For example, FIG. 20 depicts a schematic showing that high-throughput screening using the delivery and assessment platform as described herein provides for screening of multiple cargos, formats, targets, and tissues simultaneously in different models (e.g., mouse, NHP, etc.).

ii. Assessing Shuttles

Technologies described herein may be used to detect, assess, and/or characterize shuttles (e.g., peptide shuttles (e.g., variant peptide shuttles); shuttle nucleic acids (e.g., variant shuttle nucleic acids), shuttle components (e.g., variant shuttle components)). For example, in some embodiments, technologies described herein may be used for the detection, assessment, and/or characterization of one or more shuttles (e.g., in a complex mixture (e.g., in vivo). In some embodiments, provided technologies may be used, for example, to assay shuttles in complex environments (e.g., serum, blood, tissue, etc.). That is, some embodiments, a shuttle agent (e.g., shuttles (e.g., peptide shuttles (e.g., variant peptide shuttles); shuttle nucleic acids (e.g., variant shuttle nucleic acids), shuttle components (e.g., variant shuttle components))) as described herein can be detected, assessed, and/or characterized similarly to how a cargo agent (e.g., cargos (e.g., cargo polypeptides (e.g., variant cargo polypeptides); cargo nucleic acids (e.g., variant cargo nucleic acids), cargo components (e.g., variant cargo components))) is detected, assessed, and/or characterized using technologies described herein.

As described herein, a shuttle may be associated with a barcode (i.e., a barcoded shuttle). In some embodiments, a barcoded shuttle may be assayed using binding agents (e.g., phages with binders expressed on them) using methods as described herein. In some embodiments, a barcoded shuttle may be captured (e.g., using affinity reagents) on a surface (e.g., beads or plates). In some embodiments, a barcoded shuttle may be immobilized for barcode assaying. In some embodiments, a barcoded shuttle is contacted with one or more binders and subject to decoding as described herein.

In some embodiments, shuttles may be detected, assessed, and/or characterized in vitro. In some embodiments, shuttles may be detected, assessed, and/or characterized in vivo.

In some embodiments, shuttles that were previously detected, assessed, and/or characterized may be pooled together and subjected to further detection, assessment, and/or characterization, for example, using methods as described herein. In some embodiments, such further detection, assessment, and/or characterization may be performed in vitro. In some embodiments, such further detection, assessment, and/or characterization may be performed in vivo.

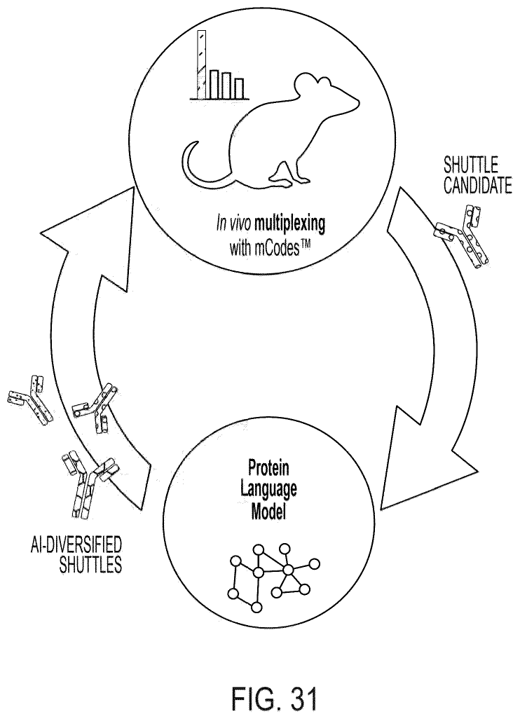

In some embodiments, shuttles that were previously detected, assessed, and/or characterized may be subjected to mutagenesis (e.g., random mutagenesis, targeted mutagenesis, or using machine learning (e.g., pre-trainer large language model)). In some embodiments, for example, such mutated shuttles (e.g., variant shuttles (e.g., variant shuttle nucleic acids, variant shuttle polypeptides) may be pooled together and subjected to further detection, assessment, and/or characterization, for example, using methods as described herein. FIG. 31 depicts a schematic of such an exemplary method. FIG. 31 shows that variants of a previously detected, assessed, and/or characterized shuttle may be generated and subjected to further detection, assessment, and/or characterization, for example, using methods as described herein. In some embodiments, such iterative methods, for example, could result in a variant shuttle that, for example, shows improved or enhanced functionality (e.g., improved developability, expression, affinity, etc.). In some embodiments, a shuttle may comprise a structural feature (e.g., length, hydrophobicity, affinity, sequence motif, etc.) that has an effect (e.g., positive or negative) on cargo function. In some embodiments, such further detection, assessment, and/or characterization may be performed in vitro. In some embodiments, such further detection, assessment, and/or characterization may be performed in vivo.

In some embodiments, shuttles may be disposed within one or more delivery particles as described herein.

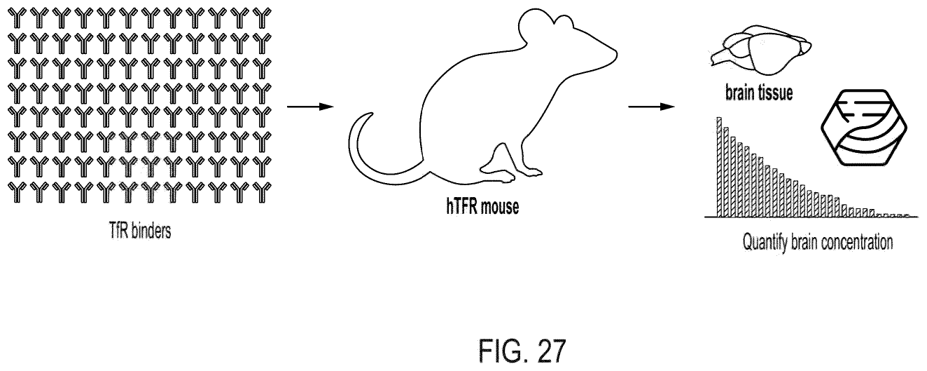

For example, FIG. 27 depicts a schematic of an exemplary method that provides for high throughput shuttle delivery, production, screening, identification, and/or characterization as described herein. FIG. 27 shows a schematic of an in vivo screen for TfR-targeted VHH shuttles in a humanized TfR mouse according to an embodiment described herein. In some embodiments, polypeptides comprising (1) a peptide shuttle and (2) a peptide barcode are administered to an animal (e.g., a mammal). In some embodiments, functional shuttles are delivered to a tissue of interest, and decoding methods are used to determine shuttles and/or delivery particles with desired properties. Alternately such delivery may also be achieved, in some embodiments, when nucleic acids comprising (1) a shuttle component whose nucleotide sequence is or comprises a sequence encoding a peptide shuttle, and (2) a barcode component whose nucleotide sequence is or comprises a sequence encoding a peptide barcode are disposed within one or more delivery particles and are administered to an animal (e.g., a mammal). In some embodiments, functional shuttles are expressed in a tissue of interest, and decoding methods are used to determine shuttles and/or delivery particles with desired properties.

In some embodiments, methods described herein determine simultaneous in vivo assessment of phenotypes of shuttles in multiple tissues. In some embodiments, a phenotype includes biodistribution information related to shuttles. In some embodiments, a phenotype includes pharmacokinetic (clearance) information of shuttles. In some embodiments, a phenotype includes half-life information of shuttles. In some embodiments, a phenotype includes tissue-mediated drug disposition (TMDD) of shuttles. In some embodiments, a phenotype includes properties related to in vivo stability of shuttles. In some embodiments, multiple phenotypes of shuttles may be determined simultaneously.

In some embodiments, methods described herein determine simultaneous in vivo tracking and/or assessment and/or quantification of different shuttles. In some embodiments, methods described herein determine simultaneous in vivo tracking and/or assessment and/or quantification of different types of delivery particles.

iii. Compositions

Among other things, the present disclosure provides compositions. In some embodiments, a composition comprises a nucleic acid as described herein. In some embodiments, a composition comprises one or more nucleic acids as described herein. In some embodiments, a composition comprises a plurality of nucleic acids as described herein. In some embodiments, when more than one construct is included in the composition, the nucleic acids are each different.

In some embodiments, a composition comprises a delivery particle as described herein. In some embodiments, a composition comprises one or more delivery particles as described herein. In some embodiments, a composition comprises a plurality of delivery particles. In some embodiments, when more than one delivery particle is included in the composition, the delivery particles are each different.

In some embodiments, a composition comprises an AAV delivery particle as described herein. In some embodiments, a composition comprises one or more AAV delivery particles as described herein. In some embodiments, a composition comprises a plurality of AAV delivery particles. In some embodiments, when more than one AAV delivery particle is included in the composition, the AAV delivery particles are each different.

In some embodiments, a composition comprises a barcode as described herein. In some embodiments, a composition comprises one or more barcodes as described herein. In some embodiments, a composition comprises a plurality of barcodes as described herein. In some embodiments, when more than one barcode is included in the composition, the barcodes are each different.

In some embodiments, a composition comprises a shuttle (e.g., peptide shuttle, shuttle component) as described herein. In some embodiments, a composition comprises one or more shuttles (e.g., peptide shuttles, shuttle components) as described herein. In some embodiments, a composition comprises a plurality of shuttles (e.g., peptide shuttles, shuttle components) as described herein. In some embodiments, when more than one shuttle (e.g., peptide shuttle, shuttle component) is included in the composition, the shuttles are each different. In some embodiments, when more than one shuttle (e.g., peptide shuttle, shuttle component) is included in the composition, the shuttles are all the same.

In some embodiments, a composition comprises a binder as described herein. In some embodiments, a composition comprises one or more binders as described herein. In some embodiments, a composition comprises a plurality of binders as described herein. In some embodiments, when more than one binder is included in the composition, the binders are each different.

In some embodiments, a composition comprises a binding agent as described herein. In some embodiments, a composition comprises one or more binding agents as described herein. In some embodiments, a composition comprises a plurality of binding agents as described herein. In some embodiments, when more than one binding agent is included in the composition, the binding agents are each different.

In some embodiments, a composition comprises a cargo polypeptide as described herein. In some embodiments, a composition comprises one or more cargo polypeptides as described herein. In some embodiments, a composition comprises a plurality of cargo polypeptides as described herein. In some embodiments, when more than one cargo polypeptide is included in the composition, the cargo polypeptides are each different. In some embodiments, when more than one cargo polypeptide is included in the composition, the cargo polypeptides are all the same.

In some embodiments, a composition comprises a shuttle-cargo as described herein. In some embodiments, a composition comprises one or more shuttle-cargos as described herein. In some embodiments, a composition comprises a plurality of shuttle-cargos as described herein. In some embodiments, when more than one shuttle-cargos is included in the composition, the shuttle-cargos are each different. In some embodiments, when more than one shuttle-cargos is included in the composition, the shuttle-cargos are all the same.

In some embodiments, a composition comprises a shuttle-cargo polypeptide as described herein. In some embodiments, a composition comprises one or more shuttle-cargo polypeptides as described herein. In some embodiments, a composition comprises a plurality of shuttle-cargo polypeptides as described herein. In some embodiments, when more than one shuttle-cargo polypeptide is included in the composition, the shuttle-cargo polypeptides are each different. In some embodiments, when more than one shuttle-cargo polypeptide is included in the composition, the shuttle-cargo polypeptides are all the same.

In some embodiments, a composition comprises a candidate therapeutic polypeptide or therapeutic polypeptide as described herein. In some embodiments, a composition comprises one or more candidate therapeutic polypeptides or therapeutic polypeptides as described herein. In some embodiments, a composition comprises a plurality of candidate therapeutic polypeptides or therapeutic polypeptides as described herein. In some embodiments, when more than one candidate therapeutic polypeptides or therapeutic polypeptides is included in the composition, the candidate therapeutic polypeptides or therapeutic polypeptides are each different.

In some embodiments, a composition is or comprises a pharmaceutical composition.

iv. Kits

Technologies as described herein may be provided in the form of a composition. For example, in some embodiments, a composition may comprise one or more elements (e.g., nucleic acid, amino acid, etc.) to produce or generate one or more barcodes and/or binders as described herein. In some embodiments, a composition may comprise one or more elements to produce or generate a set of barcodes. In some embodiments, a composition may comprise one or more elements to produce or generate a set of shuttles. In some embodiments, a composition may comprise one or more elements to produce or generate a set of binders. In some embodiments, a composition may comprise one or more elements to produce or generate a pool of barcode-binder pairs. In some embodiments, a composition may comprise one or more elements to produce or generate binding agents (e.g., phage expressing binders). In some embodiments, a composition may be a barcode composition. In some embodiments, a composition may be a shuttle composition. In some embodiments, a composition may be a shuttle-cargo composition. In some embodiments, a composition may be a shuttle-cargo polypeptide composition. In some embodiments, a composition may be a binder composition. In some embodiments, a composition may be a barcode-binder composition. In some embodiments, a composition may be a binding agent composition. In some embodiments, a composition may comprise one or more of barcodes, shuttles, binders, binding agents, and/or components thereof. In some embodiments, a composition may comprise one or more sets/pools of barcodes, shuttles, binders, binding agents, and/or components thereof.

Provided herein are compositions comprising nucleic acids, delivery particles, barcodes, shuttles, binders, binding agents, therapeutic agents, cargos, shuttle-cargos, polypeptides (e.g., cargo polypeptides, shuttle-cargo polypeptides, therapeutic polypeptides, etc.), or components thereof. In some embodiments, a composition comprises nucleic acids, delivery particles, barcodes, shuttles binders, binding agents, therapeutic agents, cargos, shuttle-cargos, polypeptides (e.g., cargo polypeptides, shuttle-cargo polypeptides, therapeutic polypeptides, etc.), components thereof and/or combinations thereof, which have been assessed, identified, characterized or assayed using methods as described herein. In some embodiments, a composition provided herein comprises one or more, two or more, three or more, four or more, five or more, six or more, seven or more, eight or more, nine or more, or ten or more nucleic acids, delivery particles, barcodes, shuttles, binders, binding agents, therapeutic agents, cargos, shuttle-cargos, polypeptides (e.g., cargo polypeptides, shuttle-cargo polypeptides, therapeutic polypeptides, etc.), components thereof and/or combinations thereof, which have been assessed, identified, characterized or assayed using methods as described herein.

In some embodiments, a composition provided herein comprises two or more, three or more, four or more, five or more, six or more, seven or more, eight or more, nine or more, or ten or more nucleic acid sequences or amino acid sequences as listed in Table 1, Table 2, Table 3, Table 4, Table 5, Table 6, or Table 7.

A composition as described herein may be formulated in various forms. For example, in some embodiments, a composition as described herein may be formulated in a powder form (e.g., lyophilized). In some embodiments, a composition as described herein may be formulated in a liquid form.

In some embodiments, compositions for use in accordance with the present disclosure are pharmaceutical compositions, e.g., for administration (e.g., topical, oral, subcutaneous, intravenous, intramuscular, intracerebral, intrathecal, rectal (e.g., rectal intubation), opthalmical, intravitreal, or suprachoroidal administration) to a subject (e.g., a mammal (e.g., a human)). In some embodiments, such compositions are administered to a subject to detect, characterize, and/or assess one or more attributes of one or more cargos administered or to be administered to the subject. Pharmaceutical compositions typically include an agent to be administered (e.g., nucleic acids, delivery particles, barcodes, shuttles, binders, binding agents, therapeutic agents, cargos, shuttle-cargos, polypeptides (e.g., cargo polypeptides, shuttle-cargo polypeptides, therapeutic polypeptides, etc.), and/or components thereof), and a pharmaceutically acceptable carrier. Certain exemplary pharmaceutically acceptable carriers include, for instance saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include topical, oral, subcutaneous, intravenous, intramuscular, intracerebral, intrathecal, rectal, (e.g., rectal intubation), opthalmical, intravitreal, or suprachoroidal administration.

In some embodiments, pharmaceutically compatible binding agents, and/or adjuvant materials can be included as part of a pharmaceutical composition. In some particular embodiments, a pharmaceutical composition can contain, e.g., any one or more of the following inactive ingredients, or compounds of a similar nature: a binder, an excipient, a lubricant, a glidant, or some similar such compound.

Compositions can be included in a kit, container, pack, or dispenser, together with instructions for administration (e.g., to a subject) or for use in a method described herein. In some embodiments, instructions may include methods to reconstitute a powder form composition to a liquid form composition for further use. In some embodiments, a kit may include instructions that allows a user to generate new set of binders for a new set of barcodes. In some embodiments, a kit comprises a set of instructions to perform sequencing of one or more phage particles bound to one or more barcodes.

In some embodiments, a kit comprises information designating peptide barcodes for each binder. In some embodiments, a kit comprises a computer readable program for decoding sequencing data.

In some embodiments, a kit comprises reagents to express a binder on a phage particle. In some embodiments, a kit comprises nucleic acids that encode one or more barcodes. In some embodiments, a kit comprises nucleic acids that encode one or more binders. In some embodiments, a kit comprises nucleic acids that encode one or more shuttles (e.g., shuttle component).

Those skilled in the art, reading the present disclosure, will appreciate that, in some embodiments, a composition (e.g., a nucleic acid composition, a delivery particle composition, a binder composition, a barcode composition, a shuttle composition, a shuttle-cargo composition, a binding agent composition, a polypeptide composition (e.g., a cargo polypeptide composition, a shuttle-cargo polypeptide composition, a therapeutic polypeptide composition), etc.) as described herein may be or comprise one or more cells, tissues, or organisms (e.g., plant or microbe cells, tissues, virus, or organisms) that produce (e.g., have produced, and/or are producing) a relevant binder, barcode, shuttle, shuttle-cargo, binding agent, and/or polypeptide (e.g., a cargo polypeptide, a shuttle-cargo polypeptide composition, a therapeutic polypeptide) as described herein.

Those skilled in the art will appreciate that, in some embodiments, technologies for preparing compositions and/or preparations, and/or for preparing (and particularly for preparing pharmaceutical compositions) may include one or more steps of assessing or characterizing a compound, preparation, or composition, e.g., as part of quality control. In some embodiments, if an assayed material does not meet pre-determined specifications for the relevant assessment, it is discarded. In some embodiments, if such assayed material does meet the pre-determined specifications, then it continues to be processed as described herein.

In some embodiments, a composition is tailored to a specific subject (e.g., a specific mammal, e.g., a patient). In some embodiments, a composition is specific for a cargo to be assessed for an individual subject (e.g., mammal (e.g., human, mouse, etc.)). In some embodiments, a composition is specific for cargos to be assessed for an individual subject (e.g., mammal (e.g., human, mouse)). In some embodiments, a composition is specific for cargos of a population of subjects (e.g., mammals (e.g., humans, mice, etc.)). Populations of subjects can include, but are not limited to: families, subjects in the same regional location (e.g., neighborhood, city, state, or country), subjects with the same disease or condition, subjects of a particular age or age range, subjects that consume a particular diet (e.g., food, food source, or caloric intake).

### EXEMPLIFICATION

### Example 1: Identification of Barcodes, Corresponding Binding Agents, and Determining Fingerprints

The present Example demonstrates methods for identifying barcodes, corresponding binding agents (e.g., binders expressed on binding agents), and determining fingerprints (e.g., barcode fingerprints) and using the information to determine the proportion of a barcode in a given mixture. The resulting materials can then be used to measure and quantify different cargos.

### Design and Synthesis of a Barcode Library:

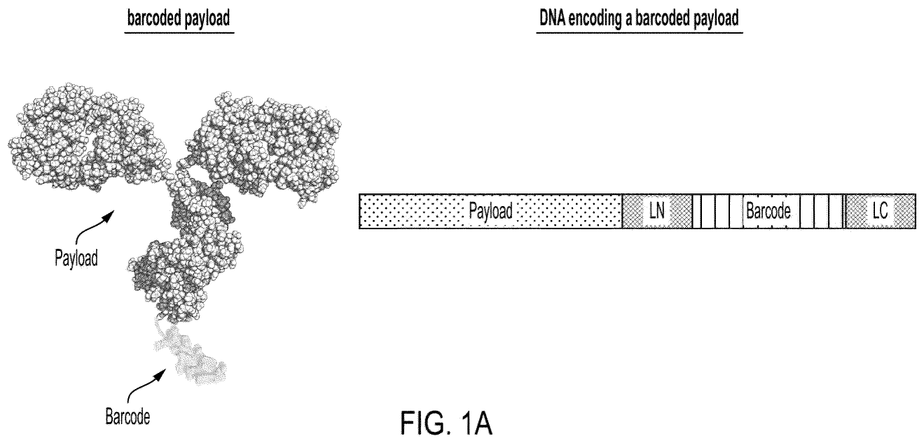

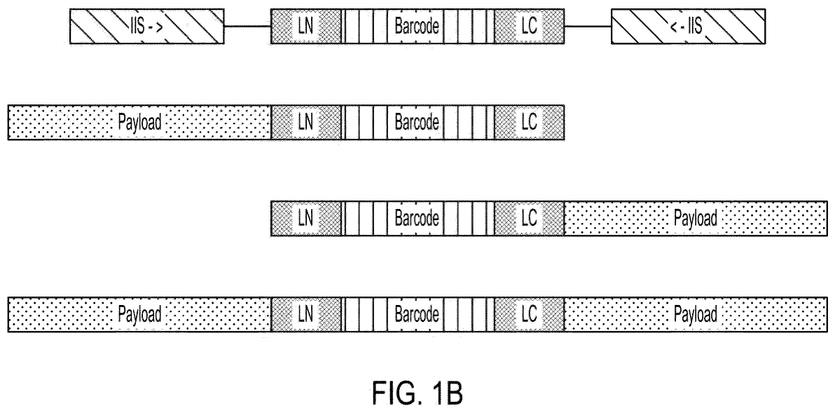

Barcode sequences were designed which contain specific sequence motifs thought to fold into a given helical or loop structure. All sequences from the Protein Data Bank (PDB) were downloaded, along with their corresponding secondary structure predictions. Sequences were selected and subsetted from the full sequence if they met the criteria of: being a contiguous helix or loop sequence for a length of 8-25 amino acids. A random subset of 100,000 of the peptide sequences matching this criteria were then ordered as an oligo pool, containing constant overhangs and type US sites for cloning into a vector (see FIG. 1B).

Cloning Barcode Library into Expression Plasmid:

The designed pool of barcodes was cloned into a pET expression vector to yield a barcode attached to a cargo protein. A plasmid was constructed containing 6×HIS-HALO-TEV-LN-IIS-LC, allowing for direct cloning of the oligo pool via golden gate assembly. LN and LC represent the constant overhangs in the oligo pool used for ligation (FIG. 1B). 1 μg of vector was predigested with BsaI at 37° C. and purified. Oligo pools were amplified via Polymerase Chain Reaction (PCR). A 1:10 molar ratio of purified vector and insert were added to a golden gate assembly reaction using NEB golden gate assembly mix (Cat: E1601S), and incubated at 37° C. for 1 hour, then heat killed at 70° C. for 5 min. This material was then purified, drop dialyzed into pure H₂O for 60 min, and electroporated into electrocompetent BL21 bacteria (lucigen). Serial dilution were plated to recover individual colonies. Colonies were then picked, grown in media containing 1% glycerol and 100 μg/ml carbenicillin, and glycerol stocked in 20% glycerol at −80° C. After cloning, a construct is generated containing a barcode attached to a protein using a linker (FIG. 1A).

### Expression of Individual Barcodes:

Expression was performed either using in vitro transcription translation (IVTT) or BL21 induction. For IVTT, PCR was performed directly from glycerol stock by adding primers specific for T7 and T7 terminator sequences in BL21. The resulting amplicon contains T7 and T7 terminator for expression, and makes the protein 8×HIS-HALO-TEV-LN-Barcode-LC. 1 μL of PCR product, containing approximately 50 ng of DNA was added to a 10 μL IVTT reaction using NEBPure (cat no: E6800S) assembled according to the manufacturer's instructions. The reaction was then incubated for 4 hours at 37° C. For Escherichia coli (E. coli) expression, cultures were grown to an OD of 0.5 at 37° C., then induced using Isopropyl β-d-1-thiogalactopyranoside (IPTG) and grown overnight at 25° C. Cells were lysed the next day using sonication in the lysis buffer, and the lysed material separated from the inclusion bodies via centrifugation, taking the supernatant containing protein. Supernatant was purified using affinity chromatography Ni-NTA resin and stored for future use.

### Capture of Individual Barcodes on HALO Magnetic Beads:

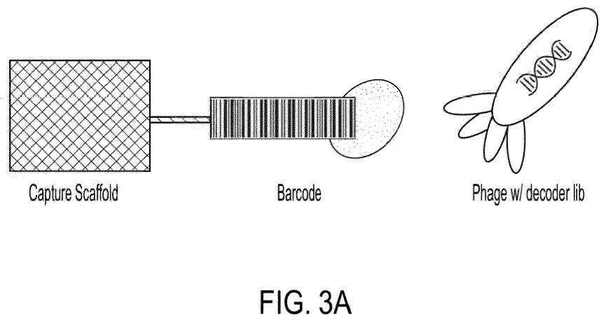

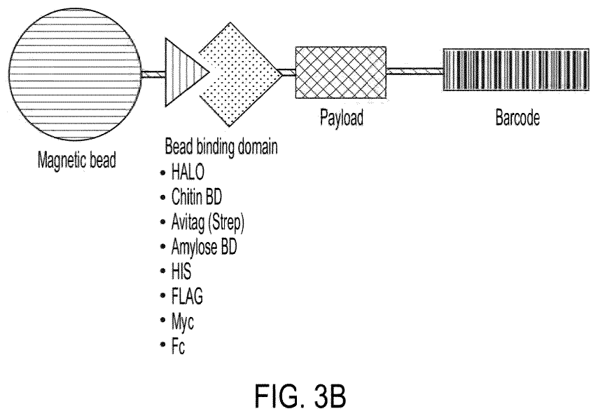

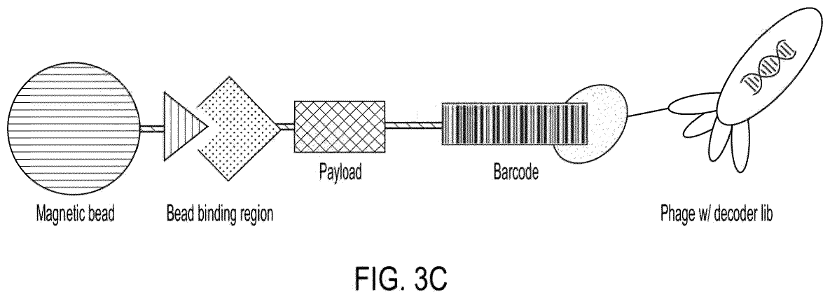

10 μL of WITT was diluted to 50 μL in PBS supplemented with BSA at 1 mg/ml. To this mixture, 30 μL of Halo tag magnetic beads (cat: G7281) were added, and incubated with shaking at 400 rpm for 2 hours, then 4° C. with shaking overnight. Beads were captured on magnetic stand, and the supernatant removed. The beads were then washed 2× with PBS-T with 0.1% Tween 20 (PBS-T). A schematic of a captured barcode is shown in FIG. 3B.

Construction of a Phage Display Library Containing Binders with Varying Affinities to Barcodes:

Binders with strong affinities to at least one barcode were generated via methods known to those skilled in the art (e.g., phage display, hybridoma, etc.). These binders were then displayed on phage as scFv fragments fused to m13 gene 3 protein (g3). Briefly, oligos containing the scFv binding sequences were generated via DNA synthesis. The oligo were cloned into a plasmid containing the constant regions of the scFv connected to G3 via a G4S linker (SEQ ID NO.: 9060) and myc tag. 30 μg of the library was electroporated into TG1 (lucigen) and plated on several 25 mm plates containing carbenecllin at 100 μg/ml and glucose 1%. Dilutions of electroporation were plated for diversity analysis. The Q trays were scraped and glycerol stocked. To produce phage, a 2 L culture was inoculated at ˜OD 0.05 and grown to OD 0.5 at 37° C. with 100 μg/ml carbenicillin and 1% glycerol. At OD 0.5, helper phage was added at a 10:1 phage:cell ratio, and incubated with shaking at 250 rpm. After 1 hour, shaking was reduced to 150 rpm and temperature to 30° C., and incubated overnight. The next day, phage was prepared via PEG precipitation (Barbas et al. 2001), resuspended in 10 mL, and titered. Phage was stored at 4° C. until use.

### Assessment of Phage-Binder-Barcode Interaction:

10 μL of the phage library prepared using methods described above was added to the captured barcode and incubated at room temperature for 2 hours, to allow binding of the phage to the barcode (FIG. 3C). After incubation, the mixture was washed by successive transfers of the magnetic beads to fresh PBS-T 3 times. Phage were eluted from beads by resuspension in PBS containing TEV protease+0.1% DTT and incubation at 37° C. for 30 min. Beads were collected on a magnetic stand, and the supernatant collected. To the supernatant, trypsin was added and further incubated for 30 min. To propagate phage, the supernatant was added to 50 mL of TG1 E. coli grown at 37° C. to an OD of 0.5 in 2×YT and incubated with shaking at 37° C. for 1 hour. Then 100 μg/ml carbenicillin and 1% glucose were added with incubation under the same conditions for 1 hr. Helper phage (cat: PH050L) was added and further incubated for 1 hour at 37° C. The culture was centrifuged and placed into new media containing 100 μg/ml carbenicillin and 50 μg/ml kanamycin, and incubated overnight at 30° C. Following this, centrifugation at 4000 g for 20 minutes was performed, and the supernatant containing phage was collected.

### Analysis and Establishment of a Fingerprint for a Given Barcode:

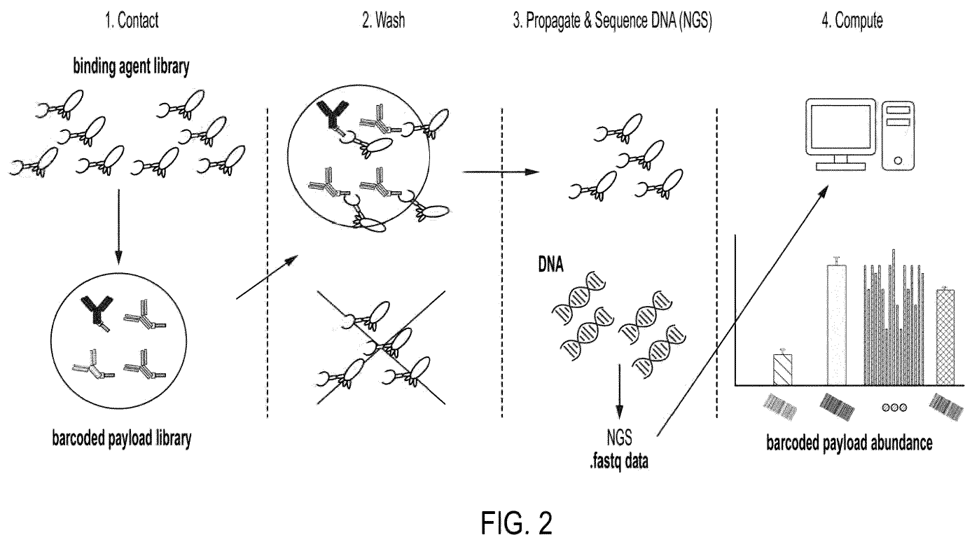

After selection was performed from the original phage pool against each barcode individually, phage-scfv (i.e., phage-binder) selectivity was analyzed via NGS. Phage were lysed via heating at 98° C. for 10 minutes, and the resulting genomes were PCRed using primers which flank the CDR regions of both the heavy CDR3 (5 prime) and light chain CDR3 (3 prime). A second round of PCR was performed to add required illumina sequences (i5/i7, sequencing primer binding region) for NGS. The result DNA was pooled, quantified and subject to NGS using an Illumina instrument. This process is shown in FIG. 2 and FIGS. 3A-3C.

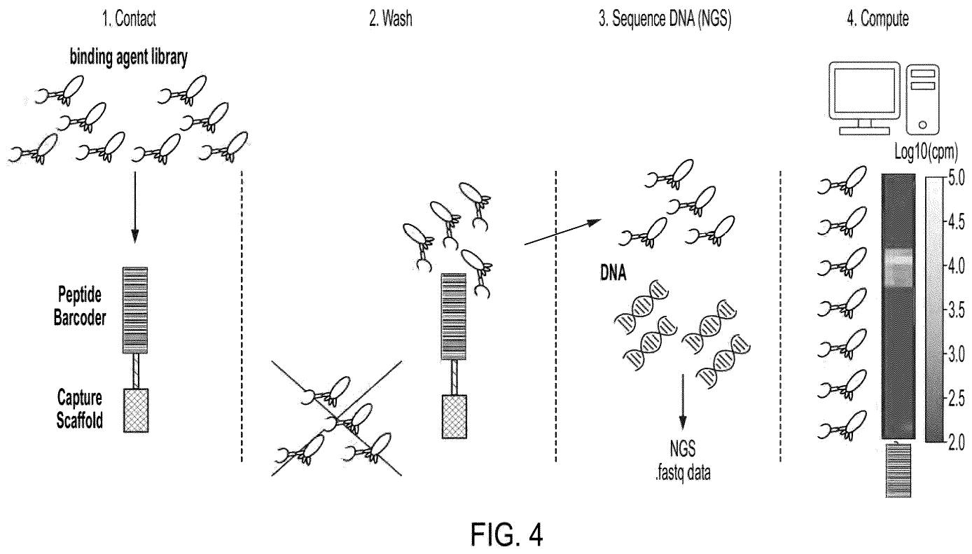

NGS reads are demultiplexed using the illumina software bcl-convert, such that each final .fastq contains the DNA sequences from a given phage CDR3 pair which correspond to the output from a given barcode well. The corresponding CDR3 sequences are then counted using a computer program, revealing the distribution of binders present for a given barcode. The fingerprint of a barcode corresponds to the vector of counts for each scFv binder within the given pool. Each fingerprint is the median of n=3 individual barcode replicates. The process and resulting fingerprint for a single barcode using the pool of phage binders is shown in FIG. 4.

### Determination of Proportion of Barcodes in a Given Mixture Using a Fingerprint Matrix:

Once the fingerprints of each barcode of a set of barcodes have been determined, the proportions of barcodes in an unknown sample were measured in the following manner. The binder-barcode interactions were assessed as described above, and the resulting NGS readout was fit to a linear combination of the known fingerprints via least squares. That is, the coefficients of the linear combination are chosen by minimizing the sum of squares of the differences between the measured NGS count and the expected NGS count for all binding species as described in Example 8. The expected NGS count is given by the matrix product of the fingerprint matrix with a set of barcode abundance coefficients. Once the coefficients have been obtained, they are normalized to sum to 1 to obtain proportions.

### Decoding of Equal Proportion Barcode Mixture to Assess Fingerprint Scaling:

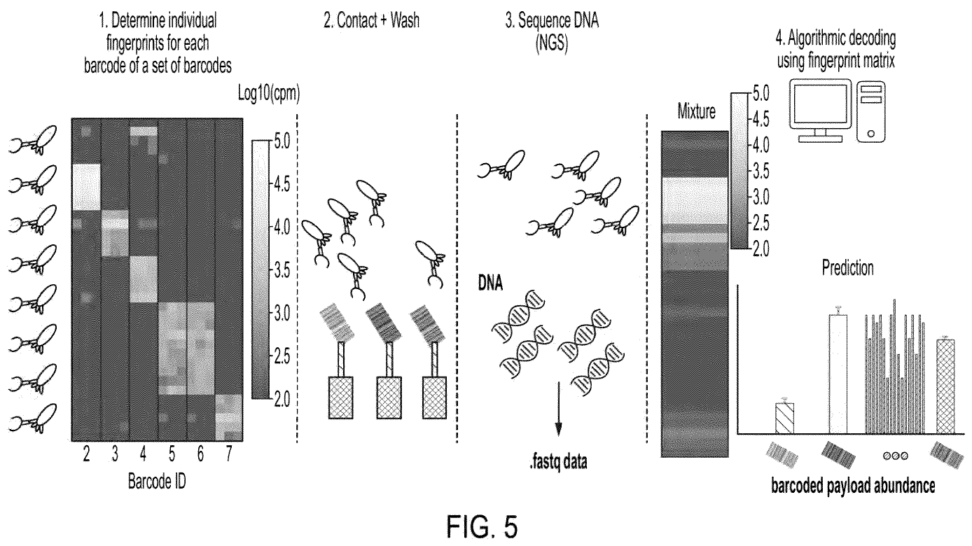

In order to determine any scaling issues that may arise due to varying affinities between binders and barcodes, a scaling factor is generated via a measurement of barcodes mixed in equal proportions. Briefly, all barcodes validated are mixed at even concentrations after production. Phage binder interactions were assessed as described above, and the resulting phage was subject to NGS. Using the fingerprints determined for individual barcodes above, the proportion of barcodes in the mixture was estimated via least squares regression as described in Example 8. The proportions predicted using the least squares form the basis for scaling factors (sf) where sf=1/p, where p=proportion predicted. The process is described in FIG. 5.

### Assessment of Mixtures of Barcodes of Known Proportions:

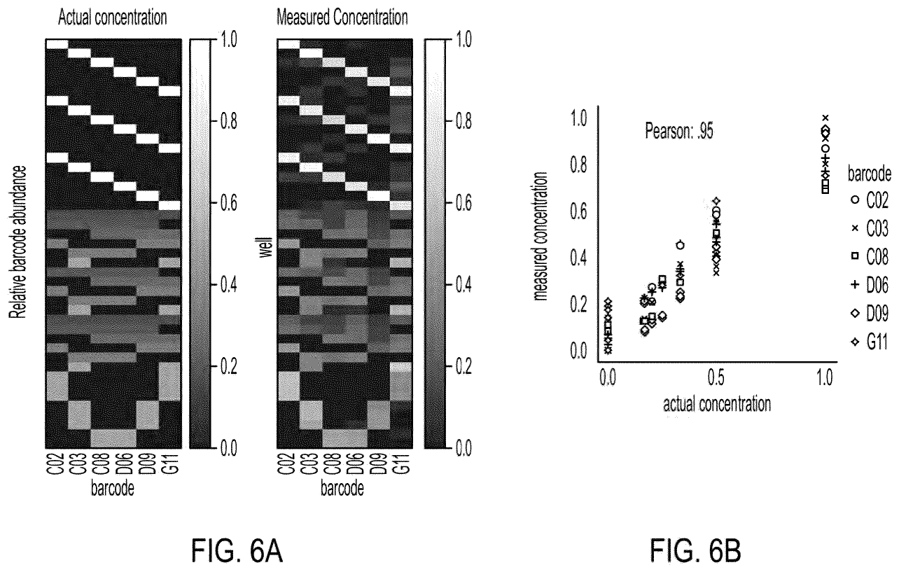

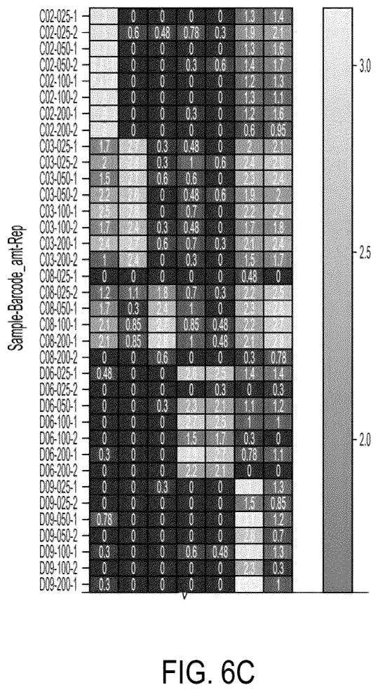

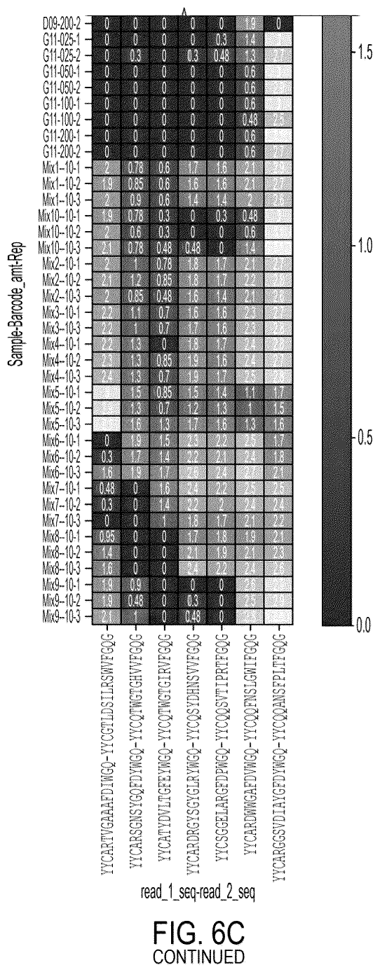

Barcodes produced above were mixed at known proportions and subject to assessment to determine their accuracy. Barcodes were mixed at different proportions (FIG. 6A), and then analysis of phage binder (i.e., binding agent) interactions was performed as described above. The NGS counts for each binder within the pool were counted. Least squares was used to determine the proportion of barcodes given the fingerprint constructed above using single barcodes. Predictions from the least squares analysis were then rescaled using the established scaling factor by p′=p*sf, where p′ is the new prediction, p is the original prediction and sf is the established scaling factor. The new predictions are then re-normalized to sum to 1.

FIG. 6B shows the accuracy of proportion measurement for this method. Six (6) different barcodes were measured, normalized via a scaling factor, and relative barcode proportions were estimated with a global pearson correlation of 0.95 across all measurements made. Measurements were across a 100-fold gradient of barcodes. FIG. 6C shows a plot of NGS count values, normalized to counts per million, for each single barcode measurement as well as mixture that were used to predict the relative abundance of each barcode within the mixture. Rows are experiments, thus all values in a row are generated from a single .fastq file and columns are binding agents.

### Example 2: In Vitro Detection of a Cargo in a Mixture Using Binder-Barcode Platform

The present example demonstrates a method of measuring the presence or absence of a given cargo in a mixture using a binder-barcode platform as described herein.

Barcodes generated in Example 1 were transferred onto a unique cargo using DNA cloning. Briefly, barcodes were amplified out of the pET 6×HIS-HALO-TEV-LN-Barcode-LC such that the LN-Barcode-LC part is amplified. The barcode insert was cloned using gibson into a new pET vector containing 6×HIS-Cargo-LN-Barcode-LC, where cargo was a unique protein of interest. Cargo-barcodes were produced in E. coli as described above. Barcoded cargo proteins are then purified via affinity chromatography using Ni NTA, washed in 500 mM NaCl, 50 mM Tris-HCl, 50 mM imidazole, and eluted using 500 mM imidazole. Barcoded cargo proteins were then subject to decoding using the phage binder library described in Example 1. FIGS. 6, 9, and 12 show experimental setups to detect cargo using barcodes previously generated in a different context, and results of such experiments thus showing these barcodes contain generalized detection properties across varying numbers of barcodes within a pool.

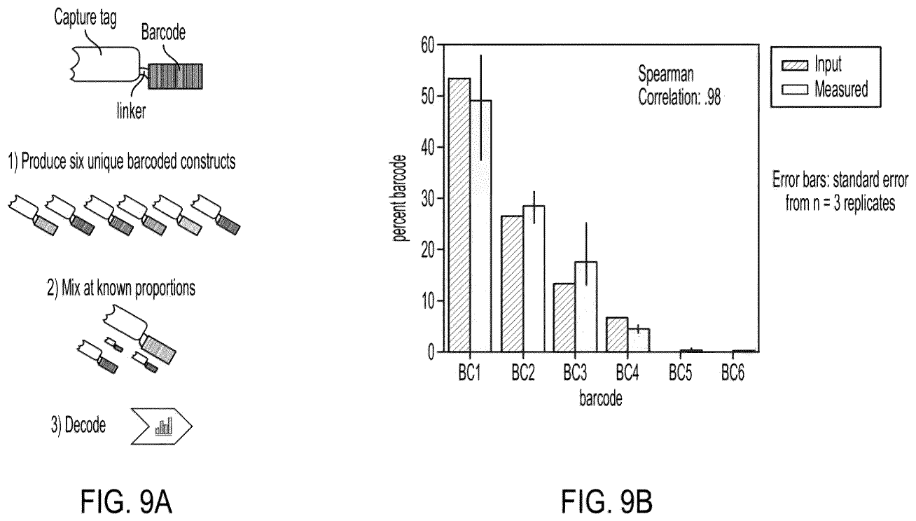

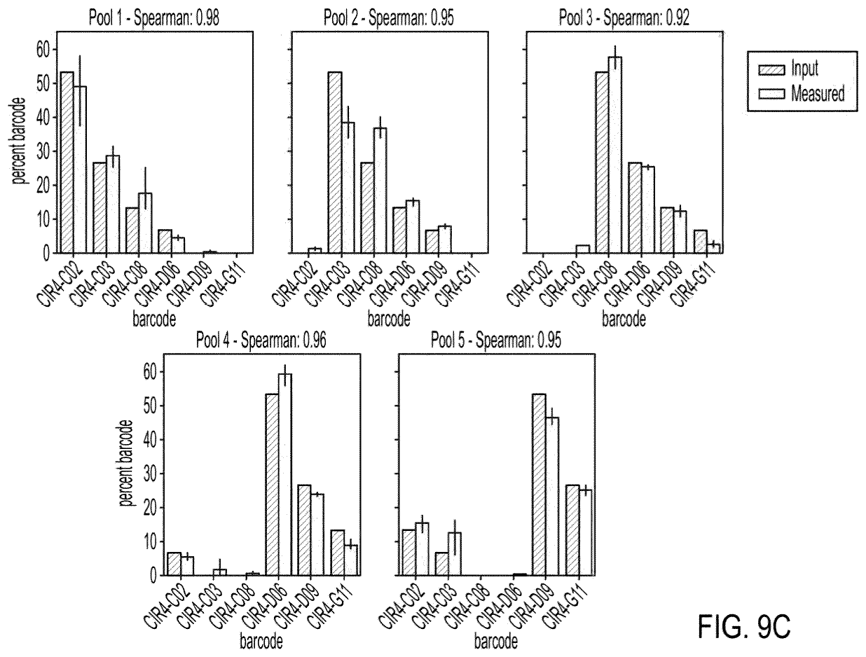

In the experiment described in FIG. 9, six unique barcodes (BC1, BC2, BC3, BC4, BC5, and BC6) were mixed at known proportions, contacted with binding agents, and subjected to decoding as described herein (FIG. 9A). Two barcodes were experimentally held out as negative controls, but prediction for these barcodes was allowed, thus allowing determination of background prediction. FIGS. 9B and 9C show data on accuracy of decoding procedure across a 10-fold range of concentrations for the 6 unique barcodes. FIG. 9B shows plot of actual data (input) and measured data obtained after decoding for one mixture of known barcode concentrations. Input known concentrations (left bar) are shown next to predictions/measured data (right bar) for each barcode across 3 replicates. FIG. 9C shows plots of actual data (input) and measured data obtained after decoding for five different mixtures (i.e., pools 1-5) of known barcode concentrations. Input known concentrations (left bar) are shown next to predictions/measured data (right bar) for each barcode across 3 replicates.

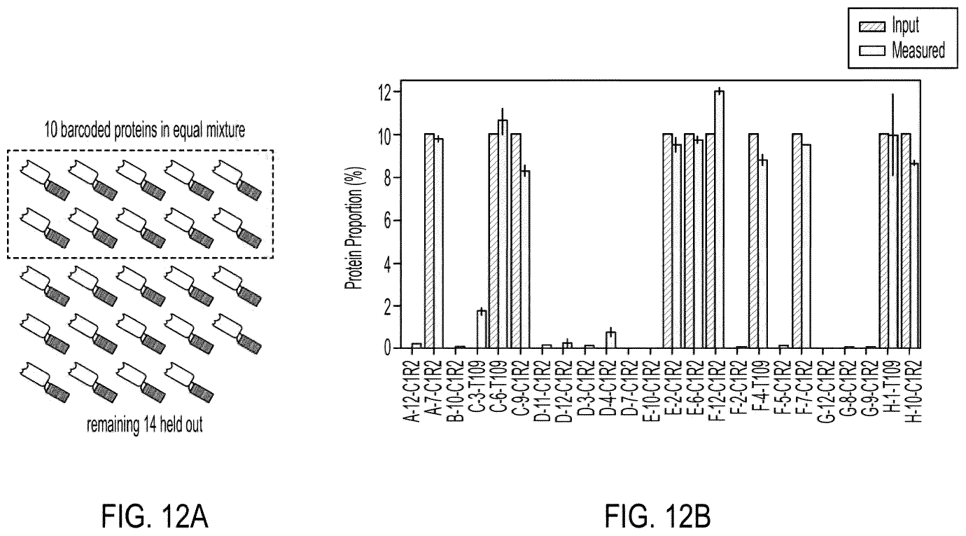

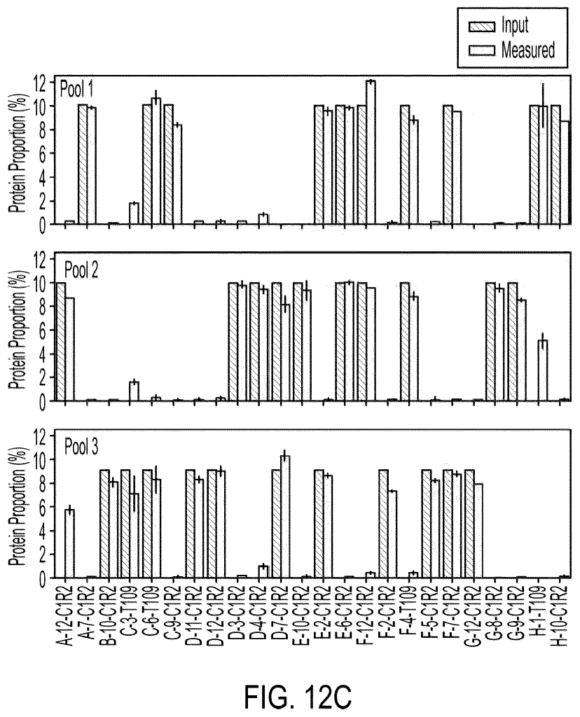

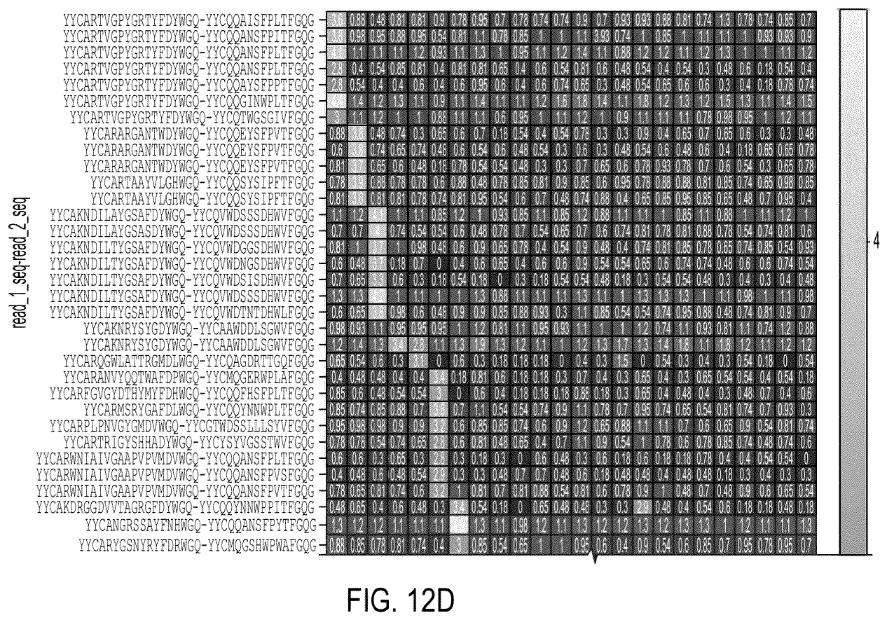

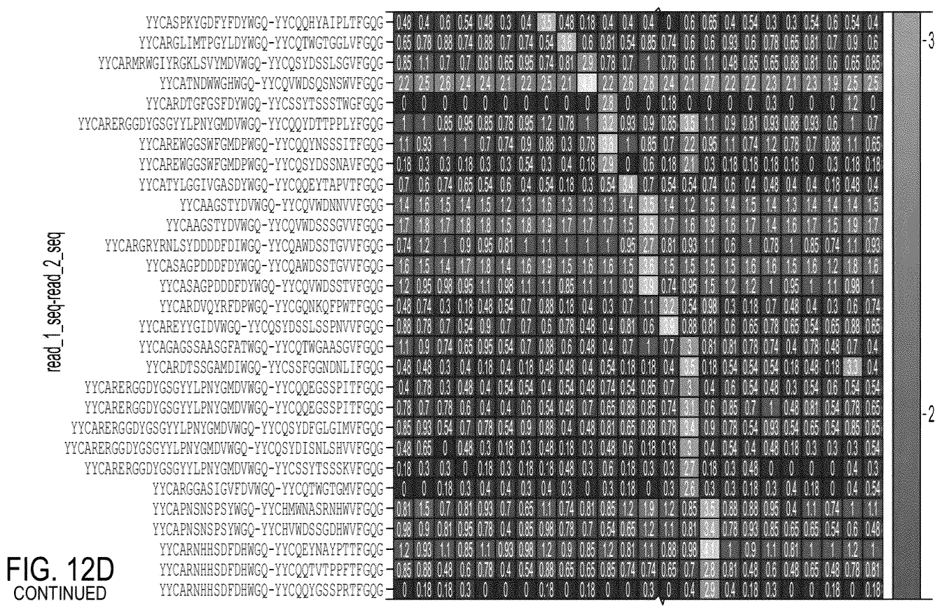

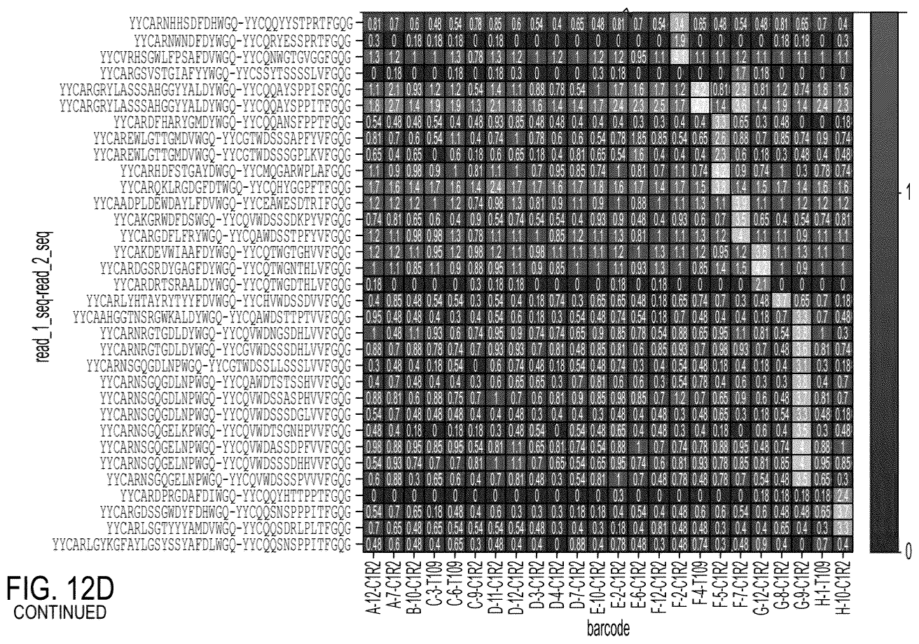

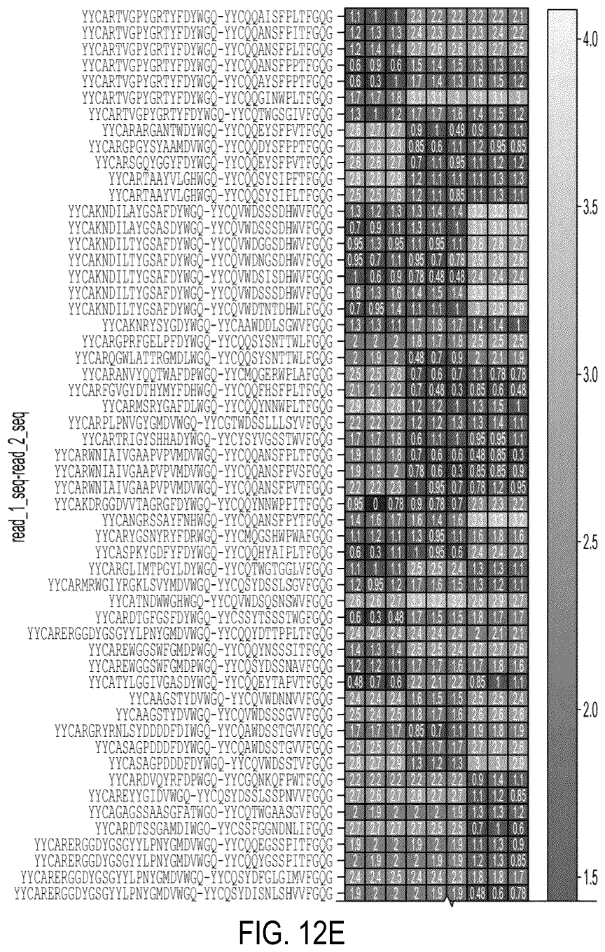

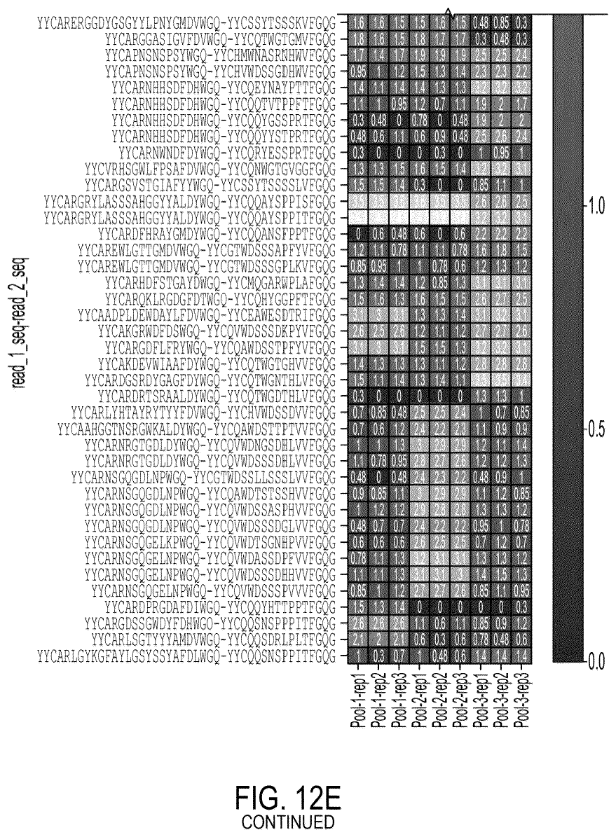

In the experiment described in FIG. 12, the quantification of twenty-four (24) barcodes contained within a single mixture was determined. FIG. 12A shows a graphical depiction of the experiment. Of 24 total barcodes the algorithm can predict, 10 were present within a mixture at equal concentrations. The rest were held out from the pool, but prediction was computationally allowed. Three (3) separate pools, which cover all possible barcodes were measured in replicate. These pools were mixed, then captured on HALO beads, described in Example 1. The immobilized barcoded cargo was then contacted with a pool of binding agents (i.e., binding agents with binders expressed on them), and decoded to CDR3 sequence counts as described in Example 1. The CDR3 sequence counts, determined via NGS, were then utilized to determine the presence and total concentration of each barcoded cargo in the sample via decoding (see Example 8). FIG. 12B shows prediction for the first pool; the input concentration (left bar) and measured concentration (right bar) are plotted for each barcode in the pool. FIG. 12C plots predictions for each of the three pools. As in FIG. 12B, the bar graphs plot input concentration on the left and measured concentration on the right. FIG. 12D shows the barcode fingerprint for each of the 24 barcodes used to computationally determine the relative abundance of the barcodes within each of the 3 pools. Columns represent barcode fingerprints, and rows represent binding agent fingerprints. FIG. 12E shows the binding agent counts from the three pools, used to computationally determine the proportion of the pools. Rows are the binding agent counts, columns are the pools, and each cell is the binding agent count within a specific pool.

### Example 3: In Vitro Assessment of Cargo Stability within a Pool of Cargos Using Binder-Barcode Platform

The present example demonstrates a method to determine the general aggregative tendencies of several cargos in a pool using barcode decoding.

A purified pool of barcoded cargos is generated using the method described in Example 2. The purified pool is then subject to size exclusion chromatography using standard methods. Different fractions are collected—corresponding to monomeric vs aggregated cargos. The general presence or absence of a given barcoded cargo within the purified pool is not known. The separated fractions, containing an unknown abundance of each barcoded cargo, are then immobilized on beads or immunosorbent assay plates, contacted with a pool of binders (i.e., binding agents with binders expressed on them), and decoded to CDR3 sequence counts as described in Example 1. The CDR3 sequence counts, determined via NGS, are then utilized to determine the presence and total concentration of barcoded cargos in each fraction. The concentration of barcoded cargos in the different fractions are then compared to determine the percent of each barcoded cargo which is monomeric vs aggregated within the purified pool.

### Example 4: In Vivo Assessment of Cargo Pharmacokinetics within a Pool of Cargos Using Binder-Barcode Platform

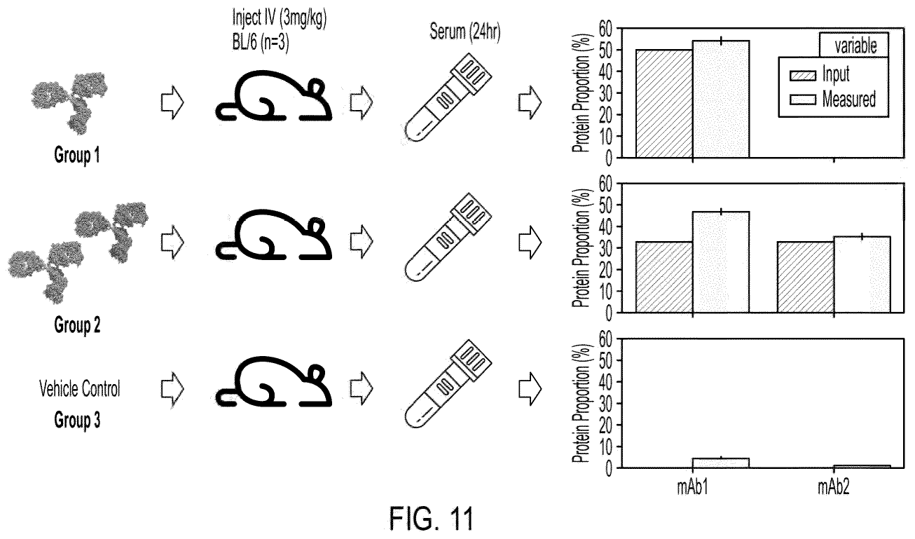

The present example demonstrates a method to determine the overall residence and clearance time of a given cargo, contained within a pool of cargos, using a mouse model, as demonstrated in FIG. 11.

Three pools of barcoded cargos were injected into three different groups (pool 1 into group 1; pool 2 into group 2, and pool 3 into group 3) of mice (n=3). Pool one contained a single barcoded antibody at 10 mg/kg. Pool two contained two barcoded antibodies pooled at equal concentrations and injected at 20 mg/kg. Pool three contained PBS only. Injection volumes were held constant at 100 μL per pool. At 24 hours after injection, blood was collected from each mouse and serum separated. 10 μL of serum was diluted 1:10 in PBS and captured using anti-human IgG magnetic beads (Ray biotech cat #801-101-1) by incubation overnight at 4° C. with mixing at 700 rpm. The immobilized barcoded cargos were washed using PBS-T three (3×) times, to remove all serum proteins not associated with the affinity reagent. The immobilized barcoded cargo was then contacted with a pool of binding agents (i.e., binding agents with binders expressed on them), and decoded to CDR3 sequence counts as described in Example 1. The CDR3 sequence counts, determined via NGS, were then utilized to determine the presence and total concentration of each barcoded cargo in the sample via decoding (see Example 8). The proportion of barcoded cargos measured at 24 hours was compared to the injected concentration to determine the relative rate of clearance for each barcoded cargo from the organism. In each of the groups, only the injected barcoded antibody was detected by decoding as evidenced by the graph plotted in FIG. 11, with high accuracy. In group 2 mice, which were injected with pool 2 that contains both antibodies at equal concentrations, slightly differing amounts are measured via decoding in the serum at 24 hours. It is hypothesized that this difference is due to differences in clearance rates between the two barcoded antibodies. As expected, the control group showed almost no antibody.

### Example 5: In Vivo Assessment of Cargo Biodistribution Using Binder-Barcode Platform

The present example demonstrates a method to determine the overall distribution of a barcoded cargo across a diverse set of tissues using a mouse model.

A pool of purified cargos is injected intravenously into a BALB-6 mouse. After at least 24 hours, different tissue samples, such as liver, lung, and brain, are taken from the organism. The tissues are then processed into a single-cell suspension via vigorous shaking with beads. The suspension is then lysed using a lysis buffer to liberate the barcoded cargos (e.g., barcoded cargo polypeptides) contained within the tissues. The lysed suspension is then purified using a universal tag affinity reagent contained within the cargos to separate the barcoded cargos. Purified barcoded cargos are then immobilized, and barcode decoding is performed according to method described in Example 1. The CDR3 sequence counts, determined via NGS, are then utilized to determine the presence and total concentration of each barcoded cargo in each sample. Cargo abundance across different tissue samples is then compared, to determine the percent of each cargo which is contained within each tissue. This data may then be used to select the best cargo with specific biodistribution properties.

### Example 6: In Vitro Demonstration of Recovering Known Mixture of Unmodified Antibodies

The present example demonstrates how a known mixture of antibody proteins with no barcode attached was quantified using the protein quantitation invention described herein.

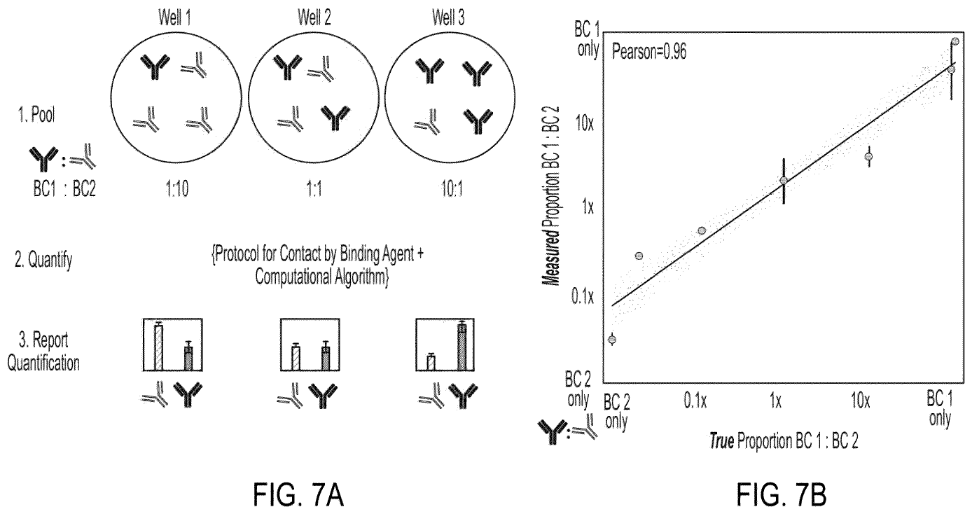

Briefly, scFv binders to the antibodies were generated using methods known to those skilled in the art. The binder were then cloned and displayed on phage as described in Example 1. The two antibodies of interest were expressed in CHO cells and purified from the media using Protein A affinity chromatography. Antibodies were mixed together in known proportions (FIG. 7A). Antibodies were then captured using 50 μL anti-Human Fc magnetic beads and incubated in PBS. The antibodies were then subject to phage assessment as described in Example 1, and the relative abundance of each antibody estimated using the algorithm described in Example 8. An accuracy of pearson 0.96 at determining the relative concentration of these two antibodies in varying proportion mixtures was calculated (FIG. 7B).

### Example 7: In Vitro Demonstration of Recovering Known Mixture of Antibodies in Presence of Serum

The present example demonstrates how a known mixture of antibody proteins with barcodes contained within internal regions of the protein sequences was quantified in mouse serum using the protein quantitation technology described herein.

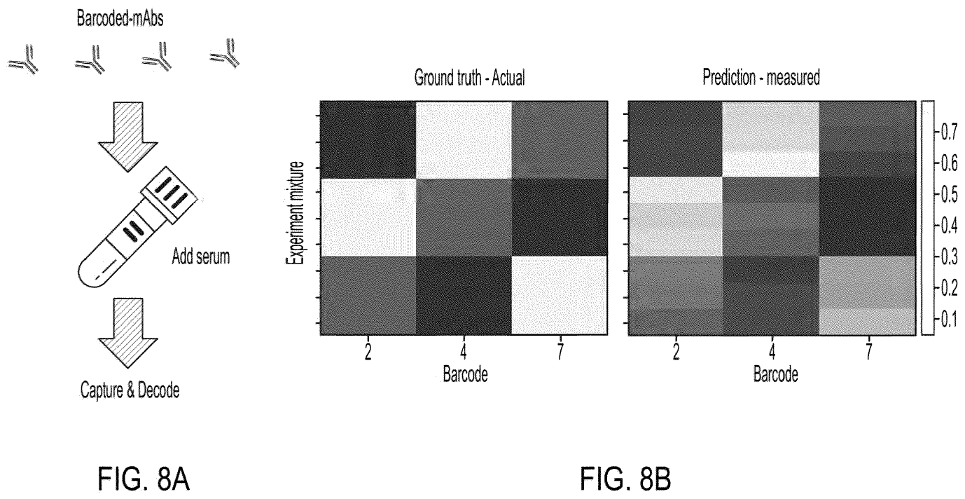

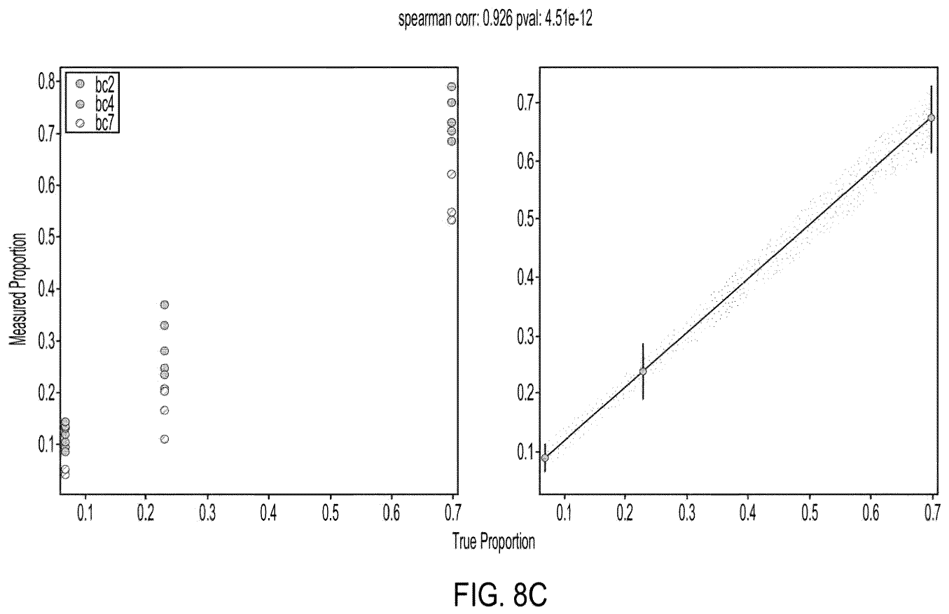

Briefly, antibodies were produced as described in Example 6, and a similar experiment was performed, except after production, the antibodies were mixed with mouse serum, incubated at 37° C. for 30 minutes, and then captured using anti-Fc magnetic beads (FIG. 8A). The immobilized barcoded antibodies were then contacted with a pool of binding agents (i.e., binding agents with binders expressed on them), and decoded to CDR3 sequence counts as described in Example 1. The CDR3 sequence counts, determined via NGS, were then utilized to determine the presence and total concentration of each barcoded antibodies in the sample via decoding (Example 8). The relative concentration of three antibodies was estimated with a spearman of 0.926 across 3 mixtures of antibody concentration as shown in FIG. 8B and FIG. 8C. These results show the ability using technologies described herein to rank the antibodies present in a sample with high accuracy after incubation with serum i.e., in a complex environment.

### Example 8: Detailed Description of the Decoding Algorithm Used to Infer Barcode Abundance

### Method of Inferring Barcode Amounts:

Before decoding an unknown sample, a set of barcodes and its interaction with a binder pool (i.e., phage binder pool) must first be characterized. This is done by decoding a set of known samples under known conditions. The binder pool and experimental conditions is held fixed between all samples.

To characterize a set of barcodes, we measure a set of fingerprints, one for each barcode. A fingerprint represents the ideal readout of an individual barcode. Roughly speaking, it is the spectrum of affinities between a given barcode and all the binder species in the pool. A fingerprint can be estimated by decoding multiple identical samples containing purely one barcode, averaging together the readouts of the replicates, and rescaling accordingly. Alternatively, fingerprints can be learned by decoding samples containing known mixtures of barcodes and deconvoluting appropriately to isolate individual fingerprints. Together, the fingerprints of a set of barcodes is known as a “fingerprint matrix”.

Once the fingerprint matrix of a set of barcodes is determined, it can be used to infer the barcode composition in an unknown sample. The decoding algorithm accomplishes this by fitting the readout of the unknown sample to a linear combination of fingerprints. This is described in further detail in the algorithmic section described herein. A key assumption of the algorithm is that the decoding process is linear: if a sample contains two barcodes mixed in equal proportions, it is assumed that its readout is equal to the sum of the fingerprints of the two barcodes (plus noise). More generally, the readout of a mixture of barcodes is assumed to be the sum of the fingerprints of each barcode, appropriately weighted by its prevalence in the mixture. This assumption has empirically been found to be true.

The task of barcode quantification has varying levels of difficulty. From easiest to hardest, these include:
Binary classification: detecting the presence or absence of a barcode in a sample
Rank-order quantification: ranking barcodes from most to least prevalent in a sample
Relative quantification: determining the ratios between barcodes in a sample
Absolute quantification: determining the absolute amount of each barcode in a sample

In this Example, absolute quantification is discussed in further detail.

### Mathematical Model of Decoding:

The decoding process can be represented with the following mathematical model, where:
x is a length-n vector representing the input sample and each entry is the amount of a barcode species in units of ng; y is a length-m vector representing the bound binder fraction and each entry is the number of particles of a binder species in units of pfu; z is a length-m vector representing the NGS readout and each entry is the number of counts for a binder species.

The bound binder fraction is modelled as a linear combination of fingerprints, and the NGS readout is modelled as multiplying the bound binder fraction by a conversion factor:

$${y}_{j} = {\sum }_{i = 1}^{n} {A}_{ji} {x}_{i} + {ϵ}_{1}$$

$${z}_{j} = {s}_{j} {y}_{j} + {ϵ}_{2}$$

where Aⱼᵢ is the fingerprint matrix, an m by n matrix with units of pfu bound per ng of barcode. The ‘ji’ entry represents the propensity for barcode i to bind binder j in the binder pool; sⱼ is the conversion factor between pfu bound and number of NGS reads for binder species j; ε₁ is noise associated with the binding steps; ε₂ is noise associated with the post-binding steps.

The model assumes that binding between barcodes and binders is linear. In other words, if a sample contains a mixture of barcodes, its readout is assumed to be equal to the sum of the fingerprints of the individual barcodes, weighted by the relative barcode abundances. In Appendix A we provide a detailed biophysical model which justifies the linear assumption under one key condition: the amount of available binder cannot be significantly depleted by binding to the barcodes in the sample. Thus, as in typical immunoassays, the binding agent must be in excess and cannot be the limiting reagent.

### Fingerprint Matrix Aⱼᵢ:

Each column of the mn-fingerprint matrix is a fingerprint. Each fingerprint represents the ideal, properly normalized readout of a pure barcode. The entry Aⱼᵢ represents the contribution of the j'th binder to the fingerprint of barcode i.

The fingerprint of a barcode depends on the binding affinity to all the binders in the pool as well as the relative abundance of each binder species in the pool. Furthermore, the fingerprint is sensitive to the binding, equilibration, and elution steps. In the simplest case, the fingerprint matrix is given by

$${A}_{ji} = {d}_{j} / {K}_{ji}$$

where dⱼ is the concentration of binder j in the binder pool, and Kⱼᵢ is the dissociation constant of the complex between barcode i and binder j (see Appendix A). In more complicated cases, A, could also include effects of adhesion to surfaces, unbinding during washing steps, etc.

The matrix product of A with a barcode mixture x gives the composition of the ideal bound binder fraction (i.e., in absence of noise), in units of number of phage particles.

The fingerprint matrix can be determined from measuring the readout of multiple known samples. Multiple replicates are performed to average over noise. In addition, the fingerprints are properly scaled, either with respect to one another or to an absolute standard (see the Normalization section).

### Conversion Factors sⱼ:

The post-binding steps introduce a conversion factor between the number of bound phage particles to the number of NGS reads. This is represented by sⱼ. In the simplest case, sⱼ is identical for all binder species and represents an overall normalization,

$$s = C / {\sum }_{j = 1}^{m} {y}_{j}$$

such that the total number of reads/counts is C. This models the situation where there is some sort of bottleneck in the processing, such as saturation of the propagation culture, such that the end result is a fixed number of reads, irrespective of the amount of bound phage. In a more complicated case, sⱼ could depend on the binder species, reflecting amplification bias or differential phage fitness in propagation.

In the case where the conversion factor is identical for all binder species, it is a single number which must be determined on a sample-by-sample basis. This can be done by using a DNA sequence spiked in at some point of the process (as described herein).

### Noise ε:

Noise sources are represented by the terms ε₁ and ε₂. In the absolute simplest case, ε₁ is absent and is ε₂ is Gaussian noise of fixed variance, in which case prediction can be done with ordinary least-squares regression. In reality, the noise arises from multiple, non-Gaussian sources, as detailed in the sections above. These include log-normal noise involved with exponential steps, such as phage propagation and PCR amplification, Poisson noise due to finite sequencing depth (and possibly stochasticity in binding/elution at low concentrations), and Gaussian noise from all sorts of other processes, such as sample degradation, etc.

Conversion from Read Counts to Phage Counts:

One feature of the NGS is that the readout is a relative measurement: it gives the ratio of abundances between different binder species, but not necessarily the absolute concentrations of binder species. To obtain an absolute readout, the raw readout must be divided by a conversion factor between the number of phage particles bound and the NGS read count.

Without knowing the conversion factor, it is only possible to determine the relative abundances of barcode in a sample (i.e., proportions). Absolute quantification requires a reference of known concentration (either barcode or binder) to be spiked into the process.

### Methods of Absolute Quantification:

Spiking a Phage Ladder into Eluate:

One method of normalizing is to add a unique binder species into the eluate at a known concentration, yₛₚᵢₖₑ₋ᵢₙ. This reference species should be distinct from the existing binders in the pool. In the subsequent steps (where the eluted phage are propagated, DNA extracted, PCR'ed, and sequenced), the reference phage will be amplified by (ideally) the same factor as the other phage in the pool, i.e.,

$${z}_{spike - in} = s \cdot {y}_{spike - in}$$

The conversion factor can be estimated by dividing the number of reads corresponding to the reference phage by the (known) concentration at which it was added, i.e.,

$$\hat{s} = {y}_{spike - in} / {z}_{spike - in}$$

A generalization is to spike multiple binder species into the eluate. Each reference species can be spiked in at a different concentration. If the concentrations are evenly spaced, this forms a “phage ladder”, in analogy with ladders used in gel electrophoresis. To estimate the conversion factor, the number of reads of each reference sequence can be compared to the (known) concentration at which it was added to the eluate. Averaging across the species then yields a more precise estimate of the conversion factor.

Spiking Barcode into Sample:

Alternatively, a reference barcode of known concentration can be added to the sample at the beginning of the decoding process. This reference barcode should be distinct from the existing barcodes in the sample. The decoding algorithm can use the raw readout to determine the proportions of all barcodes within the sample, including the reference and the sample barcodes. By dividing the reference barcode concentration by its predicted proportion, a barcode conversion factor can be determined. Multiplying all the predicted proportions by this factor then yields the absolute abundances of barcode.

Note that this method is only applicable for decoding an unknown sample after a set of properly normalized fingerprints has been determined.

### Scaling Fingerprints:

An important subtlety is that readouts have to be scaled even in the case of relative quantification. Specifically, the fingerprints of a set of barcodes must be properly scaled with respect to one another. Intuitively, this is because raw, unscaled fingerprints cannot be directly compared between barcodes; the read count of a binder has a different meaning in the context of a different barcode's fingerprint, because each barcode has a different conversion factor between bound binder count and read count. To ensure that fingerprints are measured in the same units, each unscaled fingerprint must each be scaled by (the inverse of) its conversion factor.

To illustrate this, consider a case with two barcodes. Suppose that, due to differences in binding affinity across the binder pool, the total amount of binder bound to 100 ng of barcode A is 10 times greater than that bound to 100 ng of barcode B. However, due to the nature of the method, the raw readouts end up having the same number of reads. In this example, one NGS read in barcode A's raw fingerprint corresponds to 10 reads in barcode B's raw fingerprint. The conversion factors are different. Now consider a sample containing a 1:1 mixture of the two barcodes. Due to the difference in affinity, the bound binder fraction in this sample is 10:1; consequently, the number of NGS reads corresponding to A and B would also be in a 10:1 ratio. In other words, the readout is proportional to 10a+b, where a is the raw readout of A and b is the raw readout of B. On the basis of this, one would come to the incorrect conclusion that A and B are in a 10:1 ratio. To correct for this, the raw fingerprint of barcode A must be multiplied by a factor of 10, compared to B, to obtain a correctly scaled fingerprint, a′=10a and b′=b. Now, when the two barcodes are mixed in equal proportions, the correct result is determined: the readout is an equally weighted mixture of the two correctly scaled fingerprints, a′+b′.

This example shows that the relative scaling factor between barcodes' raw fingerprints can be determined by measuring the readout of a known mixture of the barcodes. If the barcodes are mixed in equal proportions, the composition of the mixture readout will be each raw fingerprint, weighted by their scaling relative to each other. (Note that this gives only the relative conversion factor between the barcodes and not an absolute conversion factor to absolute barcode quantities.)

### The Decoding Algorithm:

There are two phases of the decoding algorithm:
“Training phase”: Learn the fingerprint matrix A, by measuring the readout of a number of known samples
“Testing phase”: Predict the barcode amounts in an unknown sample by measuring the readout and comparing it to A,

### Training Phase: Learning the Fingerprint Matrix

In the training phase, the fingerprints of a set of barcodes are determined by measuring the readouts of a set of samples with known composition. The fingerprints must be correctly scaled with respect to one another. One method of measuring the fingerprint matrix is outlined below.

First, a set of samples, each containing purely a single barcode are prepared. Each sample is decoded. The fingerprint of each barcode is estimated by taking multiple replicates of a barcode and averaging together their readouts. The error is reduced if more replicates are averaged. This yields an unscaled fingerprint for each barcode.

Next, the fingerprints are correctly scaled with respect to each other. This is done by multiplying each of the unscaled fingerprints by a scaling factor. To determine the scaling factor of each barcode, a sample consisting of all barcodes mixed in equal proportion is decoded. Theoretically, this readout of this sample should be the sum of the normalized fingerprints of all the barcodes, weighted equally. However, if we fit this mixture readout to the set of unnormalized fingerprints determined from the previous step, the weights of the barcodes would not be equal. The coefficients to the linear fit are precisely the factor by which each barcode's fingerprint should be multiplied by to obtain a correctly normalized fingerprint. By averaging together multiple replicates of this, a more precise estimate of the scaling factors can be determined.

This method is detailed further below. Let Ã be the unscaled fingerprint matrix obtained from averaging multiple unscaled measurements of single barcodes. Each column should sum to a fixed number of reads, so the units of Ã are counts per million. The correctly scaled fingerprint matrix A is obtained by multiplying each column of Ã by a rescaling factor S, such that A=ÃS, where S is a n×n diagonal “rescaling matrix”. Here Sᵢᵢ is the scaling factor for the i'th barcode, with units of pfu eluted per count per million. Let y be the readout of a mixture of known composition x. One way to determine scaling factors is to use the unscaled fingerprint matrix to infer a set of “biased” predictions as \tilde{x}=Ã+y=SA⁺y, where Ã⁺ is the pseudoinverse of Ã (see the prediction section below). The ratio of the “biased” prediction to the actual amount is an estimate of the scaling factor: Sᵢᵢ≈ [symbol] /xᵢ. Multiplying each unscaled fingerprint by this scaling factor gives our best estimate of the properly scaled fingerprint A=ÃS.

Note that there are other methods for measuring the fingerprint matrix. This present method only uses the single-barcode samples to determine (unnormalized) fingerprints, and the mixture samples to determine the scaling factors. More sophisticated methods may use uneven mixtures to determine scaling factors and/or use the information in these samples to better estimate the fingerprint (beyond just learning its scaling factor).

### Testing Phase: Predicting Barcode Amounts in Unknown Samples

In the testing phase we are supplied with the readout y of an unknown sample x and we aim to infer its composition, \hat{x}. This is done by fitting the readout to a linear combination of fingerprints, y=A\hat{x}, where A is the (properly scaled) fingerprint matrix learned from the training phase.

The fitting is done by choosing a set of coefficients xⱼ, which minimizes a loss function. The loss function measures the deviation between the expected and measured readout. The expected readout is the matrix product Aₓ, based on the determined fingerprints and proposed mixture coefficients. In the simplest case, the loss function is a sum of squared errors,

$$( \hat{x} ) = {\sum }_{j = 1}^{m} {( {\sum }_{i = 1}^{n} {A}_{ji} {\hat{x}}_{i} - {y}_{j} )}^{2} = {‖ A \hat{x} - y ‖}_{2}^{2}$$

and the inferred mixture composition is the minimizer of this loss, \hat{x}=argmin L(x). If the number of binders is greater than the number of barcodes, then Aₓ=y is an overdetermined system and there is a unique minimizer of the loss. The solution is given by

$$\hat{x} = {A}^{+} y$$

where A⁺=(A^{T}A)⁻¹A^{T} is the Moore-Penrose inverse of the fingerprint matrix. If relative abundances (proportions) are desired, the coefficients can be normalized to sum to 1.

Note that the L2 loss function above is the simplest case. It is (proportional to) the negative log likelihood in the case where ε₁ is absent (no noise in binding) and ε₂ is Gaussian noise of fixed variance. To model more realistic forms of noise, other loss functions can be chosen.

### APPENDIX A: BIOPHYSICAL MODEL OF BINDING PROCESS

Let n be the number of barcoded cargo polypeptide species in a sample, and let xᵢ, i=1, . . . , n be the concentrations of each barcode species added to the decoding well. Likewise, let m be the number of decoder species in the decoder pool, and let dⱼ, j=1, . . . , m be the concentrations of each decoder species added to the decoding well. Suppose that any decoder can interact with any barcode in a one-to-one stoichiometry to form a bound complex. A total number of nm such complexes can be formed, one for each barcode-decoder pair. Let cᵢⱼ, i=1, . . . , n, j=1, . . . , m be the concentration of a complex between barcode i and decoder j, and let Kᵢⱼ be the equilibrium dissociation constant characterizing the affinity of this interaction, with ΔG_{binding}=−RT ln Kᵢⱼ.

For any decoder species, we assume that the amount which binds to the sample and subsequently eluted is given by

$${y}_{j} ≡ {\sum }_{i = 1}^{m} {c}_{ij} ,$$

the bound complexes of that decoder summed over all possible barcode pairs.

In the simplest case, we assume that each species is an ideal solute in a dilute solution, and that the binding between barcodes and decoders is allowed to approach thermodynamic equilibrium. At equilibrium, some fraction of the decoders will bind to the barcodes, but there will still be remaining unbound barcodes, with concentrations \hat{x}ᵢ, i=1, . . . n, as well as unbound decoders, with concentrations \tilde{d}ⱼ, j=1, . . . m.

The equilibrium state is given by minimizing the overall free energy of the system. This is shown to be equivalent to solving the following set of equations:

$${x}_{i} = {\tilde{x}}_{i} + {\sum }_{j = 1}^{m} {c}_{ij} for i = 1 , … n$$

$${d}_{j} = {\tilde{d}}_{j} + {\sum }_{i = 1}^{n} {c}_{ij} for j = 1 , … m$$

$${K}_{ij} = {\tilde{x}}_{i} {\tilde{d}}_{j} / {c}_{ij} for i = 1 , … , n , j = 1 , … , m$$

The first equation ensures the conservation of mass: the total amount of a barcode species is the unbound amount plus the amounts bound in complexes with all possible decoder partners. The second equation is an analogous statement for the decoders. The last equation is the definition of an equilibrium constant of a barcode-decoder pair.

The known values are the equilibrium constants Kᵢⱼ and the xᵢ and dⱼ, representing the total concentration of each species of barcode and decoder, respectively, added to the well. The unknown variables are [symbol] , [symbol] , and cᵢⱼ. The values of the unknown variables are determined by solving the system of equations above.

In the decoding process, a decoder pool with fixed values of Kᵢⱼ and dⱼ is added to a sample of unknown barcode composition, xᵢ. The observable output of the binding process is the amount of each decoder species which binds to the sample,

$${y}_{j} ≡ {\sum }_{i = 1}^{m} {c}_{ij} .$$

A key question of the binding process is how the input, the barcode concentrations xᵢ, affects the output, the bound decoder amounts yⱼ.

In general, the system of equations above is non-linear, but when certain conditions are met, the binding process can be well approximated by a set of linear equations. The decoding process is greatly simplified if the underlying equations are linear. A linear system implies that, at the least:
1) If the concentration of a barcode is doubled, the decoders bound to that barcode are all correspondingly doubled. (no saturation)
2) If two barcodes are mixed together, the decoders bound to the mixture are the sum of the decoders bound to each barcode alone. (no competition)
   These two criteria can be thought of as linearity of single and multiple barcode detection, respectively. They are necessary, but not sufficient conditions.

To see how these criteria play in practice, consider the following binding situations which violate one or more of the criteria. Suppose that the decoder is the limiting reagent in a one-to-one stoichiometry of barcodes and decoders. Above a certain barcode concentration, all the available decoders would become saturated; an increase in xᵢ would not lead to a proportional increase in yⱼ. Thus, to avoid saturation, the barcode concentrations should be kept below the Kd of the interaction (or the decoder concentration, whichever is greater—see below).

As another example, consider a situation where two barcodes A and B both have affinity for a certain decoder D, but barcode A has a much stronger affinity than barcode B. In the absence of A, the binding of barcode B has a certain binding curve. However, if there is enough A to deplete much of the available D, the binding of barcode B to the remaining D will be significantly altered. This is a situation where A and B compete for available decoder D. Note, however, that if the binding of A did not deplete the amount of D remaining in the pool, then the binding between B and D would not be altered by the presence of A. The competitive behavior occurs only in situations where the available D is significantly consumed by binding to A—if A is highly abundant and/or the A-D affinity is strong. In both examples of non-linear situations, one or more decoders in the pool is significantly depleted by binding to barcodes.

These examples indicate that the binding process is linear when a small fraction of the decoding pool is bound to the sample. Indeed, if this condition is met, the equations above can be simplified into a simple linear system. Under this assumption, the amount of unbound decoder \tilde{d}ᵢ is well approximated by the total amount of decoder, dⱼ. If we further assume that the bound complexes deplete a small fraction of the available barcodes, then the system of equations simplifies to

$${c}_{ij} = {x}_{i} {d}_{j} / {K}_{ij}$$

and the output can be represented with a simple matrix multiplication,

$${y}_{j} = {\sum }_{i = 1}^{n} {A}_{ji} {x}_{i} with {A}_{ji} ≡ {d}_{j} / {K}_{ij}$$

### Example 9: Assessment of Absolute Cargo Abundance of a Single Test Barcode Using a Reference Barcode (or Spike-In Barcode)

The present example demonstrates a method to determine absolute cargo abundance of cargos in a pool using a reference barcode and barcode decoding.

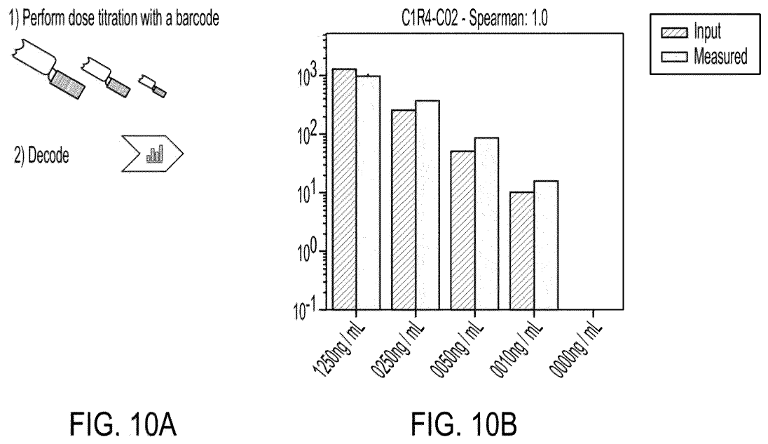

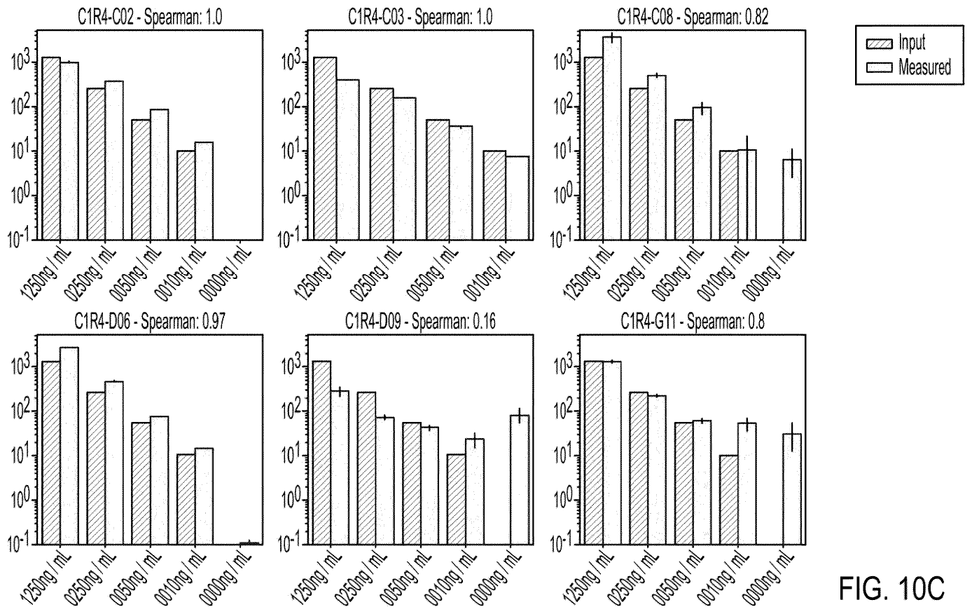

FIG. 10A shows a schematic of an experiment. A single test barcode attached to cargo was assayed at several concentrations, ranging from 0 ng/mL to 1250 ng/mL. In the same sample, a “spike-in” barcode attached to a cargo (i.e., a reference barcode) was added to each assay mixture at 250 ng/mL. Various concentrations of a test barcoded cargo were contacted with binding agents (i.e., binding agents with binders expressed on them) and decoding was performed as described herein. Prediction of a reference or “spike-in” barcode was used to determine an absolute amount of a test barcode, and by extension a barcoded cargo, being measured (see Example 8). FIG. 10B shows a plot of measured absolute quantities of the test barcode (right bar) compared to known input concentrations of the test barcode (left bar) for each titration of the test barcode. The Y-axis is a logarithm of the test barcode concentration in nanograms per milliliter (ng/mL). FIG. 10C shows results of determination of absolute concentration for 6 different barcoded cargos. The plots show known input concentrations (left bar) and measured concentration (right bar) for six (6) different barcoded cargos.

### Example 10: Simultaneous In Vivo Assessment of Phenotypes of Cargos Using Binder-Barcode Platform

The present example demonstrates a method to perform simultaneous in vivo assessment of phenotypes of cargos using a binder-barcode platform described herein. In some embodiments, a phenotype includes pharmacokinetic (or clearance) data as described herein.

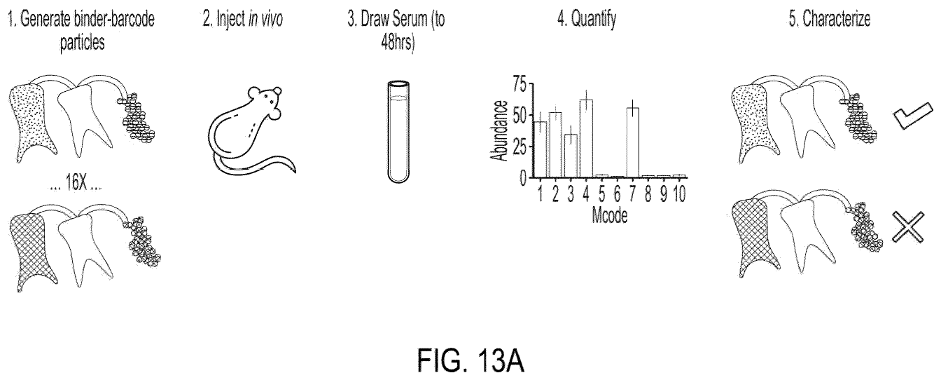

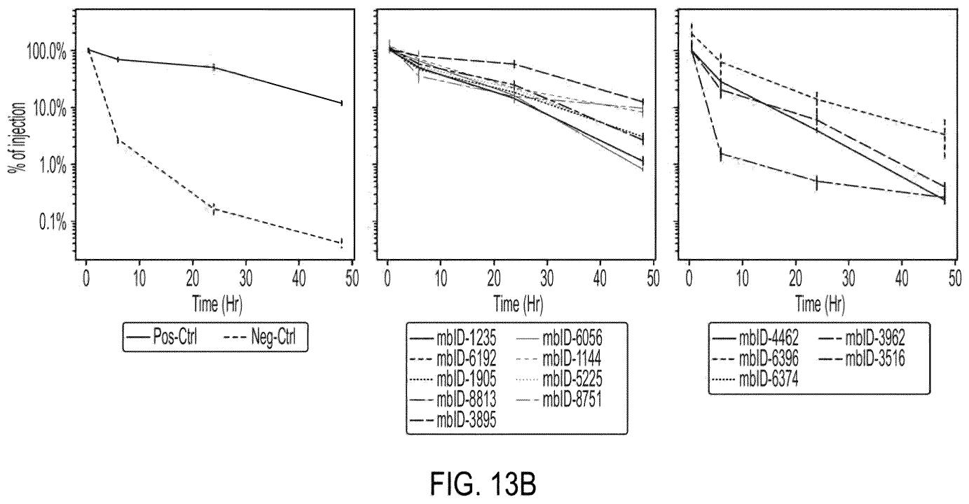

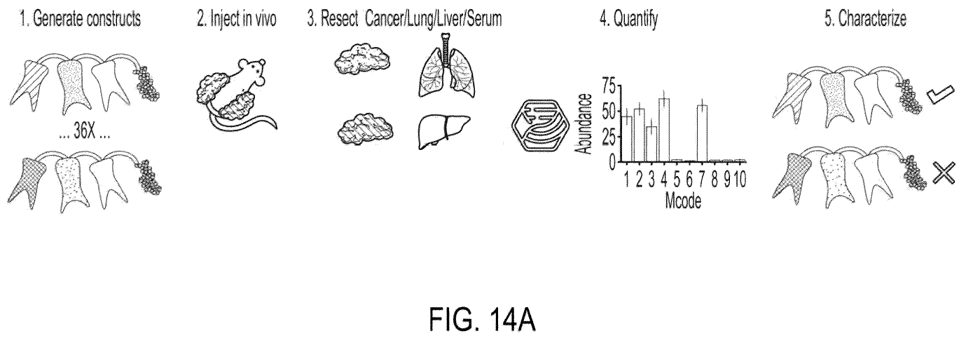

FIG. 13A is a schematic of a method to detect and/or quantify and/or characterize fourteen (14) exemplary cargos (e.g., proteins) in a pool using a binder-barcode platform described herein. Fourteen (14) exemplary binder molecules were produced with different barcodes as described herein (“binder-barcode particles”). Binder-barcode particles were injected as a pool into wild-type (wt) BALB/c mice. At timepoints 30 min, 6 hours, 24 hours, and 48 hours, blood was collected from individual mice (n=3 per timepoint), and serum was extracted. Binder-barcode particles were captured and subjected to the decoding procedure as described.

FIG. 13B shows simultaneous in vivo assessment of clearance phenotypes of cargos using a binder-barcode platform described herein. In particular, FIG. 13B shows plots of clearance of multiple cargos, measured simultaneously and grouped by rate of clearance phenotype (e.g., slow vs. fast clearance). FIG. 13B (left) depicts controls with known properties that were measured. FIG. 13B (center) depicts cargos identified as having slow clearance properties over time. FIG. 13B (right) depicts cargos identified as having fast clearance properties over time. Data was normalized to 100% of injection volume for each binder-barcode particle.

Accordingly, the present example confirms that a binder-barcode platform described herein can be used for simultaneous characterization of phenotypes in vivo.

### Example 11: Simultaneous In Vivo Assessment of Phenotypes of Cargos in Multiple Tissues Using Binder-Barcode Platform

Among other things, the present disclosure provides an insight that on-target, off-tumor toxicity is a biodistribution challenge. The present example demonstrates a method for simultaneous in vivo assessment of phenotypes of cargos in multiple tissues using a binder-barcode platform described herein. In some embodiments, a phenotype includes biodistribution data as described herein. In some embodiments, a phenotype includes pharmacokinetic (clearance) data as described herein. In some embodiments, a phenotype includes pharmacokinetic data and biodistribution data as described herein.

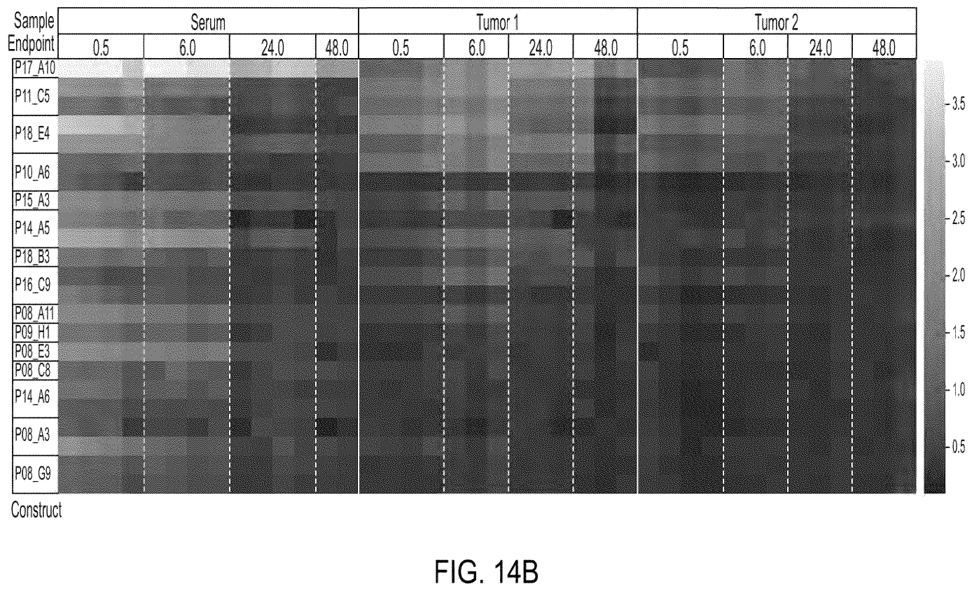

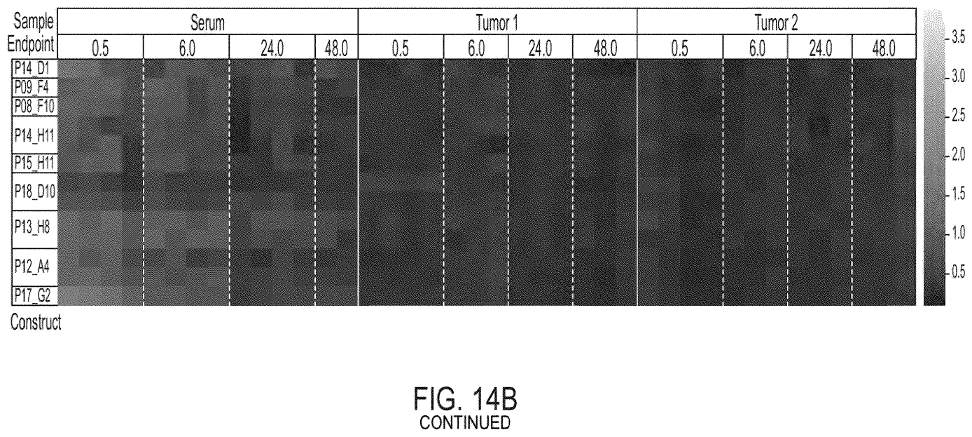

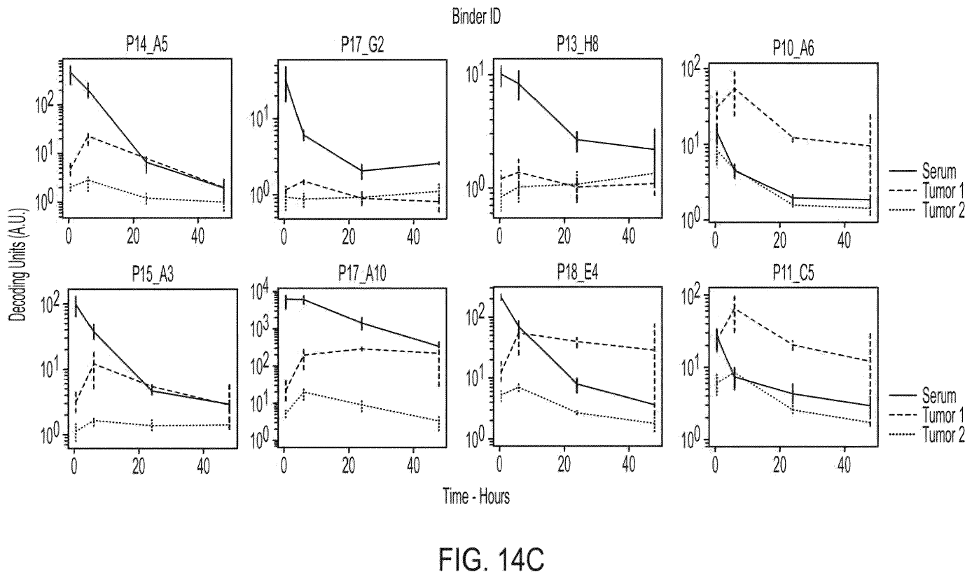

FIG. 14A is a schematic of a method to detect and/or identify and/or quantify and/or characterize thirty-six (36) exemplary cargos (e.g., proteins) in a pool using a binder-barcode platform described herein. Thirty-six (36) binder molecules were produced with different barcodes (“binder-barcode particles”). Binder-barcode particles were injected as a pool into tumor bearing NSG mice, which had been previously implanted with two tumor cell lines (Tumor 1, Tumor 2). At timepoints 30 min, 6 hours, 24 hours, and 48 hours, blood and tumor tissue was collected from individual mice (n=2-4 per timepoint). In some embodiments, other tissue can be harvested, including lung, liver, brain, etc. Tissue was lysed using standard lysis buffer. Serum was separated from blood. Binder-barcode particles were captured binder from each tissue and subjected to a decoding procedure as described herein.

FIG. 14B is a heat-map of all binder-barcode particle data collected as described herein. Rows indicate different binder constructs identifiers (IDs) that correlate to binder-barcode particles tested by the present example. Columns indicate data for a mouse across each time point for serum, Tumor 1, or Tumor 2. Color intensity indicates relative units of drug as measured via a decoding procedure described herein. Color intensity indicates a normalized readout of relative concentration as measured via next generation sequencing (NGS).

FIG. 14C depicts plots of binder-barcode particles described by FIG. 14B using a decoding procedure described herein. A diversity of properties was simultaneously measured. For example, binder-barcode particle P14_A5 was rapidly cleared from serum, with minimal accumulation in Tumor 1 or Tumor 2, while binder-barcode particle P17_A10 was more slowly cleared and maintained in tumor 1 over time.

Accordingly, the present example confirms that a binder-barcode platform described herein can be used for simultaneous characterization of phenotypes of cargos across multiple tissue types in vivo.

### Example 12: Simultaneous In Vivo Assessment of Phenotypes of Cargos Using Binder-Barcode Platform

The present example demonstrates a method for simultaneous in vivo assessment of phenotypes of cargos (e.g., proteins) using a binder-barcode platform described herein. In some embodiments, a phenotype is a half-life measurement as described herein.

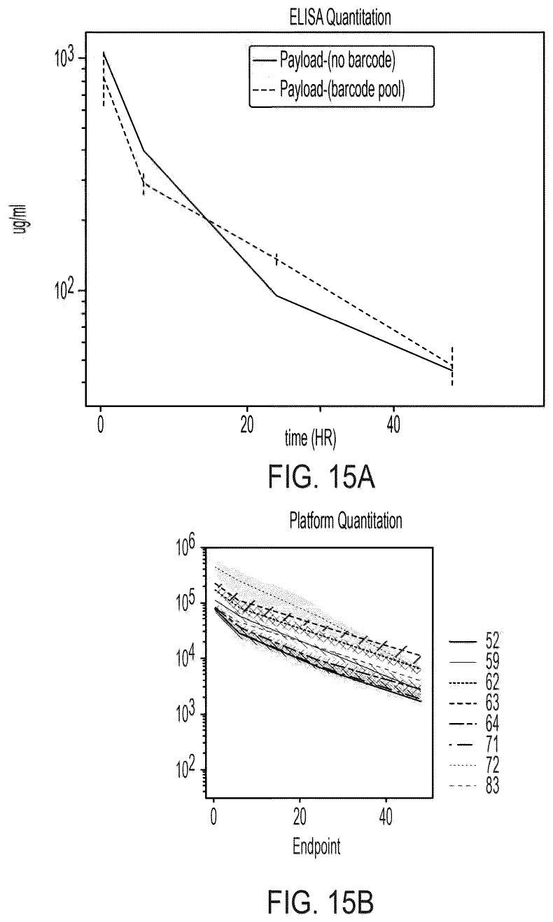

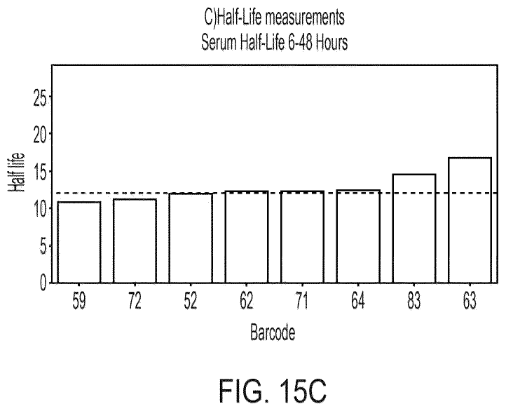

FIG. 15A depicts a plot showing ELISA quantitation of two groups of cargos (Group 1: cargo polypeptide with no barcode; Group 2: a pool of eight (8) binder-barcode particles where each particle includes the same cargo polypeptide used in Group 1, and each particle is barcoded with a different barcode). Group 1 was injected into a cohort of wild-type (wt) BALB/c mice. Group 2 was also injected into a cohort of wild-type (wt) BALB/c mice. At timepoints 6 hours, 24 hours, and 48 hours, blood was collected from individual mice (n=3 per timepoint), and serum was extracted. ELISA quantitation showed similar measurements between Group 1 and Group 2, confirming that barcode does not affect cargo function. FIG. 15B shows a plot depicting simultaneous and individual assessment of eight (8) distinct entities in Group 2 using a decoding procedure described herein. FIG. 15C shows a comparison of half-life measurements for Group 1 and Group 2. Half-life measurements for Group 1 was quantified using ELISA (dashed line). Half-life measurements for Group 2 was quantified using a decoding procedure described herein (bars). As shown in FIG. 15C, variation in half-life measurements across different binder-barcode particles within a pool can be resolved using a binder-barcode platform described herein. Moreover, ELISA cannot perform simultaneous measurements of phenotypes in one experiment.

Accordingly, the present example confirms that a binder-barcode platform described herein can be used for simultaneous, accurate characterization of phenotypes in vivo. Moreover, the present example confirms that attachment of a barcode to a cargo using a method described herein does not disrupt in vivo properties of a cargo.

### Example 13: Sensitivity and Dynamic Range of Binder-Barcode Platform

The present example demonstrates a method to determine sensitivity and dynamic range of a binder-barcode platform described herein.

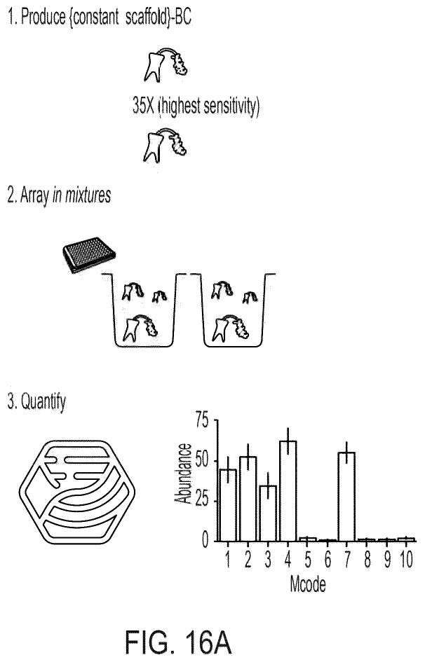

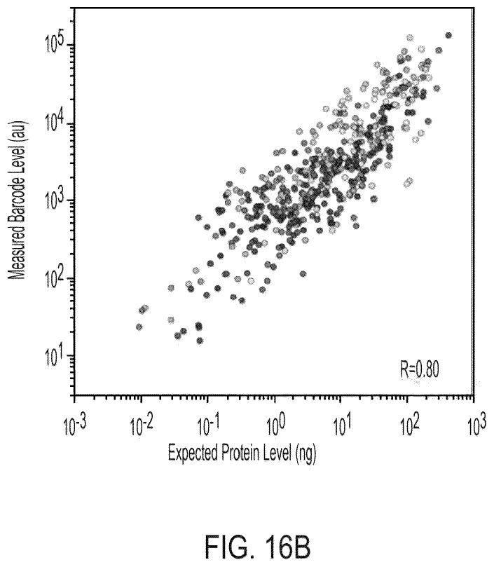

An array of ninety-six (96) mixtures comprising ten (10) to thirty-five (35) barcoded cargos (e.g., proteins), with each barcoded cargo having a known concentration between 1 picogram (pg) and 1 microgram (μg), was designed to determine sensitivity and dynamic range of a binder-barcode platform described herein (FIGS. 16A-16B). Each data point in FIG. 16B represents a comparison between a known concentration of a barcoded cargo particle from one of the ninety-six (96) distinct mixtures and a concentration determined by a decoding procedure described herein. As shown in FIG. 16B, cargos were quantified across a 10,000-fold range of concentrations. FIG. 16B also shows cargos down to 0.1 nanograms (ng) were also quantified.

Accordingly, the present example confirms that a binder-barcode platform described herein can be used for simultaneous, sensitive characterization of barcoded cargos across a broad dynamic range of concentrations in diverse mixtures.

### Example 14: Comparison of In Vitro and In Vivo Assessment of Phenotypes of Cargos Using Binder-Barcode Platform

The present example provides an insight that in vitro models sometimes fail to accurately model in vivo environments. Among other things, the present disclosure provides an insight that certain in vitro systems can be limited (e.g., with respect to their modeling of in vivo performance) by one or more of the following: two-dimensional monolayer of in vitro models versus complex three-dimensional architecture of in vivo environments, differences in target post translation modification or accessibility, differences in gene expression, absence of stromal cells and/or extracellular matrix, lack of circulation and diffusion from vasculature, or off-target and antigen sink effects not modeled in in vitro models.

Binder-barcode particles tested in Example 11 were also tested in an in vitro T cell activation assay (data not shown). A binder-barcode particle that exhibited highest T cell activation in vitro exhibited poor tumor tissue accumulation and rapid clearance in vivo. Moreover, a binder-barcode particle that exhibited modest T cell activation in vitro exhibited high tumor accumulation and slow clearance in vivo. Accordingly, the present example confirms that a binder-barcode platform described herein can be used to identify binders with unexpected in vivo properties. Moreover, the present example confirms that a binder-barcode platform described herein can be used to select therapeutic candidates exhibiting desirable phenotypes in vivo.

### Example 15: Barcode does not Significantly Affect Cargo Properties

The present example confirms that a barcode has relatively little or no effect on cargo (e.g., protein) properties. In some embodiments, a property includes cargo affinity as described herein. In some embodiments, a property includes cargo production (or yield) as described herein.

Affinity and production (or yield) of a cargo (e.g., protein) with and without a barcode were assessed using biolayer interferometry (BLI). Ten different barcodes were tested. Affinity of a barcoded cargo exhibited similar affinity of a cargo without a barcode, as measured by BLI (data not shown). Moreover, cargo production (or yield) of a barcoded cargo exhibited similar cargo production (or yield) of a cargo without a barcode, as measured by BLI (data not shown).

Accordingly, the present example confirms that cargo performance is not significantly impacted by a barcode. Moreover, the present example confirms that cargo production is not significantly impacted by a barcode.

### Example 16: Assessment and Quantification of Expression and/or Localization of Barcoded Cargo Polypeptides Delivered Using Multiple Serotypes of AAV Delivery Particles

The present example demonstrates a multiplexed, high-throughput method of measuring a barcoded cargo polypeptide as described herein. Moreover, the present example demonstrates that a binder-barcode platform described herein can be used to quantify effects (e.g., phenotypes such as localization, expression, etc.) that result from modifying cargo polypeptides as described herein. Further, the present example demonstrates that a binder-barcode platform described herein can be used to quantify effects (e.g., phenotypes such as localization, expression, etc.) that result from a type of delivery particle that is used to deliver a cargo component as described herein. In particular, the present example demonstrates effects of a secretion tag on expression and/or localization of cargo polypeptides using three different serotypes of AAV delivery particles (AAV2, AAV9, AAV.PHPB) in a pooled mixture. The present example demonstrates that expression and/or tropism of cargo polypeptides can be quantified using a binder-barcode platform as described herein.

Other types of cargo components and modifications to a cargo polypeptide can be assessed using a binder-barcode platform as described herein. Phenotypes other than expression and localization can also be quantified and/or assessed using a binder-barcode platform as described herein.

### Method

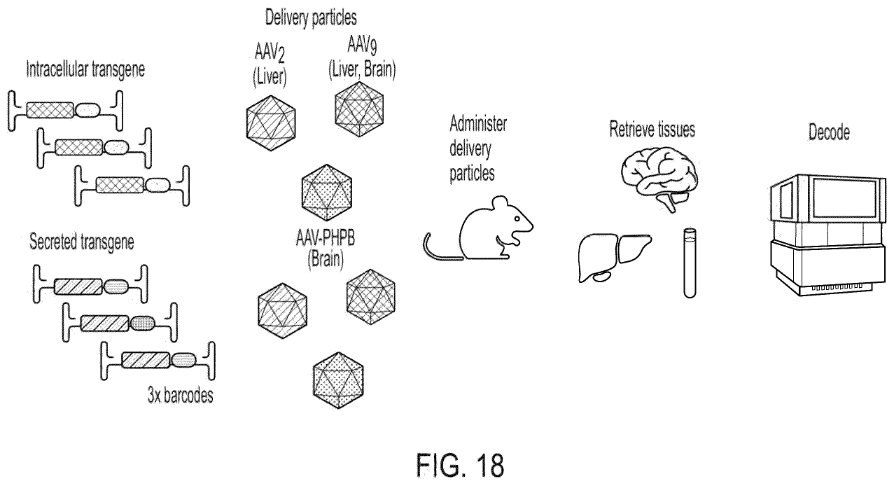

FIG. 18 depicts a schematic of an exemplary method described herein that provides for tracking and/or identification and/or assessment and/or quantification of different nucleic acids encoding a cargo component disposed within different types of delivery particles (e.g., AAV delivery particles, e.g., AAV2, AAV9, AAV.PHPB) that exhibit different tissue tropisms.

Two constructs comprising barcoded cargo components were synthesized. A first construct was formatted as Sec-ALFA-GOI-BC (“Construct 1”), where Sec is a nucleotide sequence encoding a secretory signal peptide, ALFA is a nucleotide sequence encoding a universal capture tag, GOI is a cargo component whose nucleotide sequence is or comprises a sequence encoding a cargo polypeptide of interest, and BC is a barcode component whose nucleotide sequence is or comprises a sequence encoding a peptide barcode. A second construct was formatted as ALFA-GOI-BC (“Construct 2”). A difference between Construct 1 and Construct 2 was that Construct 2 lacked a secretion signal peptide (Sec). For Construct, six (6) different versions were synthesized, and for Construct 2 nine (9) different version were synthesized, each associated with a different barcode (see Table 8). Each version of Construct 1 and Construct 2 were cloned into a backbone containing human cytomegalovirus (HCMV) promoter and an inverse terminal repeat (ITR) sequence for packaging in an adeno associate virus (AAV). The resulting plasmids were then used to produce AAV using methods known in the art. Three different AAV serotypes were used to package constructs, as shown in Table 8, namely AAV2, AAV9, and AAV-PHPB. Each version of Construct 1 and Construct 2 were individually packaged into each serotype of AAV. It is noted that AAV2 was only measured with a secretion tag. Table 8 shows experimental designs for Construct 1 and Construct 2.

TABLE 8
Different AAV variants produced and their content

| Construct # | Secretion tag | Barcode ID | AAV Serotype |
|---|---|---|---|
| 1 | Yes | 1 | AAV-2 |
| 1 | Yes | 2 | AAV-2 |
| 1 | Yes | 3 | AAV-2 |
| 1 | Yes | 4 | AAV-9 |
| 1 | Yes | 5 | AAV-9 |
| 1 | Yes | 6 | AAV-9 |
| 1 | Yes | 7 | AAV-PHPB |
| 1 | Yes | 8 | AAV-PHPB |
| 1 | Yes | 9 | AAV-PHPB |
| 2 | No | 10 | AAV-9 |
| 2 | No | 11 | AAV-9 |
| 2 | No | 12 | AAV-9 |
| 2 | No | 13 | AAV-PHPB |
| 2 | No | 14 | AAV-PHPB |
| 2 | No | 15 | AAV-PHPB |

AAV delivery particles were titered via qPCR to determine genome copies per mL (GC/mL). AAV delivery particles were pooled such that GC/mL was equal across all variants.

C57 female mice (n=15) were injected via tail vein with 1×10¹¹ total genome copies of the pooled AAV delivery particles. Three (3) mice were then euthanized at each time point (1 day, 2 days, 7 days, 14 days, and 21 days) and brain, liver and serum tissues were collected. Tissues were lysed using Radioimmunoprecipitation Assay (RIPA) buffer with a HALT protease inhibitor cocktail and two (2) rounds of bead beating for 60 seconds at 25 Hz, and barcodes were subject to decoding as previously described herein.

### Results

A person of ordinary skill in the art would understand tropisms conferred by AAV serotypes, such as AAV2, AAV9, AAV-PHPB. For example, AAV2 generally has poor expression and primarily infects liver, AAV9 has mostly high liver expression and low levels of expression in the brain, and AAV.PHPB infects and expresses cargo polypeptides in neuron cells (see Weinmann et al, Nature Communications, 11, 5432 (2020)). However, these results are either usually performed as single measurements when looking at protein, or via RNA and/or DNA sequencing of the viral genomes when looking in a pool. Furthermore, effects of incorporating one or more components such as a secretion tag on expression and/or localization of a cargo polypeptide and assessing such effects is unknown. Moreover, other technologies would not be able to assess effects on delivered cargo polypeptides using different types of delivery particles in a multiplexed and high-throughput format.

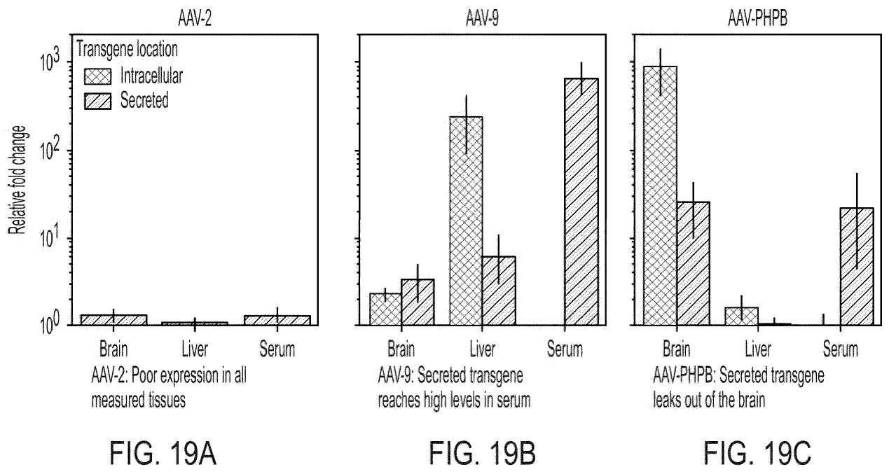

FIGS. 19A, 19B, and 19C show data for three replicate mice at 14 days, averaged across barcode and mouse replicates. It is noted that data from other time points look similar. The Y-axis in each of the graphs of FIGS. 19A-19C show a fold-change in decoding measurement for a given barcoded cargo component and serotype pair as compared to a background measurement. The X-axis in each of FIGS. 19A-19C shows various tissues analyzed for each of the three AAV serotypes. Green bars correspond to Construct 2 (i.e., constructs without a secretion tag), and orange bars correspond to Construct 1 (i.e., constructs with a secretion tag. AAV2, which was only measured with a secretion tag, had general poor expression across tissues collected compared to other serotypes (see FIG. 19A). Strikingly, when AAV9 was used for packaging, a cargo polypeptide was expressed and localized over 100-fold more in serum compared to liver when a secretion tag was associated with a cargo polypeptide (see FIG. 19B). Additionally, as shown in FIG. 19B, cargo polypeptides were not localized in serum without a secretion tag. Further, AAV9 cargo polypeptides were expressed in brain tissue, which was detected marginally over background for both Construct 1 and Construct 2. AAV.PHPB is known to infect and express exclusively from neurons. However, when a secretion tag was associated with a cargo polypeptide and packaged in AAV.PHPB, almost equal amounts of cargo polypeptide localized in serum and brain tissue (see FIGS. 19A-19C). This finding indicates that a secretion tag is leading to leakage from brain tissue. Conversely, when no secretion tag is added to a cargo polypeptide, no cargo polypeptide was seen in serum and very high levels of cargo polypeptide expression was seen in brain tissue (see FIG. 19C).

### Discussion

As described herein, peptide barcodes were used to assess effects of localization and/or expression of cargo polypeptides delivered by a variety of AAV serotypes in a single mixture. This workflow would have been previously impossible to perform and would not previously be explicitly capable of measuring delivered cargo polypeptides directly. If a skilled person were to look only at DNA and RNA of AAV, a skilled person would obtain results that are not well correlated with actual localization or expression of a cargo polypeptide. Accordingly, the present example demonstrates quantification of expression and/or localization of different types of cargo polypeptides using different types delivery particles in a single experiment using a binder-barcode platform described herein.

It is an insight of the present disclosure that other forms of delivery vehicles (e.g., viral particles, lipid-based particles ((e.g., cell-produced or not cell-produced), lipid nanoparticles (LNPs), liposomes, micelles, extracellular vesicles (e.g., exosomes, microparticles, etc.)), polymer-based particles (e.g., PGLA-based), polysaccharide-based particles, etc.) and/or nucleic acids (e.g., RNA) may be used in accordance with the Examples described herein.

Moreover, delivery particles and/or nucleic acids (e.g., RNA) may be designed and/or tested via a binder-barcode platform to assess and/or quantify nucleic acids using a binder-barcode platform described herein to address challenges with correlating an amount of nucleic acid in a tissue with functional performance (e.g., expression). Furthermore, the present example may be used to assess large pools of therapeutic polypeptides delivered simultaneously to cells, tissues, or organs. This workflow reduces a number of burdens of conventional methods, including production, assessment, and ability to capture functional readouts of cargo polypeptides and/or delivery particles in a high-throughput and multiplexed format.

### Example 17: In Vivo Discovery and Assessment of Barcoded Cargo Polypeptides that Cross the Blood-Brain Barrier (BBB) Using AAV Delivery Particles

The present example demonstrates a multiplexed, high-throughput method of measuring a barcoded cargo polypeptide as described herein. Moreover, the present example demonstrates that a binder-barcode platform described herein can be used to identify and/or quantify cargo polypeptides for certain phenotypes such as expression and/or localization. For example, in some embodiments, cargo polypeptides can be identified and/or assessed for ability to cross the blood-brain barrier (BBB) as described by this example.

### Method

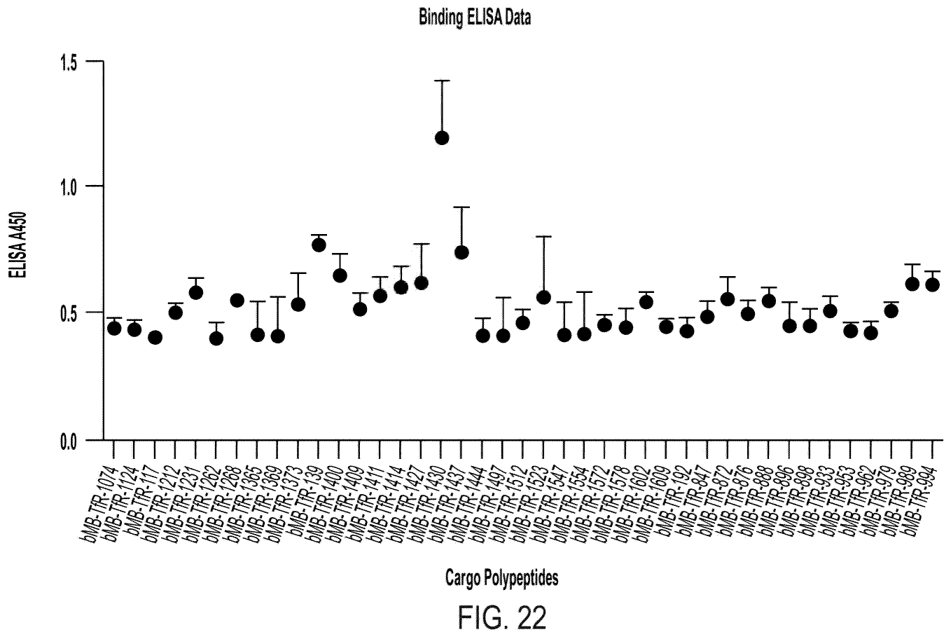

A set of cargo polypeptides were measured via octet biolayer interferometry (BLI) to determine their respective dissociation against the transferrin receptor (TfR). Dissociation against TfR for another set of cargo polypeptides were determined via ELISA according to standard methods (see, e.g., Engvall, E., Clinical Chemistry, Volume 56, Issue 2, 2010, Pages 319-320). Results of an ELISA assay are shown in FIG. 22. Each cargo component encoding a cargo polypeptide was then associated with a unique barcode component Each peptide barcode encoded by a barcode component is associated (e.g., covalently or not covalently) with the C-terminus of the cargo polypeptide via a linker. Nucleic acids (or constructs) were synthesized with cargo components including a sequence encoding a cargo polypeptide, promoter elements, terminator elements, and/or ITR elements for AAV packaging according to standard methods. Table 9 below shows experimental designs for constructs tested in this example.

TABLE 9
Different AAV delivery particles produced and their content

| | | Barcode | |
|---|---|---|---|
| Construct # | Cargo | ID | AAV Serotype |
| 1 | bMB-TfR-116 | bc-28 | AAV-DJ |
| 2 | bMB-TfR-541 | bc-211 | AAV-DJ |
| 3 | bMB-TfR-305 | bc-100 | AAV-DJ |
| 4 | bMB-TfR-359 | bc-170 | AAV-DJ |
| 5 | bMB-TfR-085 | bc-248 | AAV-DJ |
| 6 | bMB-TfR-002 | bc-266 | AAV-DJ |
| 7 | bMB-TfR-353 | bc-189 | AAV-DJ |
| 8 | bMB-TfR-231 | bc-280 | AAV-DJ |
| 9 | bMB-TfR-267 | bc-238 | AAV-DJ |
| 10 | bMB-TfR-143 | bc-8 | AAV-DJ |
| 11 | bMB-TfR-300 | bc-62 | AAV-DJ |
| 12 | bMB-TfR-299 | bc-84 | AAV-DJ |
| 13 | bMB-TfR-248 | bc-20 | AAV-DJ |
| 14 | bMB-TfR-106 | bc-318 | AAV-DJ |
| 15 | bMB-TfR-352 | bc-161 | AAV-DJ |
| 16 | bMB-TfR-139 | bc-155 | AAV-DJ |
| 17 | bMB-TfR-152 | bc-27 | AAV-DJ |
| 18 | bMB-TfR-154 | bc-141 | AAV-DJ |
| 19 | bMB-TfR-158 | bc-135 | AAV-DJ |
| 20 | bMB-TfR-160 | bc-283 | AAV-DJ |
| 21 | bMB-TfR-326 | bc-43 | AAV-DJ |
| 22 | bMB-TfR-354 | bc-79 | AAV-DJ |
| 23 | bMB-TfR-144 | bc-224 | AAV-DJ |
| 24 | bMB-TfR-121 | bc-187 | AAV-DJ |
| 25 | bMB-TfR-160 | bc-33 | AAV-DJ |
| 26 | bMB-TfR-117 | bc-340 | AAV-DJ |
| 27 | bMB-TfR-136 | bc-276 | AAV-DJ |
| 28 | bMB-TfR-318 | bc-180 | AAV-DJ |
| 29 | bMB-TfR-122 | bc-86 | AAV-DJ |
| 30 | bMB-TfR-236 | bc-228 | AAV-DJ |
| 31 | bMB-TfR-140 | bc-45 | AAV-DJ |
| 32 | bMB-TfR-343 | bc-130 | AAV-DJ |
| 33 | bMB-TfR-329 | bc-4 | AAV-DJ |
| 34 | bMB-TfR-193 | bc-22 | AAV-DJ |
| 35 | bMB-TfR-559 | bc-10 | AAV-DJ |
| 36 | bMB-TfR-086 | bc-167 | AAV-DJ |
| 37 | bMB-TfR-265 | bc-251 | AAV-DJ |
| 38 | bMB-TfR-324 | bc-304 | AAV-DJ |
| 39 | bMB-TfR-313 | bc-292 | AAV-DJ |
| 40 | bMB-TfR-356 | bc-73 | AAV-DJ |
| 41 | bMB-TfR-357 | bc-168 | AAV-DJ |
| 42 | bMB-TfR-379 | bc-162 | AAV-DJ |
| 43 | bMB-TfR-345 | bc-32 | AAV-DJ |
| 44 | bMB-TfR-016 | bc-7 | AAV-DJ |
| 45 | bMB-TfR-847 | bc-278 | AAV-DJ |
| 46 | bMB-TfR-994 | bc-150 | AAV-DJ |
| 47 | bMB-TfR-525 | bc-17 | AAV-DJ |
| 48 | bMB-TfR-387 | bc-195 | AAV-DJ |
| 49 | bMB-TfR-365 | bc-249 | AAV-DJ |
| 50 | bMB-TfR-334 | bc-142 | AAV-DJ |
| 51 | bMB-TfR-151 | bc-226 | AAV-DJ |
| 52 | bMB-TfR-136 | bc-122 | AAV-DJ |
| 53 | bMB-TfR-310 | bc-196 | AAV-DJ |
| 54 | bMB-TfR-295 | bc-93 | AAV-DJ |
| 55 | bMB-TfR-302 | bc-59 | AAV-DJ |
| 56 | bMB-TfR-312 | bc-25 | AAV-DJ |
| 57 | bMB-TfR-321 | bc-145 | AAV-DJ |
| 58 | bMB-TfR-142 | bc-40 | AAV-DJ |
| 59 | bMB-TfR-989 | bc-41 | AAV-DJ |
| 60 | fc5_vhh | bc-270 | AAV-DJ |
| 61 | lag16 | bc-53 | AAV-DJ |
| 62 | fc5_vhh | bc-116 | AAV-DJ |
| 63 | lag16 | bc-52 | AAV-DJ |
| 64 | fc5_vhh | bc-327 | AAV-DJ |
| 65 | lag16 | bc-97 | AAV-DJ |
| 66 | bMB-TfR-113 | bc-28 | AAV-DJ |
| 67 | bMB-TfR-896 | bc-211 | AAV-DJ |
| 68 | bMB-TfR-962 | bc-100 | AAV-DJ |
| 69 | bMB-TfR-309 | bc-170 | AAV-DJ |
| 70 | bMB-TfR-876 | bc-248 | AAV-DJ |
| 71 | bMB-TfR-064 | bc-266 | AAV-DJ |
| 72 | bMB-TfR-215 | bc-189 | AAV-DJ |
| 73 | bMB-TfR-141 | bc-280 | AAV-DJ |
| 74 | bMB-TfR-147 | bc-238 | AAV-DJ |
| 75 | bMB-TfR-979 | bc-8 | AAV-DJ |
| 76 | bMB-TfR-341 | bc-62 | AAV-DJ |
| 77 | bMB-TfR-383 | bc-84 | AAV-DJ |
| 78 | bMB-TfR-278 | bc-20 | AAV-DJ |
| 79 | bMB-TfR-445 | bc-318 | AAV-DJ |
| 80 | bMB-TfR-264 | bc-161 | AAV-DJ |
| 81 | bMB-TfR-109 | bc-155 | AAV-DJ |
| 82 | bMB-TfR-157 | bc-27 | AAV-DJ |
| 83 | bMB-TfR-151 | bc-141 | AAV-DJ |
| 84 | bMB-TfR-933 | bc-135 | AAV-DJ |
| 85 | bMB-TfR-157 | bc-283 | AAV-DJ |
| 86 | bMB-TfR-125 | bc-43 | AAV-DJ |
| 87 | bMB-TfR-306 | bc-79 | AAV-DJ |
| 88 | bMB-TfR-192 | bc-224 | AAV-DJ |
| 89 | bMB-TfR-320 | bc-187 | AAV-DJ |
| 90 | bMB-TfR-126 | bc-33 | AAV-DJ |
| 91 | bMB-TfR-360 | bc-340 | AAV-DJ |
| 92 | bMB-TfR-953 | bc-276 | AAV-DJ |
| 93 | bMB-TfR-363 | bc-180 | AAV-DJ |
| 94 | bMB-TfR-250 | bc-86 | AAV-DJ |
| 95 | bMB-TfR-898 | bc-228 | AAV-DJ |
| 96 | bMB-TfR-888 | bc-45 | AAV-DJ |
| 97 | bMB-TfR-328 | bc-130 | AAV-DJ |
| 98 | bMB-TfR-348 | bc-4 | AAV-DJ |
| 99 | bMB-TfR-119 | bc-22 | AAV-DJ |
| 100 | bMB-TfR-296 | bc-10 | AAV-DJ |
| 101 | bMB-TfR-293 | bc-167 | AAV-DJ |
| 102 | bMB-TfR-350 | bc-251 | AAV-DJ |
| 103 | bMB-TfR-335 | bc-304 | AAV-DJ |
| 104 | bMB-TfR-155 | bc-292 | AAV-DJ |
| 105 | bMB-TfR-315 | bc-73 | AAV-DJ |
| 106 | bMB-TfR-373 | bc-168 | AAV-DJ |
| 107 | bMB-TfR-301 | bc-162 | AAV-DJ |
| 108 | bMB-TfR-872 | bc-32 | AAV-DJ |
| 109 | bMB-TfR-280 | bc-7 | AAV-DJ |
| 110 | bMB-TfR-140 | bc-278 | AAV-DJ |
| 111 | bMB-TfR-149 | bc-150 | AAV-DJ |
| 112 | bMB-TfR-126 | bc-17 | AAV-DJ |
| 113 | bMB-TfR-162 | bc-195 | AAV-DJ |
| 114 | bMB-TfR-143 | bc-249 | AAV-DJ |
| 115 | bMB-TfR-337 | bc-142 | AAV-DJ |
| 116 | bMB-TfR-123 | bc-226 | AAV-DJ |
| 117 | bMB-TfR-112 | bc-122 | AAV-DJ |
| 118 | bMB-TfR-393 | bc-196 | AAV-DJ |
| 119 | bMB-TfR-377 | bc-93 | AAV-DJ |
| 120 | bMB-TfR-107 | bc-59 | AAV-DJ |
| 121 | bMB-TfR-252 | bc-25 | AAV-DJ |
| 122 | bMB-TfR-319 | bc-145 | AAV-DJ |
| 123 | bMB-TfR-137 | bc-40 | AAV-DJ |
| 124 | bMB-TfR-098 | bc-41 | AAV-DJ |

DNA of the aforementioned nucleic acid constructs were pooled in equimolar ratios to form two non-overlapping pools. These pools were then used to construct AAV delivery particles according to standard methods. Two pools were then titered using a qPCR assay to determine the genome copies present per ml (GC/mL). Titers for the two pools are shown in a table below.

TABLE 10
Table showing GC/mL for two pools AAV

| Pool # | AAV Library | GC/mL |
|---|---|---|
| 1 | AAV-library-1 | 7.48 × 10¹² GC/mL |
| 2 | AAV-library-2 | 1.82 × 10¹³ GC/mL |

AAV delivery particles were diluted to 5×10¹¹ GC/mL and 200 μL of each of the two AAV libraries was injected into transgenic female C57 containing a humanized transferrin receptor allele (Mouse strain C57BU/6-Tfr1tm1TFR1/Bcgen). Each AAV library was injected into 15 individual mice (n=15). Three (3) mice are euthanized at each time point (7 days, 14 days, 21 days, and 28 days). Mice are perfused with 2× blood volumes of Phosphate buffered Saline (PBS). Blood is then collected and serum is separated from blood. After perfusion, brain, liver, muscle, skin, heart, lung, kidney, stomach, intestine tissues are all collected. Tissue is then lysed and subjected to decoding as described herein.

### Results

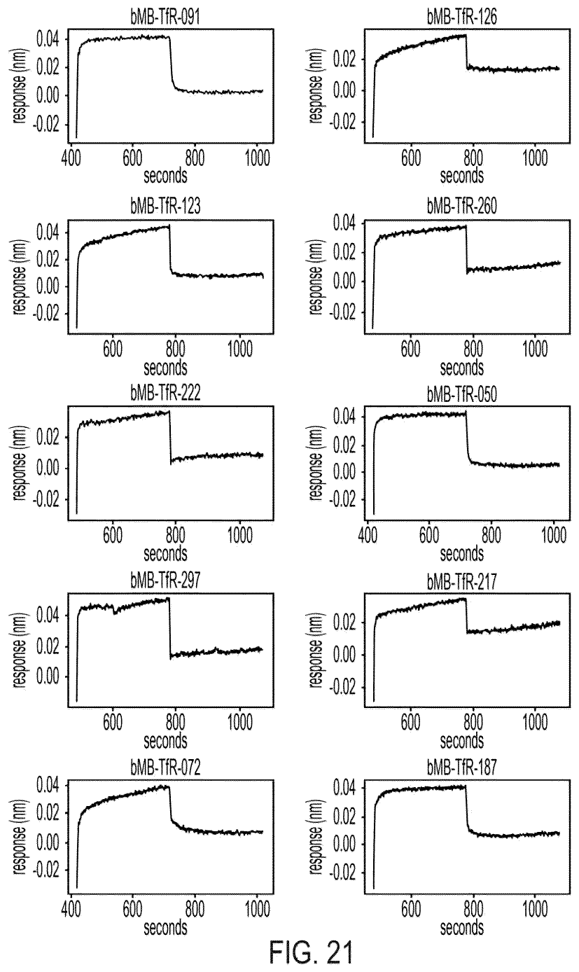

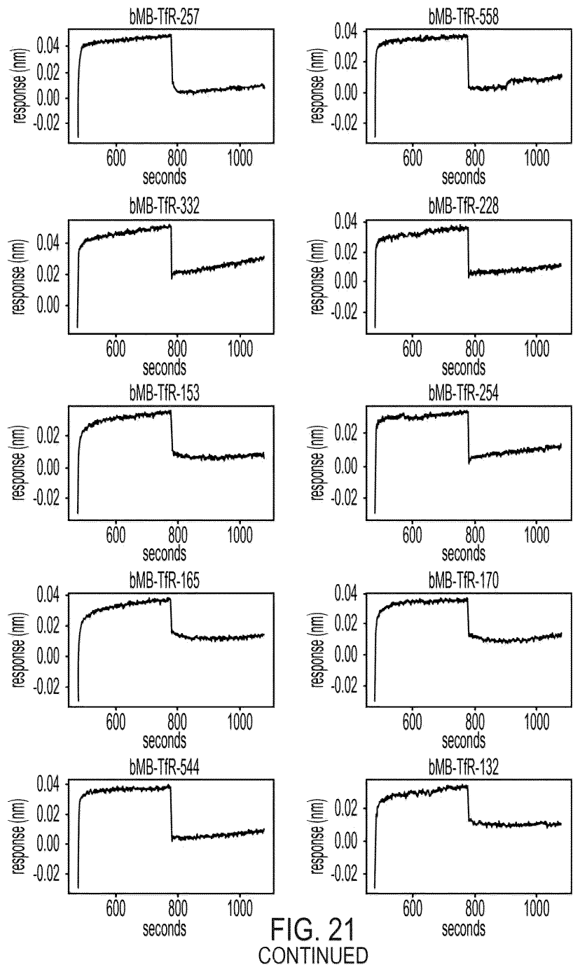

The present example demonstrates that a binder-barcode platform as described herein can quantify localization and/or expression of cargo polypeptide in certain tissues, including brain tissue, and can determine if said cargo polypeptide is also localizing and/or being expressed in other tissue (such as serum), including a ratio of amount of cargo polypeptide being expressed across different tissue types. For example, data in FIG. 21 shows respective dissociation of cargo polypeptides against the transferrin receptor (TfR) according to embodiments described herein.

Moreover, delivery particles, including AAV delivery particles, provide continuous expression of cargo polypeptides, which increases overall sensitivity of a binder-barcode platform as described herein. Ability to quantify potent and functional delivery of cargo polypeptides across different tissues can help identify potent cargo polypeptides that not only cross the BBB but are retained and/or more significantly expressed in brain tissue compared to other tissues.

It is an insight of the present disclosure that other forms of delivery vehicles (e.g., viral particles, lipid-based particles ((e.g., cell-produced or not cell-produced), lipid nanoparticles (LNPs), liposomes, micelles, extracellular vesicles (e.g., exosomes, microparticles, etc.)), polymer-based particles (e.g., PGLA-based), polysaccharide-based particles, etc.) and/or nucleic acids (e.g., RNA) may be used in accordance with the Examples described herein.

Accordingly, the present example demonstrates a multiplexed and high-throughput approach that can be used to identify and quantify cargo polypeptides that cross the BBB.

### Example 18: In Vivo Discovery and Assessment of Barcoded Cargo Polypeptides that Target Tumors Using AAV Delivery Particles

The present example demonstrates a multiplexed method of measuring pooled barcoded cargo polypeptides to assess tumor targeting using AAV delivery particles and a binder-barcode platform as described herein.

### Method

A set of cargo polypeptides is measured via octet biolayer interferometry (BLI) to determine their respective dissociation against a tumor target. Another set of cargo polypeptides is determined via ELISA according to standard methods (see, e.g., Engvall, E., Clinical Chemistry, Volume 56, Issue 2, 2010, Pages 319-320). Each cargo component encoding a cargo polypeptide was then associated with a unique barcode component. Each peptide barcode encoded by a barcode component is associated (e.g., covalently or not covalently) with the C-terminus of the cargo polypeptide via a linker. Nucleic acids (or constructs) were synthesized with cargo components including a sequence encoding a cargo polypeptide, promoter elements, terminator elements, and/or ITR elements for AAV packaging according to standard methods. Table 11 below shows experimental designs for constructs tested in this example.

TABLE 11
Different AAV delivery particles produced and their content

| Construct | | | AAV |
|---|---|---|---|
| # | Cargo | Barcode ID | Serotype |
| 1 | bMB-N4-3 | 1 | AAV-8 |
| 2 | bMB-N4-4 | 2 | AAV-8 |
| 3 | bMB-N4-8 | 3 | AAV-8 |
| 4 | bMB-N4-11 | 4 | AAV-8 |
| 5 | bMB-N4-13 | 5 | AAV-8 |
| 6 | bMB-N4-34 | 6 | AAV-8 |
| 7 | bMB-N4-40 | 7 | AAV-8 |
| 8 | bMB-N4-52 | 8 | AAV-8 |
| 9 | bMB-N4-53 | 9 | AAV-8 |
| 10 | bMB-N4-59 | 10 | AAV-8 |
| 11 | bMB-N4-69 | 11 | AAV-8 |
| 12 | bMB-N4-79 | 12 | AAV-8 |
| 13 | bMB-N4-82 | 13 | AAV-8 |
| 14 | bMB-N4-86 | 14 | AAV-8 |
| 15 | bMB-N4-87 | 15 | AAV-8 |
| 16 | bMB-N4-94 | 16 | AAV-8 |
| 17 | bMB-N4-98 | 17 | AAV-8 |
| 18 | bMB-N4-99 | 18 | AAV-8 |
| 19 | bMB-N4-102 | 19 | AAV-8 |
| 20 | bMB-N4-103 | 20 | AAV-8 |
| 21 | bMB-N4-108 | 21 | AAV-8 |
| 22 | bMB-N4-111 | 22 | AAV-8 |
| 23 | bMB-N4-113 | 23 | AAV-8 |
| 24 | bMB-N4-115 | 24 | AAV-8 |
| 25 | bMB-N4-119 | 25 | AAV-8 |
| 26 | bMB-N4-122 | 26 | AAV-8 |
| 27 | bMB-N4-126 | 27 | AAV-8 |
| 28 | bMB-N4-127 | 28 | AAV-8 |
| 29 | bMB-N4-131 | 29 | AAV-8 |
| 30 | bMB-N4-135 | 30 | AAV-8 |
| 31 | bMB-N4-138 | 31 | AAV-8 |
| 32 | bMB-N4-143 | 32 | AAV-8 |
| 33 | bMB-N4-146 | 33 | AAV-8 |
| 34 | bMB-N4-147 | 34 | AAV-8 |
| 35 | bMB-N4-148 | 35 | AAV-8 |
| 36 | bMB-N4-154 | 36 | AAV-8 |
| 37 | bMB-N4-155 | 37 | AAV-8 |
| 38 | bMB-N4-160 | 38 | AAV-8 |
| 39 | bMB-N4-162 | 39 | AAV-8 |
| 40 | bMB-N4-166 | 40 | AAV-8 |
| 41 | bMB-N4-170 | 41 | AAV-8 |
| 42 | bMB-N4-179 | 42 | AAV-8 |
| 43 | bMB-N4-183 | 43 | AAV-8 |
| 44 | bMB-N4-185 | 44 | AAV-8 |
| 45 | bMB-N4-190 | 45 | AAV-8 |
| 46 | bMB-N4-193 | 46 | AAV-8 |
| 47 | bMB-N4-194 | 47 | AAV-8 |
| 48 | bMB-N4-198 | 48 | AAV-8 |
| 49 | bMB-N4-199 | 49 | AAV-8 |
| 50 | bMB-N4-208 | 50 | AAV-8 |
| 51 | bMB-N4-209 | 51 | AAV-8 |
| 52 | bMB-N4-215 | 52 | AAV-8 |
| 53 | bMB-N4-216 | 53 | AAV-8 |
| 54 | bMB-N4-219 | 54 | AAV-8 |
| 55 | bMB-N4-220 | 55 | AAV-8 |
| 56 | bMB-N4-221 | 56 | AAV-8 |
| 57 | bMB-N4-222 | 57 | AAV-8 |
| 58 | bMB-N4-223 | 58 | AAV-8 |
| 59 | bMB-N4-234 | 59 | AAV-8 |
| 60 | bMB-N4-236 | 60 | AAV-8 |
| 61 | bMB-N4-240 | 61 | AAV-8 |
| 62 | bMB-N4-248 | 62 | AAV-8 |
| 63 | bMB-N4-251 | 63 | AAV-8 |
| 64 | bMB-N4-253 | 64 | AAV-8 |
| 65 | bMB-N4-264 | 65 | AAV-8 |
| 66 | bMB-N4-295 | 66 | AAV-8 |
| 67 | bMB-N4-297 | 67 | AAV-8 |
| 68 | bMB-N4-314 | 68 | AAV-8 |
| 69 | bMB-N4-325 | 69 | AAV-8 |
| 70 | bMB-N4-327 | 70 | AAV-8 |
| 71 | bMB-N4-349 | 71 | AAV-8 |
| 72 | bMB-N4-351 | 72 | AAV-8 |
| 73 | bMB-N4-356 | 73 | AAV-8 |
| 74 | bMB-N4-359 | 74 | AAV-8 |
| 75 | bMB-N4-364 | 75 | AAV-8 |
| 76 | bMB-N4-377 | 76 | AAV-8 |
| 77 | bMB-N4-385 | 77 | AAV-8 |
| 78 | bMB-N4-387 | 78 | AAV-8 |
| 79 | bMB-N4-388 | 79 | AAV-8 |
| 80 | bMB-N4-389 | 80 | AAV-8 |
| 81 | bMB-N4-390 | 81 | AAV-8 |
| 82 | bMB-N4-394 | 82 | AAV-8 |
| 83 | bMB-N4-398 | 83 | AAV-8 |
| 84 | bMB-N4-400 | 84 | AAV-8 |
| 85 | bMB-N4-401 | 85 | AAV-8 |
| 86 | bMB-N4-409 | 86 | AAV-8 |
| 87 | bMB-N4-446 | 87 | AAV-8 |
| 88 | bMB-N4-448 | 88 | AAV-8 |
| 89 | bMB-N4-467 | 89 | AAV-8 |
| 90 | bMB-N4-483 | 90 | AAV-8 |
| 91 | bMB-N4-559 | 91 | AAV-8 |
| 92 | bMB-N4-627 | 92 | AAV-8 |
| 93 | bMB-N4-638 | 93 | AAV-8 |
| 94 | bMB-N4-768 | 94 | AAV-8 |
| 95 | bMB-N4-772 | 95 | AAV-8 |
| 96 | bMB-N4-775 | 96 | AAV-8 |
| 97 | bMB-N4-785 | 97 | AAV-8 |
| 98 | bMB-N4-787 | 98 | AAV-8 |
| 99 | bMB-N4-788 | 99 | AAV-8 |
| 100 | bMB-N4-789 | 100 | AAV-8 |
| 101 | bMB-N4-790 | 101 | AAV-8 |
| 102 | bMB-N4-796 | 102 | AAV-8 |
| 103 | bMB-N4-797 | 103 | AAV-8 |
| 104 | bMB-N4-810 | 104 | AAV-8 |
| 105 | bMB-N4-819 | 105 | AAV-8 |
| 106 | bMB-N4-820 | 106 | AAV-8 |
| 107 | bMB-N4-822 | 107 | AAV-8 |
| 108 | bMB-N4-846 | 108 | AAV-8 |
| 109 | bMB-N4-857 | 109 | AAV-8 |
| 110 | bMB-N4-860 | 110 | AAV-8 |
| 111 | bMB-N4-861 | 111 | AAV-8 |
| 112 | bMB-N4-870 | 112 | AAV-8 |
| 113 | bMB-N4-876 | 113 | AAV-8 |
| 114 | bMB-N4-877 | 114 | AAV-8 |
| 115 | bMB-N4-879 | 115 | AAV-8 |
| 116 | bMB-N4-881 | 116 | AAV-8 |
| 117 | bMB-N4-883 | 117 | AAV-8 |
| 118 | bMB-N4-890 | 118 | AAV-8 |
| 119 | bMB-N4-922 | 119 | AAV-8 |
| 120 | bMB-N4-934 | 120 | AAV-8 |
| 121 | bMB-N4-940 | 121 | AAV-8 |
| 122 | bMB-N4-942 | 122 | AAV-8 |
| 123 | bMB-N4-955 | 123 | AAV-8 |
| 124 | bMB-N4-960 | 124 | AAV-8 |
| 125 | bMB-N4-975 | 125 | AAV-8 |
| 126 | bMB-N4-985 | 126 | AAV-8 |
| 127 | bMB-N4-988 | 127 | AAV-8 |
| 128 | bMB-N4-997 | 128 | AAV-8 |
| 129 | bMB-N4-1000 | 129 | AAV-8 |
| 130 | bMB-N4-1003 | 130 | AAV-8 |
| 131 | bMB-N4-1004 | 131 | AAV-8 |
| 132 | bMB-N4-1007 | 132 | AAV-8 |
| 133 | bMB-N4-1009 | 133 | AAV-8 |
| 134 | bMB-N4-1011 | 134 | AAV-8 |
| 135 | bMB-N4-1023 | 135 | AAV-8 |
| 136 | bMB-N4-1025 | 136 | AAV-8 |
| 137 | bMB-N4-1031 | 137 | AAV-8 |
| 138 | bMB-N4-1032 | 138 | AAV-8 |
| 139 | bMB-N4-1033 | 139 | AAV-8 |
| 140 | bMB-N4-1037 | 140 | AAV-8 |
| 141 | bMB-N4-1050 | 141 | AAV-8 |
| 142 | bMB-N4-1052 | 142 | AAV-8 |
| 143 | bMB-N4-1059 | 143 | AAV-8 |
| 144 | bMB-N4-1060 | 144 | AAV-8 |
| 145 | bMB-N4-1063 | 145 | AAV-8 |
| 146 | bMB-N4-1065 | 146 | AAV-8 |
| 147 | bMB-N4-1068 | 147 | AAV-8 |
| 148 | bMB-N4-1069 | 148 | AAV-8 |
| 149 | bMB-N4-1071 | 149 | AAV-8 |
| 150 | bMB-N4-1072 | 150 | AAV-8 |
| 151 | bMB-N4-1093 | 151 | AAV-8 |
| 152 | bMB-N4-1097 | 152 | AAV-8 |
| 153 | bMB-N4-1098 | 153 | AAV-8 |
| 154 | bMB-N4-1103 | 154 | AAV-8 |
| 155 | bMB-N4-1106 | 155 | AAV-8 |
| 156 | bMB-N4-1124 | 156 | AAV-8 |
| 157 | bMB-N4-1131 | 157 | AAV-8 |
| 158 | bMB-N4-1132 | 158 | AAV-8 |
| 159 | bMB-N4-1133 | 159 | AAV-8 |
| 160 | bMB-N4-1135 | 160 | AAV-8 |
| 161 | bMB-N4-1137 | 161 | AAV-8 |
| 162 | bMB-N4-1141 | 162 | AAV-8 |
| 163 | bMB-N4-1143 | 163 | AAV-8 |
| 164 | bMB-N4-1145 | 164 | AAV-8 |
| 165 | bMB-N4-1161 | 165 | AAV-8 |
| 166 | bMB-N4-1162 | 166 | AAV-8 |
| 167 | bMB-N4-1167 | 167 | AAV-8 |
| 168 | bMB-N4-1172 | 168 | AAV-8 |
| 169 | bMB-N4-1177 | 169 | AAV-8 |
| 170 | bMB-N4-1180 | 170 | AAV-8 |
| 171 | bMB-N4-1184 | 171 | AAV-8 |
| 172 | bMB-N4-1193 | 172 | AAV-8 |
| 173 | bMB-N4-1196 | 173 | AAV-8 |
| 174 | bMB-N4-1205 | 174 | AAV-8 |
| 175 | bMB-N4-1206 | 175 | AAV-8 |
| 176 | bMB-N4-1210 | 176 | AAV-8 |
| 177 | bMB-N4-1215 | 177 | AAV-8 |
| 178 | bMB-N4-1217 | 178 | AAV-8 |
| 179 | bMB-N4-1223 | 179 | AAV-8 |
| 180 | bMB-N4-1230 | 180 | AAV-8 |
| 181 | bMB-N4-1239 | 181 | AAV-8 |
| 182 | bMB-N4-1250 | 182 | AAV-8 |
| 183 | bMB-N4-1251 | 183 | AAV-8 |
| 184 | bMB-N4-1252 | 184 | AAV-8 |
| 185 | bMB-N4-1257 | 185 | AAV-8 |
| 186 | bMB-N4-1259 | 186 | AAV-8 |
| 187 | bMB-N4-1277 | 187 | AAV-8 |
| 188 | bMB-N4-1278 | 188 | AAV-8 |
| 189 | bMB-N4-1279 | 189 | AAV-8 |
| 190 | bMB-N4-1284 | 190 | AAV-8 |
| 191 | bMB-N4-1292 | 191 | AAV-8 |
| 192 | bMB-N4-1293 | 192 | AAV-8 |
| 193 | bMB-N4-1307 | 193 | AAV-8 |
| 194 | bMB-N4-1310 | 194 | AAV-8 |
| 195 | bMB-N4-1312 | 195 | AAV-8 |
| 196 | bMB-N4-1313 | 196 | AAV-8 |
| 197 | bMB-N4-1330 | 197 | AAV-8 |
| 198 | bMB-N4-1338 | 198 | AAV-8 |
| 199 | bMB-N4-1357 | 199 | AAV-8 |
| 200 | bMB-N4-1360 | 200 | AAV-8 |
| 201 | bMB-N4-1361 | 201 | AAV-8 |
| 202 | bMB-N4-1362 | 202 | AAV-8 |
| 203 | bMB-N4-1363 | 203 | AAV-8 |
| 204 | bMB-N4-1367 | 204 | AAV-8 |
| 205 | bMB-N4-1371 | 205 | AAV-8 |
| 206 | bMB-N4-1379 | 206 | AAV-8 |
| 207 | bMB-N4-1381 | 207 | AAV-8 |
| 208 | bMB-N4-1388 | 208 | AAV-8 |
| 209 | bMB-N4-1398 | 209 | AAV-8 |
| 210 | bMB-N4-1407 | 210 | AAV-8 |
| 211 | bMB-N4-1408 | 211 | AAV-8 |
| 212 | bMB-N4-1414 | 212 | AAV-8 |
| 213 | bMB-N4-1416 | 213 | AAV-8 |
| 214 | bMB-N4-1417 | 214 | AAV-8 |
| 215 | bMB-N4-1425 | 215 | AAV-8 |
| 216 | bMB-N4-1436 | 216 | AAV-8 |
| 217 | bMB-N4-1443 | 217 | AAV-8 |
| 218 | bMB-N4-1447 | 218 | AAV-8 |
| 219 | bMB-N4-1452 | 219 | AAV-8 |
| 220 | bMB-N4-1467 | 220 | AAV-8 |

DNA of the aforementioned nucleic acid constructs are pooled in equimolar ratios to form two non-overlapping pools. These pools are then used to construct AAV delivery particles according to standard methods. Two pools are then titered using a qPCR assay to determine the genome copies present per ml (GC/mL).

AAV delivery particles are diluted to about 5×10¹¹ GC/mL and 200 μL of each of the two AAV libraries is injected via tail vein into transgenic NSG mice. In parallel, the mice are injected subcutaneously with 5*10⁶ SW780 cells, a cell line exhibiting epithelial morphology that was isolated from the urinary bladder of a human patient with transitional cell carcinoma, in the left flank, and 5*10⁶ HCT116 cells, a cell line that was isolated from the colon of an adult human male with colon cancer, in the right flank in a 200 μL volume composed of 50% Matrigel (Corning® Matrigel® Matrix) and 50% DMEM. When tumors reach 200-250 mm³ in size, mice are euthanized. Mice are perfused with 2× blood volumes of Phosphate buffered Saline (PBS). Blood is then collected and serum is separated from blood. After perfusion, brain, liver, muscle, skin, heart, lung, kidney, stomach, intestine tissues are all collected. Tissue is then lysed and subjected to decoding as described previously herein.

### DISCUSSION

The present example demonstrates that a binder-barcode platform as described herein can quantify localization and/or expression of cargo polypeptide in certain tissues, including tumor tissue. Moreover, the present example demonstrates that a binder-barcode platform as described herein can determine if said cargo polypeptide is also localizing and/or being expressed in other tissue (such as serum), including a ratio of amount of cargo polypeptide being expressed across different tissue types.

Moreover, delivery particles, including AAV delivery particles, provide continuous expression of cargo polypeptides, which increases overall sensitivity of a binder-barcode platform as described herein. Ability to quantify targeted and functional delivery of cargo polypeptides across different tissues can help determine potent cargo polypeptides that not only specifically target tumor tissues but are retained and/or more significantly expressed in tumor tissue compared to other tissues.

It is an insight of the present disclosure that other forms of delivery vehicles (e.g., viral particles, lipid-based particles ((e.g., cell-produced or not cell-produced), lipid nanoparticles (LNPs), liposomes, micelles, extracellular vesicles (e.g., exosomes, microparticles, etc.)), polymer-based particles (e.g., PGLA-based), polysaccharide-based particles, etc.) and/or nucleic acids (e.g., RNA) may be used in accordance with the Examples described herein.

Accordingly, the present example demonstrates a multiplexed and high-throughput approach that can be used to identify and quantify cargo polypeptides that target tumor tissue.

### Example 19: In Vivo Discovery and Assessment of Barcoded Cargo Polypeptides Delivered Using Lipid Nanoparticles (LNPs)

The present example demonstrates a multiplexed method of measuring barcoded polypeptides administered in a pooled mixture using a lipid nanoparticle delivery particles (LNPs) and a binder-barcode platform as described herein.

### Method

LNPs containing a barcoded cargo component are synthesized according to methods known in the art. 1 mg of LNP for each barcoded cargo component is generated. A table describing different variations of LNPs and/or cargo components is provided below. LNP Formulation “A” refers to an exemplary formulation that may be used in accordance with embodiments of the present disclosure. In other embodiments, other types of formulations may be used.

TABLE 12
Different LNP variants produced and their content

| Construct # | Cargo | Barcode ID | LNP Formulation |
|---|---|---|---|
| LNP1 | ALFA-Sec-GOI | 1 | A |
| LNP2 | ALFA-Sec-GOI | 2 | A |
| LNP3 | ALFA-Sec-GOI | 3 | A |
| LNP4 | ALFA-GOI | 4 | A |
| LNP5 | ALFA-GOI | 5 | A |
| LNP6 | ALFA-GOI | 6 | A |

LNPs are pooled equally by mass and 200 μg of the LNP pool is injected via tail vein into female C57 mice (n=10). At a 24 hour time point, mice are euthanized and brain, liver, muscle, skin, heart, lung, kidney, stomach, intestine tissues are collected. Tissue is then lysed and subjected to decoding as described herein.

### Discussion

Accordingly, the present example demonstrates that multiple cargo polypeptides can be delivered using pooled LNPs and assessed in parallel. An amount of each barcoded component delivered to each tissue of the mouse is readily assessed using a binder-barcode platform. Moreover, a functional readout, including expression of each cargo polypeptide, is assessed and quantified using a binder-barcode platform described herein.

### Example 20: In Vivo Discovery and Assessment of Tissue-Specific Barcoded Cargo Polypeptides Using AAV Delivery Particles

The present example demonstrates a multiplexed method of measuring a barcoded polypeptide to assess tissue-specific expression of a cargo polypeptide using a binder-barcode platform as described herein. Moreover, the present example describes methods for quantifying and/or assessing a library of cargo components that encode a cargo polypeptide. As described herein, in some embodiments, each cargo component may further comprise a tissue-specific promoter. As described herein, in some embodiments, each cargo component may further comprise an untranslated region (UTR). As described herein, in some embodiments, each cargo component may further comprise a tissue-specific promoter and a UTR. In such an embodiment, the present example describes demonstrates quantification of effects of promoters on cargo polypeptide expression and/or localization. A binder-barcode platform described herein is used to quantify effects of cargo polypeptide localization and/or expression based on additional variables included in the cargo component As such, a binder-barcode platform as described herein can be used to identify cargo components based on desired phenotypes of cargo polypeptides.

Other types of cargo components and modifications to a cargo polypeptide can be assessed using a binder-barcode platform as described herein. Phenotypes other than expression and localization can also be quantified and/or assessed using a binder-barcode platform as described herein.

### Method

Different nucleic acids comprising barcoded cargo components were designed as follows. Cargo components that encode a cargo polypeptide can also include other components such as promoters, UTRs, or combinations thereof. Each cargo component encoding a cargo polypeptide was then associated with a unique barcode component. Each peptide barcode encoded by a barcode component is associated (e.g., covalently or not covalently) with the C-terminus of the cargo polypeptide via a linker. Nucleic acids (or constructs) were synthesized with cargo components including a sequence encoding a cargo polypeptide, promoter elements, terminator elements, and/or ITR elements for packaging into AAV delivery particles. A table describing different variations of AAV delivery particles and/or cargo components is provided below.

TABLE 13
Different variants produced and their content

| Construct | | Barcode | AAV | | |
|---|---|---|---|---|---|
| # | Cargo | ID | Serotype | Promoter | UTR |
| 1 | ALFA-Sec-GOI | 1 | AAV-9 | CMV | WPRE |
| 2 | ALFA-Sec-GOI | 2 | AAV-9 | CMV + intron | WPRE |
| 3 | ALFA-Sec-GOI | 3 | AAV-9 | EFIA | WPRE |
| 4 | ALFA-Sec-GOI | 4 | AAV-9 | EFS | WPRE |
| 5 | ALFA-Sec-GOI | 5 | AAV-9 | CAG | WPRE |
| 6 | ALFA-Sec-GOI | 6 | AAV-9 | CBh | WPRE |
| 7 | ALFA-Sec-GOI | 7 | AAV-9 | CBA | WPRE |
| 8 | ALFA-Sec-GOI | 8 | AAV-9 | SFFV | WPRE |
| 9 | ALFA-Sec-GOI | 9 | AAV-9 | MSCV | WPRE |
| 10 | ALFA-Sec-GOI | 10 | AAV-9 | SV40 | WPRE |
| 11 | ALFA-Sec-GOI | 11 | AAV-9 | mPGK | WPRE |
| 12 | ALFA-Sec-GOI | 12 | AAV-9 | hPGK | WPRE |
| 13 | ALFA-Sec-GOI | 13 | AAV-9 | UBC | WPRE |
| 14 | ALFA-Sec-GOI | 14 | AAV-9 | Mammalian | WPRE |
| | | | | Tissue- | |
| | | | | Specific | |
| | | | | Promoters | |
| 15 | ALFA-Sec-GOI | 15 | AAV-9 | Name | WPRE |
| 16 | ALFA-Sec-GOI | 16 | AAV-9 | Nanog | WPRE |
| 17 | ALFA-Sec-GOI | 17 | AAV-9 | Nes | WPRE |
| 18 | ALFA-Sec-GOI | 18 | AAV-9 | Tuba1a | WPRE |
| 19 | ALFA-Sec-GOI | 19 | AAV-9 | Camk2a(long) | WPRE |
| 20 | ALFA-Sec-GOI | 20 | AAV-9 | Camk2a(short) | WPRE |
| 21 | ALFA-Sec-GOI | 21 | AAV-9 | SYN1 | WPRE |
| 22 | ALFA-Sec-GOI | 22 | AAV-9 | Hb9 | WPRE |
| 23 | ALFA-Sec-GOI | 23 | AAV-9 | Th | WPRE |
| 24 | ALFA-Sec-GOI | 24 | AAV-9 | Thy1 | WPRE |
| 25 | ALFA-Sec-GOI | 25 | AAV-9 | NSE | WPRE |
| 26 | ALFA-Sec-GOI | 26 | AAV-9 | GFAP(long) | WPRE |
| 27 | ALFA-Sec-GOI | 27 | AAV-9 | GFAP(short) | WPRE |
| 28 | ALFA-Sec-GOI | 28 | AAV-9 | Iba1 | WPRE |
| 29 | ALFA-Sec-GOI | 29 | AAV-9 | ProA1 | WPRE |
| 30 | ALFA-Sec-GOI | 30 | AAV-9 | hRHO | WPRE |
| 31 | ALFA-Sec-GOI | 31 | AAV-9 | hBEST1 | WPRE |
| 32 | ALFA-Sec-GOI | 32 | AAV-9 | Grm6 | WPRE |
| 33 | ALFA-Sec-GOI | 33 | AAV-9 | Grm6(short) | WPRE |
| 34 | ALFA-Sec-GOI | 34 | AAV-9 | Grm6(long) | WPRE |
| 35 | ALFA-Sec-GOI | 35 | AAV-9 | Prnp | WPRE |
| 36 | ALFA-Sec-GOI | 36 | AAV-9 | Cnp | WPRE |
| 37 | ALFA-Sec-GOI | 37 | AAV-9 | K14 | WPRE |
| 38 | ALFA-Sec-GOI | 38 | AAV-9 | K19 | WPRE |
| 39 | ALFA-Sec-GOI | 39 | AAV-9 | BK5 | WPRE |
| 40 | ALFA-Sec-GOI | 40 | AAV-9 | mTyr | WPRE |
| 41 | ALFA-Sec-GOI | 41 | AAV-9 | cTnT | WPRE |
| 42 | ALFA-Sec-GOI | 42 | AAV-9 | αMHC(long) | WPRE |
| 43 | ALFA-Sec-GOI | 43 | AAV-9 | αMHC(short) | WPRE |
| 44 | ALFA-Sec-GOI | 44 | AAV-9 | Myog | WPRE |
| 45 | ALFA-Sec-GOI | 45 | AAV-9 | ACTA1 | WPRE |
| 46 | ALFA-Sec-GOI | 46 | AAV-9 | MHCK7 | WPRE |
| 47 | ALFA-Sec-GOI | 47 | AAV-9 | SM22a | WPRE |
| 48 | ALFA-Sec-GOI | 48 | AAV-9 | EnSM22a | WPRE |
| 49 | ALFA-Sec-GOI | 49 | AAV-9 | Runx2 | WPRE |
| 50 | ALFA-Sec-GOI | 50 | AAV-9 | OC | WPRE |
| 51 | ALFA-Sec-GOI | 51 | AAV-9 | Col1a1 | WPRE |
| 52 | ALFA-Sec-GOI | 52 | AAV-9 | Col2a1 | WPRE |
| 53 | ALFA-Sec-GOI | 53 | AAV-9 | aP2 | WPRE |
| 54 | ALFA-Sec-GOI | 54 | AAV-9 | Adipoq | WPRE |
| 55 | ALFA-Sec-GOI | 55 | AAV-9 | Tie1 | WPRE |
| 56 | ALFA-Sec-GOI | 56 | AAV-9 | Cd144 | WPRE |
| 57 | ALFA-Sec-GOI | 57 | AAV-9 | CD68(short) | WPRE |
| 58 | ALFA-Sec-GOI | 58 | AAV-9 | CD68(long) | WPRE |
| 59 | ALFA-Sec-GOI | 59 | AAV-9 | CD11b | WPRE |
| 60 | ALFA-Sec-GOI | 60 | AAV-9 | Afp | WPRE |
| 61 | ALFA-Sec-GOI | 61 | AAV-9 | Alb | WPRE |
| 62 | ALFA-Sec-GOI | 62 | AAV-9 | TBG | WPRE |
| 63 | ALFA-Sec-GOI | 63 | AAV-9 | TBG + intron | WPRE |
| 64 | ALFA-Sec-GOI | 64 | AAV-9 | MMTV | WPRE |
| 65 | ALFA-Sec-GOI | 65 | AAV-9 | Wap | WPRE |
| 66 | ALFA-Sec-GOI | 66 | AAV-9 | HIP | WPRE |
| 67 | ALFA-Sec-GOI | 67 | AAV-9 | Pdx1 | WPRE |
| 68 | ALFA-Sec-GOI | 68 | AAV-9 | Ins2 | WPRE |
| 69 | ALFA-Sec-GOI | 69 | AAV-9 | Hcn4 | WPRE |
| 70 | ALFA-Sec-GOI | 70 | AAV-9 | NPHS2 | WPRE |
| 71 | ALFA-Sec-GOI | 71 | AAV-9 | SPB | WPRE |
| 72 | ALFA-Sec-GOI | 72 | AAV-9 | CD144 | WPRE |
| 73 | ALFA-Sec-GOI | 73 | AAV-9 | TERT | WPRE |
| 74 | ALFA-Sec-GOI | 74 | AAV-9 | CMV | oPRE |
| 75 | ALFA-Sec-GOI | 75 | AAV-9 | CMV + intron | oPRE |
| 76 | ALFA-Sec-GOI | 76 | AAV-9 | EF1A | oPRE |
| 77 | ALFA-Sec-GOI | 77 | AAV-9 | EFS | oPRE |
| 78 | ALFA-Sec-GOI | 78 | AAV-9 | CAG | oPRE |
| 79 | ALFA-Sec-GOI | 79 | AAV-9 | CBh | oPRE |
| 80 | ALFA-Sec-GOI | 80 | AAV-9 | CBA | oPRE |
| 81 | ALFA-Sec-GOI | 81 | AAV-9 | SFFV | oPRE |
| 82 | ALFA-Sec-GOI | 82 | AAV-9 | MSCV | oPRE |
| 83 | ALFA-Sec-GOI | 83 | AAV-9 | SV40 | oPRE |
| 84 | ALFA-Sec-GOI | 84 | AAV-9 | mPGK | oPRE |
| 85 | ALFA-Sec-GOI | 85 | AAV-9 | hPGK | oPRE |
| 86 | ALFA-Sec-GOI | 86 | AAV-9 | UBC | oPRE |
| 87 | ALFA-Sec-GOI | 87 | AAV-9 | Mammalian | oPRE |
| | | | | Tissue- | |
| | | | | Specific | |
| | | | | Promoters | |
| 88 | ALFA-Sec-GOI | 88 | AAV-9 | Name | oPRE |
| 89 | ALFA-Sec-GOI | 89 | AAV-9 | Nanog | oPRE |
| 90 | ALFA-Sec-GOI | 90 | AAV-9 | Nes | oPRE |
| 91 | ALFA-Sec-GOI | 91 | AAV-9 | Tuba1a | oPRE |
| 92 | ALFA-Sec-GOI | 92 | AAV-9 | Camk2a(long) | oPRE |
| 93 | ALFA-Sec-GOI | 93 | AAV-9 | Camk2a(short) | oPRE |
| 94 | ALFA-Sec-GOI | 94 | AAV-9 | SYN1 | oPRE |
| 95 | ALFA-Sec-GOI | 95 | AAV-9 | Hb9 | oPRE |
| 96 | ALFA-Sec-GOI | 96 | AAV-9 | Th | oPRE |
| 97 | ALFA-Sec-GOI | 97 | AAV-9 | Thy1 | oPRE |
| 98 | ALFA-Sec-GOI | 98 | AAV-9 | NSE | oPRE |
| 99 | ALFA-Sec-GOI | 99 | AAV-9 | GFAP(long) | oPRE |
| 100 | ALFA-Sec-GOI | 100 | AAV-9 | GFAP(short) | oPRE |
| 101 | ALFA-Sec-GOI | 101 | AAV-9 | Iba1 | oPRE |
| 102 | ALFA-Sec-GOI | 102 | AAV-9 | ProA1 | oPRE |
| 103 | ALFA-Sec-GOI | 103 | AAV-9 | hRHO | oPRE |
| 104 | ALFA-Sec-GOI | 104 | AAV-9 | hBEST1 | oPRE |
| 105 | ALFA-Sec-GOI | 105 | AAV-9 | Grm6 | oPRE |
| 106 | ALFA-Sec-GOI | 106 | AAV-9 | Grm6(short) | oPRE |
| 107 | ALFA-Sec-GOI | 107 | AAV-9 | Grm6(long) | oPRE |
| 108 | ALFA-Sec-GOI | 108 | AAV-9 | Prnp | oPRE |
| 109 | ALFA-Sec-GOI | 109 | AAV-9 | Cnp | oPRE |
| 110 | ALFA-Sec-GOI | 110 | AAV-9 | K14 | oPRE |
| 111 | ALFA-Sec-GOI | 111 | AAV-9 | K19 | oPRE |
| 112 | ALFA-Sec-GOI | 112 | AAV-9 | BK5 | oPRE |
| 113 | ALFA-Sec-GOI | 113 | AAV-9 | mTyr | oPRE |
| 114 | ALFA-Sec-GOI | 114 | AAV-9 | cTnT | oPRE |
| 115 | ALFA-Sec-GOI | 115 | AAV-9 | αMHC(long) | oPRE |
| 116 | ALFA-Sec-GOI | 116 | AAV-9 | αMHC(short) | oPRE |
| 117 | ALFA-Sec-GOI | 117 | AAV-9 | Myog | oPRE |
| 118 | ALFA-Sec-GOI | 118 | AAV-9 | ACTA1 | oPRE |
| 119 | ALFA-Sec-GOI | 119 | AAV-9 | MHCK7 | oPRE |
| 120 | ALFA-Sec-GOI | 120 | AAV-9 | SM22a | oPRE |
| 121 | ALFA-Sec-GOI | 121 | AAV-9 | EnSM22a | oPRE |
| 122 | ALFA-Sec-GOI | 122 | AAV-9 | Runx2 | oPRE |
| 123 | ALFA-Sec-GOI | 123 | AAV-9 | OC | oPRE |
| 124 | ALFA-Sec-GOI | 124 | AAV-9 | Col1a1 | oPRE |
| 125 | ALFA-Sec-GOI | 125 | AAV-9 | Col2a1 | oPRE |
| 126 | ALFA-Sec-GOI | 126 | AAV-9 | aP2 | oPRE |
| 127 | ALFA-Sec-GOI | 127 | AAV-9 | Adipoq | oPRE |
| 128 | ALFA-Sec-GOI | 128 | AAV-9 | Tie1 | oPRE |
| 129 | ALFA-Sec-GOI | 129 | AAV-9 | Cd144 | oPRE |
| 130 | ALFA-Sec-GOI | 130 | AAV-9 | CD68(short) | oPRE |
| 131 | ALFA-Sec-GOI | 131 | AAV-9 | CD68(long) | oPRE |
| 132 | ALFA-Sec-GOI | 132 | AAV-9 | CD11b | oPRE |
| 133 | ALFA-Sec-GOI | 133 | AAV-9 | Afp | oPRE |
| 134 | ALFA-Sec-GOI | 134 | AAV-9 | Alb | oPRE |
| 135 | ALFA-Sec-GOI | 135 | AAV-9 | TBG | oPRE |
| 136 | ALFA-Sec-GOI | 136 | AAV-9 | TBG + intron | oPRE |
| 137 | ALFA-Sec-GOI | 137 | AAV-9 | MMTV | oPRE |
| 138 | ALFA-Sec-GOI | 138 | AAV-9 | Wap | oPRE |
| 139 | ALFA-Sec-GOI | 139 | AAV-9 | HIP | oPRE |
| 140 | ALFA-Sec-GOI | 140 | AAV-9 | Pdx1 | oPRE |
| 141 | ALFA-Sec-GOI | 141 | AAV-9 | Ins2 | oPRE |
| 142 | ALFA-Sec-GOI | 142 | AAV-9 | Hcn4 | oPRE |
| 143 | ALFA-Sec-GOI | 143 | AAV-9 | NPHS2 | oPRE |
| 144 | ALFA-Sec-GOI | 144 | AAV-9 | SPB | oPRE |
| 145 | ALFA-Sec-GOI | 145 | AAV-9 | CD144 | oPRE |
| 146 | ALFA-Sec-GOI | 146 | AAV-9 | TERT | oPRE |

DNA of the aforementioned nucleic acid constructs is pooled in equimolar ratios. This is then used to construct AAV delivery particles according to standard methods. Pools are then titered using a qPCR assay to determine the genome copies present per ml (GC/mL).

AAV delivery particles are diluted to about 5×10¹¹ GC/mL and 200 μL of the AAV libraries is injected into transgenic mice.

Mice are euthanized at 7 days, 14 days, 21 days, and 28 days. At these endpoints, blood is collected and serum is separated from blood. Afterwards, mice are perfused with 2× blood volumes of Phosphate buffered Saline (PBS) and brain, liver, muscle, skin, heart, lung, kidney, stomach and intestine tissues are all collected. Tissue is then lysed and subjected to decoding as described herein.

In some embodiments, nucleic acids (constructs) are also delivered to mice via other types of delivery particles, including LNPs (see, e.g., Example 4). Mice are euthanized at 7, 14, 21 and/or 28 days. Blood is collected and serum is separated from blood. Then, mice are perfused with PBS and brain, liver, muscle, skin, heart, lung, kidney, stomach and intestine tissues are all collected. Tissue is then lysed and subjected to decoding as described herein.

### Discussion

Accordingly, the present example demonstrates the ability to measure localization and/or expression of multiple cargo polypeptides delivered through different delivery particles (e.g., AAV, lipid-based, etc.) in parallel. Accordingly, multiple cargo component designs can be assessed in parallel across multiple tissues of interest as described herein.

### Example 21: In Vivo Inducible Expression of Barcoded Cargo Polypeptides Delivered Using AAV Delivery Particles

The present example demonstrates a multiplexed method of measuring a barcoded cargo polypeptide to assess inducible expression of the cargo polypeptide delivered using delivery particles as described herein.

Other types of cargo components and modifications to a cargo polypeptide can be assessed using a binder-barcode platform as described herein. Phenotypes other than expression and localization can also be quantified and/or assessed using a binder-barcode platform as described herein.

### Method

Different nucleic acids comprising barcoded cargo components were designed as follows. Cargo components that encode a cargo polypeptide, can include other components such as inducible promoters. In some embodiments, inducible promoters require an exogenous signal (e.g., Doxycycline (DOX)) to be activated and drive expression of a cargo polypeptide. Each cargo component encoding a cargo polypeptide was then associated with a unique barcode component. Each peptide barcode encoded by a barcode component is associated (e.g., covalently or not covalently) with the C-terminus of the cargo polypeptide via a linker. Nucleic acids (or constructs) were synthesized with cargo components including a sequence encoding a cargo polypeptide, promoter elements, terminator elements, and/or ITR elements for packing into AAV delivery particles. A table describing different variations of AAV delivery particles and/or cargo components is provided below.

TABLE 14
Different variants produced and their content

| | | | | | Exogenous |
|---|---|---|---|---|---|
| Construct | AAV | | | | Signal |
| # | Serotype | Cargo | Barcode ID | Promotor | Required |
| 1 | AAV-9 | ALFA-Sec-GOI | 1 | Tert | Tetracycline |
| 2 | AAV-9 | ALFA-Sec-GOI | 2 | Dox | Doxycycline |

DNA of the aforementioned nucleic acid constructs is pooled in equimolar ratios. This is then used to construct AAV delivery particles according to standard methods. Pools are then titered using a qPCR assay to determine the genome copies present per ml (GC/mL).

AAV delivery particles are diluted to about 5×10¹¹ GC/mL and 200 μL of the AAV libraries is injected into transgenic mice. Mice are euthanized at 7 days, 14 days, 21 days, and 28 days. At these endpoints, blood is collected and serum is separated from blood. Afterwards, mice are perfused with 2× blood volumes of Phosphate buffered Saline (PBS) and brain, liver, muscle, skin, heart, lung, kidney, stomach and intestine tissues are all collected. Tissue is then lysed and subjected to decoding as described herein.

In some embodiments, nucleic acids (constructs) are also delivered to mice via other types of delivery particles, including LNPs (see, e.g., Example 4). Mice are euthanized at 7, 14, 21 and/or 28 days. Blood is collected and serum is separated from blood. Then, mice are perfused with PBS and brain, liver, muscle, skin, heart, lung, kidney, stomach and intestine tissues are all collected. Tissue is then lysed and subjected to decoding as described herein.

### Discussion

Accordingly, the present example demonstrates the ability to measure localization and/or expression of multiple cargo polypeptides delivered through different delivery particles (e.g., AAV, lipid-based, etc.) in parallel. Accordingly, multiple cargo component designs can be assessed in parallel across multiple tissues of interest as described herein.

### Example 22: In Vivo Discovery and Assessment of Tissue-Specific Barcoded Cargo Polypeptides Using Lentivirus Delivery Particles

Example 20 is repeated using lentivirus delivery particles.

### Example 23: In Vivo Multiplexed Screening of TfR Shuttles that Cross the Blood-Brain Barrier (BBB)

The present example demonstrates a multiplexed, high-throughput method of screening shuttles (e.g., shuttle moieties) as described herein. The present example also demonstrates that a binder-barcode platform described herein can be used to assess inducible expression of a delivered cargo polypeptide. Moreover, the present example demonstrates that a binder-barcode platform described herein can be used to identify and/or quantify cargo polypeptides and/or components thereof for certain phenotypes such as expression and/or localization. For example, in some embodiments, shuttles (e.g., shuttle moieties) can be identified and/or assessed for ability to cross the blood-brain barrier (BBB) (e.g., by binding to a BBB surface receptor and inducing transcytosis into the brain) as described by this example. In some embodiments, a shuttle is or comprises a shuttle moiety.

Other types of cargo components can be assessed using a binder-barcode platform as described herein. Phenotypes other than expression and localization can also be quantified and/or assessed using a binder-barcode platform as described herein. Exemplary phenotypes include epitope properties, affinity, thermostability, pH sensitivity, or other phenotypes described herein.

### Method

Transferrin receptor (TfR) was selected as an initial brain target to be targeted by a shuttle moiety as described herein.

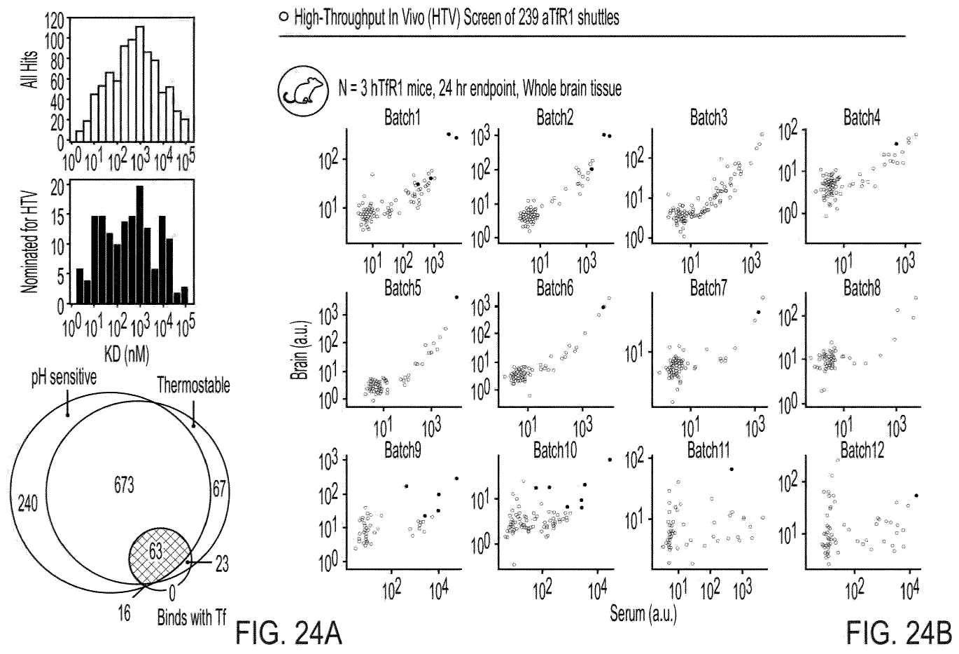

Following phage-display discovery, 239 unique anti-TfR VHHs with unique properties including: epitope, affinity, thermostability, and pH sensitivity, were nominated for screening in vivo (see FIG. 24A). Protein language models (not shown in FIG. 24A) and phage-based readouts were used to inform this selection.

The 239 VHHs were simultaneously screened in vivo in sets of 15 to 96, at doses ranging from 0.5 to 1 mg/kg, depending on batch size (see FIG. 24B). Brain, serum, cell-free fraction (parenchyma), and muscle tissue abundances were quantified using barcodes at 24 hours (1 day). Hits demonstrating favorable brain uptake and stable PK (robust serum levels) were advanced for further screening and validation.

### Example 24: Multiplexed In Vivo Assessment of Phenotypes of Shuttles Using Binder-Barcode Platform

The present example demonstrates a method to perform multiplexed in vivo assessment of phenotypes of shuttles (e.g., brain shuttle moieties) using a binder-barcode platform described herein. In some embodiments, a phenotype includes pharmacokinetic (or clearance) data as described herein.

### Method

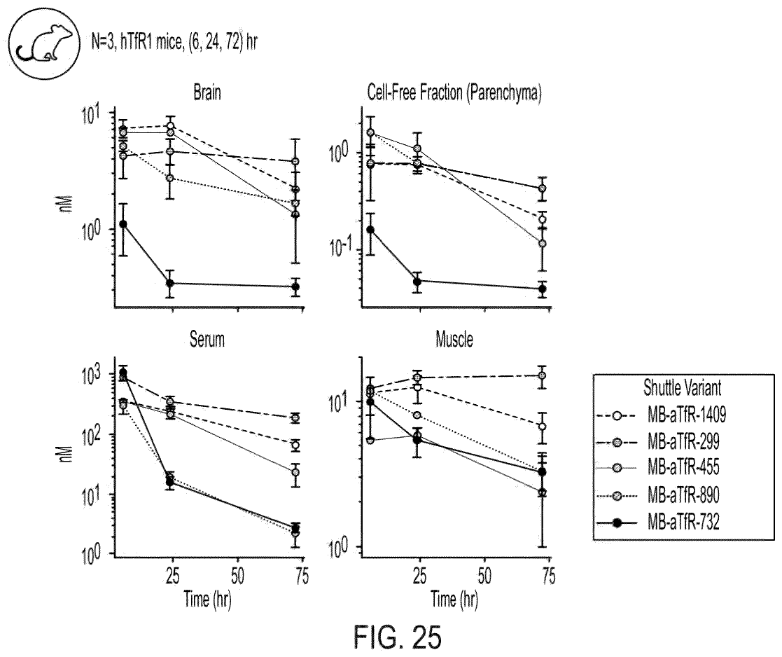

Select candidates from a TfR screen performed in Example 23 were analyzed in a multiplexed PK analysis. Results of this experiment are shown in FIG. 25. As shown in FIG. 25, “shuttle” candidates showed a diversity of different PK properties across blood, brain, and peripheral tissues.

### Example 25: In Vivo Multiplexed Screening of TfR Shuttles that Cross the Blood-Brain Barrier (BBB)

The present example demonstrates a multiplexed, high-throughput method of screening shuttles (e.g., shuttle agents or shuttle moieties) as described herein. The present example also demonstrates that a binder-barcode platform described herein can be used to assess inducible expression of a delivered cargo polypeptide. Moreover, the present example demonstrates that a binder-barcode platform described herein can be used to identify and/or quantify cargo polypeptides and/or shuttle-cargo polypeptides and/or shuttles and/or components thereof for certain phenotypes such as expression and/or localization. For example, in some embodiments, shuttles (e.g., shuttle moieties) can be identified and/or assessed for ability to cross the blood-brain barrier (BBB) (e.g., by binding to a BBB surface receptor and inducing transcytosis into the brain) as described by this example. In some embodiments, a shuttle is or comprises a shuttle moiety.

Other types of cargo components can be assessed using a binder-barcode platform as described herein. Phenotypes other than expression and localization can also be quantified and/or assessed using a binder-barcode platform as described herein. Exemplary phenotypes include epitope properties, affinity, thermostability, pH sensitivity, or other phenotypes described herein.

### Method

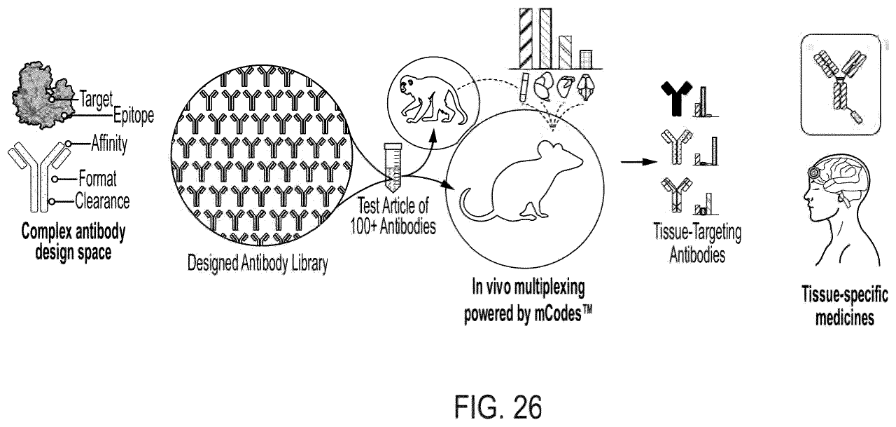

Transferrin receptor (TfR) was selected as an initial brain target to be targeted by a shuttle moiety as described herein. See, e.g., FIGS. 26-27.

### Molecule Construction and Production:

Following phage-display discovery, 250 unique anti-TfR VHHs with unique binding properties including: epitope, affinity, thermostability, and pH sensitivity, were nominated for screening in vivo. These anti-TfR VHHs were produced as C-terminal Fc fusions.

Plasmid backbone, VHH, or barcode gene fragment was PCR amplified using 10 ng of template DNA. Template DNA, primers (500 nM final concentration), and water were combined with Q5 High-Fidelity Master Mix (1× final concentration, NEB) and amplified for 30 cycles. Plasmid backbone PCR product was purified with Nucleospin PCR Cleanup Kit (Machery Nagel). 10 μg purified, linear plasmid backbone was digested for 2 hours at 37° C. with FastDigest DpnI (Invitrogen) before heat inactivation and SPRI purification (Bulldog Bio). 25 ng backbone DNA were combined 1:3 with insert DNA and HiFi Assembly Master Mix (1× final concentration, NEB). Assembly reaction was incubated for 1 hour at 50° C. 2.5 ng assembled plasmid product was used as the template for PCR amplification. 500 ng PCR amplified DNA was complexed with Expifectamine293 according to manufacturer recommendations (Gibco) in a transfection volume of 800 μL in 12 replicates. Transfected cells were harvested by centrifugation on day 5. 800 μL of cell culture supernatant was transferred to a fresh 96-deep well plate containing 12.5 μL MagSepharose PrismA beads (Cytiva). Supernatants and beads were incubated at 36.5° C. for 1 hour at 1,000 RPM. Beads were collected, washed, and purified antibodies eluted into 120 μL 100 mM glycine, 150 mM sodium chloride, pH 2.0 using the KingFisher Purification System (Thermo Fisher) and neutralized with 15 μL 1 M Sodium Phosphate, pH 7. Purified antibodies were quantified by BCA before pooling at the appropriate mass to achieve 1, 2.5, or 5 mg/kg dosing (IgG equivalent) and concentrated using centrifugal concentrators (10 kDa MWCO, MilliporeSigma) to a final volume of 1050 μL. Final concentration of the pooled antibodies was measured by BCA. Antibody pools were snap frozen in liquid nitrogen before dosing.

### Animals:

All procedures in animals were performed in adherence to ethical regulations and protocols approved by Avastus Institutional Animal Care and Use Committee (IACUC). Mice were housed under a 12-hour light/dark cycle and had access to water and standard rodent diet (LabDiet #25502, Irradiated) ad libitum.

hTfR mice (C57BL/6-Tfr1tm1TFR1/Bcgen, B-hTFR1, 110861) were obtained from Biocytogen. Mice were randomly assigned to groups based on predetermined sample sizes. Experimenters were not blinded to the sample groups.

### In Vivo Screen:

Antibodies were pooled into test articles in groups of 50-100 and were injected at 1 mg/kg/antibody. A total of 3 mice per TA group were systemically dosed via tail vein. After 24 hours, animals were sacrificed and blood was collected in serum separator tubes via cardiac puncture. Prior to organ collection, mice were transcardially perfused with PBS. The brain was divided into two hemispheres and along with muscle, liver, heart and lungs, all tissues were snap frozen and stored at −80° C.

### Tissue Processing:

One hemisphere of brain tissues (right side) was lysed (750 μL) using RIPA buffer (ThermoFisher) with Halt protease inhibitor (ThermoFisher, 1:100) in a TissueLyzer (Qiagen) on at 30 rpm for 7 min using a stainless-steel bead of 5 mm (Qiagen). Upon ensuring that the sample is lysed, tubes are centrifuged at 7000 g at 4° C. for 20 min. The supernatant was collected representing the whole brain fraction and was subjected to quantification using decoding protocol described herein.

For parenchymal extraction, the other brain hemisphere (left) was minced into small pieces with a scalpel in an HBSS buffer containing 10 mM HEPES. The sample was homogenized using a long 21 G cannula (0.8 mm×120 mm) mounted on a 2 mL syringe by aspirating and pushing out 20 times. Homogenate was transferred to 2 mL tubes and centrifuged at 2000 g at 4° C. for 10 min. Supernatant here represents the whole parenchyma while the pellet, when lysed, represents the myelin and vessels fraction. Whole parenchyma was further processed into cell free and cell-associated fractions. For other tissues (muscle, heart, liver and lung), an above-mentioned lysis protocol was followed. All supernatants post-lysis were collected and subjected for quantification through decoding protocol described herein.

### Quantification:

Molecules were quantified using decoding protocol described herein. Briefly, lysed tissue samples were incubated with magnetic beads coated with ALFAtag single-domain antibody (ALFA Selector ST, Nanotag Biotechnologies, Germany) added at 10 μL per well and incubated shaking at 900 rpm to ensure thorough mixing of beads for 1.5 hours at room temperature (RT), plates were then moved to 4° C. to continue capturing overnight. Beads were washed once with 1×PBS and the appropriate scFv decoder phage library (1E10 pfu/mL) was added to bind the barcodes for 2 hours at RT, shaking at 900 rpm. Beads were then washed three times to remove non-specifically binding phages in 1×PBST (0.1% Tween-20) using an extractor shield to carefully transfer the beads to each wash plate. After the final wash, beads were incubated in Trypsin (0.1 μg/mL, Sigma Aldrich) for 1 hour at 37° C. to elute phages off of the beads. Eluate was then added to E. coli TG1 cells (LGC, Biosearch Technologies) in exponential phase (OD600=0.4) and incubated for 1 hour at 37° C., shaking at 1000 rpm. Carbenicillin (120 μg/mL, Sigma Aldrich) and Glucose (1%) were added to LB media (Sigma Aldrich) and 1 mL of this prepared media was added to the bacterial cells infected with phages and incubated shaking at 1000 rpm for 16 hours at 37° C. Samples were then subjected to NGS library preparation where 1 μL of cells were added to Q5 High-Fidelity 2× Master Mix (NEB) with appropriate forward and reverse primers (0.75 μM final concentration) to amplify the scFv decoder insert and libraries were amplified with 13 cycles of PCR. DNA libraries were then indexed with unique dual index primers (i5 and i7) to enable sequencing and the libraries were pooled together post-indexing. Libraries were then purified using SPRI bead purification (CleanNGS DNA purification magnetic beads) using an amount appropriate for the desired size selection and DNA was eluted in a buffer containing 10 mM Tris-HCl (pH 8.0) with 0.1 mM EDTA. DNA libraries were then sequenced with Illumina's NextSeq 2000 sequencer, generating paired end reads of 150 bp. Precise quantification of barcode amounts in the tissue samples from NGS data was enabled by including a standard curve that was decoded with known amounts of protein in commercially purchased (Transnetyx) strain-matched control tissues lysed with the above-mentioned lysis protocol under a larger scale (GentleMacs). A known amount of control phage was also added to every well for normalization of the NGS signal across all the wells thereby enabling accurate and meaningful quantification of barcode amounts in the wells.

### Results

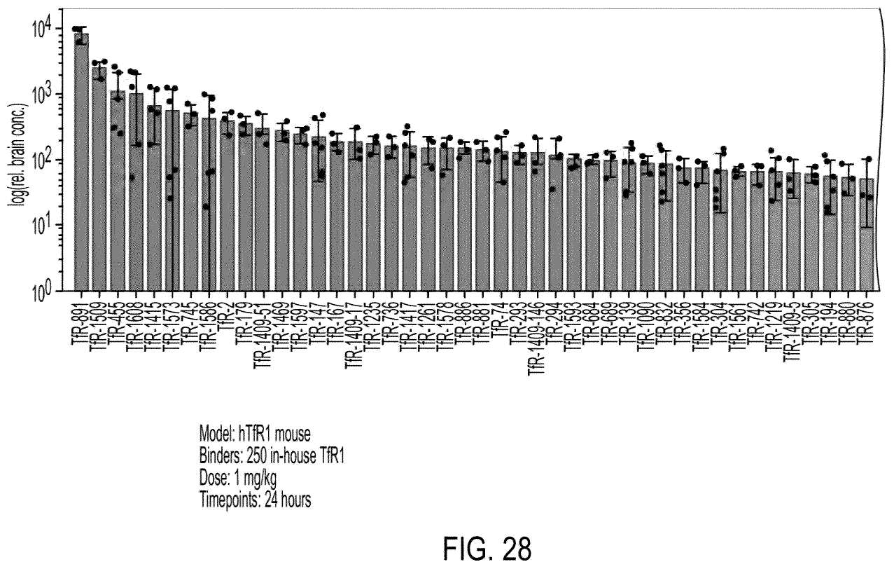

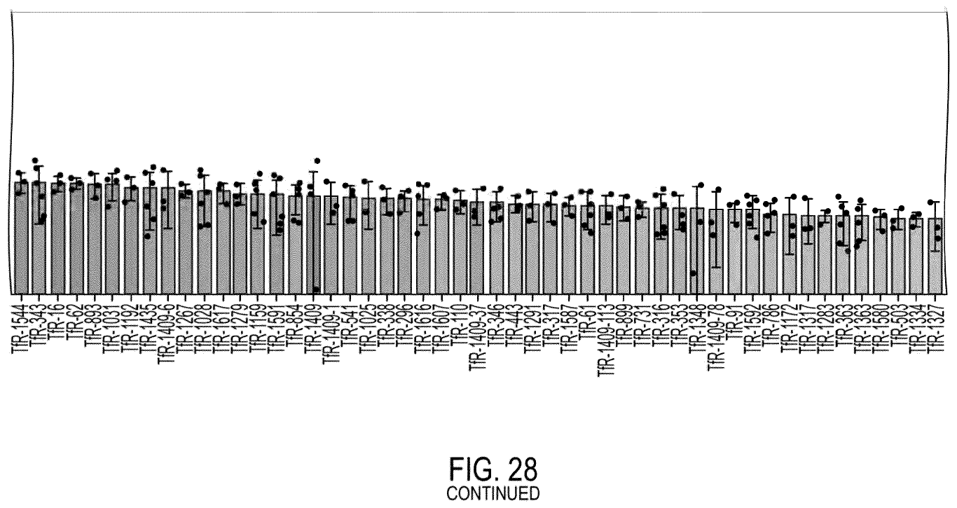

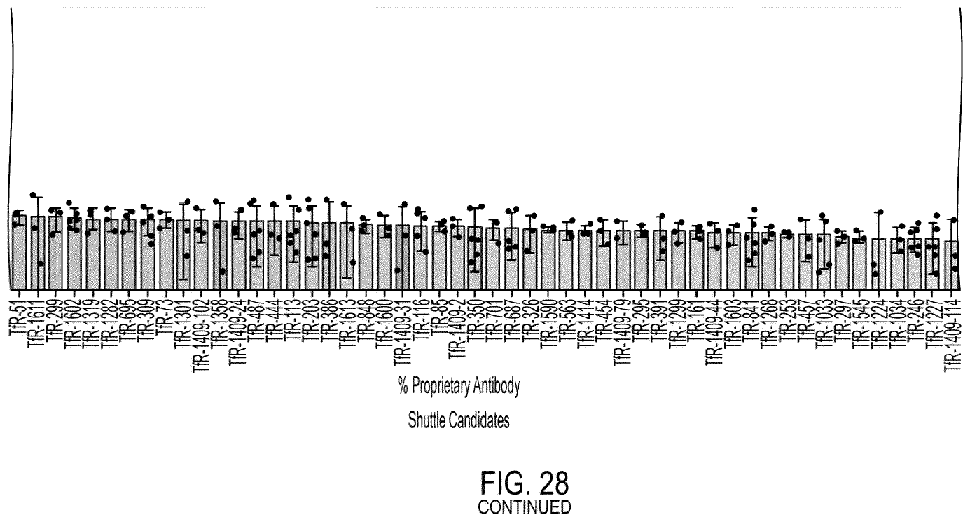

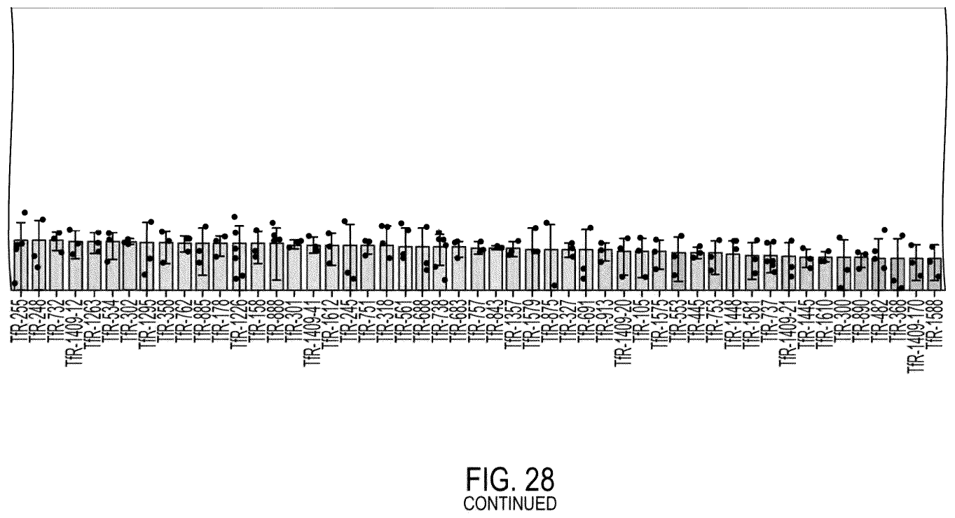

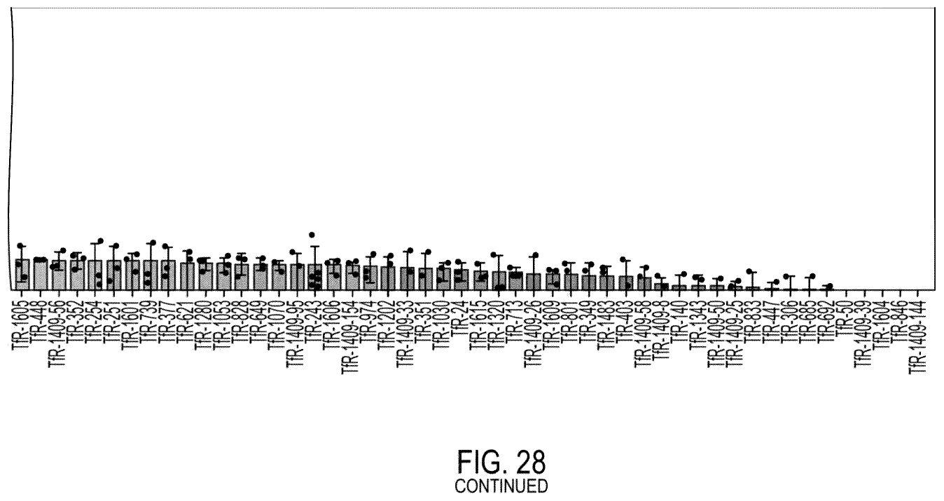

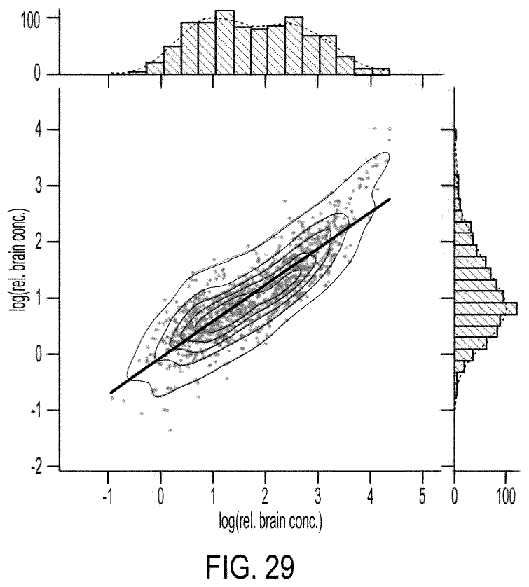

FIG. 28 shows results of quantification across all 250 binders tested in a humanized mouse. Relative concentrations as measured via NGS quantification depict several log order variations in brain concentration, with top tested molecules being enriched 4 log orders more than the lowest concentration molecules. FIG. 29 shows a scatter plot comparing relative serum concentration of relative brain concentration for the same set of molecules. Using the combination of this data, a set of molecules was manually curated which contained high brain uptake relative to serum abundance.

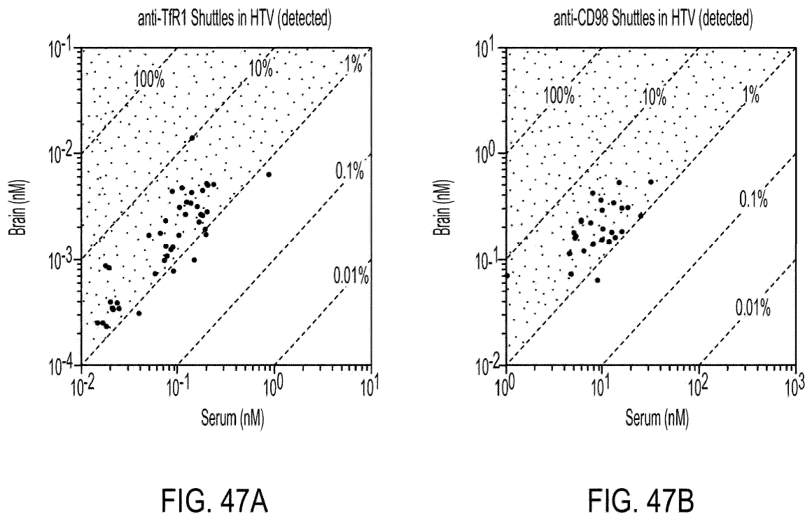

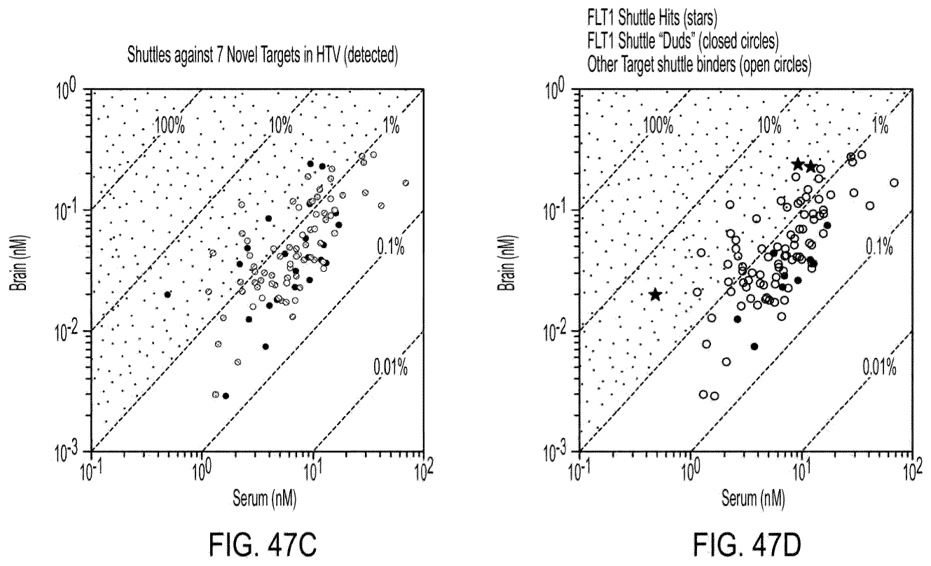

FIGS. 47A-47D depicts plots showing multiplexed in vivo screening data of shuttle candidates. The diagonal lines with percentages denote brain/serum ratios. FIG. 47A depicts plots related to protein-barcoded anti-TfR1 nanobodies screened in multiplex in humanized TfR1 mice (n=3), with takedown and analysis of serum and whole-brain tissue conducted at 24 hrs post-injection. A total of 250 total molecules were screened and those nanobodies detected in brain are shown.

### Example 26: Multiplexed In Vivo Assessment of Phenotypes of Shuttles Using Binder-Barcode Platform

The present example demonstrates a method to perform multiplexed in vivo assessment of phenotypes of shuttles (e.g., brain shuttle moieties) using a binder-barcode platform described herein. In some embodiments, a phenotype includes pharmacokinetic (or clearance) data as described herein.

### Method

### Molecule Construction and Production:

Plasmid backbone, VHH, or Barcode gene fragment was PCR amplified using 10 ng of template DNA. Forward and reverse primer (500 nM final concentration) was combined with Q5 High-Fidelity Master Mix (Ix final concentration, NEB) and amplified for 30 cycles. Plasmid backbone PCR product was purified with Nucleospin PCR Cleanup Kit (Machery Nagel). 10 μg purified, linear plasmid backbone was digested for 2 hours at 37° C. with FastDigest DpnI (Invitrogen) before heat inactivation and SPRI purification (Bulldog Bio). 25 ng backbone DNA were combined 1.3 with insert DNA and HiFi Assembly Master Mix (1× final concentration, NEB). Assembly reaction was incubated for 1 hour at 50° C. 2.5 ng assembled plasmid product was used as the template for PCR amplification. 12.5 Mg PCR amplified DNA was complexed with Expifectamine293 according to manufacturer recommendations (Gibco) in a transfection volume of 25 mL. Transfected cells were harvested by centrifugation on day 5. 30 μL cell culture supernatant was diluted 1:4 in 1×PBS, 0.5% BSA, 0.05% Tween20 and quantified by Octet by interpolating the initial slope against a linear, point-to-point curve of the standard curve (200, 100, 50, 25, 12.5, 6.25, 3.125, 0 μg/mL). Harvested supernatant from each antibody transfection was combined equimolar by dosing group for pooled purification. Purification was performed on an Akta Pure25 with a HitTrap MabSelect SuRe pcc column at 5 mL/min. Pooled cell culture supernatant was loaded onto the column washed with 25 mM sodium phosphate, 150 mM sodium chloride, 10% glycerol pH 6.0 and eluted with 100 mM glycine, 500 mM sodium chloride, 10% glycerol, pH 3.2 directly into 10% volume of 1M sodium phosphate, pH 6.0. Peak fractions were pooled, concentrated using centrifugal concentrators (10 kDa MWCO, MilliporeSigma) and diluted to a final concentration of ˜0.3 mg/mL/antibody to dose at 5 mg/kg/antibody (IgG equivalent). Antibody pools were snap frozen in liquid nitrogen and thawed before dosing.

### In Vivo Screening:

Antibodies were pooled into test articles in groups of 5-10 and were injected at 5 mg/kg/antibody. A total of 3 mice per TA group per time point were systemically dosed via tail vein. After 6, 24 and 72 hours, animals were sacrificed and blood was collected in serum separator tubes via cardiac puncture. Prior to organ collection, mice were transcardially perfused with PBS. The brain was divided into two hemispheres and along with muscle, liver, heart and lungs, all tissues were snap frozen and stored at −80° C.

### Tissue Processing:

One hemisphere of brain tissues (right side) was lysed (750 μL) using RIPA buffer (ThermoFisher) with Halt protease inhibitor (ThermoFisher, 1:100) in a TissueLyzer (Qiagen) on at 30 rpm for 7 min using a stainless steel bead of 5 mm (Qiagen). Upon ensuring that the sample is lysed, tubes are centrifuged at 7000 g at 4° C. for 20 min. The supernatant was collected representing the whole brain fraction and was subjected to quantification using decoding protocol described herein.

For parenchymal extraction, the other brain hemisphere (left) was minced into small pieces with a scalpel in an HBSS buffer containing 10 mM HEPES. The sample was homogenized using a long 21 G cannula (0.8 mm×120 mm) mounted on a 2 mL syringe by aspirating and pushing out 20 times. Homogenate was transferred to 2 mL tubes and centrifuged at 2000 g at 4° C. for 10 min. Supernatant here represents the whole parenchyma while the pellet, when lysed, represents the myelin and vessels fraction. The whole parenchyma was further processed into cell free and cell-associated fractions. For other tissues (muscle, heart, liver and lung), the above-mentioned lysis protocol was followed. All supernatants post-lysis were collected and subjected for quantification through decoding protocol described herein.

### Quantification:

Molecules were quantified using decoding protocol described herein. Briefly, lysed tissue samples were incubated with magnetic beads coated with ALFAtag single-domain antibody (ALFA Selector ST, Nanotag Biotechnologies, Germany) added at 10 μL per well and incubated shaking at 900 rpm to ensure thorough mixing of beads for 1.5 hours at room temperature (RT), plates were then moved to 4° C. to continue capturing overnight. Beads were washed once with 1×PBS and the appropriate scFv decoder phage library (1E10 pfu/mL) was added to bind the barcodes for 2 hours at RT, shaking at 900 rpm. Beads were then washed three times to remove non-specifically binding phages in 1×PBST (0.1% Tween-20) using an extractor shield to carefully transfer the beads to each wash plate. After the final wash, beads were incubated in Trypsin (0.1 μg/mL, Sigma Aldrich) for 1 hour at 37° C. to elute phages off of the beads. The eluate was then added to E. coli TG1 cells (LGC, Biosearch Technologies) in exponential phase (OD600=0.4) and incubated for 1 hour at 37° C., shaking at 1000 rpm. Carbenicillin (120 μg/mL, Sigma Aldrich) and Glucose (1%) were added to LB media (Sigma Aldrich) and 1 mL of this prepared media was added to the bacterial cells infected with phages and incubated shaking at 1000 rpm for 16 hours at 37° C. Samples were then subjected to NGS library preparation where 1 μL of cells were added to Q5 High-Fidelity 2× Master Mix (NEB) with appropriate forward and reverse primers (0.75 μM final concentration) to amplify the scFv decoder insert and libraries were amplified with 13 cycles of PCR. DNA libraries were then indexed with unique dual index primers (i5 and 17) to enable sequencing and the libraries were pooled together post-indexing. Libraries were then purified using SPRI bead purification (CleanNGS DNA purification magnetic beads) using an amount appropriate for the desired size selection and DNA was eluted in a buffer containing 10 mM Tris-HCl (pH 8.0) with 0.1 mM EDTA. DNA libraries were then sequenced with Illumina's NextSeq 2000 sequencer, generating paired end reads of 150 bp. Precise quantification of barcode amounts in the tissue samples from NGS data was enabled by including a standard curve that was decoded with known amounts of protein in commercially purchased (Transnetyx) strain-matched control tissues lysed with the above-mentioned lysis protocol under a larger scale (GentleMacs). A known amount of control phage was also added to every well for normalization of the NGS signal across all the wells thereby enabling accurate and meaningful quantification of barcode amounts in the wells.

### Results

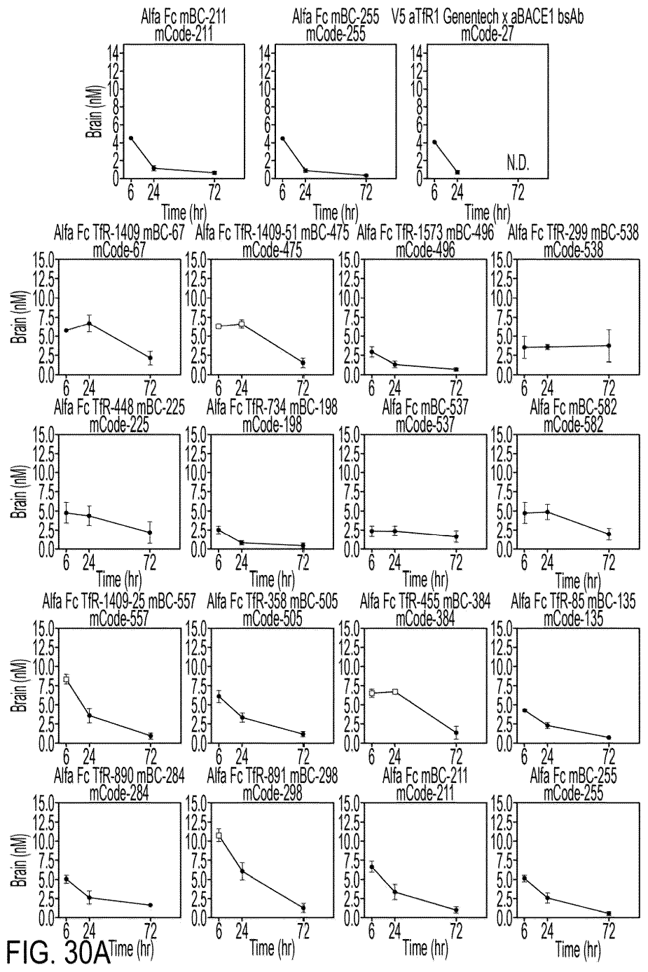

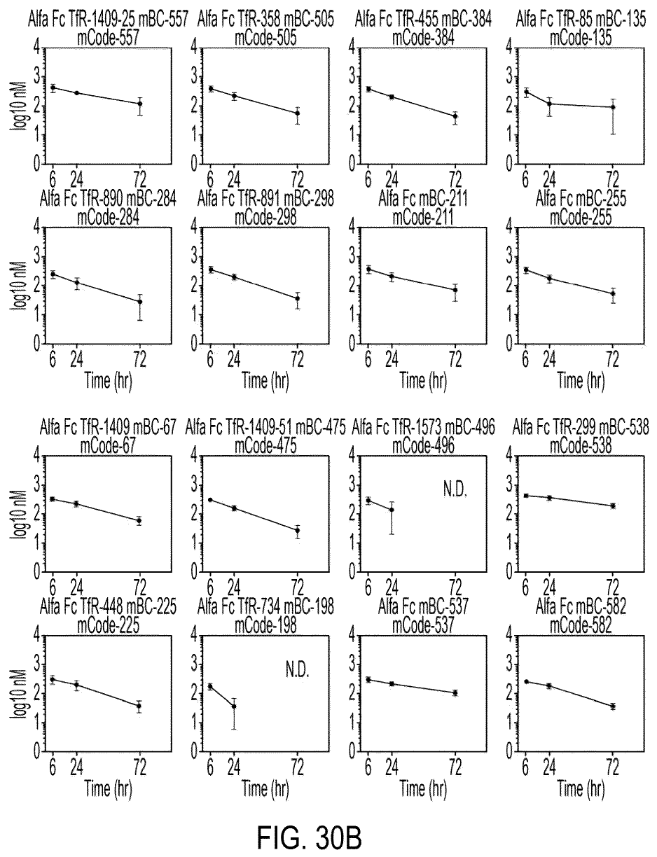

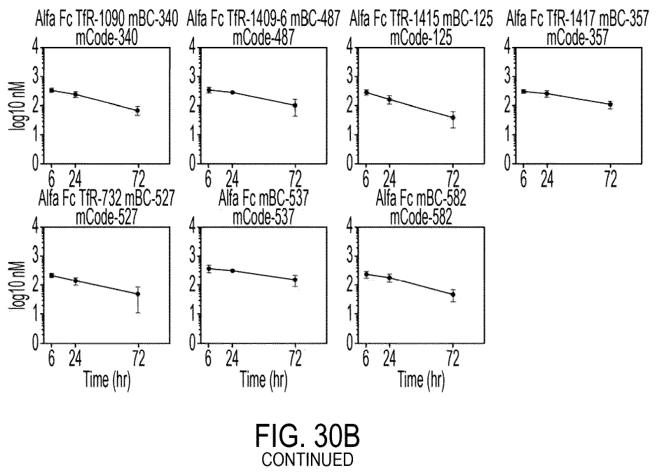

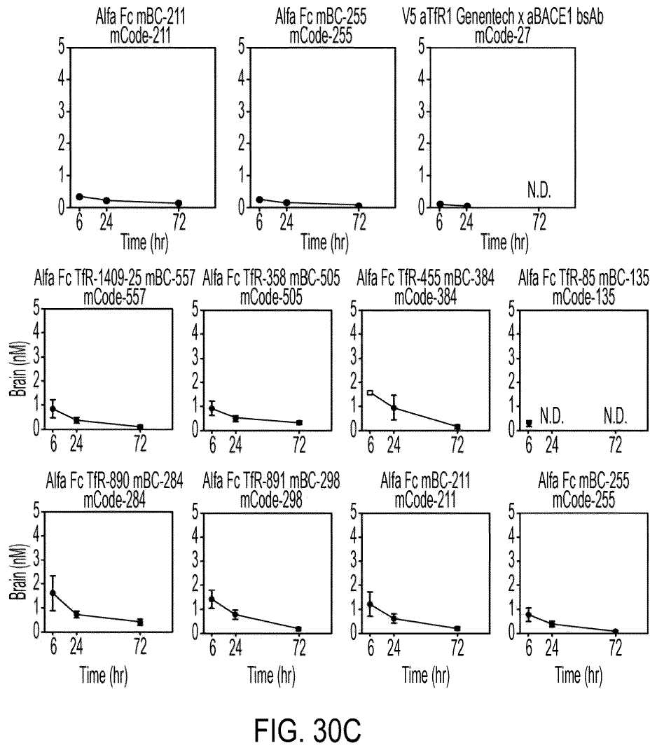

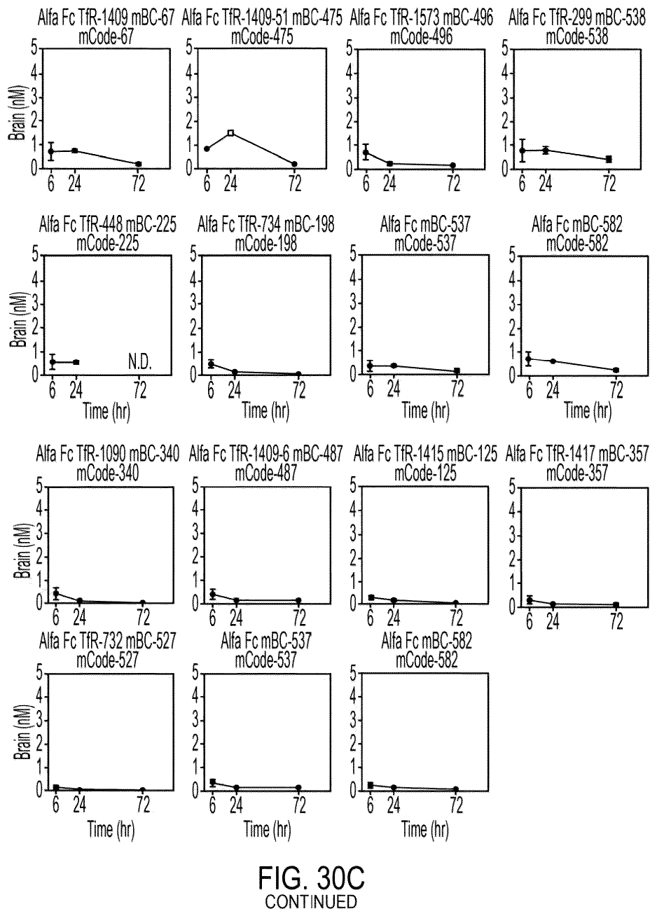

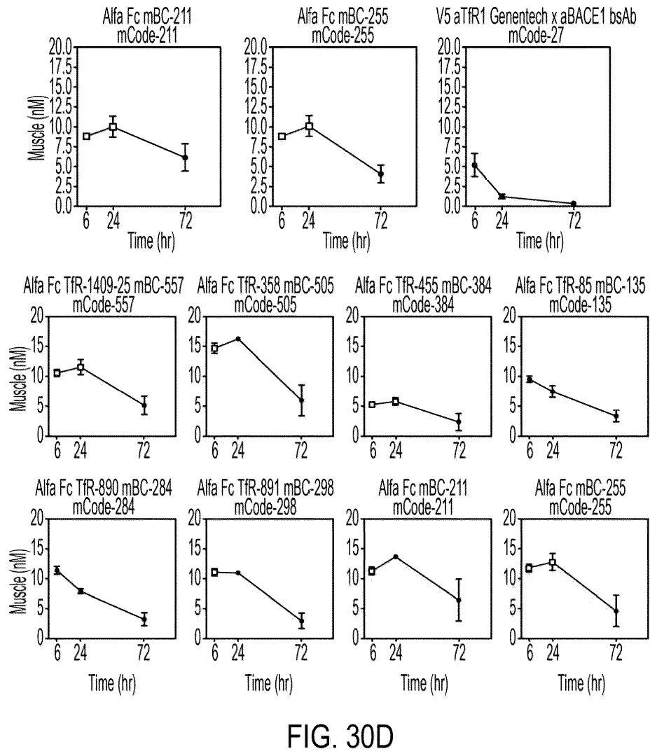

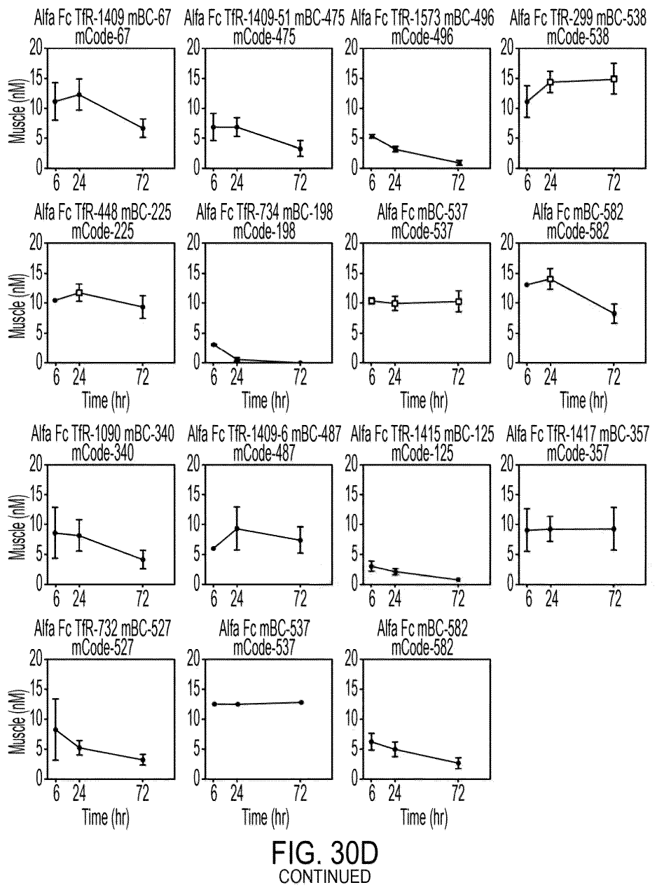

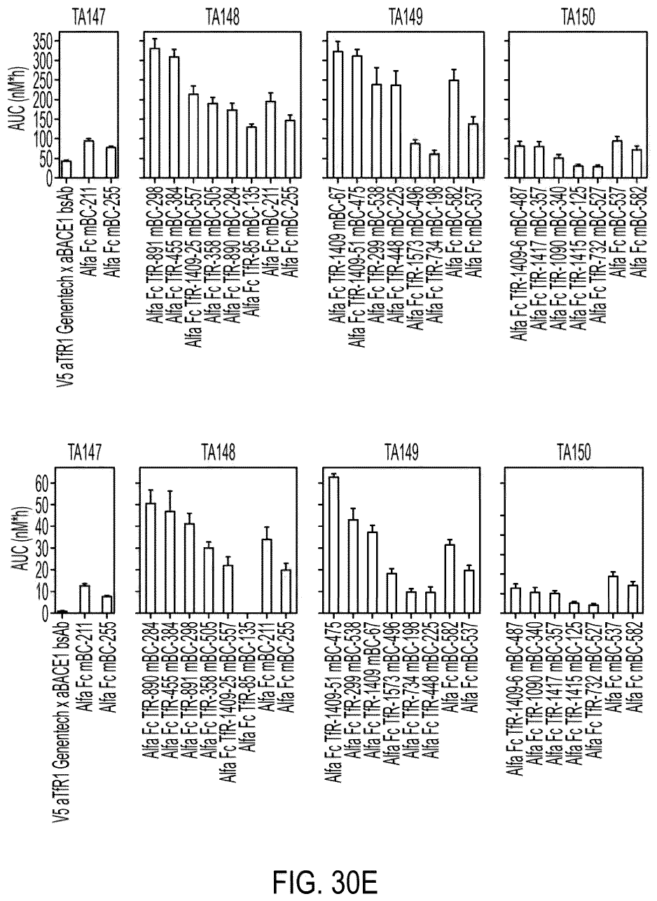

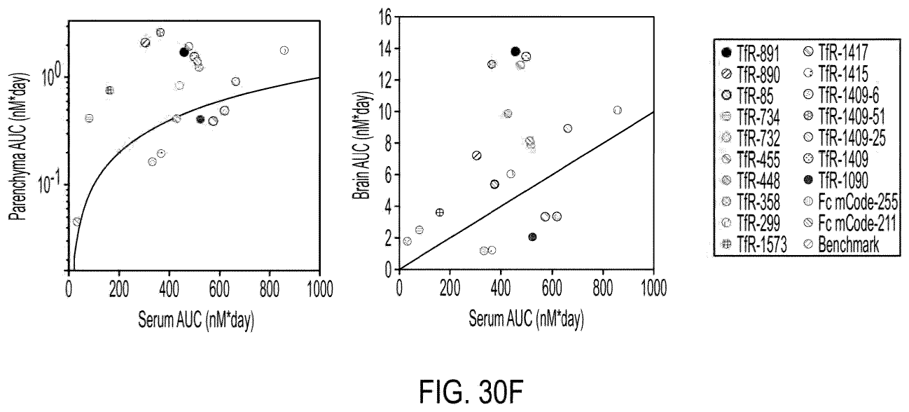

FIG. 30A-FIG. 30D show brain, serum, parenchyma and muscle pharmacokinetics for all measured molecules selected for follow up from an in vivo screen described by Example 25. Notably, a wide variety of phenotypes were observed. TfR shuttle 1409 (i.e., TfR-1409) and its variant TfR-1409-51, and TfR shuttle 891 (i.e., TfR-891) have strong brain uptake when compared to molecules without shuttles, or the VHH shuttle selected for low brain uptake, TfR-732.

FIG. 30E shows AUCs for molecules over time, highlighting variant TfR-1409-51, which has the best accumulation compared to other molecules tested.

FIG. 30F, shows data comparing serum AUC to both brain and parenchyma AUC, highlighting variation across molecules. As shown in FIG. 30f, variant TfR-1409-51 exhibited a highest ratio of brain concentration comparatively to serum data.

Accordingly, the present example identifies variant shuttle moieties that exhibit improved properties relative to other shuttle moieties in a multiplexed format.

### Example 27: Design of Improved Variants of TfR and FLT1 Shuttles Using Protein Language Model (PLM) Diversification

The present example demonstrates design of improved variants of shuttles (e.g., brain shuttle moieties). In some embodiments, an improved variant of a shuttle exhibits improved phenotypes of shuttles. In some embodiments, a phenotype includes pharmacokinetic (or clearance) data as described herein.

### Method

It is an insight of the present disclosure that a shuttle can be improved upon via a shallow, directed mutational search with the use of protein language models (PLMs). In recent publications (P. Emami et al., 2023; B. L. Hie et al., 2023), PLMs including ESM-2 (Z. Lin et al., 2022) and ProtBERT (A. Elnaggar et al., 2022) have been shown to possess a zero-shot, or naïve understanding of protein functionality across many families of proteins. It is the insight of the present disclosure that an already good High Throughput in Vivo screening (HTV)-informed hit can be further optimized and diversified using AI (e.g., PLMs), and tested rapidly in vivo again using the technologies disclosed herein (see FIG. 31). This process repeated more and more times can generate a rich dataset to fine-tune existing PLMs to engineer shuttles with optimal properties. In some embodiments, this is referred to as “learning the shuttle-protein-manifold”.

Initially, in silico experiments were conducted using ESM-2 and ProtBERT to generate scores for every possible single mutation in the sequence of TfR-1409. These scores for mutations were compared across the TfR-1409 sequence to frequencies of those amino acids in publicly available unpaired VH and VHH sequences in the OAS database (T. H. Olsen et al., 2022). It was concluded that ProtBERT scores were better aligned with beneficial VHH mutations.

In silico sampling was performed with ProtBERT of variant sequences of TfR-1409 at maximum mutational distances in the set {1, 2, 3, . . . , 10}. For each sampling step, the gradient of the score for that sequence was computed with respect to the amino acid identities of each residue, and a new mutation with a probability determined by a categorical distribution derived from this gradient was proposed. Such gradient-based sampling was performed with a modified version of the code provided by P. Emami et al (2023). Notably, while the Emami paper uses gradient-directed MCMC for sampling, implementation of the same herein drops the accept/reject step of MCMC, effectively resulting in a noisy gradient ascent. For more information on the sampling algorithm used to generate variants, see Algorithm 1 in P. Emami et al (2023).

After generating around 10,000 variants of TfR-1409 and scoring the sequences in silico, duplicate sequences were dropped and the 20 top-scoring variants from each of the 10 possible maximum mutational distances were selected, resulting in a set of 200 variants. Binding curves for 196 of the resulting 200 variants were assayed via BLI. The top 50 binding curves, sorted by fit quality, were selected for advancement to multiplexed in vivo screening (HTV) using the binder barcode platform described herein. Of these 50, 33 variants, including wildtype TfR-1409, were ultimately assayed in vivo. The best-performing variant in vivo was TfR-1409-51.

Protein language models guided amutagenesis approach to design variants that preserve protein stability and proper folding. A library of AI-designed variants were generated and evaluated using yeast surface display to assess levels of display (as a proxy for stability), target binding, and polyreactivity. Species cross-reactivity and pH sensitivity was also screened at this stage. Next-generation sequencing of the sorted yeast populations was used to select for promising variants that exhibited desired properties (e.g., low polyreactivity, range of affinities) for in vivo screening.

### Results

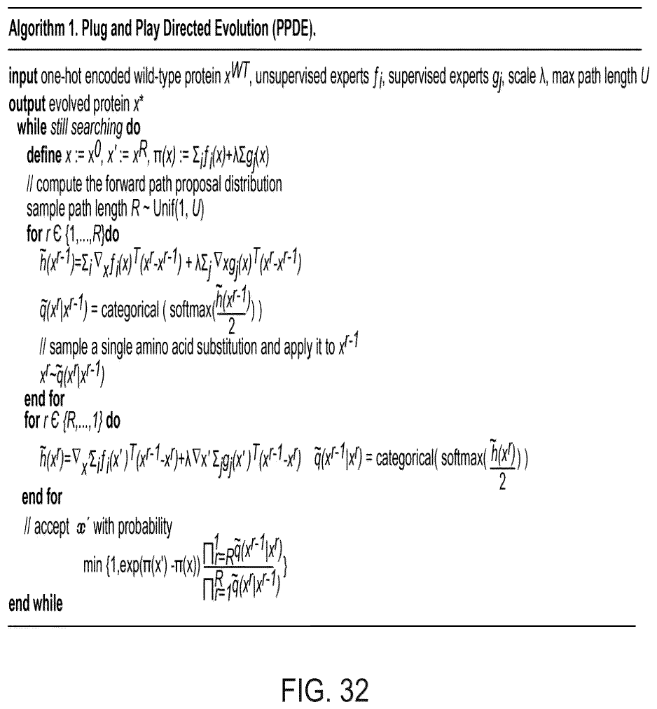

FIG. 31 shows a general overview of a described process above. Shuttles such as TfR-1409 were selected from in vivo screening campaigns such as described herein, and iterated through a protein language model, where they are then put back in vivo for further assessment (see also FIG. 32).

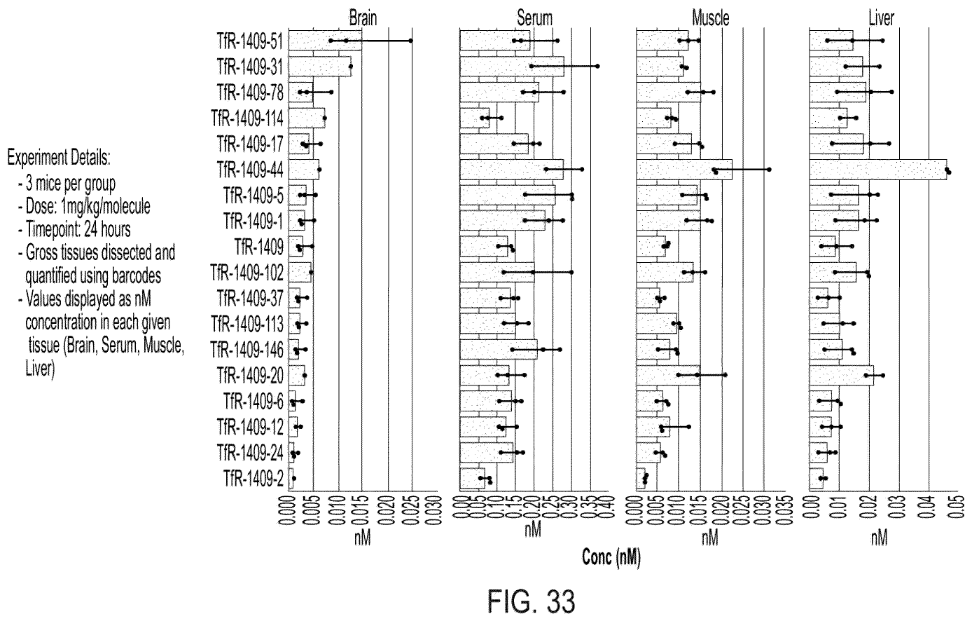

FIG. 33 depicts results of a screen for tested molecules, as described herein, but focusing on just the molecules which are AI improved variants of TfR-1409. As shown in FIG. 33, TfR-1409-51 exhibited relatively increased concentration in brain update (e.g., by 4-times compared to other tested molecules). Several of AI-identified molecules perform worse than the parent sequence by the metric of brain uptake. Unexpectedly, the platform described herein provides actual in vivo performance metrics to identify variant molecules (e.g., shuttles) with improved, desirable properties as described herein.

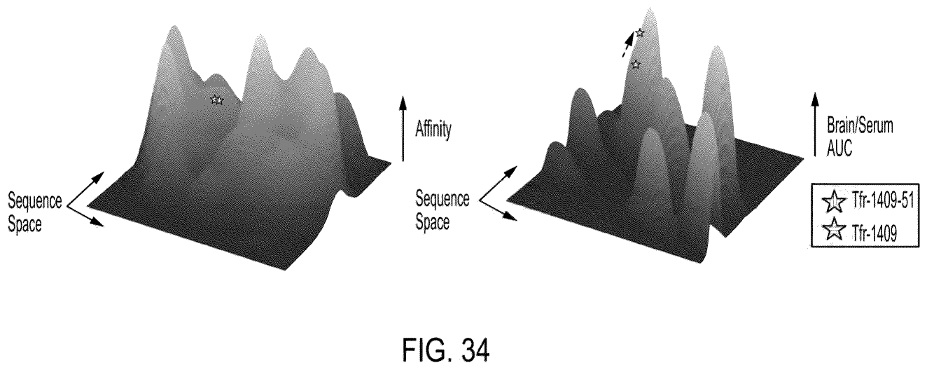

FIG. 34 depicts a comparison of in vivo vs in vitro data. X axis and Y axis are an approximation of sequence space as fit with UMAP, while Y represents measure affinity, or brain over serum AUC as described herein. These two scores are poorly correlated, indicating that in vivo data is required for identifying desirable cargo for in vivo outcomes.

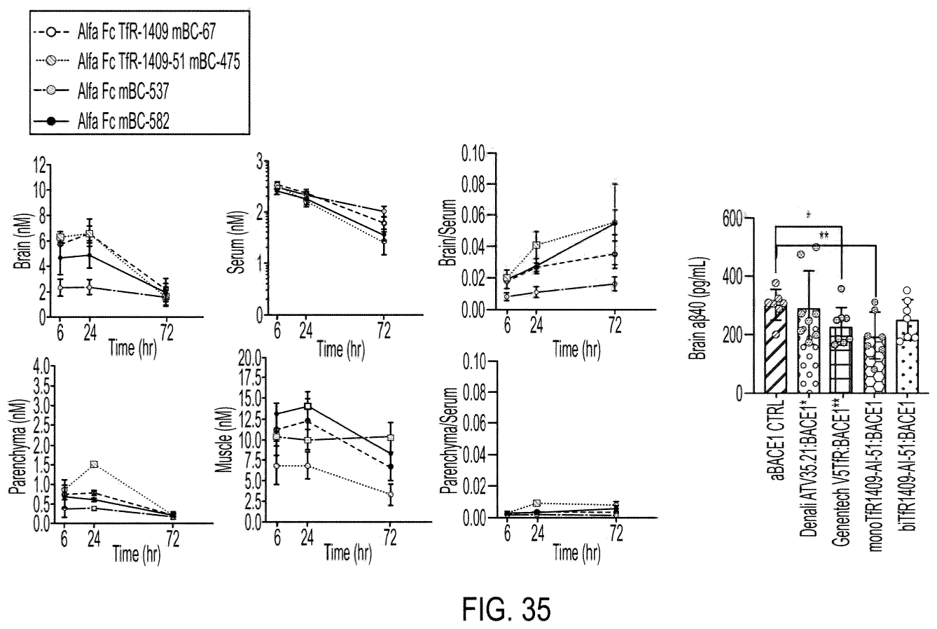

FIG. 35 highlights an observed variation from a validation PK experiment described in Example 26 for TfR-1409 and TfR-1409-51. FIG. 35 shows an increased brain uptake as well as increased parenchymal abundance, highlighting an improvement made with the AI-based approach described herein and a positive correlation with in vivo screening results.

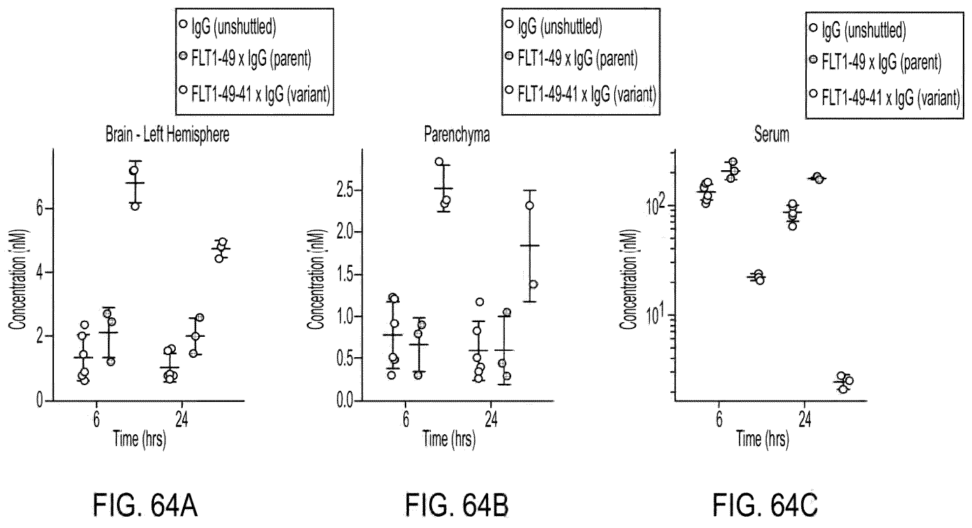

FIG. 64 depicts plots comparing levels of an IgG either alone (unshuttled) versus fused to shuttle FLT1-49 (parent) or an affinity-optimized variant FLT1-49-41 as quantified by decode analysis. Constructs were examined at 1 mg/kg in a humanized FLT1 mouse model at 6 hr and 24 hr timepoints in a) whole brain preparations, b) parenchymal fractionated samples, and c) serum. These data demonstrate that affinity optimized shuttle variant FLT1-49-41 shows enhanced brain uptake in humanized FLT-1 mouse model.

### Example 28: In Vivo Characterization and Screening of TfR Shuttle- IgG Lecanemab Payload Fusions

The present example demonstrates a method of screening and characterizing shuttle-payload fusions as described herein. The present example also demonstrates that a binder-barcode platform described herein can be used to assess abundance of a delivered shuttle-cargo polypeptide, for example TfR-IgG lecanemab fusions. Moreover, the present example demonstrates that a binder-barcode platform described herein can be used to identify and/or quantify cargo polypeptides and/or components thereof for certain phenotypes such as expression and/or localization. For example, in some embodiments, shuttle-payload (e.g., shuttle-cargo polypeptides) can be identified and/or assessed for ability to cross the blood-brain barrier (BBB) (e.g., by binding to a BBB surface receptor and inducing transcytosis into the brain) as described by this example. In some embodiments, a shuttle is or comprises a shuttle moiety.

Other types of cargo components can be assessed using a binder-barcode platform as described herein. Phenotypes other than expression and localization can also be quantified and/or assessed using a binder-barcode platform as described herein. Exemplary phenotypes include epitope properties, affinity, thermostability, pH sensitivity, or other phenotypes described herein.

### Method

### Molecule Construction and Production:

Heavy and light chain plasmid DNA was combined at a 1:1.5 ratio for symmetrical antibodies or 1:1:3 heavy (knob):heavy (hole):light chain plasmid to a total mass of 40 μg combined with 10 μg salmon sperm DNA. DNA was complexed with Expifectamine293 according to manufacturer recommendations (Gibco) in a transfection volume of 50 mL. Transfected cells were harvested by centrifugation on day 5. 1 mL cell culture supernatant was transferred to a fresh 96-deep well plate containing 10 μL MagSepharose PrismA beads (Cytiva) in three replicates. Supernatants and beads were incubated at 36.5° C. for 1 hour at 1,000 RPM. Beads were collected, washed, and purified antibodies eluted into 120 μL 100 mM glycine, 150 mM sodium chloride, pH 3.2 using the KingFisher Purification System (Thermo Fisher) and neutralized with 15 μL 1 M Sodium Phosphate, pH 7. Purified antibodies were quantified by BCA and evaluated for aggregation by size exclusion chromatography. Antibodies of sufficient quality were combined equimolar by dosing group for pooled purification. Purification was performed on an Akta Pure25 with a HitTrap MabSelect SuRe pcc column at 5 mL/min. Pooled cell culture supernatant was loaded onto the column washed with 20 mM sodium phosphate, 150 mM sodium chloride, 1 pH 7.0 and eluted with 100 mM glycine, 150 mM sodium chloride, pH 3.2 directly into 10% volume of 1M sodium phosphate, pH 7.0. Peak fractions were pooled, concentrated using centrifugal concentrators (10 kDa MWCO, MilliporeSigma). Concentrated antibodies were dialyzed into 20 mM sodium phosphate, 150 mM sodium chloride and diluted to a final concentration of ˜0.5 mg/mL/antibody to dose at 5 mg/kg/antibody (IgG equivalent). Antibody pools were snap frozen in liquid nitrogen and thawed before dosing.

### In Vivo Screening and Characterization:

Antibodies were pooled into test articles in groups of 5-10 and were injected at 5 mg/kg/antibody. A total of 3 mice per TA group per time point were systemically dosed via tail vein. After 6, 24 and 72 hours, animals were sacrificed and blood was collected in serum separator tubes via cardiac puncture. Prior to organ collection, mice were transcardially perfused with PBS. The brain was divided into two hemispheres and along with muscle, liver, heart and lungs, all tissues were snap frozen and stored at −80° C.

### Tissue Processing:

One hemisphere of brain tissues (right side) was lysed using TPER (ThermoFisher) buffer with Halt protease inhibitor (ThermoFisher, 1:100) where 1 mL buffer was added to every 150 mg tissue in a TissueLyzer (Qiagen) on at 30 rpm for 7 min using a stainless steel bead of 5 mm (Qiagen). Upon ensuring that the sample is lysed, tubes are centrifuged at 7000 g at 4° C. for 20 min. The supernatant was collected representing the whole brain fraction and was subjected to quantification using decoding protocol described herein.

For parenchymal extraction, the other brain hemisphere (left) was minced into small pieces with a scalpel in an HBSS buffer containing 10 mM HEPES. The sample was homogenized using a long 21 G cannula (0.8 mm×120 mm) mounted on a 2 mL syringe by aspirating and pushing out 20 times. Homogenate was transferred to 2 mL tubes and centrifuged at 2000 g at 4° C. for 10 min. The supernatant here represents the whole parenchyma while the pellet, when lysed, represents the myelin and vessels fraction. The whole parenchyma was further processed into cell free and cell-associated fractions. For other tissues (muscle, heart, liver and lung), the above-mentioned lysis protocol was followed. All supernatants post-lysis were collected and subjected for quantification through decoding protocol described herein.

### Quantification:

Molecules were quantified using decoding protocol described herein. Briefly, lysed tissue samples were incubated with the appropriate scFv decoder phage library (1E10 pfu/mL) to bind the barcodes for 2 hours at room temperature (RT) shaking at 600 rpm. Magnetic beads coated with ALFAtag single-domain antibody (ALFA Selector ST, Nanotag Biotechnologies, Germany) were then added at 10 μL per well and incubated shaking at 900 rpm to ensure thorough mixing of beads for 1.5 hours at RT. Beads were then washed four times to remove non-specifically binding phages in 1×PBST (0.10% Tween-20) using a KingFisher bead washing station, after which they were incubated in Trypsin (0.1 μg/mL, Sigma Aldrich) for 1 hour at 37° C. to elute phages off of the beads. The eluate was then added to E. coli TG1 cells (LGC, Biosearch Technologies) in exponential phase (OD600=0.4) and incubated for 1 hour at 37° C., shaking at 1000 rpm. Carbenicillin (120 μg/mL, Sigma Aldrich) and Glucose (1%) were added to LB media (Sigma Aldrich) and 1 mL of this prepared media was added to the bacterial cells infected with phages and incubated shaking at 1000 rpm for 16 hours at 37° C. Samples were then subjected to NGS library preparation where 1 μL of cells were added to Q5 High-Fidelity 2× Master Mix (NEB) with appropriate forward and reverse primers (0.75 μM final concentration) to amplify the scFv decoder insert and libraries were amplified with 13 cycles of PCR. DNA libraries were then indexed with unique dual index primers (i5 and i7) to enable sequencing and the libraries were pooled together post-indexing. Libraries were then purified using SPRI bead purification (CleanNGS DNA purification magnetic beads) using an amount appropriate for the desired size selection and DNA was eluted in a buffer containing 10 mM Tris-HCl (pH 8.0) with 0.1 mM EDTA. DNA libraries were then sequenced with Illumina's NextSeq 2000 sequencer, generating paired end reads of 150 bp. Precise quantification of barcode amounts in the tissue samples from NGS data was enabled by including a standard curve that was decoded with known amounts of protein in commercially purchased (Transnetyx) strain-matched control tissues lysed with the above-mentioned lysis protocol under a larger scale (GentleMacs). A known amount of control phage was also added to every well for normalization of the NGS signal across all the wells thereby enabling accurate and meaningful quantification of barcode amounts in the wells.

### Results

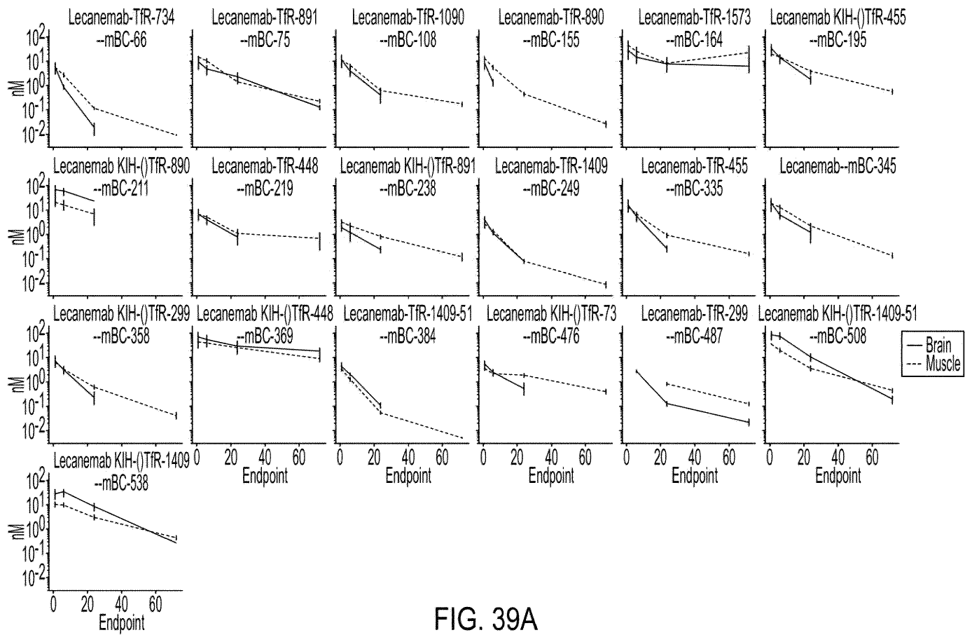

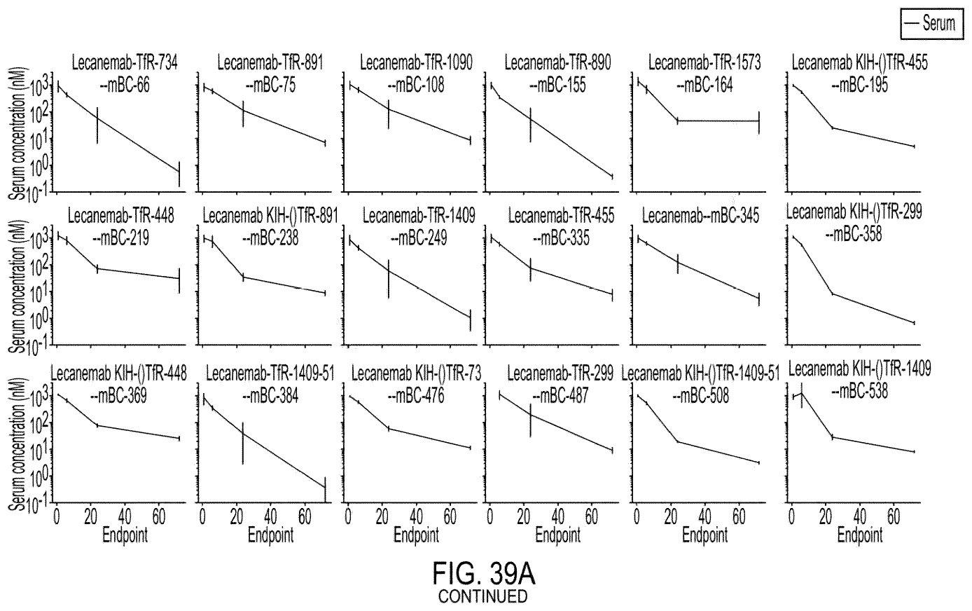

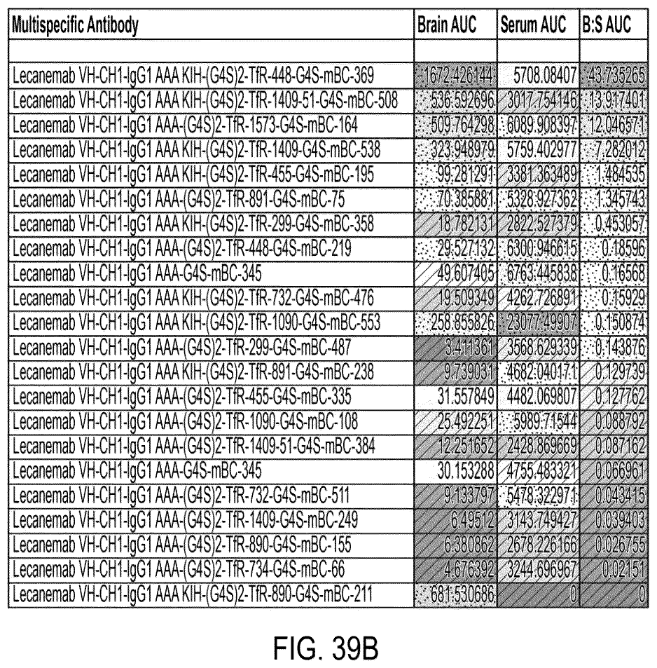

FIG. 39A shows brain, muscle and serum PK for TfR shuttle variants when fused to lecanemab. Notably, monovalent format (marked as KiH) for 14090-51 improves brain PK and AUC, increasing brain AUC by 10-fold over lecanemab without shuttle. Importantly, several variants which showed strong performance on the Fc-only constructs in Examples 25-27 show poor performance on lecanemab, indicating the importance of testing all shuttle constructs on the payload of interest. The present Example demonstrates that sequences such as those listed in Table 5 can be assessed in accordance with methods described herein.

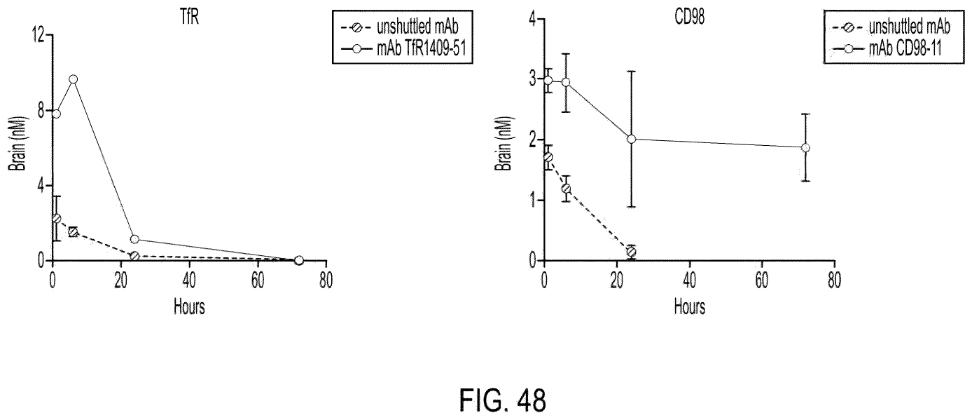

FIG. 48 depicts plots showing in vivo PK validation of TfR1 and CD98hc shuttles with a common anti-amyloid beta (Aβ) antibody payload. Shown are shuttled and unshuttled anti-aβ mAbs with timepoints measured at 1, 6, 24, and 72 hours post-injection. The left plot shows kinetics of shuttle TfR-1409-51 with anti-aβ measured in humanized TfR1 mice.

### Example 29: In Vivo Characterization and Screening of TfR Shuttle- aBACE1 Payload Fusions

The present example demonstrates a method of screening and characterizing shuttle-payload fusions as described herein. The present example also demonstrates that a binder-barcode platform described herein can be used to assess abundance of a delivered shuttle-cargo polypeptide, for example TfR-aBACE1 fusions. Moreover, the present example demonstrates that a binder-barcode platform described herein can be used to identify and/or quantify cargo polypeptides and/or components thereof for certain phenotypes such as expression and/or localization. For example, in some embodiments, shuttle-payload (e.g., shuttle-cargo polypeptides) can be identified and/or assessed for ability to cross the blood-brain barrier (BBB) (e.g., by binding to a BBB surface receptor and inducing transcytosis into the brain) as described by this example. In some embodiments, a shuttle is or comprises a shuttle moiety.

Other types of cargo components can be assessed using a binder-barcode platform as described herein. Phenotypes other than expression and localization can also be quantified and/or assessed using a binder-barcode platform as described herein. Exemplary phenotypes include epitope properties, affinity, thermostability, pH sensitivity, or other phenotypes described herein.

### Method

### Molecule Construction and Production:

Heavy and light chain plasmid DNA was combined at a 1:1.5 ratio for symmetrical antibodies or 1:1:3 heavy (knob):heavy (hole):light chain plasmid to a total mass of 0.8 μg per mL of transfection combined with 0.2 μg salmon sperm DNA per mL of transfection. DNA was complexed with Expifectamine293 (Gibco) according to manufacturer recommendations. Transfected cells were harvested by centrifugation on day 5. Purification was performed on an Akta Pure25 with a HitTrap MabSelect SuRe pcc column at 5 mL/min. Cell culture supernatant was loaded onto the column washed with 20 mM sodium phosphate, 150 mM sodium chloride, 1 pH 7.0 and eluted with 100 mM glycine, 150 mM sodium chloride, pH 3.2 directly into 10% volume of 1M sodium phosphate, pH 7.0. Peak fractions were pooled, concentrated using centrifugal concentrators (10 kDa MWCO, MilliporeSigma). Concentrated antibodies were dialyzed into 20 mM sodium phosphate, 150 mM sodium chloride and diluted to a final concentration of ˜5 mg/mL to dose at 50 mg/kg/antibody (IgG equivalent). Antibodies were snap frozen in liquid nitrogen and thawed before dosing.

### Dosing, Blood, and Tissue Collections:

All mouse studies utilized hTfR females (Biocytongen). Mice were IV dosed via the tail vein with 30 mg/kg or 50 mg/kg of test articles.

At terminal sacrifice, blood (˜500 μL) was collected via cardiac puncture. Blood was collected in EDTA tubes to prevent clotting and spun at 14000 g for 10 min. After blood collection, mice were transcardially perfused with PBS. Fresh-frozen brain tissue was homogenized using a Qiagen TissueLyser with 5 mm steel beads for 7 min at 30 Hz in 500 uL of RIPA lysis buffer+HALT protease inhibitors (100×) for 150 mg of brain tissue.

### Human IgG ELISA:

IgG antibody concentrations in mouse plasma and brain lysates were quantified using a generic human IgG assay (ab195215. AbCam) following the manufacturer's instructions. Plasma was diluted 1:20000 and 1:40000 in a sample diluent provided with the ELISA kit. Similarly, brain lysates were diluted 1:2 and 1:5 for analysis of huIgG concentration. Briefly, samples and standard curves were incubated with an antibody cocktail for 40 minutes at room temperature on a plate shaker set to 400 rpm. After washes, a TMB Development Solution was added to each well and the reaction was stopped with the addition of a Stop Solution to each well. Absorbance was measured at 450 nm in a plate reader. The standard curve was fit using a linear logistic regression.

### Mouse Aβ40 ELISA:

For PD evaluation, brains were extracted after perfusion and hemi-brains were isolated for homogenization in 8× by tissue weight of 5 M GuHCl and incubated under rotation for 3 hours at 4 C. These lysates were further diluted 1:10 with dilution Buffer for Guanidine. Samples were then spun at 14,000 rpm for 20 min at 4 C, and supernatants were isolated for mouse Abeta-40 measurements. MaxiSorp plate was coated overnight with the polyclonal capture antibody specific for the C-terminus of the Aβ40 peptide (Millipore #AB5074P) at 4 C. After blocking was performed with 200 μL of blocking buffer (2% BSA, 7.5 g/L glycine in PBS) for 1 h at room temperature. Diluted guanidine brain lysates samples and peptide standards were loaded on the plate and added concurrently with the biotinylated primary β-Amyloid, 1-16 (Biolegend 805703) antibody followed by a High Sensitivity Streptavidin-HRP (Thermo Fisher P121130) secondary antibody. Following another four washes, 100 μL of TMB was added to each well. Plates were incubated in the dark for 30 min at room temperature, then the reaction was terminated by the addition of 50 μL of H2SO4 (20% v/v) (Stop Solution). Absorbance values at 450 nm were measured using a Bio-Rad Microplate Reader. Assay sensitivity was <300 pg/mL for both Aβ40 and Aβ42.

### Results

FIG. 35 shows quantification of aB-40 (i.e., Aβ40) in the brain at 24 hours for the measured constructs. Notably, monovalent TfR-1409-51 shows a performance with desirable properties as compared to benchmarks or its bivalent version.

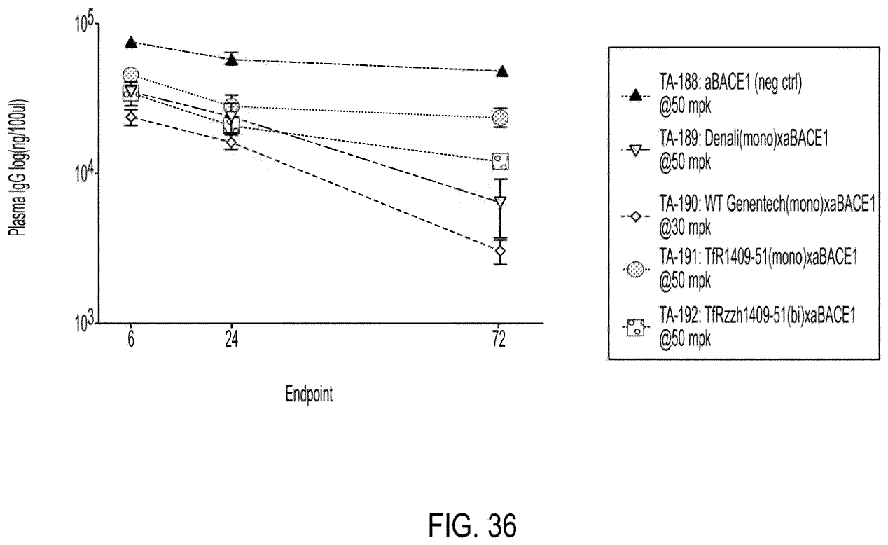

FIG. 36 highlights that monovalent TfR-1409-51-aBACE1 has improved PK properties in addition to brain uptake relative to other molecules tested.

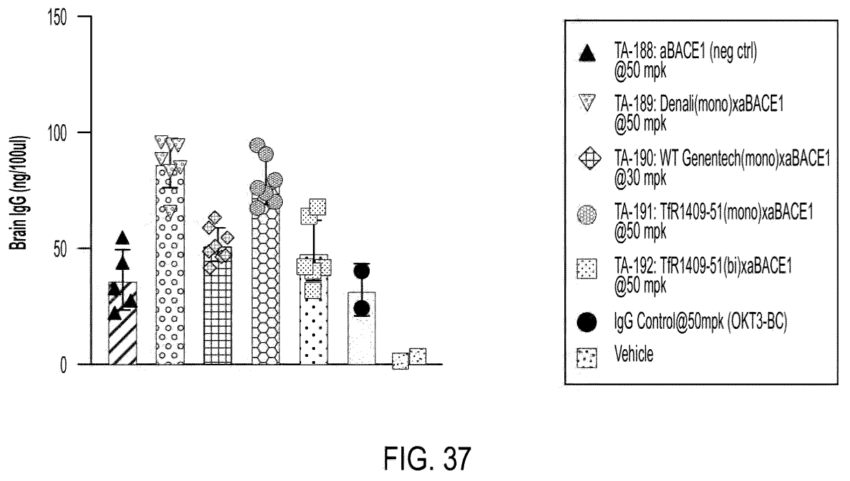

FIG. 37 shows brain IgG concentration at 24 hours, interestingly Denali(mono)aBACE1 has high brain uptake, but does not significantly knock down aβ40.

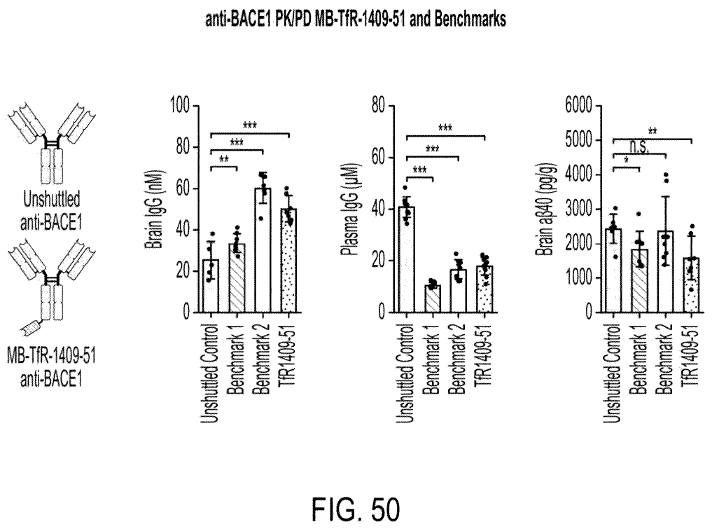

FIG. 50 shows plots of brain uptake and functional activity of an anti-BACE1 antibody alone or shuttled by MB-TfR-1409-51 versus benchmarks in humanized TfR1 mice at 24 hrs post-injection. Column 1 from left shows format of unshuttled anti-BACE1 control mAb and the shuttled MB-TfR-1409-51 construct. Column 2 from left shows levels of antibody for each construct measured in whole brain using ELISA. Column 3 from left shows levels of antibody for each construct measured in plasma using ELISA. Column 4 from left shows levels of Aβ40 for each construct measured in brain using ELISA.

### Example 30: In Vivo Detection of TfR Shuttle-aBACE1 Payload Fusions

The present example demonstrates in vivo characterization of shuttle-payload fusions as described herein. Specifically, the present example demonstrates immunofluorescence detection of the TfR-1409-51-aBACE1 construct in specific brain regions.

### Method

### In Vivo Detection:

hTfR mice were IV dosed via the tail vein with 30 mg/kg or 50 mg/kg of test articles. 24 hours after dosing, animals were perfused by PBS and brains were extracted, drop-fixed in 4% paraformaldehyde overnight at 4 C followed by 30% sucrose overnight at 4 C. Brains were then sectioned at 35 μm on a sliding cryostat, blocked in 5% NDS (Normal Donkey Serum), 10% of Triton in PBS for 1 hour at room temperature and stained with NeuN (Rabbit monoclonal anti-NeuN antibody (1:500, Abcam ab177487) overnight at 4 C in blocking buffer. After overnight incubation, sections were washed in PBS and and incubation with Alexa Fluor® 594 AffiniPure Donkey Anti-Rabbit IgG (H+L) (1:200) Jackson 711-585-152) and Alexa Fluor® 488 AffiniPure Donkey Anti-Human IgG (H+L) (1:200) (Jackson 709-545-149) at room temperature for 2 hours.

Brain sections were washed in PBS then mounted on slides using Prolong Glass Antifade Mountant and imaged using a Zeiss Axioscan scope (20×/0.8 air objective).

### Results

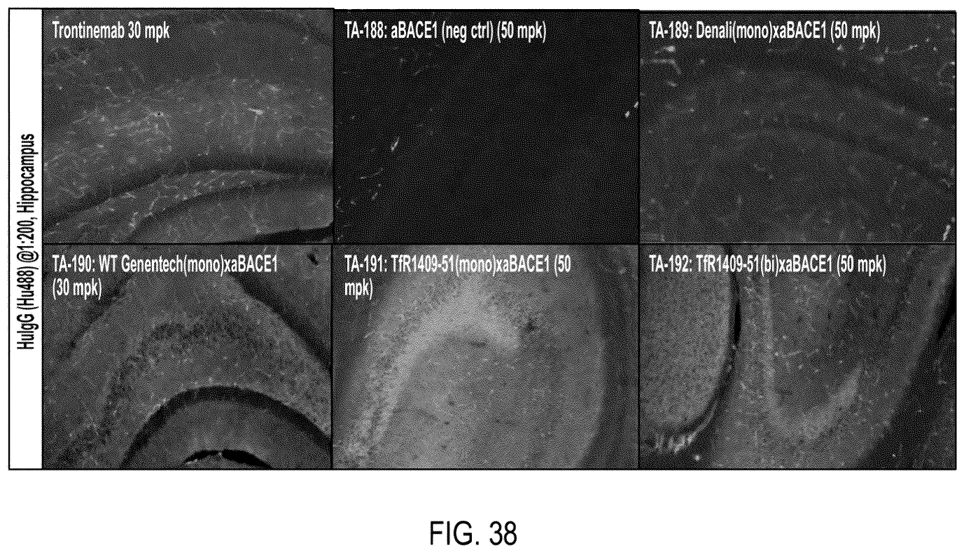

FIG. 38 shows that TfR-1409-51-aBACE1 monovalent at 24 hours exhibits the highest global brain staining when directly compared head-to-head to several TfR benchmark molecules.

### Example 31: Multiplexed In Vivo Screening and Assessment of Phenotypes of Shuttles to Different Targets Using Binder-Barcode Platform

The present example demonstrates a method to perform multiplexed in vivo screening and assessment of phenotypes of shuttles (e.g., brain shuttle moieties) designed to target multiple different targets (or “cell-receptor agents” or “portals”) of interest in a high-throughput format using a binder-barcode platform described herein. For example, in some embodiments, multiple shuttles (e.g., shuttle moieties) can be identified and/or assessed simultaneously using a binder-barcode platform described herein as described by this example. In some embodiments, targets, for example, as identified in this Example, may be nominated by a sophisticated nomination criteria including public scRNA-seq datasets for specific cell types (e.g., brain cell). Additionally, in some embodiments, targets, for example, as identified in this Example, may be nominated by computational predictions of homology, cell surface expression, and/or other proprietary determinants. In some embodiments, a shuttle is or comprises a shuttle moiety. In some embodiments, a phenotype includes pharmacokinetic (or clearance) data as described herein.

### Method

### Molecule Construction and Production:

350 VHH molecules selected for the binding properties to seven different targets namely CD98, FLT1, LDLR1, InsulinR, IGF1R, SCARB1, LRP1 were produced as C-terminal Fc fusions. A person having ordinary skill in the art would appreciate that other molecules can be selected for binding to a variety of targets in a multiplexed, high-throughput screen according to embodiments of the present disclosure.

Plasmid backbone, VHH, or Barcode gene fragment was PCR amplified using 5 ng of template DNA. Forward and reverse primer (250 nM final concentration) was combined with Q5 High-Fidelity Master Mix (1× final concentration, NEB) and amplified for 30 cycles. Plasmid backbone PCR product was purified with Nucleospin PCR Cleanup Kit (Machery Nagel). 7 μg purified, linear plasmid backbone was digested for 2 hours at 37° C. with FastDigest DpnI (Invitrogen) before heat inactivation and SPRI purification (Bulldog Bio). 25 ng backbone DNA were combined 1:3 with insert DNA and HiFi Assembly Master Mix (lx final concentration, NEB). Assembly reaction was incubated for 1 hour at 50° C. 2.5 ng assembled plasmid product was used as the template for PCR amplification. 500 ng PCR amplified DNA was complexed with Expifectamine293 according to manufacturer recommendations (Gibco) in a transfection volume of 800 μL in 12 replicates. Transfected cells were harvested by centrifugation on day 5. 800 μL of cell culture supernatant was transferred to a fresh 96-deep well plate containing 10 μL MagSepharose PrismA beads (Cytiva). Supernatants and beads were incubated at 36.5° C. for 1 hour at 1,000 RPM. Beads were collected, washed, and purified antibodies eluted into 120 μL 100 mM glycine, 150 mM sodium chloride, pH 3.2 using the KingFisher Purification System (Thermo Fisher) and neutralized with 12.5 μL 1 M Sodium Phosphate, pH 7. Purified antibodies were quantified by BCA before pooling at the appropriate mass to achieve 1, 2.5, or 5 mg/kg dosing (IgG equivalent) and concentrated using centrifugal concentrators (10 kDa MWCO, MilliporeSigma) to a final volume of 1050 μL. Final concentration of the pooled antibodies was measured by BCA. Antibody pools were snap frozen in liquid nitrogen and thawed before dosing.

### In Vivo Screening:

Antibodies were pooled into test articles in groups of 50-100 and were injected at 1 mg/kg/antibody. A total of 3 mice per TA group were systemically dosed via tail vein. After 72 hours, animals were sacrificed and blood was collected in serum separator tubes via cardiac puncture. Prior to organ collection, mice were transcardially perfused with PBS. The brain was divided into two hemispheres and along with muscle, liver, heart and lungs, all tissues were snap frozen and stored at −80° C.

### Tissue Processing:

One hemisphere of brain tissues (right side) was lysed using TPER (ThermoFisher) buffer with Halt protease inhibitor (ThermoFisher, 1:100) where 1 mL buffer was added to every 150 mg tissue in a TissueLyzer (Qiagen) on at 30 rpm for 7 min using a stainless steel bead of 5 mm (Qiagen). Upon ensuring that the sample is lysed, tubes are centrifuged at 7000 g at 4° C. for 20 min. The supernatant was collected representing the whole brain fraction and was subjected to quantification using decoding protocol described herein.

For parenchymal extraction, the other brain hemisphere (left) was minced into small pieces with a scalpel in an HBSS buffer containing 10 mM HEPES. The sample was homogenized using a long 21 G cannula (0.8 mm×120 mm) mounted on a 2 mL syringe by aspirating and pushing out 20 times. Homogenate was transferred to 2 mL tubes and centrifuged at 2000 g at 4° C. for 10 min. The supernatant here represents the whole parenchyma while the pellet, when lysed, represents the myelin and vessels fraction. The whole parenchyma was further processed into cell free and cell-associated fractions. For other tissues (muscle, heart, liver and lung), the above-mentioned lysis protocol was followed. All supernatants post-lysis were collected and subjected for quantification through decoding protocol described herein.

### Quantification:

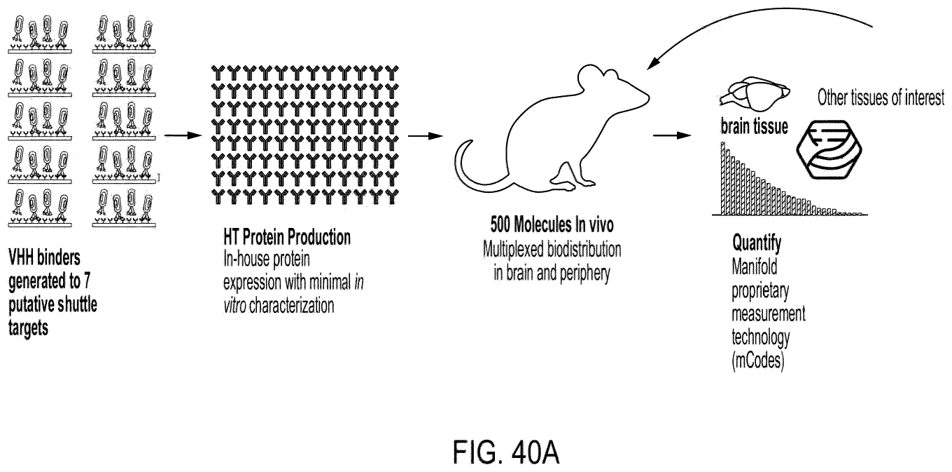

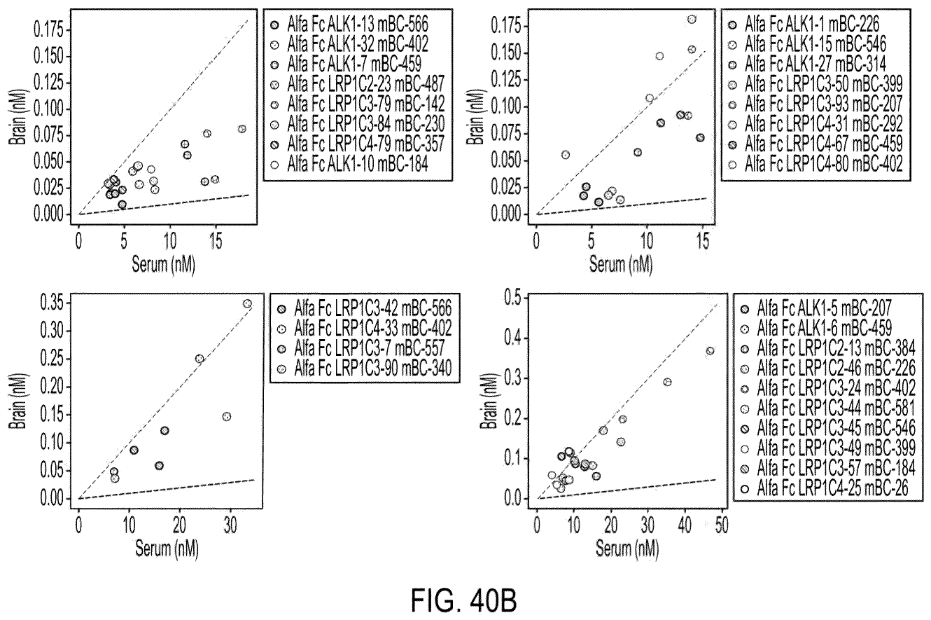

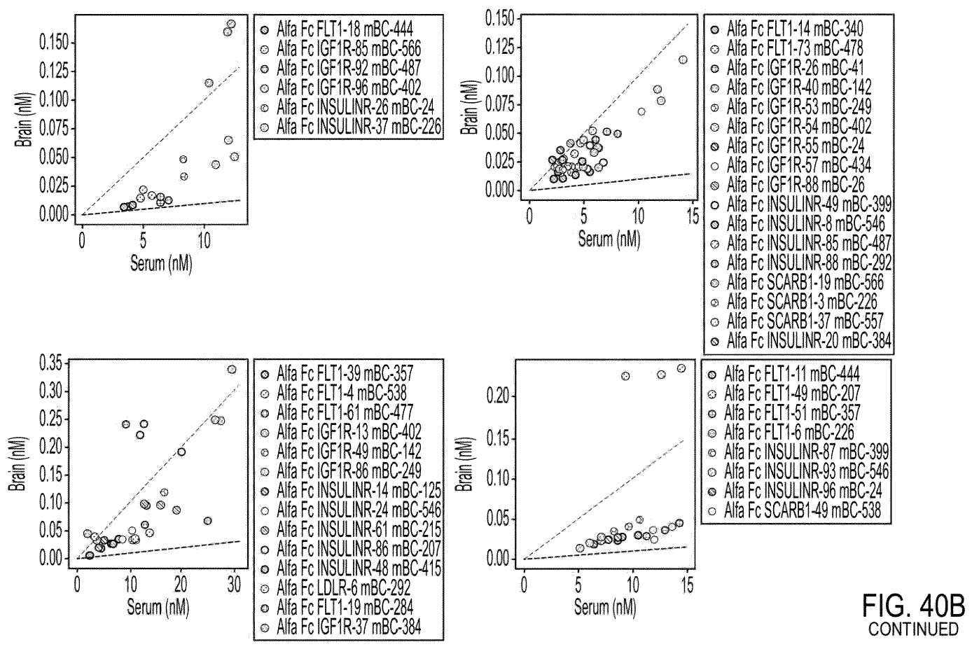

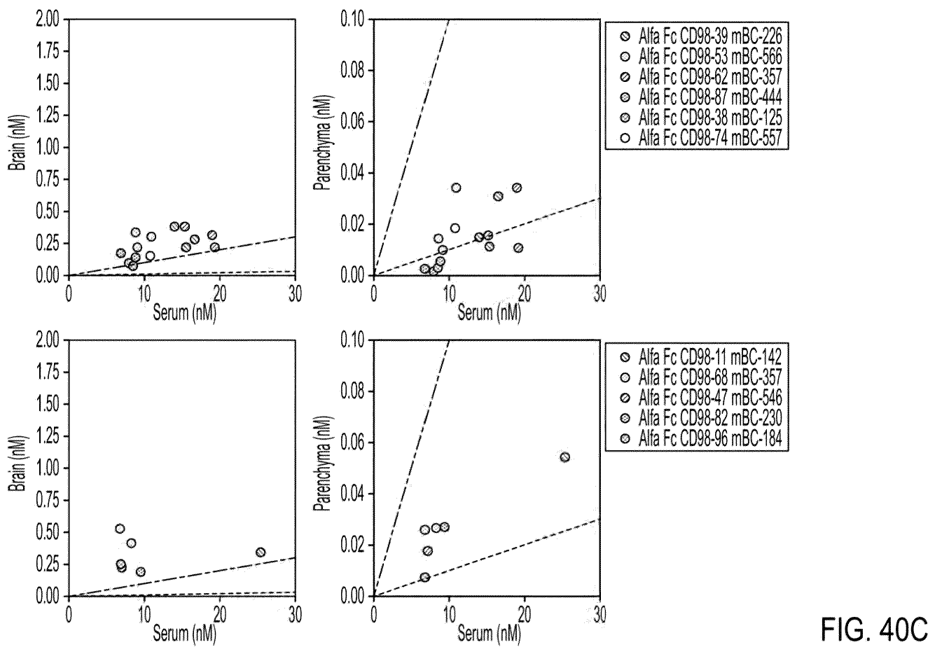

Molecules were quantified using decoding protocol described herein. Briefly, lysed tissue samples were incubated with magnetic beads coated with ALFAtag single-domain antibody (ALFA Selector ST, Nanotag Biotechnologies, Germany) added at 10 μL per well and incubated shaking at 900 rpm to ensure thorough mixing of beads for 1.5 hours at room temperature (RT), plates were then moved to 4° C. to continue capturing overnight. Beads were washed once with 1×PBS and the appropriate scFv decoder phage library (1E10 pfu/mL) was added to bind the barcodes for 2 hours at RT, shaking at 900 rpm. Beads were then washed three times to remove non-specifically binding phages in 1×PBST (0.1% Tween-20) using an extractor shield to carefully transfer the beads to each wash plate. After the final wash, beads were incubated in Trypsin (0.1 μg/mL, Sigma Aldrich) for 1 hour at 37° C. to elute phages off of the beads. The eluate was then added to E. coli TG1 cells (LGC, Biosearch Technologies) in exponential phase (OD600=0.4) and incubated for 1 hour at 37° C., shaking at 1000 rpm. Carbenicillin (120 μg/mL, Sigma Aldrich) and Glucose (1%) were added to LB media (Sigma Aldrich) and 1 mL of this prepared media was added to the bacterial cells infected with phages and incubated shaking at 1000 rpm for 16 hours at 37° C. Samples were then subjected to NGS library preparation where 1 μL of cells were added to Q5 High-Fidelity 2× Master Mix (NEB) with appropriate forward and reverse primers (0.75 μM final concentration) to amplify the scFv decoder insert and libraries were amplified with 13 cycles of PCR. DNA libraries were then indexed with unique dual index primers (i5 and i7) to enable sequencing and the libraries were pooled together post-indexing. Libraries were then purified using SPRI bead purification (CleanNGS DNA purification magnetic beads) using an amount appropriate for the desired size selection and DNA was eluted in a buffer containing 10 mM Tris-HCl (pH 8.0) with 0.1 mM EDTA. DNA libraries were then sequenced with Illumina's NextSeq 2000 sequencer, generating paired end reads of 150 bp. Precise quantification of barcode amounts in the tissue samples from NGS data was enabled by including a standard curve that was decoded with known amounts of protein in commercially purchased (Transnetyx) strain-matched control tissues lysed with the above-mentioned lysis protocol under a larger scale (GentleMacs). A known amount of control phage was also added to every well for normalization of the NGS signal across all the wells thereby enabling accurate and meaningful quantification of barcode amounts in the wells. See FIG. 40A.

### Acute Symptom Evaluation:

Wild-type (C57), hTfR1, or hVEGFR1 mice were administered a single dose of respective treatment and returned to their home cage for acute sign observations by a blinded experimenter. Observations were recorded for up to 30 minutes post-injection, focusing on clinical symptoms such as post-dose lethargy, rough coat appearance, hunching, and occasional limb or whole-body spastic movements. After the 30-minute observation period, mice were returned to their racks, and a second round of observations began 3 hours post-dose. Any symptoms noted during the initial round of observations were re-evaluated during this second round, and these were completely reversed at this time.

### Results

FIG. 40B depicts brain concentration vs serum for a subset of molecules screened. The bottom line depicts 0.1% brain/serum concentration, and the upper line displays 1%. Hits are selected for increased brain uptake properties from the given comparisons.

FIG. 40C show data related to CD98, using a humanized CD98 mouse. Hits were selected similarly as above based on ratio of brain/serum concentration.

FIG. 47A-47D depicts plots showing multiplexed in vivo screening data of shuttle candidates. The diagonal lines with percentages denote brain/serum ratios. FIG. 47A depicts plots related to protein-barcoded anti-TfR1 nanobodies screened in multiplex in humanized TfR1 mice (n=3), with takedown and analysis of serum and whole-brain tissue conducted at 24 hrs post-injection. A total of 250 total molecules were screened and those nanobodies detected in brain are shown. FIG. 47B depicts plots related to protein-barcoded anti-CD98hc nanobodies screened in multiplex in humanized CD98hc mice (n=3), with takedown and analysis of serum and whole-brain tissue conducted at 72 hrs post-injection. FIG. 47C depicts plots related to 295 protein-barcoded nanobodies directed to additional unique (e.g., not previously known) targets (n=7) screened in multiplex in C57 mice, with takedown and analysis of serum and whole-brain tissue conducted at 72 hrs post-injection. Each color represents a unique (e.g., not previously known) target. FIG. 47D depicts plots related to 30 protein-barcoded nanobodies against FLT1 screened in multiplex in C57 mice (n=3), with takedown and analysis of serum and whole-brain tissue conducted at 72 hrs post-injection. Three putative shuttle hits (green stars) were identified with a brain/serum ratio greater than 1%, while the remaining FLT1 shuttle candidates showed little/no brain exposure (red circles). Shuttle candidates against other targets (open circles) are left for reference.

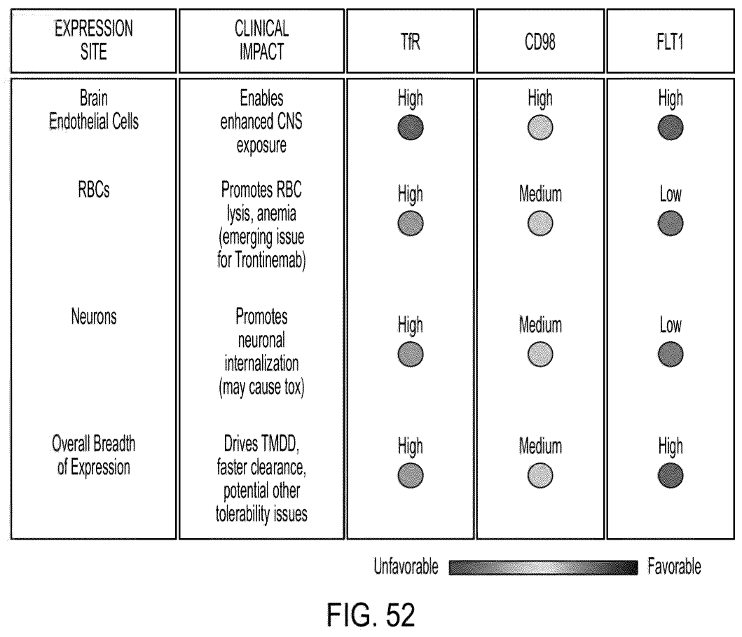

FIG. 52 shows a summary of TfR, CD98, and FLT1 expression profiles. The profiles show that FLT1 and CD98 are supportive of improved tolerability and efficacy for shuttled anti-aβ cargos.

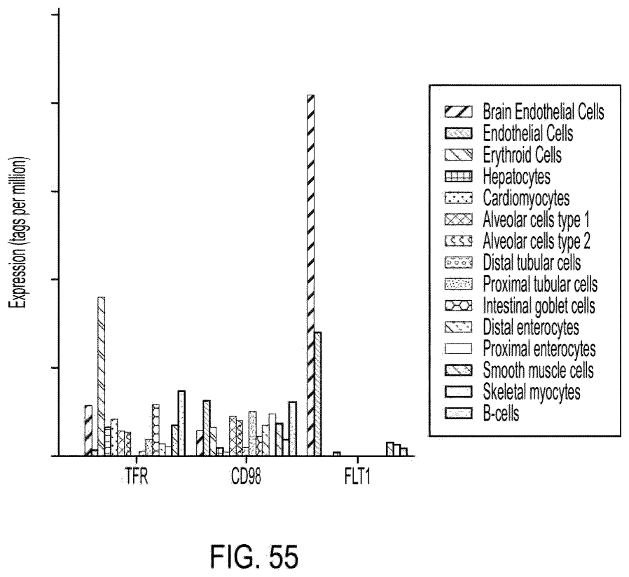

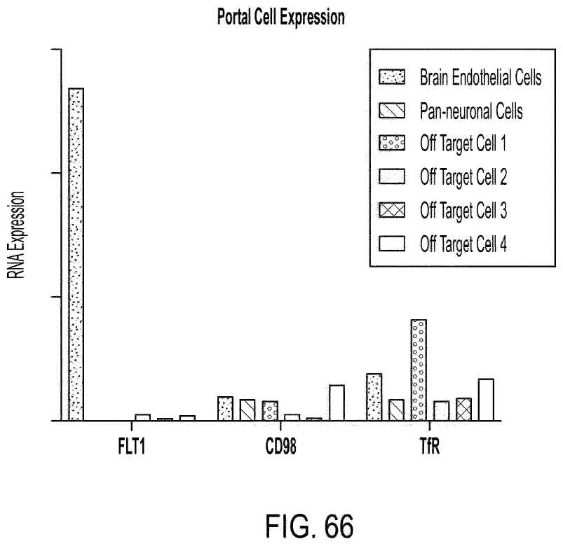

FIG. 55 shows gene expression profiles across various cell types and tissues determined using publicly available scRNA-Seq datasets. Exemplary targets (TfR, CD98, and FLT1) for brain shuttling were nominated based on favorable gene expression profiles, including enrichment on brain endothelial cells plus relatively low expression on peripheral targets (see also FIG. 66).

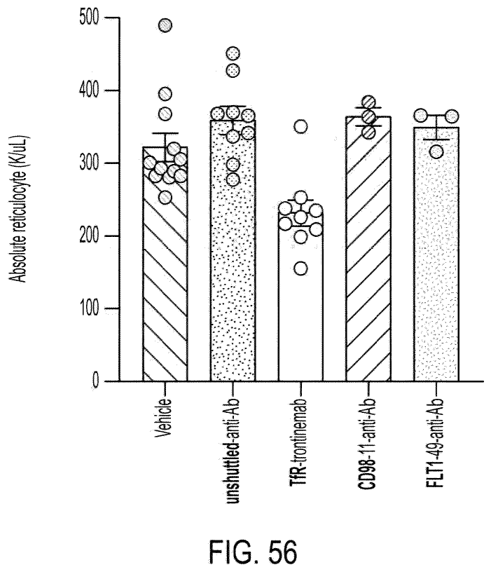

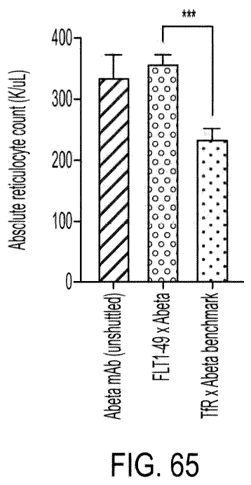

FIG. 56 shows a plot of total reticulocyte count 24 hours after intravenous injection of constructs at 5 mg/kg (n=3-8 mice per group). Anti-Aβ antibody shuttled with FLT1-49 or CD98-11 does not impact reticulocyte counts, relative to unshuttled anti-Aβ antibody (see also FIG. 65).

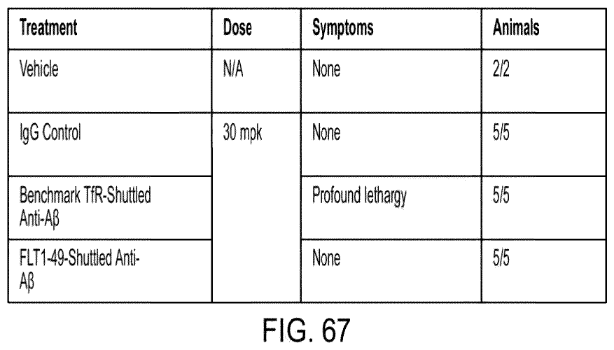

FIG. 67 shows a comparison of acute symptoms in mice following administration of the indicated treatment at the indicated dose level. Wild-type (C57), hTfR1, or hVEGFR1 mice were administered a single dose of Vehicle, IgG (30 mg/kg), Benchmark TfR-shuttled Anti-Aβ(30 mg/kg) or FLT1-49-shuttled Anti-Aβ (30 mg/kg) and observed for acute symptoms by a blinded experimenter.

### Example 32: Multiplexed In Vivo Characterization of Shuttle-IgG Lecanemab Fusions to Different Targets Using Binder-Barcode Platform

The present example demonstrates a method to perform multiplexed in vivo characterization of shuttle-IgG fusions (e.g., shuttle-cargo polypeptides as described herein) designed to target seven different targets of interest using a binder-barcode platform described herein. For example, in some embodiments, multiple shuttle-IgG fusions (e.g., shuttle-cargo polypeptides as described herein) can be identified and/or assessed simultaneously using a binder-barcode platform described herein as described by this example. For example, shuttle-IgG fusions can be identified and/or assessed for brain enrichment. Shuttle-IgG fusions can be selected based on increased concentration in brain cell(s) relative to other cell(s) (e.g., serum). In some embodiments, a shuttle is or comprises a shuttle moiety.

### Method

### Molecule Construction and Production:

Heavy and light chain plasmid DNA was combined at a 1:1.5 ratio for heavy:light chain plasmid to a total mass of 68 μg combined with 12 μg salmon sperm DNA. DNA was complexed with Expifectamine293 according to manufacturer recommendations (Gibco) in a transfection volume of 85 mL. Transfected cells were harvested by centrifugation on day 5. 1 mL cell culture supernatant was transferred to a fresh 96-deep well plate containing 10 μL MagSepharose PrismA beads (Cytiva) in three replicates. Supernatants and beads were incubated at 36.5° C. for 1 hour at 1,000 RPM. Beads were collected, washed, and purified antibodies eluted into 120 μL 100 mM glycine, 150 mM sodium chloride, pH 3.2 using the KingFisher Purification System (Thermo Fisher) and neutralized with 15 μL 1 M Sodium Phosphate, pH 7. Purified antibodies were quantified by BCA and evaluated for aggregation by size exclusion chromatography. Antibodies of sufficient quality were combined equimolar by dosing group for pooled purification. Purification was performed on an Akta Pure25 with a HitTrap MabSelect SuRe pcc column at 5 mL/min. Pooled cell culture supernatant was loaded onto the column washed with 20 mM sodium phosphate, 150 mM sodium chloride, 1 pH 7.0 and eluted with 100 mM glycine, 150 mM sodium chloride, pH 3.2 directly into 10% volume of 1M sodium phosphate, pH 7.0. Peak fractions were pooled, concentrated using centrifugal concentrators (10 kDa MWCO, MilliporeSigma). Concentrated antibodies were dialyzed into 20 mM sodium phosphate, 150 mM sodium chloride and diluted to a final concentration of ˜0.5 mg/mL/antibody to dose at 5 mg/kg/antibody (IgG equivalent). Antibody pools were snap frozen in liquid nitrogen and thawed before dosing.

### CD98 Binding Kinetics:

Purified bispecific antibodies were diluted to a final concentration to 5 μg/mL in 1×PBS, 0.5% BSA, 0.05 Tween20 (kinetics buffer) and transferred to the assay plate. HIS-tagged human CD98 was prepared in a 7-point, 2-fold serial dilution starting at 500 nM to 7.8125 nM with a 0 nM control) and transferred to the assay plate. Anti-human IgG Capture biosensors (Version 2, Sartorius) were pre-equilibrated in kinetics buffer for 20 minutes before the start of the assay. At the start of the assay, baseline signal was measured for 1 minute in kinetics buffer before capturing diluted antibodies for 5 minutes. Antibody coated sensors were re-equilibrated in kinetics buffer before measuring binding association for 5 minutes in the serial dilution of CD98 and dissociation for 5 minutes in kinetics buffer. Binding kinetics were fit using a 1:1 binding model and global fit across all concentrations.

### In Vivo Screening:

Antibodies were pooled into test articles in groups of 10-20 and were injected at 5 mg/kg/antibody. A total of 3 mice per TA group per time point were systemically dosed via tail vein. After 6, 24 and 72 hours, animals were sacrificed and blood was collected in serum separator tubes via cardiac puncture. Prior to organ collection, mice were transcardially perfused with PBS. The brain was divided into two hemispheres and along with muscle, liver, heart and lungs, all tissues were snap frozen and stored at −80° C.

### Tissue Processing:

One hemisphere of brain tissues (right side) was lysed using TPER (ThermoFisher) buffer with Halt protease inhibitor (ThermoFisher, 1:100) where 1 mL buffer was added to every 150 mg tissue in a TissueLyzer (Qiagen) on at 30 rpm for 7 min using a stainless steel bead of 5 mm (Qiagen). Upon ensuring that the sample is lysed, tubes are centrifuged at 7000 g at 4° C. for 20 min. The supernatant was collected representing the whole brain fraction and was subjected to quantification using decoding protocol described herein.

For parenchymal extraction, the other brain hemisphere (left) was minced into small pieces with a scalpel in an HBSS buffer containing 10 mM HEPES. The sample was homogenized using a long 21 G cannula (0.8 mm×120 mm) mounted on a 2 mL syringe by aspirating and pushing out 20 times. Homogenate was transferred to 2 mL tubes and centrifuged at 2000 g at 4° C. for 10 min. Supernatant here represents the whole parenchyma while the pellet, when lysed, represents the myelin and vessels fraction. Whole parenchyma was further processed into cell free and cell-associated fractions. For other tissues (muscle, heart, liver and lung), the above-mentioned lysis protocol was followed. All supernatants post-lysis were collected and subjected for quantification through decoding protocol described herein.

### Quantification:

Molecules were quantified using decoding protocol described herein. Briefly, lysed tissue samples were incubated with the appropriate scFv decoder phage library (1E10 pfu/mL) to bind the barcodes for 2 hours at room temperature (RT) shaking at 600 rpm. Magnetic beads coated with ALFAtag single-domain antibody (ALFA Selector ST, Nanotag Biotechnologies, Germany) were then added at 10 μL per well and incubated shaking at 900 rpm to ensure thorough mixing of beads for 1.5 hours at RT. Beads were then washed four times to remove non-specifically binding phages in 1×PBST (0.1% Tween-20) using a KingFisher bead washing station, after which they were incubated in Trypsin (0.1 μg/mL, Sigma Aldrich) for 1 hour at 37° C. to elute phages off of the beads. The eluate was then added to E. coli TG1 cells (LGC, Biosearch Technologies) in exponential phase (OD600=0.4) and incubated for 1 hour at 37° C., shaking at 1000 rpm. Carbenicillin (120 μg/mL, Sigma Aldrich) and Glucose (1%) were added to LB media (Sigma Aldrich) and 1 mL of this prepared media was added to the bacterial cells infected with phages and incubated shaking at 1000 rpm for 16 hours at 37° C. Samples were then subjected to NGS library preparation where 1 μL of cells were added to Q5 High-Fidelity 2× Master Mix (NEB) with appropriate forward and reverse primers (0.75 μM final concentration) to amplify the scFv decoder insert and libraries were amplified with 13 cycles of PCR. DNA libraries were then indexed with unique dual index primers (i5 and i7) to enable sequencing and the libraries were pooled together post-indexing. Libraries were then purified using SPRI bead purification (CleanNGS DNA purification magnetic beads) using an amount appropriate for the desired size selection and DNA was eluted in a buffer containing 10 mM Tris-HCl (pH 8.0) with 0.1 mM EDTA. DNA libraries were then sequenced with Illumina's NextSeq 2000 sequencer, generating paired end reads of 150 bp. Precise quantification of barcode amounts in the tissue samples from NGS data was enabled by including a standard curve that was decoded with known amounts of protein in commercially purchased (Transnetyx) strain-matched control tissues lysed with the above-mentioned lysis protocol under a larger scale (GentleMacs). A known amount of control phage was also added to every well for normalization of the NGS signal across all the wells thereby enabling accurate and meaningful quantification of barcode amounts in the wells.

### Results

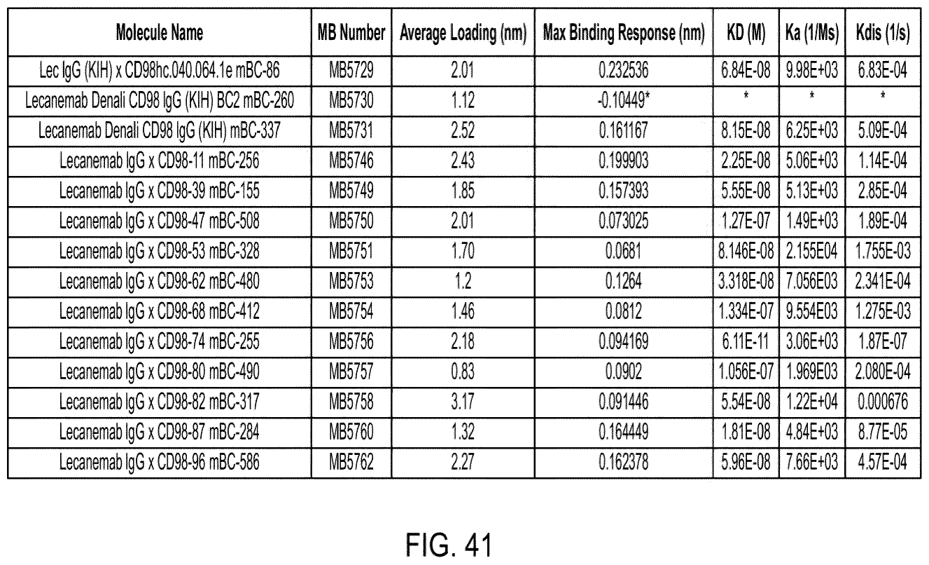

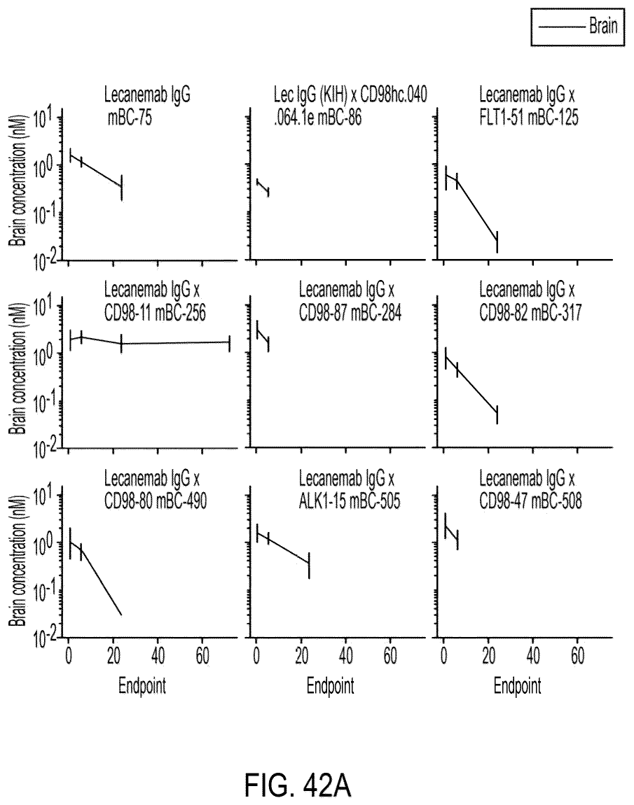

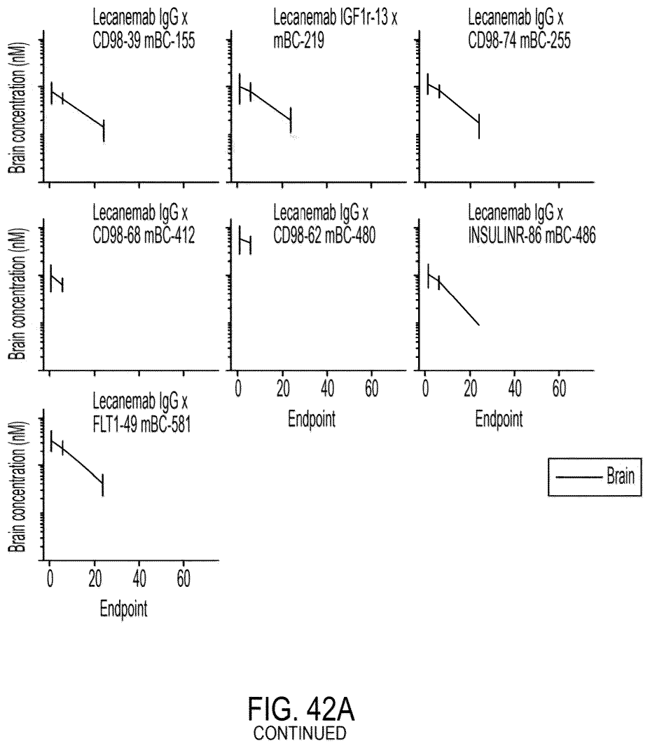

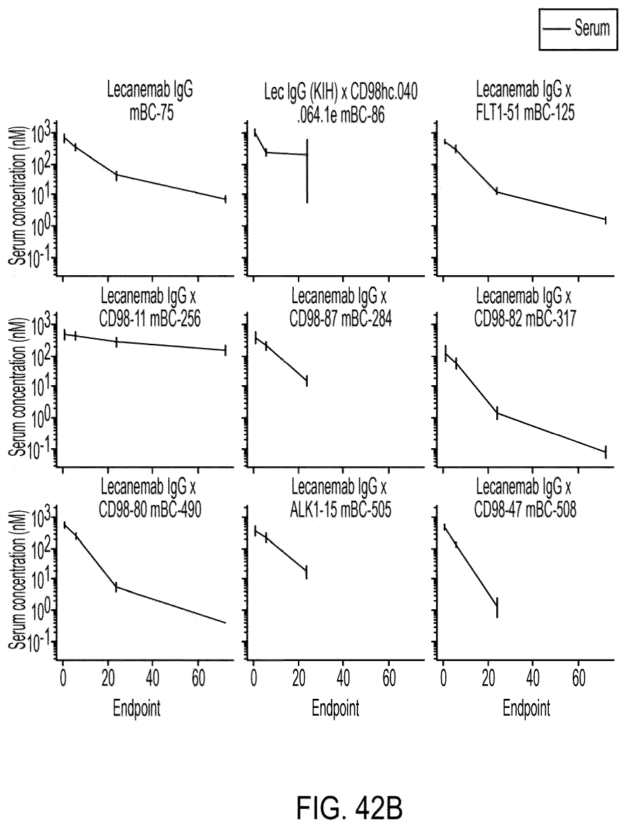

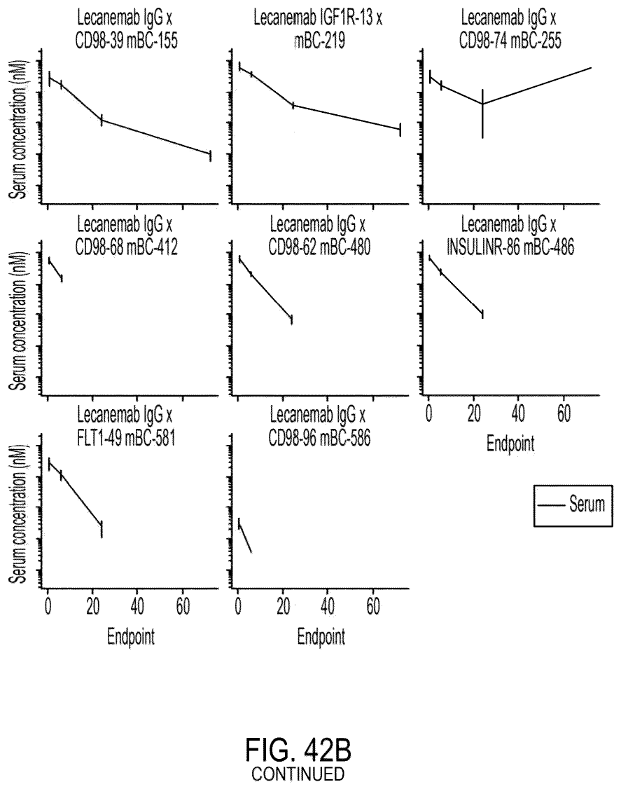

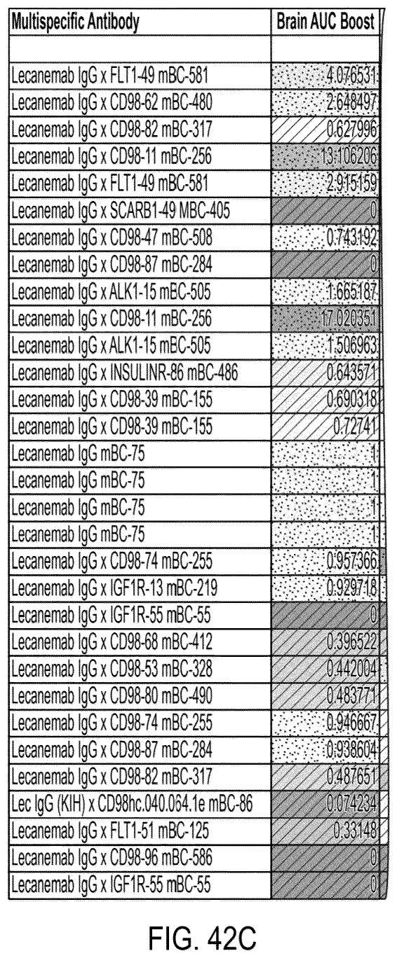

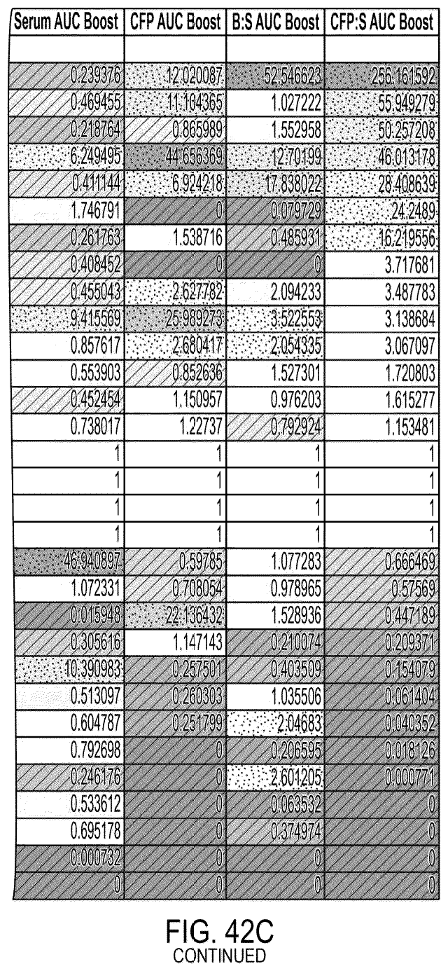

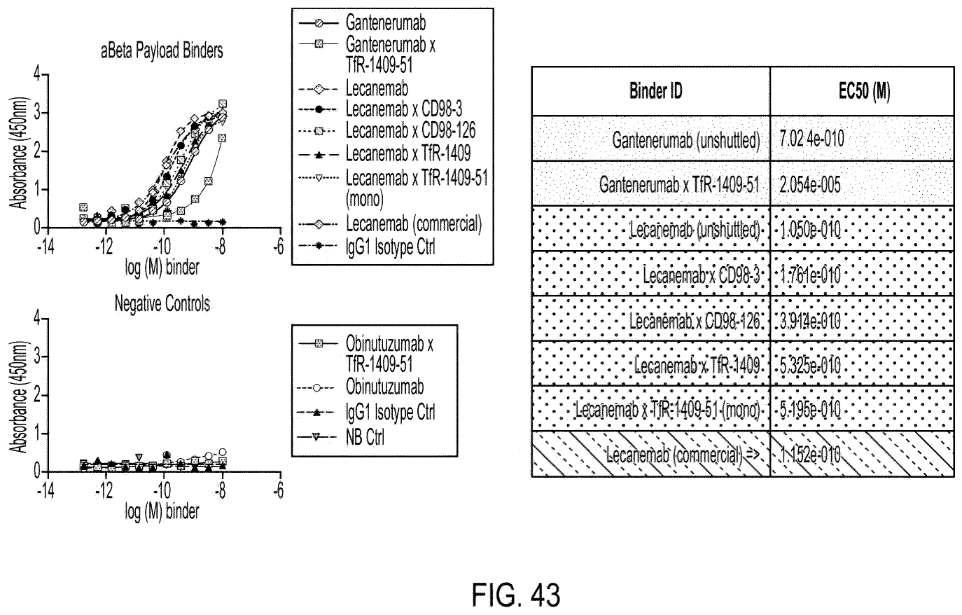

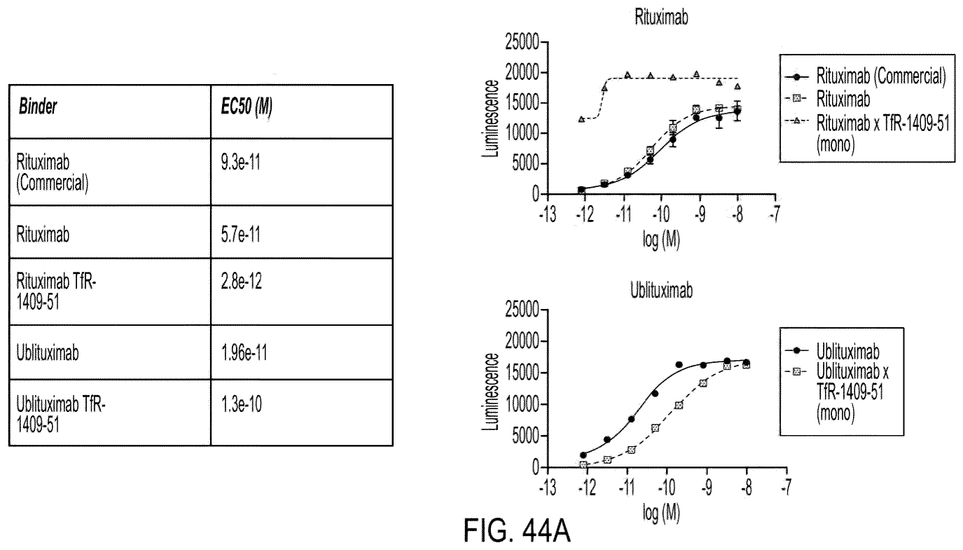

FIG. 41 shows affinities for CD98 targets. Notably, affinity is poorly correlated with the PK for molecules shown in FIG. 43. Lecanemab when fused to shuttle CD98-11 showed increased PK in the serum as well as increased brain uptake, with a brain residence time. Other interesting molecules identified by the present Example included FLT1-49, a shuttle targeting FLT1, which when fused to lecanemab showed approximately 4-fold improved AUC when compared to lecanemab alone. FIGS. 42A-42C depicts plots showing PK data for tested fusion proteins (e.g., shuttle-cargo polypeptides). FIG. 42A depicts plots showing PK data for shuttle-cargo fusion proteins for brain tissue (X-axis shows time in hours and Y-axis shows PK concentration in nM). FIG. 42B depicts plots showing PK data for shuttle-cargo fusion proteins for serum (X-axis shows time in hours and Y-axis shows PK concentration in nM). FIG. 42C shows AUC values for the tested shuttle-cargo fusion proteins to un-shuttled lecanemab. Several molecules showed varied phenotypes when compared to the Fc based constructs screen, likely due to the effects lecanemab has on PK, and highlighting an important feature of in vivo quantification across several shuttle constructs on the payload of interest.

FIG. 48 depicts plots showing in vivo PK validation of TfR1 and CD98hc shuttles with a common anti-amyloid beta (Aβ) antibody payload. Shown are shuttled and unshuttled anti-aβ mAbs with timepoints measured at 1, 6, 24, and 72 hours post-injection. The right plot shows kinetics of shuttle CD98-11 with anti-aβ measured in humanized CD98hc mice.

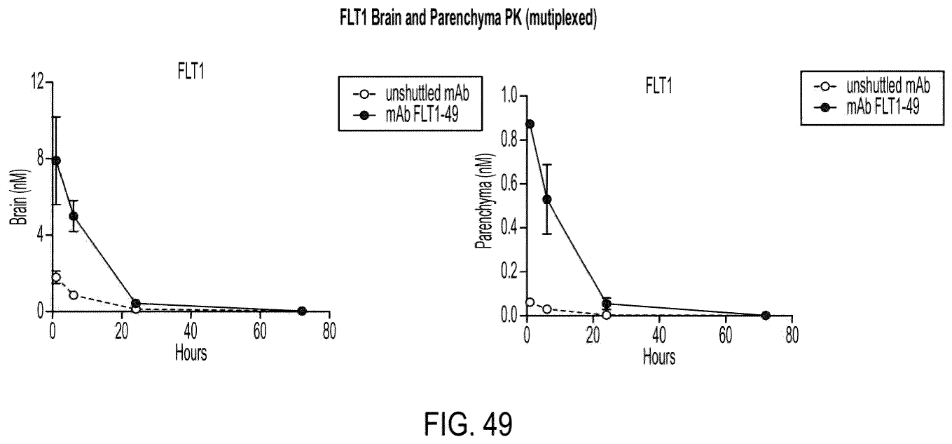

FIG. 49 depicts plots showing in vivo PK validation of a unique (e.g., not previously known) target FLT1 using shuttle FLT1-49 in C57 mice. Shown are shuttled and unshuttled anti-aβ mAbs with timepoints measured at 1, 6, 24, and 72 hours post-injection. The left plot shows quantitation of FLT1-49 in whole-brain. The right plot shows quantitation of FLT1-49 in parenchymal-extracted tissues. This data demonstrates that FLT1 is safer and more effective than other shuttle Aβ therapies. Additionally, FLT1, a BBB endothelial cell membrane protein appears to possess the optimal properties for shuttling an anti-Aβ antibody cargo to the brain to treat various diseases, including Alzheimer's. For example, in some embodiments, its properties include enriched on brain endothelial cells, low expression on other brain cells, low expression on other common sites of toxicity (e.g., erythroid precursors), and fast Parenchymal uptake (see FIG. 49 right plot).

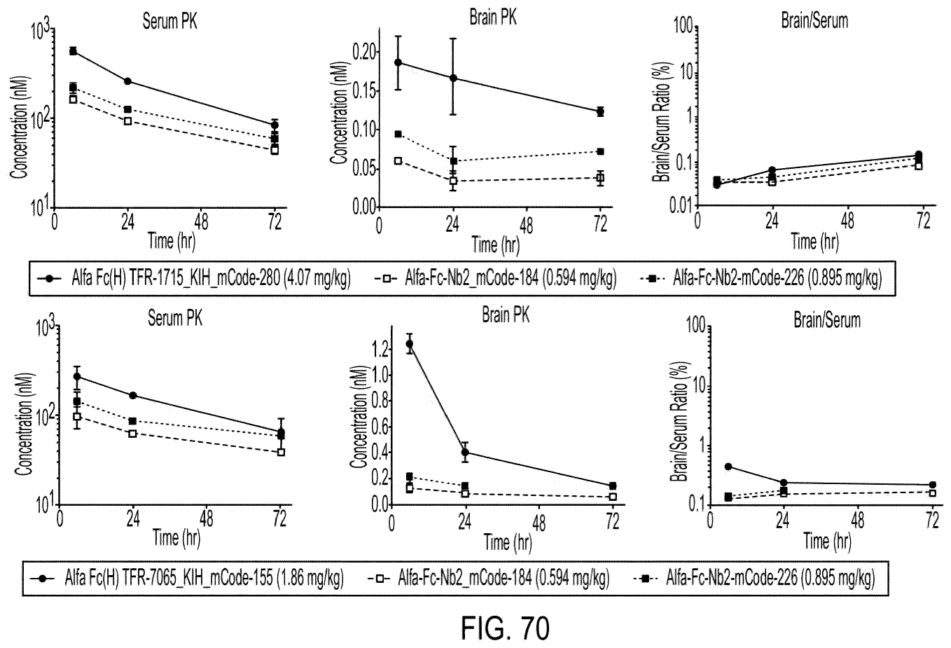

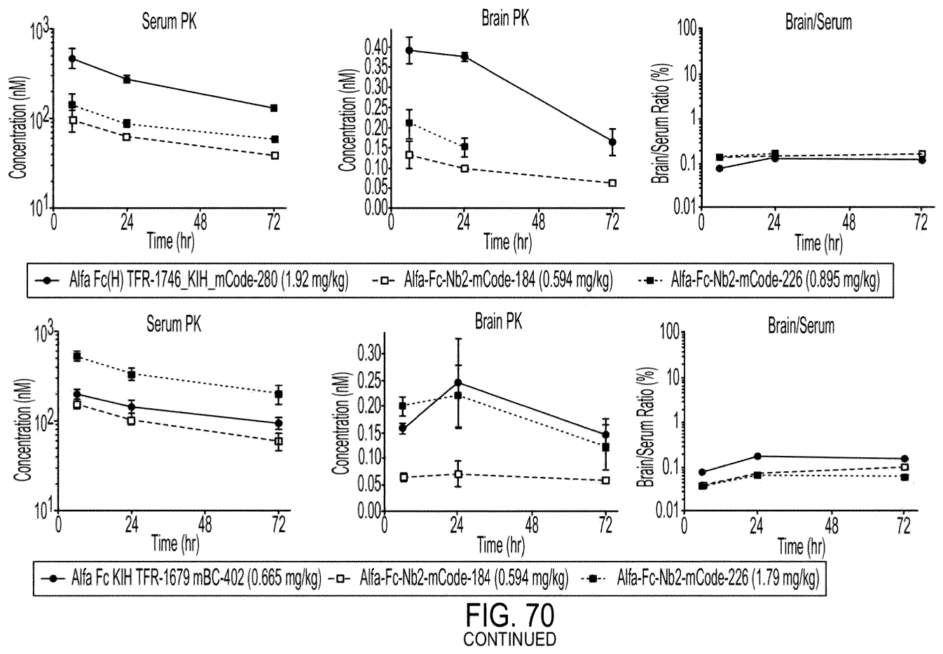

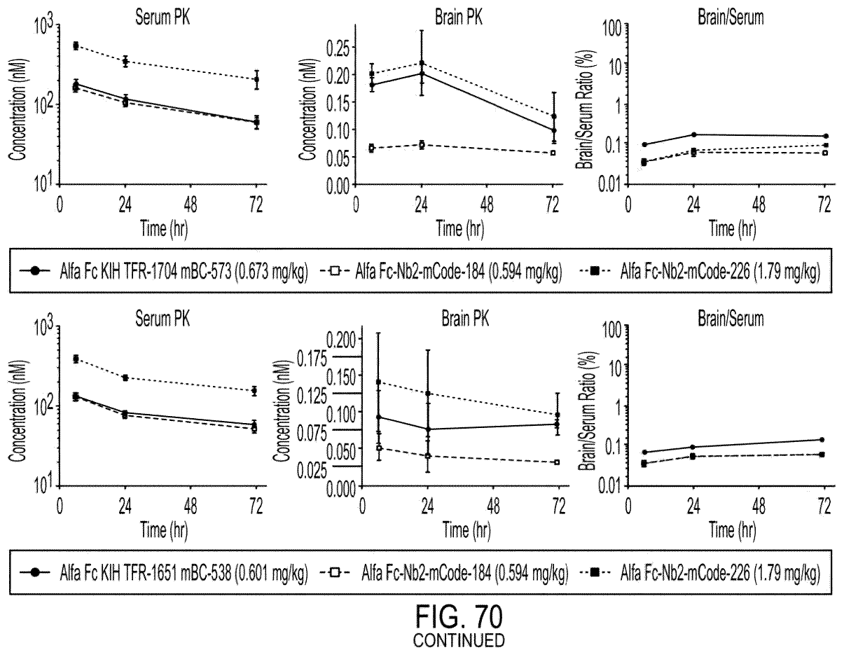

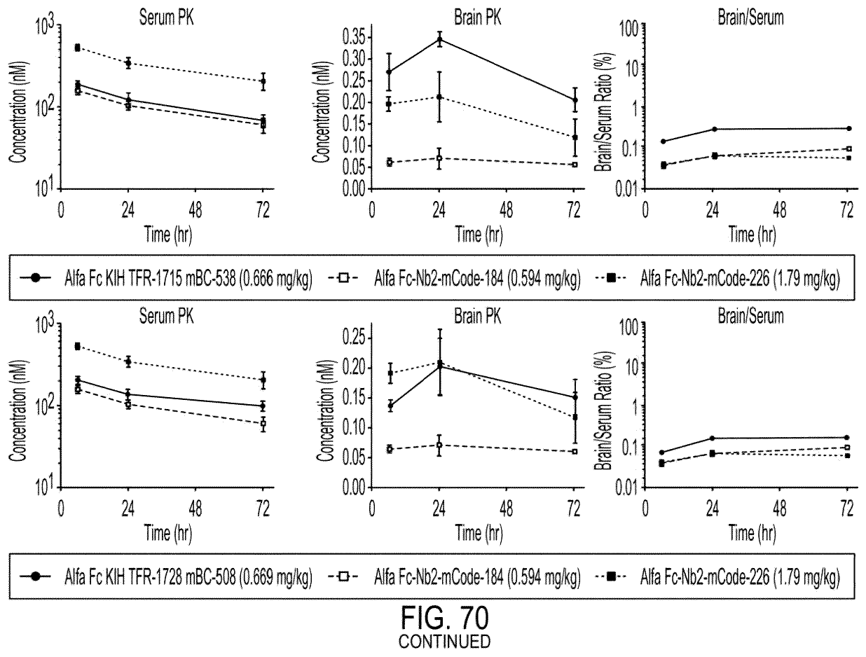

FIG. 70 depicts plots showing pharmacokinetic (PK) profiles of selected TfR shuttles (blue) vs two unshuttled controls (gray and black) across serum, brain tissue, and corresponding brain to serum ratio as measured in humanized TfR mice. These data demonstrate that multiple exemplary TfR shuttles show brain enrichment vs unshuttled controls in humanized TfR mice.

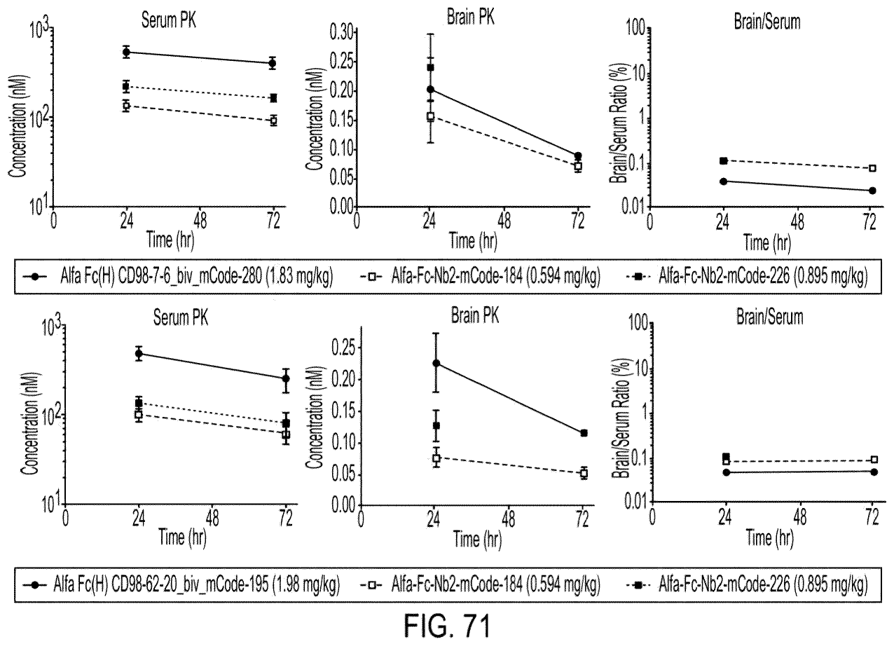

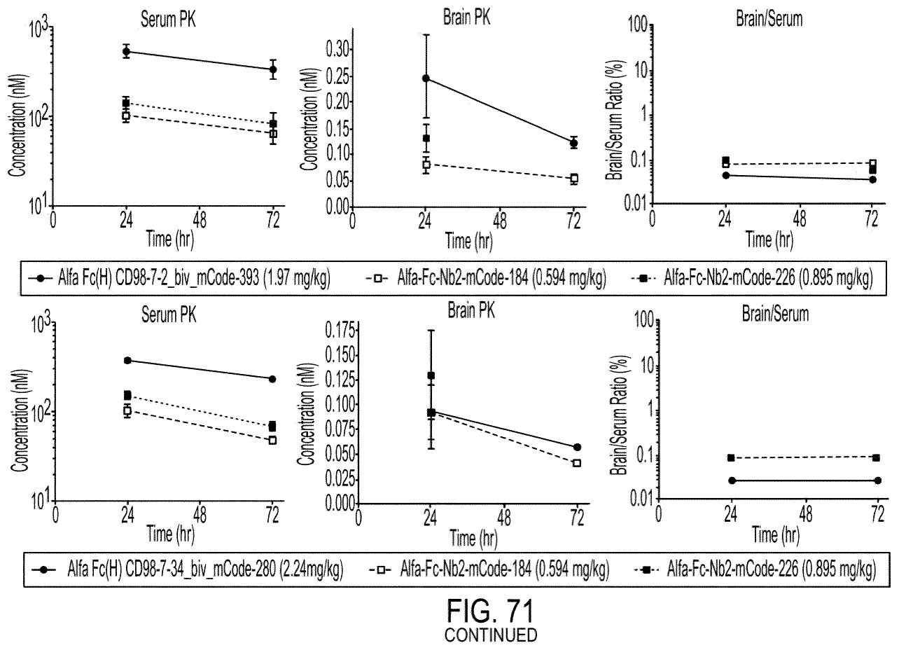

FIG. 71 depicts plots showing pharmacokinetic (PK) profiles of selected CD98 shuttles (blue) vs two unshuttled controls (gray and black) across serum, brain tissue, and corresponding brain to serum ratio as measured in humanized CD98 mice. These data demonstrate that multiple exemplary CD98 shuttles show brain enrichment vs unshuttled controls in humanized CD98 mice.

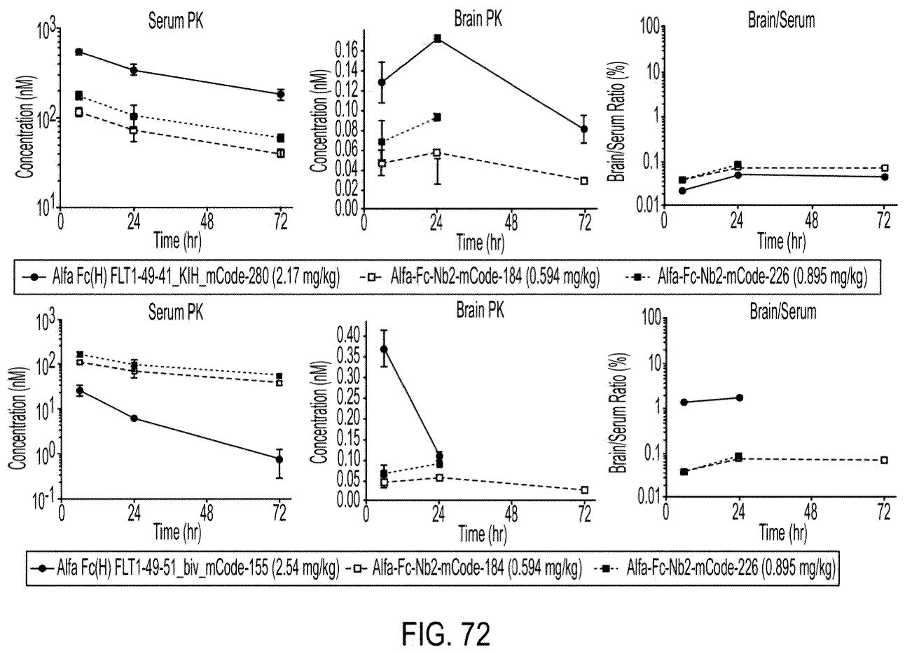

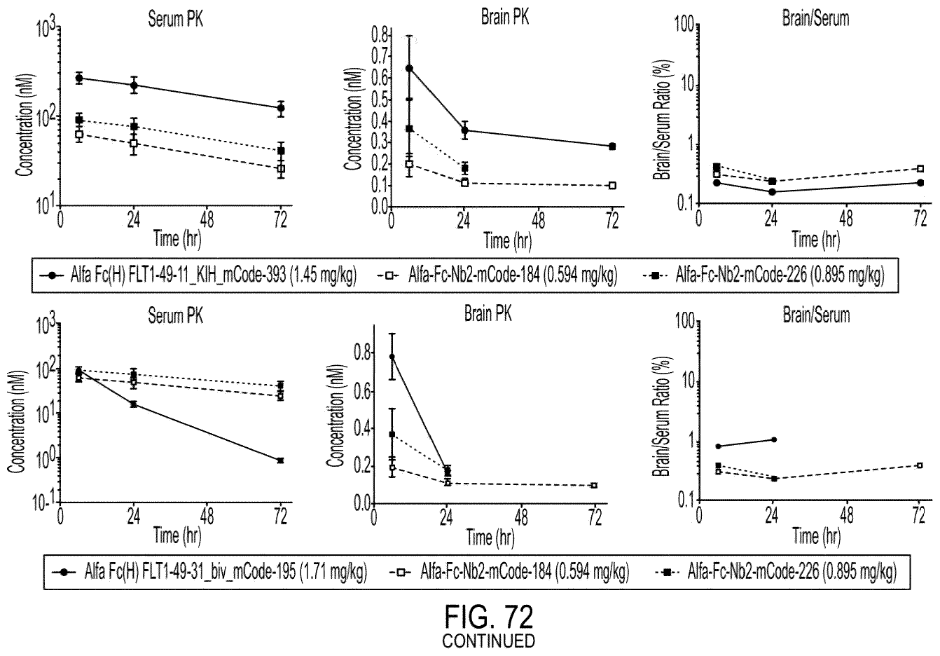

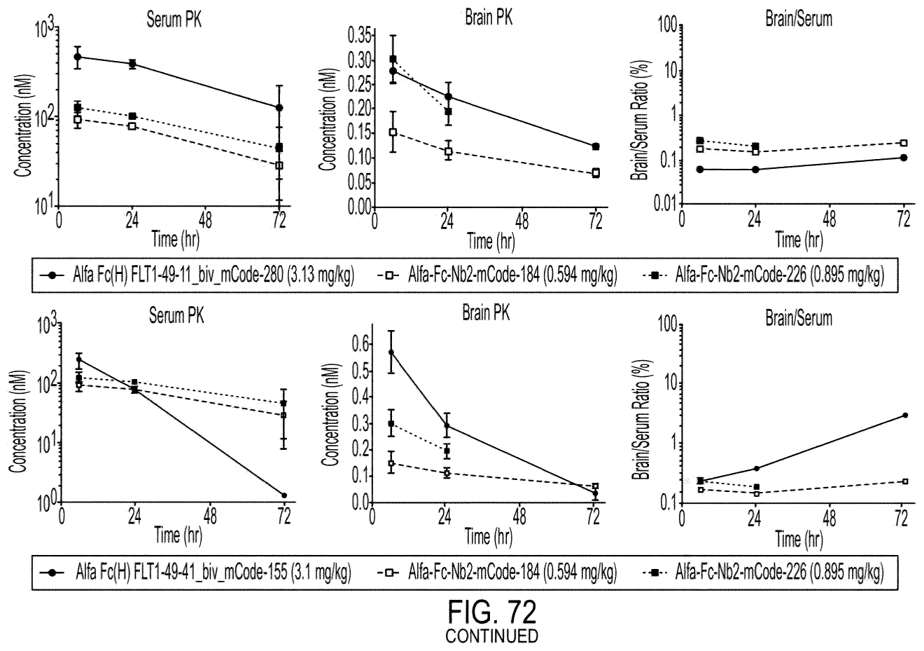

FIG. 72 depicts plots showing pharmacokinetic (PK) profiles of selected FLT1 shuttles (blue) vs two unshuttled controls (gray and black) across serum, brain tissue, and corresponding brain to serum ratio as measured in humanized FLT1 mice. These data demonstrate that multiple exemplary FLT1 shuttles show brain enrichment vs unshuttled controls in humanized FLT1 mice.

### Example 33: Characterization of Shuttle-aBACE1 Fusion Competition with Transferrin (Tf) for Binding to TfR Using Binder-Barcode Platform

The present example demonstrates a method to characterize shuttle-cargo polypeptides in a competition assay.

### Method

### Molecule Construction and Production:

Heavy and light chain plasmid DNA was combined at a 1:1.5 heavy:light chain plasmid ratio for symmetrical antibodies or 1:1:3 heavy (knob):heavy (hole):light chain plasmid ratio for asymmetrical antibodies to a total mass of 0.8 μg per mL of transfection, combined with 0.2 μg salmon sperm DNA per mL of transfection. DNA was complexed with Expifectamine293 (Gibco) according to manufacturer recommendations. Transfected cells were harvested by centrifugation on day 5. 800 μL of cell culture supernatant was transferred to a fresh 96-deep well plate containing 12.5 μL MagSepharose PrismA beads (Cytiva). Supernatants and beads were incubated at 36.5° C. for 1 hour at 1,000 RPM. Beads were collected, washed, and purified antibodies eluted into 120 μL 100 mM glycine, 150 mM sodium chloride, pH 3.2 using the KingFisher Purification System (Thermo Fisher) and neutralized with 15 μL 1 M Sodium Phosphate, pH 7. Purified antibodies were quantified by BCA, snap frozen in liquid nitrogen, and thawed prior to use.

### TfR Competition ELISAs:

For the study showing variant TfR-1409-51 binding to TfR, rh-TfR was added to a Maxisorp Nunc plate at a concentration of 5 μg/ml and at a volume of 100 μL/well, then incubated at 4 C on plate shaker overnight. After decanting and washing 3× with PBST, the plate was blocked by adding 100 μL of PBST (0.1% Tween)+2% BSA to each well for 1 h at RT on the plate shaker. A TfR-1409-51×aBACE1 bispecific antibody construct was prepared in either PBST (0.05% Tween) or PBST+1 μM transferrin. TfR×aBACE1 was prepared at a starting concentration of 1 μM and serially diluted 1:3 in the relevant buffer solution. Following plate blocking, plates were decanted and washed 3× with PBST prior to addition of TfR×aBACE1 dilutions and incubation on the plate shaker for 1 h at RT. After decanting and washing 3× with PBST, aIgG-HRP was added at a dilution of 1:2000 for 1 h at RT. ELISA plates were developed via TMB substrate and absorbance read at 450 nm.

For the study showing Tf binding to TfR, rh-TfR was added to a Maxisorp Nunc plate at a concentration of 5 μg/ml and at a volume of 100 μL/well, then incubated at 4 C on plate shaker overnight. After decanting and washing 3× with PBST, the plate was blocked by adding 100 μL of PBST+2% BSA to each well for 1 h at RT on the plate shaker. A TfR-1409-51×aBACE1 bispecific antibody construct was prepared in PBS at a concentration of 1 uM. Following plate blocking, plates were decanted and washed 3× with PBST prior to addition of either 100 μL TfR×aBACE1 solution or 100 μL PBS control, then incubated on the plate shaker for 1 h at RT. Biotinylated Tf was prepared at a starting concentration of 400 nM and serially diluted 1:3 in PBS. 5 ul of biotinylated Tf dilutions were directly added ELISA wells without washing, for ˜20× dilution and a maximum concentration of 20 nM biotinylated Tf, then incubated on the plate shaker for 1 h at RT. After decanting and washing 3× with PBST, streptavidin-HRP was added at a dilution of 1:4000 for 1 h at RT. ELISA plates were developed via TMB substrate and absorbance read at 450 nm.

### Results

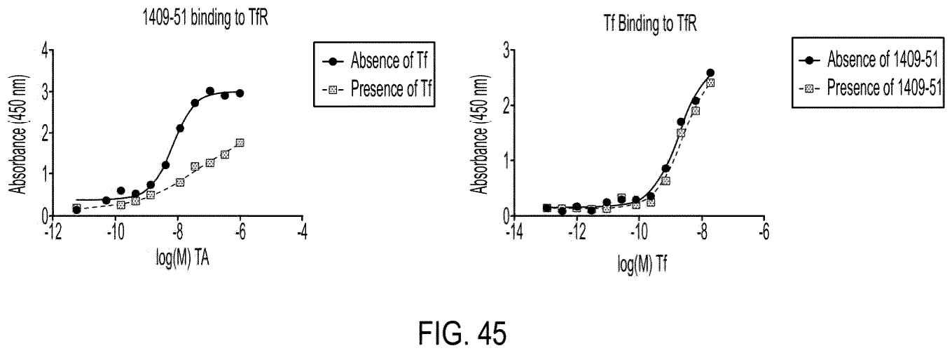

FIG. 45 shows that binding to TfR by TfR-1409-51 was reduced in the presence of Tf, however the inverse was not true. This model demonstrated that Tf should remain bioactive in the presence of TfR-1409-51, reducing the risk of toxicity, but that TfR-1409-51 may share an overlapping epitope with Tf. This partial competition of Tf may be responsible for in vivo phenotypes described herein.

### Example 34: Multiplexed In Vitro Evaluation of Therapeutic Antibodies Associated with Shuttles

The present example demonstrates multiplexed in vitro evaluation of multiple therapeutic antibodies associated multiple shuttles (e.g., shuttle moieties). In some embodiments, a shuttle is or comprises a shuttle moiety.

### Method

### Molecule Construction and Production:

Heavy and light chain plasmid DNA was combined at a 1:1.5 ratio for symmetrical antibodies or 1:1:3 heavy (knob):heavy (hole):light chain plasmid to a total mass of 0.8 μg per mL of transfection combined with 0.2 μg salmon sperm DNA per mL of transfection. DNA was complexed with Expifectamine293 (Gibco) according to manufacturer recommendations. Transfected cells were harvested by centrifugation on day 5. 800 μL of cell culture supernatant was transferred to a fresh 96-deep well plate containing 12.5 μL MagSepharose PrismA beads (Cytiva). Supernatants and beads were incubated at 36.5° C. for 1 hour at 1,000 RPM. Beads were collected, washed, and purified antibodies eluted into 120 μL 100 mM glycine, 150 mM sodium chloride, pH 3.2 using the KingFisher Purification System (Thermo Fisher) and neutralized with 15 μL 1 M Sodium Phosphate, pH 7. Purified antibodies were quantified by BCA, snap frozen in liquid nitrogen, and thawed prior to use.

aBeta (Aβ) Binding ELISA:

Pre-formed aBeta fibrils were purchased and diluted to a concentration of 2.5 μg/ml prior to addition to Maxisorp Nunc plates, then incubated on a plate shaker for 2 h at RT. After decanting and washing 3× with PBST, the plate was blocked by adding 200 μL of PBST+2% BSA to each well for 1 h at RT on the plate shaker. Test constructs were prepared at a starting concentration of 10 nM and serially diluted 1:3 in PBST+2% BSA. Following plate blocking, plates were decanted and washed 3× with PBST prior to addition of test construct solutions and incubated on the plate shaker for 1 h at RT. After decanting and washing 3× with PBST, aIgG-HRP was added at a dilution of 1:2000 for 1 h at RT. ELISA plates were developed via TMAB substrate and absorbance read at 450 nm.

### ADCC Assays:

The ADCC reporter bioassay kit from Promega, based on an NFAT luciferase readout, was used to evaluate the activity of test constructs. Assay buffer was comprised of RPMI 1640+4% low IgG serum. Raji cells were plated at 12,500 cells/well on white 96 well assay plates in 25 μL assay buffer/well. Test construct serial dilutions were prepared at a starting concentration of 10 nM and serially diluted 1:3 in 25 μL assay buffer/well. 75,000 effector Jurkat cells were subsequently added in 25 μL assay buffer. Plates were incubated at 37 C+5% CO2 for 3 h. To develop and read luminescence signal, assay plates were removed from the incubator and equilibrated to RT for 15 min. 75 ul of Bio-Glo was added to each well and incubated at RT for 5 min, then luminescence read on a plate reader, using the setting for opaque 96 well plates (integration of 500 ms and gain of 120-135).

### Results

FIG. 43 shows aBeta binding of two different anti-aBeta antibodies (gantenerumab and lecanemab) fused to brain shuttle VHH domains. While binding EC50 was significantly reduced for gantenerumab following fusion with TfR-1409-51, lecanemab fusion affinities remained within ˜5-fold of parental lecanemab across all evaluated VHH fusion domains. No binding was detected in the absence of aBeta antibodies.

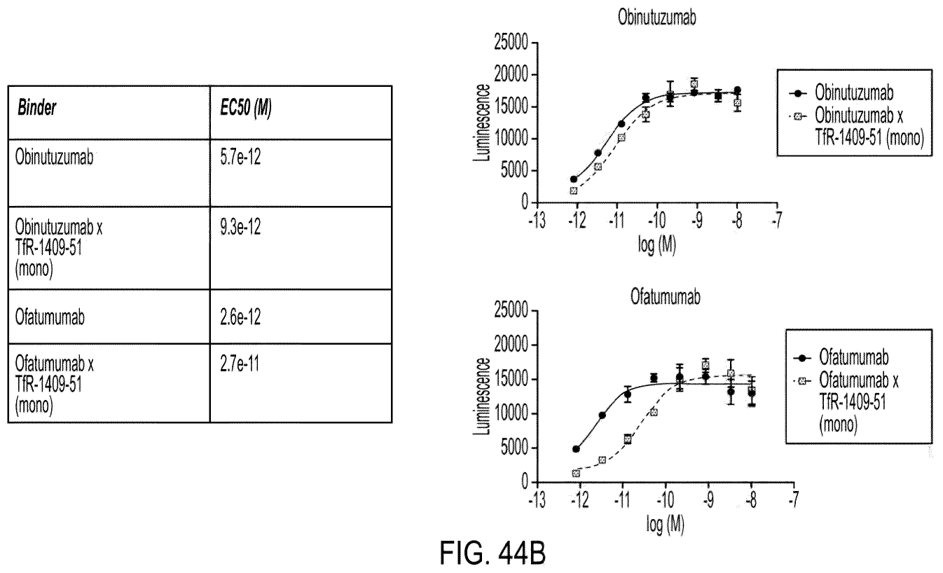

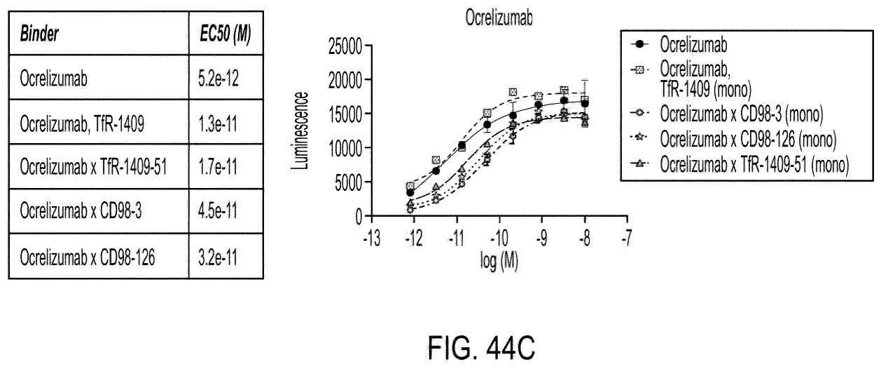

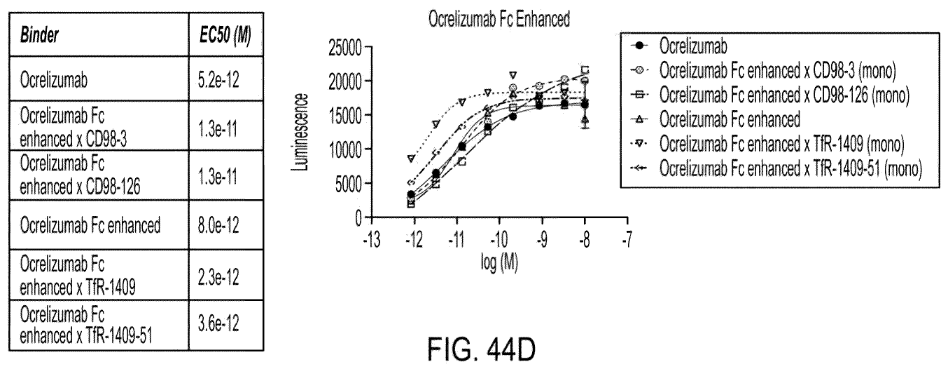

FIGS. 44A- FIG. 44D show the potency of anti-CD20 antibodies (rituximab, ublituximab, obinutuzumab, ofatumumab, and ocrelizumab) fused to brain shuttle VHH domains. Multiple VHH domains (TfR-1409, TfR-1409-51, CD98-3, and CD98-126) as well as Fc enhancing mutations (S239D/1332E) were evaluated with ocrelizumab, while just TfR-1409-51 and WT Fc was evaluated with the other anti-CD20 antibodies. The shift in ocrelizumab fusion ADCC EC50 relative to WT ocrelizumab ADCC ranged from 2.5-fold to 8.7-fold depending on the evaluated VHH domain. Fc enhancement did not significantly impact the activity of ocrelizumab. Of the anti-CD20 antibodies evaluated with TfR-1409-51, rituximab-TfR-1409-51 demonstrated a significant increase in luminescence signal relative to WT rituximab, the ADCC EC50 of ublituximab-TfR-1409-51 shifted 6.6-fold relative to WT ublituximab, the ADCC EC50 of obinutuzumab-TfR-1409-51 shifted 1.6-fold relative to WT obinutuzumab, and the ADCC EC50 of ofatumumab-TfR-1409-51 shifted 10.4-fold relative to WT ofatumumab. In all cases, the parental antibody remained functional following fusion to VHH domains.

### Example 35: Multiplexed In Vivo Screening and Assessment of Phenotypes of TfR Shuttles and Shuttle-IgG Lecanemab Fusions in Cynomolgus Monkeys

The present example demonstrates a multiplexed, high-throughput method of screening shuttles (e.g., shuttle agents or shuttle moieties (e.g., TfR shuttles)) and shuttle-IgG fusions (e.g., shuttle-cargo polypeptides) as described herein. The present example also demonstrates that a binder-barcode platform described herein can be used to assess inducible expression of a delivered cargo polypeptide in monkeys. Moreover, the present example demonstrates that a binder-barcode platform described herein can be used to identify and/or quantify cargo polypeptides and/or shuttle-cargo polypeptides and/or shuttles and/or components thereof for certain phenotypes such as expression and/or localization. For example, in some embodiments, shuttles (e.g., shuttle moieties) can be identified and/or assessed for ability to cross the blood-brain barrier (BBB) (e.g., by binding to a BBB surface receptor and inducing transcytosis into the brain) as described by this example. For example, shuttle-IgG fusions can be identified and/or assessed for brain enrichment. Shuttle-IgG fusions can be selected based on increased concentration in brain cell(s) relative to other cell(s) (e.g., serum). In some embodiments, a shuttle is or comprises a shuttle moiety.

Other types of cargo components can be assessed using a binder-barcode platform as described herein. Phenotypes other than expression and localization can also be quantified and/or assessed using a binder-barcode platform as described herein. Exemplary phenotypes include epitope properties, affinity, thermostability, pH sensitivity, or other phenotypes described herein.

### Method

### Molecule Construction and Production:

Heavy and light chain plasmid DNA was combined at a 1:1.5 ratio for symmetrical antibodies or 1:1:3 heavy (knob):heavy (hole):light chain plasmid to a total mass of 40 μg combined with 10 μg salmon sperm DNA. DNA was complexed with Expifectamine293 according to manufacturer recommendations (Gibco) in a transfection volume of 50 mL. Transfected cells were harvested by centrifugation on day 5. 1 mL cell culture supernatant was transferred to a fresh 96-deep well plate containing 10 μL MagSepharose PrismA beads (Cytiva) in three replicates. Supernatants and beads were incubated at 36.5° C. for 1 hour at 1,000 RPM. Beads were collected, washed, and purified antibodies eluted into 120 μL 100 mM glycine, 150 mM sodium chloride, pH 3.2 using the KingFisher Purification System (Thermo Fisher) and neutralized with 15 μL 1 M Sodium Phosphate, pH 7. Purified antibodies were quantified by BCA and evaluated for aggregation by size exclusion chromatography. Antibodies of sufficient quality were combined equimolar by dosing group for pooled purification. Purification was performed on an Akta Pure25 with a HitTrap MabSelect PrismA column at 5 mL/min. Pooled cell culture supernatant was loaded onto the column washed with 20 mM sodium phosphate, 150 mM sodium chloride, 1 pH 7.0 and eluted with 100 mM glycine, 150 mM sodium chloride, pH 3.2 directly into 10% volume of 1M sodium phosphate, pH 7.0. Peak fractions were pooled and dialyzed for 20 hours against 2×4 L of 20 mM sodium phosphate, 150 mM sodium chloride. Dialyzed antibodies were diluted to a final concentration of ˜0.5 mg/mL/antibody to dose at 5 mg/kg/antibody (IgG equivalent). Antibody pools were snap frozen in liquid nitrogen and thawed before dosing.

### In Vivo Pharmacokinetic Multiplex Study in Ported Cynomolgus Monkeys:

In vivo multiplexing studies in non-human primates (NHP) provided an opportunity to characterize pharmacokinetics (PK), biodistribution, and other properties of shuttles and shuttle-cargo polypeptides. Moreover, in vivo multiplexing were used to perform head-to-head comparisons of shuttles and shuttle-cargo polypeptides within individual animals.

In an initial experiment, antibodies were pooled into a test article of 9 and were injected at around 1.6 mg/kg/antibody for a total dose of 14.4 mg/kg. A total of 2 non-naive cynomolgus monkeys receive a single intravenous (IV) infusion over 30 minutes of test material on Day 0 followed by 0.5 mL of saline to flush the dose from the IV catheter. The end time of dose administration was used to determine target times for blood and Cerebrospinal fluid (CSF) sample collection time points. Blood samples, processed to obtain serum and plasma, and CSF samples were collected at various time points; Days −5, −3, 1, 2, 3, 5, 7, 10, 14, and 21.

In a second experiment, 37 shuttles—each with a unique barcode—were individually expressed and purified, pooled into a single test article, and examined in NHP across multiple tissues and timepoints using compositions and methods described herein.

### Results

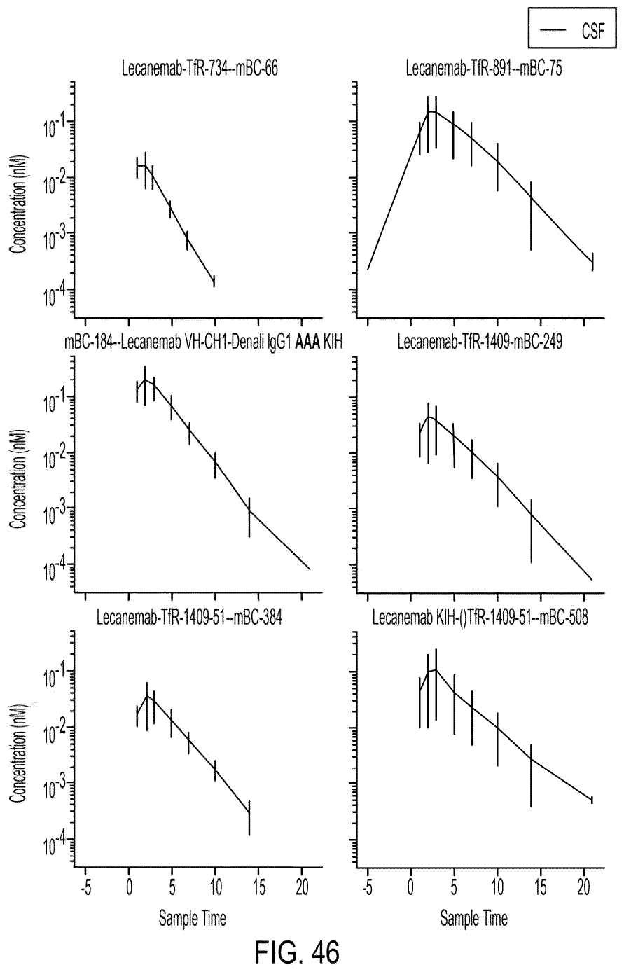

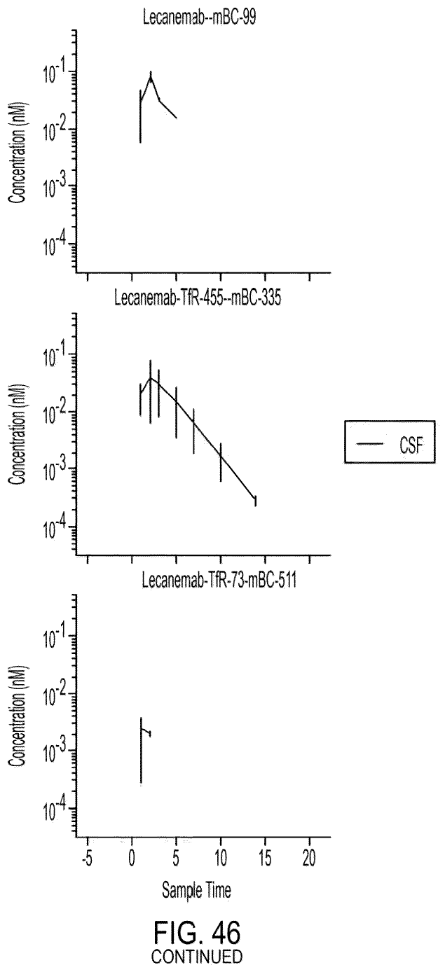

FIG. 46 shows CSF PK for several shuttle variants and shuttle-IgG fusions (e.g., shuttle-cargo polypeptides as described herein) tested in Cynomolgus monkeys. As observed from the plots, monovalent TfR-1409-51 and bivalent TfR-891 were both compatible with the Denali control molecule. Shuttle-cargo fusion Lecanemab-TfR-734, which was previously shown to have poor brain uptake in mouse showed similar low uptake in NHP. Lecanemab by itself has poor visibility and is only seen from 1-5 days but is below limit of detection for the rest of the study.

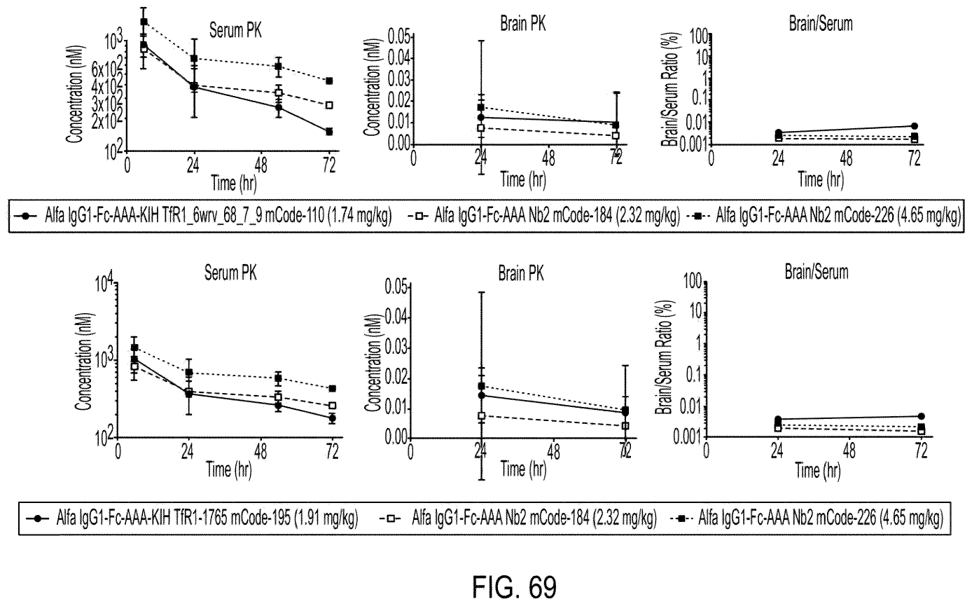

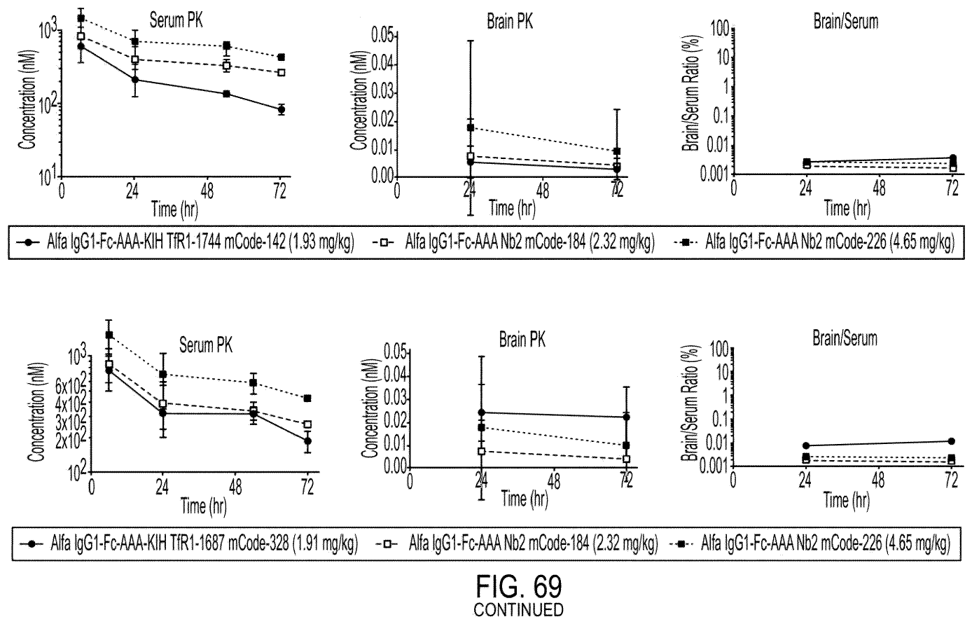

FIG. 69 shows plots of pharmacokinetic (PK) profiles of selected TfR shuttles (blue) vs two unshuttled controls (gray and black) across serum, brain tissue, and corresponding brain to serum ratio as measured in non-human primates (NHP). These data demonstrate that multiple exemplary TfR shuttles show brain enrichment vs unshuttled control in a multiplexed NHP study.

### Example 36: Characterization of TfR-1409-51 Shuttle and Shuttle-IgG Lecanemab Fusions

The present example demonstrates a method to characterize shuttles and shuttle-IgG fusions (e.g., shuttle-cargo polypeptides) as described herein. For example, the present examples demonstrates a method to characterize TfR-1409-51 shuttle and TfR-1409-51-Lecanemab fusion.

### Method

### Molecule Construction and Production:

Heavy and light chain plasmid DNA are combined at a 1:1.5 ratio for symmetrical antibodies or 1:1:3 heavy (knob):heavy (hole):light chain plasmid to a total mass of 40 μg which is combined with 10 μg salmon sperm DNA. DNA is complexed with Expifectamine293 according to manufacturer recommendations (Gibco) in a transfection volume of 50 mL. Transfected cells are harvested by centrifugation on day 5. 1 mL cell culture supernatant is transferred to a fresh 96-deep well plate containing 10 μL MagSepharose PrismA beads (Cytiva) in three replicates. Supernatants and beads are incubated at 36.5° C. for 1 hour at 1,000 RPM. Beads are collected, washed, and purified antibodies eluted into 120 μL 100 mM glycine, 150 mM sodium chloride, pH 3.2 using the KingFisher Purification System (Thermo Fisher) and neutralized with 15 μL 1 M Sodium Phosphate, pH 7. Purified antibodies are quantified by BCA and evaluated for aggregation by size exclusion chromatography. Antibodies of sufficient quality are combined equimolar by dosing group for pooled purification. Purification is performed on an Akta Pure25 with a HitTrap MabSelect SuRe pcc column at 5 mL/min. Pooled cell culture supernatant is loaded onto the column, washed with 20 mM sodium phosphate, 150 mM sodium chloride, 1 pH 7.0 and eluted with 100 mM glycine, 150 mM sodium chloride, pH 3.2 directly into 10% volume of 1M sodium phosphate, pH 7.0. Peak fractions are pooled, concentrated using centrifugal concentrators (10 kDa MWCO, MilliporeSigma). Concentrated antibodies are dialyzed into 20 mM sodium phosphate, 150 mM sodium chloride and diluted to a final concentration of ˜0.5 mg/mL/antibody to dose at 5 mg/kg/antibody (IgG equivalent). Antibody pools are snap frozen in liquid nitrogen and thawed before dosing.

### In Vivo Screening:

Mouse studies utilized hTfR females (Biocytongen). Mice are IV dosed via the tail vein with 5 mg/kg test article (singplex-one antibody). After 1, 6, 24 and 72 hours, animals are sacrificed, and blood (˜500 μL) is collected via cardiac puncture in serum separator tubes and spun at 14000 g for 10 min. Prior to organ collection mice are transcardially perfused with PBS. The brain is divided into two hemispheres and along with muscle, liver, heart and lungs, all tissues are snap frozen and stored at −80° C. Fresh-frozen brain tissue is homogenized using a Qiagen TissueLyser with 5 mm steel beads for 7 min at 30 Hz in 500 uL of RIPA lysis buffer+HALT protease inhibitors (100×) for 150 mg of brain tissue.

### Human IgG ELISA:

IgG antibody concentrations in mouse plasma and brain lysates are quantified using a generic human IgG ELISA (ab195215. AbCam) following the manufacturer's instructions. Plasma is diluted 1:10000, 1:20000, 1:40000 in a sample diluent provided with the ELISA kit. Similarly, brain lysates are diluted 1:5, 1:10 and 1:20 for analysis of huIgG concentration. Briefly, samples and standard curves are incubated with an antibody cocktail for 40 minutes at room temperature on a plate shaker set to 400 rpm. After washes, a TMB Development Solution is added to each well and the reaction is stopped with the addition of a Stop Solution to each well. Absorbance is measured at 450 nm in a plate reader. The standard curve is fit using a linear logistic regression.

### Example 37: In Vivo Detection of Shuttle-Antibody Payload Fusions

The present example demonstrates in vivo characterization of shuttle-payload fusions as described herein. Specifically, the present example demonstrates immunofluorescence detection of a TfR-1409-51-anti-Aβ mAb construct in specific brain regions. In addition, specifically, the present example demonstrates immunofluorescence detection of a FLT1-49 anti-Aβ mAb construct in both specific brain regions and whole brain images. Furthermore, the present example demonstrates that FLT1-49 increases the uptake and distribution of an anti-Abeta mAb payload throughout the mouse brain and shuttles an anti-Abeta mAb payload through the neurovasculature. Moreover, specifically, the present example demonstrates immunofluorescence detection of a CD98-11 anti-Aβ mAb construct in whole-brain images and shows that CD98-11 shuttle causes a fundamental shift in biodistribution of an anti-Abeta mAb.

### Method

### In Vivo Detection:

hTfR mice were IV dosed via the tail vein with 5 mg/kg of test articles. 24 hours after dosing, animals were perfused by PBS and brains were extracted, drop-fixed in 4% paraformaldehyde overnight at 4 C followed by 30% sucrose overnight at 4 C. Brains were then sectioned at 35 μm on a sliding cryostat, blocked in 5% NDS (Normal Donkey Serum), 10% of Triton in PBS for 1 hour at room temperature and stained with NeuN (Rabbit monoclonal anti-NeuN antibody (1:500, Abcam ab177487) overnight at 4 C in blocking buffer. After overnight incubation, sections were washed in PBS and and incubation with Alexa Fluor® 594 AffiniPure Donkey Anti-Rabbit IgG (H+L) (1:200)(Jackson 711-585-152) and Alexa Fluor®488 AffiniPure Donkey Anti-Human IgG (H+L) (1:200) Jackson 709-545-149) at room temperature for 2 hours.

Brain sections were washed in PBS then mounted on slides using Prolong Glass Antifade Mountant and imaged using a Zeiss Axioscan scope (20×/0.8 air objective).

ELISA was performed on samples obtained from mice using IgG Human SimpleStep ELISA® kit according to the protocol provided by the manufacturer.

### Immunohistochemical Staining:

Six-month-old 5×FAD mice (n=2 per group) were intravenously injected via tail with either an unshuttled anti-Aβ or FLT1-49-shuttled anti-Aβ at a dose of 50 mg/kg. Twenty-four hours post-injection, mice were perfused with cold PBS, and their brains were extracted and post-fixed in 4% paraformaldehyde solution overnight (24 hours). Following fixation, brains were dehydrated by immersion in 30% sucrose solution in PBS for at least 48 hours or until the brains had fully soaked in the solution. Brains were then frozen in OCT, and 30-μm-thick floating sections were obtained using a cryostat. These sections were stored in PBS with 2% sodium azide (NaN3) at 4° C.

For immunohistochemical staining, brain sections were washed three times for 10 minutes each with PBS+1% Triton X-100 to remove sodium azide (450 mL PBS+50 mL Triton X-100). Subsequently, sections were blocked in a solution containing 5% normal donkey serum (NDS), 10% Triton X-100 (1% final concentration), and 85% PBS for 1 hour at room temperature. The samples were then incubated overnight with shaking at 4° C. in blocking solution containing purified anti-β-Amyloid 1-16 antibody 6E10 (1 mg/mL; 1:1000 dilution). After washing with PBS, sections were incubated for 2 hours with secondary antibodies: Donkey anti-Mouse IgG (H+L) Highly Cross-Adsorbed Alexa Fluor™ 488 (1:200, Invitrogen) and Donkey anti-Human IgG (H+L) Highly Cross-Adsorbed Cy3 (1:200, Jackson Laboratory). Following a final wash with PBST three times for 10 minutes and once with PBS for 30 minutes, sections were mounted on slides using Prolong Gold DAPI Mounting Media.

Twenty-four hours after mounting, samples were imaged using a Zeiss AxioObserver 7 equipped with a 10× air objective, and images were captured using Zen software. Exposure settings were determined automatically by Zen software and applied consistently across all images. Exposure times were recorded for each experiment.

### Results

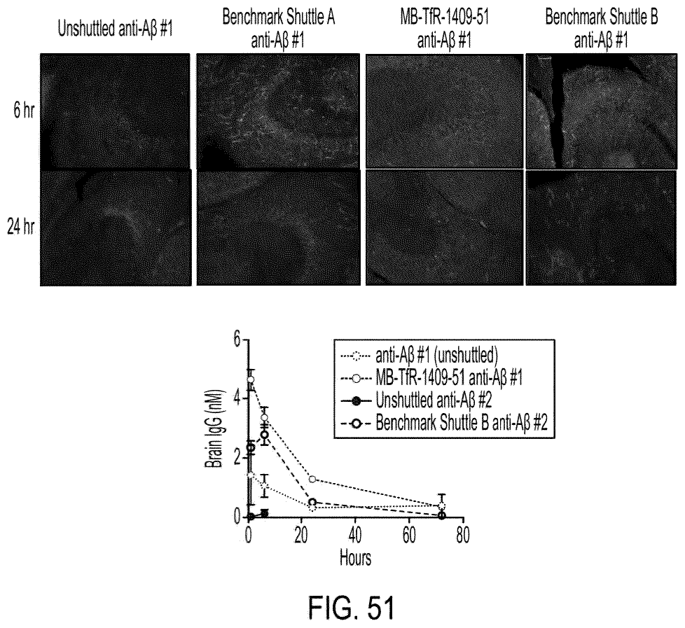

FIG. 51 shows micrographs of immunofluorescence imaging of an anti-aβ antibody alone versus MB-TfR-1409-51 and multiple benchmark shuttles. Each construct was assayed in humanized TfR mice at 5 mg/kg dose and imaged using constant exposure time at 4600 ms (Cy3). The micrographs show the in vivo biodistribution of constructs in mouse hippocampus sections stained for IgG at 6 and 24 hrs post-injection. The plot of FIG. 51 shows four of the shuttled and unshuttled antibodies shown in the micrographs, in an ELISA readout for total IgG concentration in the brain (nM) at 1, 6, 24, and 72 hours. MB-TfR-1409-51 is differentiated by faster and more total uptake in the brain relative to the Benchmark Shuttle B and unshuttled controls tested.

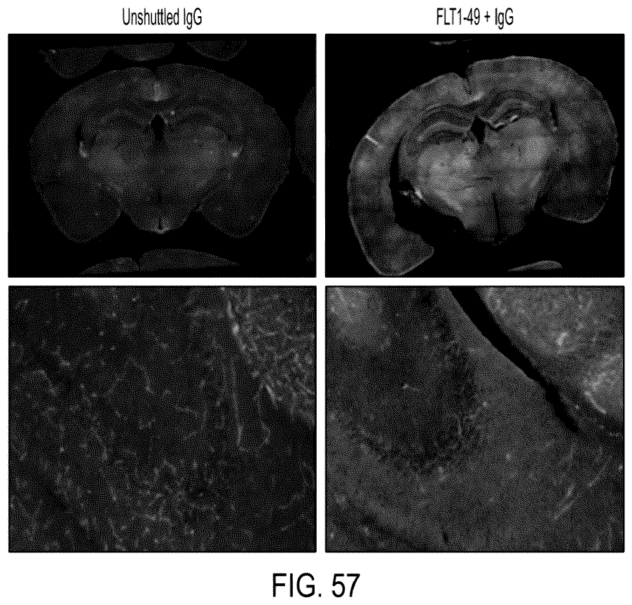

FIG. 57 shows micrographs of immunofluorescence imaging of an exemplary antibody alone versus shuttled antibody (IgG+FLT1-49). Each construct was assayed in C57 mice at 5 mg/kg. Shown here is distribution of IgG constructs in coronal brain sections and in the CA3 region of the hippocampus (20×) at 24 hours post-injection.

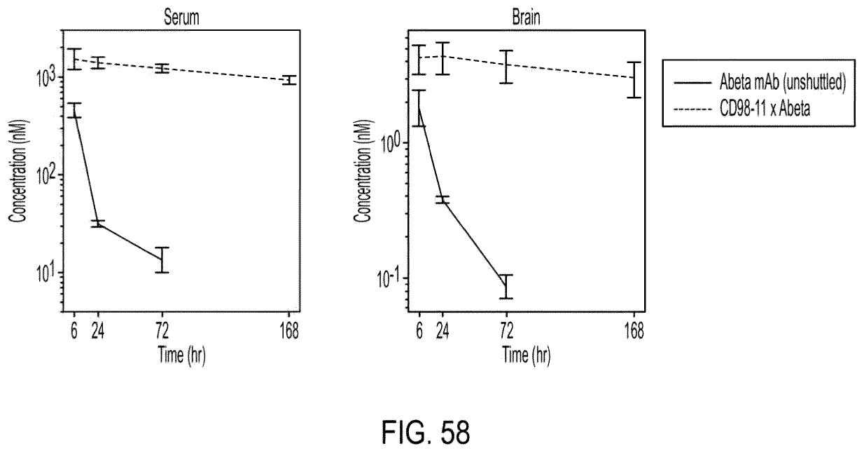

FIG. 58 depicts plots showing levels of an anti-Abeta mAb alone or fused to shuttle CD98-11. Levels were assessed in serum and whole brain by ELISA at 6, 24, 72, and 168 hr timepoints post-injection. Constructs were examined at 30 mg/kg in a humanized CD98 mouse. These data demonstrate that shuttle CD98-11 extends serum half-life and brain retention of an anti-Abeta mAb payload.

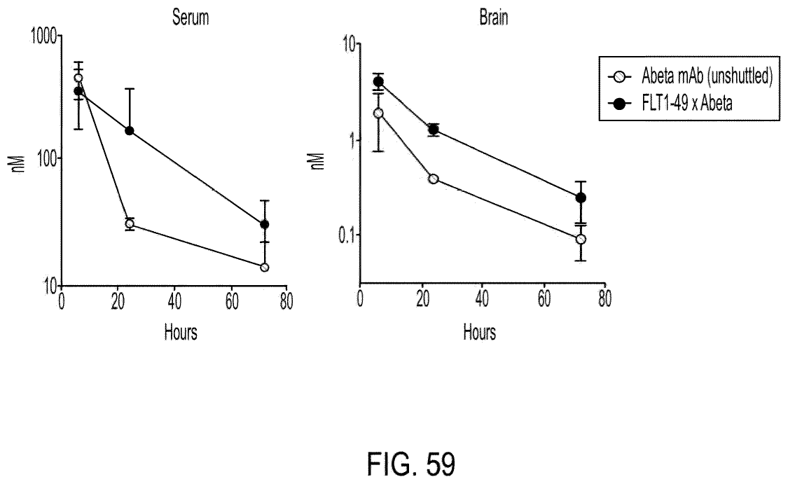

FIG. 59 depicts plots showing levels of an anti-Abeta mAb alone or fused to shuttle FLT1-49. Levels were assessed in serum and brain by ELISA at 6, 24, 72, and 168 hr timepoints post-injection. Constructs were examined at 30 mg/kg in C57 mice. These data demonstrate that shuttle FLT1-49 improves uptake of an anti-Abeta mAb payload.

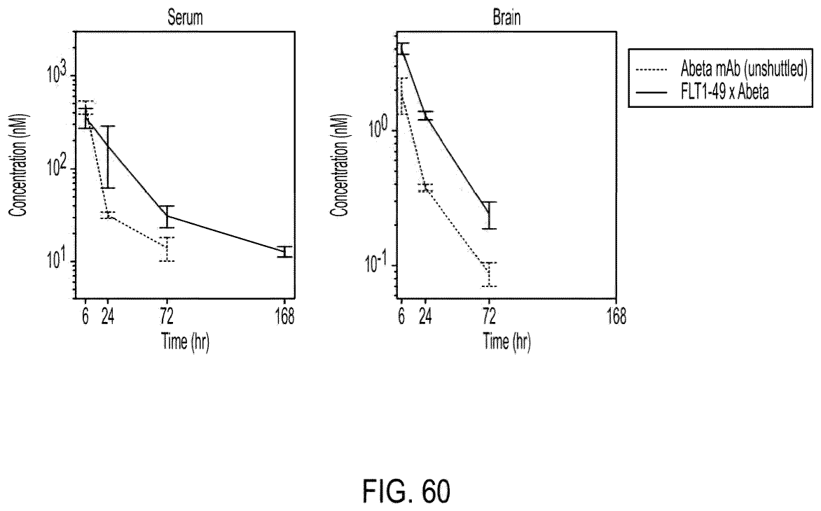

FIG. 60 depicts plots showing levels of an anti-Abeta mAb alone or fused to shuttle FLT1-49. Levels were assessed in serum and brain by ELISA at 6, 24, 72, and 168 hr timepoints post-injection. Constructs were examined at 30 mg/kg in C57 mice. These data demonstrate that shuttle FLT1-49 improves uptake of an anti-Abeta mAb payload.

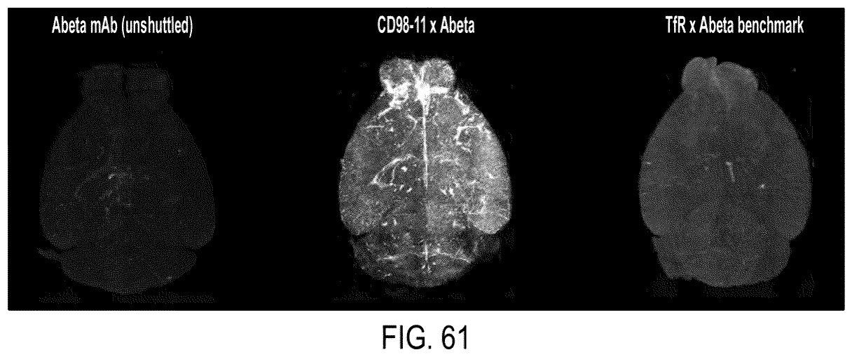

FIG. 61 shows representative 3D whole brain images generated using light sheet fluorescence microscopy (LSFM) and whole-organ immunohistochemistry (iDISCO) following treatment with an anti-Abeta mAb alone (unshuttled), versus fused to shuttle CD98-11, or a TfR-shuttled Abeta control. Constructs were examined at 50 mg/kg in humanized CD98 or humanized TfR mice at 24 hr post-injection. These data demonstrate that shuttle CD98-11 causes a fundamental shift in biodistribution of an anti-Abeta mAb.

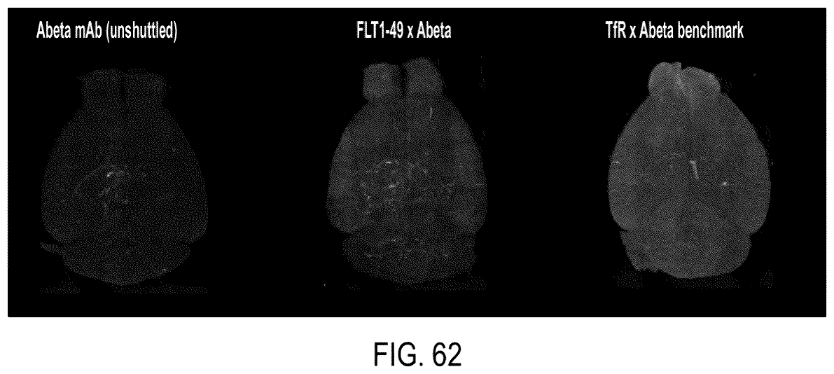

FIG. 62 shows representative 3D whole brain images generated using light sheet fluorescence microscopy (LSFM) and whole-organ immunohistochemistry (iDISCO) following treatment with an anti-Abeta mAb alone (unshuttled), versus fused to shuttle FLT1-49, or a TfR-shuttled Abeta control. Constructs were examined at 50 mg/kg in C57 mice or humanized TfR mice at 24 hr post-injection. These data demonstrate that shuttle FLT1-49 increases uptake and distribution of an anti-Abeta mAb payload throughout the mouse brain.

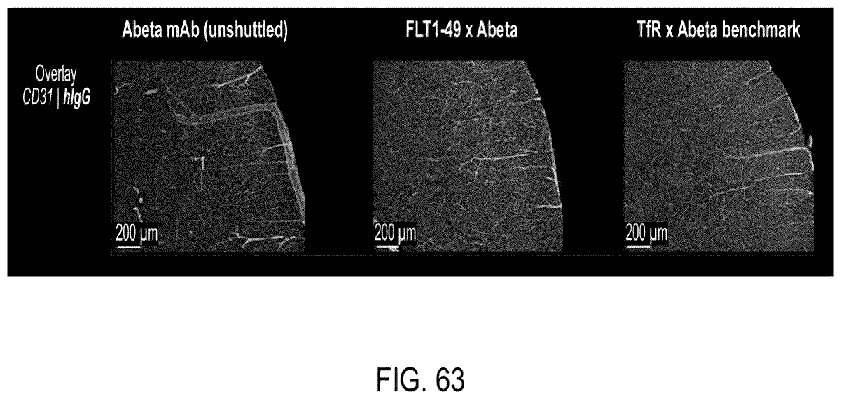

FIG. 63 shows representative 2D sections from magnified areas of whole brains scans acquired using Light sheet fluorescence microscopy (LSFM). Immunostaining for CD31 and hIgG are displayed in the same brain in teal and glow, respectively. Scale bars are 200 μm. Constructs were examined at 50 mg/kg in C57 mice or humanized TfR mice at 24 hr post-injection. These data demonstrate that FLT1-49 shuttles an anti-Abeta mAb payload through neurovasculature.

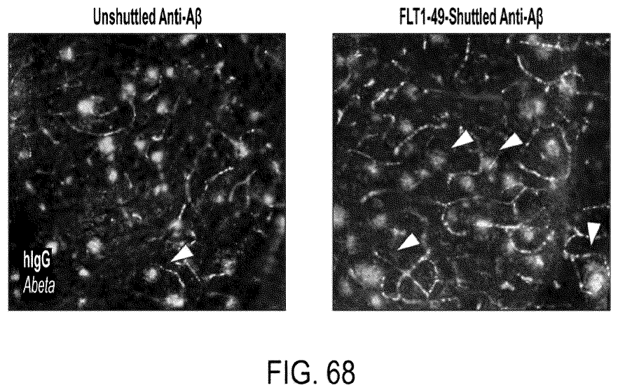

FIG. 68 shows representative immunofluorescence images from brain sections twenty-four hours post-injection of either unshuttled anti-Aβ or FLT1-49 shuttled anti-Aβ. Six-month-old 5×FAD mice (n=2 per group) were intravenously injected via tail with constructs at a dose of 50 mg/kg. Abeta plaques are displayed in green and antibodies are displayed in red.

### Example 38: In Vitro RNA Targeting Using a Shuttle-Conjugated ASO

The present example demonstrates in vitro RNA targeting via a shuttle-conjugated ASO as described herein. Specifically, the present example demonstrates MALAT1 knockdown in TfR-expressing cells in response to TfR-1409-51-conjugated MALAT1 ASO.

### Method

### Antisense Oligonucleotide Sequence:

Antisense oligonucleotide (ASO) targeting a long-noncoding RNA (lncRNA) MALAT1 was selected as an exemplary RNA target that can be used in accordance with embodiments of the present disclosure. An ASO targeting lncRNA MALAT1 was ordered from Integrated DNA Technologies (IDT). Sequences were comprised of nucleotides linked by phosphorothioate backbone chemistry and were capped at the 5′ and 3′ ends by phosphorothioate containing locked nucleic acids. Sequences also contained a 5′ azide modification linked by NHS ester chemistry as a handle for click chemistry mediated oligo conjugation (Click #2). Other click chemistry techniques can be used in accordance with embodiments of the present disclosure.

Exemplary ASO Sequence 1 that Targets MALAT1

(SEQ ID NO: 9061)
/5AzideN/+G*+G*+C*+A*+T*A*T*G*C*A*G*A*T*A*A*+T*+
G*+T*+T*+C
“/5 AzideN/”-5' azide modification linked
through NHS chemistry
“+”-Locked nucleic acid chemistry
“*”-Phosphorothioate linkage

### Protein Constructs:

To generate Fc-containing constructs with a single ASO attachment site, and to generate constructs containing only a single shuttle moiety, a Knob/Hole strategy was employed. For ASO conjugation, a carboxy-terminal sequence containing a GGGGS linker (SEQ ID NO: 9060), sortase A site and hexahistadine tag (SEQ ID NO: 9064) (GGGGGSLPETGGHHHHHH (SEQ ID NO: 9062)) was employed into a “knob” construct. TfR1409-51 was additionally appended to the amino-terminus of this “knob” construct. Control molecule constructs were designed similarly, for an unshuttled control construct a “knob” side is as described, but without TfR1409-51 at the amino-terminus. Benchmark #1 is constructed with a shuttling moiety within a Fc region as described herein.

### Protein Expression and Purification:

Plasmid DNA for knob and hole constructs was combined at a 1:1 ratio for transfection into Expi293F cells (Thermo Fisher), using 1 μg total plasmid mass per mL of transfection. DNA was sterile filtered and complexed with Expifectamine293, 1 L transfection volumes were used for each construct. Transfected cells were harvested by centrifugation four days after transfection. For each construct, supernatant was collected and sterile filtered using 0.2 μm filters before application on a 5 mL HiTrap MabSelect PrismA columns (Cytiva). Unbound material was washed from the column using 200 mL of 1×PBS before elution with 30 mL of elution buffer (100 mM glycine pH 3.2; 150 mM NaCl). Eluted protein was brought to neutral pH by addition of 3.5 mL 1M NaPO4 pH 7.0. Purified protein was concentrated to 300-400 μM using 10 kDa MWCO Amicon centrifugal filters (Millipore). Concentrated proteins were supplemented with glycerol to a final concentration of 20% before snap freezing in liquid nitrogen and storage at −80° C.

### Sortase Mediated Conjugation of “Click #1” to Protein Constructs:

Purified proteins were desalted into Click #1 conjugation buffer (30 mM HEPES pH 7.0; 250 mM NaCl) using PD-10 desalting columns (Cytiva). Click #1 sortase mediated conjugation reactions contained 50 μM desalted Fc construct; 50 mM HEPES pH 7.0; 10 mM CaCl2; 5 μM sortase A enzyme; 700 μM DBCO-PEG4-Gly-Gly-Gly (Vector Labs). Reactions were incubated at room temperature for 4 hours. Reactions were then supplemented with 2M imidazole pH 7.0 and 5M NaCl to final concentrations of 15 mM imidazole; 750 mM NaCl before addition of Ni Sepharose HP resin (Cytiva) to remove unreacted Fc constructs and sortase. Nickel resin was incubated with completed Click #1 reactions for 15 minutes, resin removed by centrifugation and the supernatant containing completed reactions was collected and filtered using 0.2 μm syringe filters. To remove excess PEG4-Gly-Gly-Gly, constructs were desalted into 20 mM NaPO4 pH 7.0; 150 mM NaCl.

### Click #2 ASO Conjugation and Size Exclusion Purification:

Desalted Click #1 reactions were combined with azide-containing Exemplary ASO 1 at final concentrations of 100 μM Click #1 reactions, 150 μM Exemplary ASO 1. Samples incubated at room temperature for 18 hours. DNA conjugation efficacy was assessed by running samples on 4-12% gradient SDS-PAGE gels, which were then stained with 1:10,000 sybrGold DNA stain reagent (Thermo Fisher) and imaged. Same gels were then stained with Coomassie and imaged. DNA conjugated proteins were then further purified by size exclusion chromatography using a superdex200 column (Cytiva) to remove excess ASO. Final purified product was then concentrated, and flash frozen at −80° C.

### MALAT1 Knockdown:

96-well nunclon delta treated plates (Thermo Fisher) were coated with poly-D lysine (Gibco) according to manufacture instructions. Wells were seeded with 20,000 cells/well using HEK293 cells overexpressing TfR Seeded cells were allowed to attach overnight and the next day media was aspirated and replaced with media containing 0-500 nM ASO conjugated constructs or ASO alone. Treated cells were then incubated for 72 hours before harvest and RNA extraction. RNA extraction was conducted using the RNeasy-96 kit (Qiagen) according to manufacture instructions. 2 μL of extracted RNA (5-10 ng/uL) was used for the qRT-PCR assay using the QuantiNova Probe RT-PCR kit (Qiagen). FAM-labeled TaqMan probes (Thermo Fisher) for human MALAT1 and human GAPDH were used in separate reactions. MALAT1 fold-change was calculated using the ΔΔCt method using GAPDH as a reference gene and normalized to untreated cells.

### Results

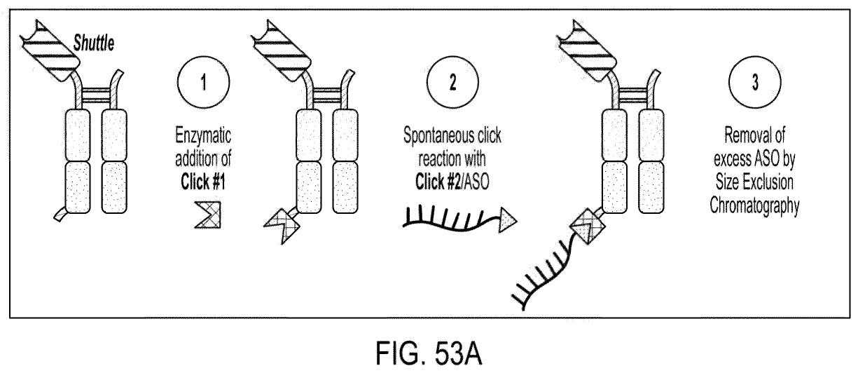

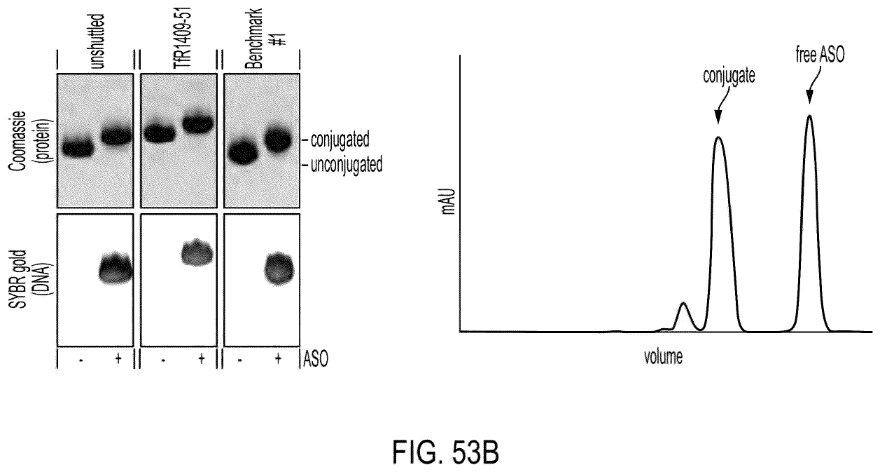

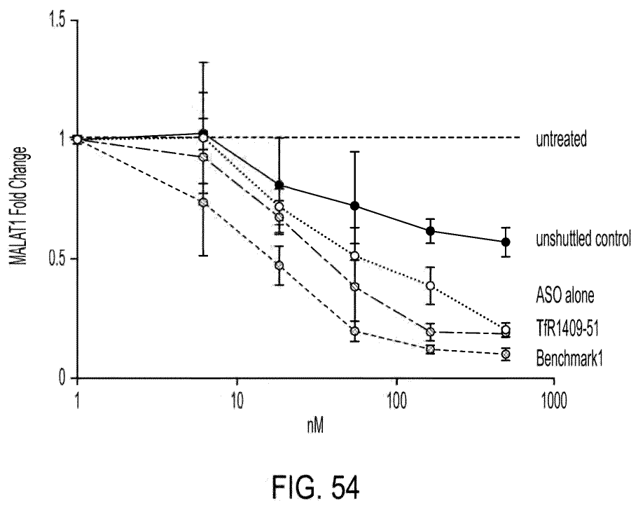

FIG. 53A illustrates an exemplary two-step conjugation strategy for ASO attachment described herein. FIG. 53B shows that ASO conjugation efficiency for each construct exceeded 90% as visualized by Coomassie staining of SDS-PAGE gels. DNA staining demonstrated that Coomassie-shifted bands are indeed conjugated to ASO. Size exclusion chromatography showed that excess ASO can be separated from ASO conjugated protein products (see FIG. 53B). FIG. 54 shows knockdown of MALAT1 in HEK293 cells overexpressing TfR as measured by qRT-PCR. Error bars illustrate standard deviation across three separate experiments. ASOs conjugated to Fc molecules containing TfR shuttles (TfR1409-51 and Benchmark #1) reduced the expression of their ASO target (MALAT1) to a greater degree than an unshuttled control.

Other types of RNAi strategies can be used in accordance with embodiments described by this Example.

### REFERENCES

Towbin H, Staehelin T, Gordon J. Electrophoretic transfer of proteins from polyacrylamide gels to nitrocellulose sheets: procedure and some applications. Proc Natl Acad Sci USA. 1979; 76(9):4350-4354. Doi:10.1073/pnas.76.9.4350
Engvall E, Perlmann P. Enzyme-linked immunosorbent assay, Elisa. 3. Quantitation of specific antibodies by enzyme-labeled anti-immunoglobulin in antigen-coated tubes. J Immunol. 1972 July; 109(1):129-35. PMID: 4113792.
Elshal M F, McCoy J P Multiplex bead array assays: performance evaluation and comparison of sensitivity to ELISA. Methods. 2006 April; 38(4):317-23. Doi: 10.1016/j.ymeth.2005.11.010. PMID: 16481199; PMCID: PMC1534009.
Shendure J, Porreca G J, Reppas N B, Lin X, McCutcheon J P, Rosenbaum A M, Wang M D, Zhang K, Mitra R D, Church G M. Accurate multiplex polony sequencing of an evolved bacterial genome. Science. 2005 Sep. 9; 309(5741):1728-32. Doi: 10.1126/science.1117389. Epub 2005 Aug. 4. PMID: 16081699.
Trads J B, Tørring T, Gothelf K V. Site-Selective Conjugation of Native Proteins with DNA. Acc Chem Res. 2017 Jun. 20; 50(6):1367-1374. Doi: 10.1021/acs.accounts.6b00618. Epub 2017 May 9. PMID: 28485577.
Egloff P, Zimmermann I, Arnold F M, Hutter C A J, Morger D, Opitz L, Poveda L, Keserue H A, Panse C, Roschitzki B, Seeger M A. Engineered peptide barcodes for in-depth analyses of binding protein libraries. Nat Methods. 2019 May; 16(5):421-428. Doi: 10.1038/s41592-019-0389-8. Epub 2019 Apr. 22. PMID: 31011184; PMCID: PMC7116144.
Pollock S B, Hu A, Mou Y, Martinko A J, Julien O, Hornsby M, Ploder L, Adams J J, Geng H, Müschen M, Sidhu S S, Moffat J, Wells J A. Highly multiplexed and quantitative cell-surface protein profiling using genetically barcoded antibodies. Proc Natl Acad Sci USA. 2018 Mar. 13; 115(11):2836-2841. Doi: 10.1073/pnas.1721899115. Epub 2018 Feb. 23. PMID: 29476010; PMCID: PMC5856557.
Mohan D, Wansley D L, Sie B M, Noon M S, Baer A N, Laserson U, Larman H B. PhIP-Seq characterization of serum antibodies using oligonucleotide-encoded peptidomes. Nat Protoc. 2018 September; 13(9):1958-1978. Doi: 10.1038/s41596-018-0025-6. Erratum in: Nat Protoc. 2018 Oct. 25; PMID: 30190553; PMCID: PMC6568263.
Barbas, C. F., Burton, D. R., Scott, J. K., & Silverman, G. J. Phage Display: A Laboratory Manual. 2001. Cold Spring Harbor Laboratory Press.
Yu Y. J, Zhang Y, Kenrick M., Hoyte K., Luk W., Lu Y., et al. Boosting brain uptake of a therapeutic antibody by reducing its affinity for a transcytosis target. Sci Transl Med 3, 84ra44 (2011).
P. Emami et al. Plug & play directed evolution of proteins with gradient-based discrete MCMC. Mach. Learn.: Sci. Technol. 4 025014 (2023).
B. L. Hie et al. Efficient evolution of human antibodies from general protein language models. Nat Biotechnol 42, 275-283 (2023).
Z. Lin et al. Language models of protein sequences at the scale of evolution enable accurate structure prediction. bioRxiv (2022).
A. Elnaggar et al. ProtTrans: Toward Understanding the Language of Life Through Self-Supervised Learning. IEEE Transactions on Pattern Analysis and Machine Intelligence, 44.10, 7112-7127 (2022).
T. H. Olsen et al. Observed Antibody Space: A diverse database of cleaned, annotated, and translated unpaired and paired antibody sequences. Protein Sci. 31.1, 141-146 (2022)

### Other Embodiments

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes as described herein. The publications, websites and other reference materials referenced herein to describe the background of the invention and to provide additional detail regarding its practice, including those listed in the above References section, are hereby incorporated by reference in their entireties.

It is to be understood that while embodiments of the invention have been described in conjunction with the detailed description thereof, the foregoing description is intended to illustrate and not limit the scope of the invention, which is defined by the scope of the appended claims. Other aspects, advantages, and modifications are within the scope of the following claims.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

Lengthy table referenced here
US20260158147A1-20260611-T00004
Please refer to the end of the specification for access instructions.

Lengthy table referenced here
US20260158147A1-20260611-T00005
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260158147A1). An electronic copy of the table will also be available from the USPTO upon request and payment of the fee set forth in 37 CFR 1.19(b)(3).

1. A peptide shuttle, wherein the peptide shuttle has or comprises an amino acid sequence having at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs: 8813-8894.

2. The peptide shuttle of claim 1, wherein the peptide shuttle has or comprises an amino acid sequence according to any one of SEQ ID NOs: 8813-8894.

3. The peptide shuttle of claim 1, wherein the peptide shuttle has or comprises an amino acid sequence according to SEQ ID NO: 8873.

4. The peptide shuttle of claim 1, wherein the peptide shuttle has or comprises an amino acid sequence according to any one of SEQ ID NOs: 8891-8894.

5. The peptide shuttle of claim 1, wherein the peptide shuttle has a length of 20 to 200 amino acids.

6.-10. (canceled)

11. A shuttle component, wherein the shuttle component has or comprises a nucleic acid sequence having at least 80%, at least 85%, at least 90%, at least 91%, at least 92%, at least 93%, at least 94%, at least 95%, at least 96%, at least 97%, at least 98%, At least 99%, or 100% identity to any one of SEQ ID NOs: 8753-8812.

12.-20. (canceled)

21. A polypeptide comprising:
(a) a peptide shuttle; and
(b) a cargo polypeptide,
wherein the peptide shuttle is operably linked to the cargo polypeptide.

22.-25. (canceled)

26. The polypeptide of claim 21, wherein the peptide shuttle has or comprises an amino acid sequence having at least 80%, a leas 85%, at least 90%, at least 91%, at least 92%, at least 93%, at least 94%, at least 95%, at least 96%, at least 97%, at least 98%, at least 99%, or 100% identity to any one of SEQ ID NOs: 8813-8894.

27. The polypeptide of claim 21, wherein the peptide shuttle has or comprises an amino acid sequence according to SEQ ID NO: 8873.

28. The polypeptide of claim 21, wherein the peptide shuttle has or comprises a nucleic acid sequence according to any one of SEQ ID NOs: 8891-8894.

29. (canceled)

30. The polypeptide of claim 21, wherein the peptide shuttle has a length of 20 to 200 amino acids.

31.-35. (canceled)

36. The polypeptide of claim 21, wherein the cargo polypeptide is or comprises a therapeutic polypeptide.

37.-59. (canceled)

60. A polynucleotide comprising:
(a) a shuttle component whose nucleotide sequence is or comprises a sequence encoding a peptide shuttle; and
(b) a cargo component whose nucleotide sequence is or comprises a sequence encoding a cargo polypeptide.

61.-116. (canceled)

117. A composition comprising:
(a) a shuttle; and
(b) a cargo (e.g., a cargo polypeptide, e.g., a cargo nucleotide (e.g., a cargo DNA, e.g., a cargo RNA, e.g., a cargo DNA/RNA)),
wherein the shuttle is operably linked to the cargo.

118. The composition of claim 117, wherein the shuttle is a peptide shuttle.

119. (canceled)

120. The composition of claim 118, wherein the peptide shuttle has or comprises an amino acid sequence having at least 80%, a leas 85%, at least 90%, at least 91%, at least 92%, at least 93%, at least 94%, at least 95%, at least 96%, at least 97%, at least 98%, at least 99%, or 100% identity to any one of SEQ ID NOs: 8813-8894.

121. The composition of claim 118, wherein the peptide shuttle has or comprises an amino acid sequence according to SEQ ID NO: 8873.

122. The composition of claim 118, wherein the peptide shuttle has or comprises an amino acid sequence according to any one of SEQ ID NOs: 8891-8894.

123.-124. (canceled)

125. The composition of claim 117, wherein the cargo is a therapeutic agent.

126. (canceled)

127. The composition of claim 117, wherein the cargo is or comprises an antibody or a fragment thereof or a variant thereof.

128.-130. (canceled)

131. The composition of claim 127, wherein the fragment has or comprises a sequence having at least 80%, at least 85%, at least 90%, at least 91%, at least 92%, at least 93%, at least 94%, at least 95%, at least 96%, at least 97%, at least 98%, at least 99%, or 100% identity to any one more sequences (e.g., VL, VH, CDR sequences) associated with rituximab, ocrelizumab, ofatumumab, ublituximab, binutuzumab, aducanumab, lecanemab, gantenerumab, donanemab, prasinezumab, anti-BACE-1 antibody 6266, anti-BACE-1 antibody YW412.8.31, Lu AF82422, or MEDI1341.

132.-137. (canceled)

138. The composition of claim 118, wherein the peptide shuttle has a length of 20 to 200 amino acids.

139.-166. (canceled)

167. A cell comprising the peptide shuttle of claim 1, the shuttle component of claim 11, the polypeptide of claim 21, the polynucleotide of claim 60, the composition of claim 117; or population thereof.

168. (canceled)

169. A pharmaceutical composition comprising peptide shuttle of claim 1, the shuttle component of claim 11, the polypeptide of claim 21, the polynucleotide of claim 60, the composition of claim 117, or any combination thereof.

170. A kit comprising a set of peptide shuttles, wherein each peptide shuttle of the set is according to claim 1.

171.-193. (canceled)

194. A method for identifying a therapeutic polypeptide or a target polypeptide to treat a disease, disorder, or condition comprising steps of:
(a) subjecting a population of barcoded shuttle-cargo polypeptides to an assessment, wherein each barcoded shuttle-cargo polypeptide comprises:
(i) a peptide shuttle,
(ii) a peptide barcode, and
(iii) a cargo polypeptide
wherein the peptide shuttle is operably linked to the cargo polypeptide, and the cargo polypeptide is operably linked to the peptide barcode;
(b) separating those members of the population that satisfy the assessment from those that do not, so that a positive population or a negative population, or both, is identified;
(c) contacting the positive population, or the negative population, or each population separately from the other, with a set of binders which includes at least one binder specific for each barcode in the population; and
(d) determining which binders bind to the separated members, thereby determining which barcoded shuttle-cargo polypeptides are present in the contacted population(s).

195.-270. (canceled)

271. A method of delivering a cargo to a tissue (e.g., the brain) comprising:
administering the cargo, or characteristic portion thereof, wherein the cargo is associated (e.g., covalently, e.g., non-covalently) with a peptide shuttle of claim 1.

272. (canceled)

273. The method of claim 271, wherein the method is a method of treatment.

274. The method of claim 271, wherein the therapeutic polypeptide comprises a peptide shuttle.

275. The method of claim 271, wherein the peptide shuttle is at a higher concentration in a brain cell, or population thereof, relative to a control cell or population thereof (e.g., serum).

276. The method of claim 271, wherein the tissue is or comprises a brain tissue.

277. The method of claim 271, wherein the method comprises treating a disease, disorder, or condition, wherein the disease, disorder, or condition is brain cancer (e.g., glioblastoma, metastatic brain cancers), Alzheimer's Disease (AD), Parkinson's Disease (PD), Huntington's Disease (HD), Multiple Sclerosis (MS), Bell's palsy, Cerebral palsy, Progressive Supranuclear Palsy (PSP), epilepsy, Motor Neuron Disease (MND), Amyotrophic Lateral Sclerosis (ALS), spinal muscular atrophy, Multiple System Atrophy (MSA), ataxia (e.g., spinocerebellar, Friedreich), Frontotemporal Dementia (FTD), or Lewy body disease.
